(19) United States
(12) Patent Application Publication
(10) Pub. No.: US 2025/0009905 A1
(43) Pub. Date: Jan. 9, 2025

(54) METHODS FOR EVALUATING TREATMENTS FOR BESTROPHINOPATHIES

(71) Applicant: The Trustees of the University of Pennsylvania, Philadelphia, PA (US)

(72) Inventors: Karina E. Guziewicz, Broomall, PA (US); Artur V. Cideciyan, Lafayette Hill, PA (US); William A. Beltran, Wynnewood, PA (US); Samuel G. Jacobson, Penn Valley, PA (US); Gustavo D. Aguirre, Media, PA (US)

(73) Assignee: The Trustees of the University of Pennsylvania, Philadelphia, PA (US)

(21) Appl. No.: 18/687,571

(22) PCT Filed: Sep. 1, 2022
(86) PCT No.: PCT/US2022/075815

§ 371 Date: Feb. 28, 2024

Related U.S. Application Data
(60) Provisional application No. 63/239,751, filed on Sep. 1, 2021.

(51) Int. Cl. A61K 48/00; A61B 3/10; A61B 5/00; A61B 5/398; A61P 27/02; C12N 15/86

(52) U.S. Cl. CPC A61K 48/0058; A61B 3/102; A61B 5/398; A61B 5/4848; A61P 27/02; C12N 15/86; C12N 2750/14143; C12N 2750/14145; C12N 2750/14171

(57) ABSTRACT

Methods for assessing efficacy of a treatment for a bestrophinopathy in a subject are provided. In certain embodiments, the subject has two mutant BEST1 alleles. In certain embodiments, the subject has at least one mutant BEST1 allele.

### STATEMENT OF GOVERNMENT SUPPORT

This invention was made with government support under EY006855 awarded by the National Institutes of Health. The government has certain rights in the invention.

### BACKGROUND OF THE INVENTION

Mutations in the human BEST1 (hBEST1) gene result in a spectrum of retinal disease phenotypes collectively termed bestrophinopathies associated with pathognomonic macular lesions. To date, nearly 300 either monoallelic or biallelic mutations in hBEST1 have been identified and associated with inherited visual defects of variable onset, severity, and progression. The broad spectrum of clinical presentations in bestrophinopathies ranges from the widespread symptoms affecting peripheral retina and vitreous in a rare condition of vitreoretinochoroidopathy (ADVIRC) to the well-defined clinical abnormalities often limited to macula and paramacular areas in the central retina like in Best Vitelliform Macular Dystrophy (BVMD) and more extensive in autosomal recessive bestrophinopathy (ARB). BVMD, inherited as an autosomal dominant trait with incomplete penetrance, and the recessive form (ARB) are the most common and best explored juvenile macular dystrophies among bestrophinopathies, characterized by a markedly abnormal electrooculogram (EOG) accompanied by an excessive accumulation of lipofuscin material within cells of the retinal pigment epithelium (RPE), formation of focal and multifocal subretinal lesions, and consequently, loss of central vision.

While bestrophinopathies were first described in 1905, understanding of their pathological mechanism as well as any progress in the development of treatment has been hampered by the dearth of reliable animal models to carry out the mechanistic studies. Recent identification of spontaneous animal models of BEST1-associated retinopathies has proven crucial in the investigation of disease mechanisms and development of new therapeutic strategies. The spontaneous canine BEST1 disease model (cBEST; canine multifocal retinopathy, cmr) is a naturally occurring autosomal recessive disorder in dogs, which is caused by the same genetic defects as human bestrophinopathies, and captures the full range of clinical manifestations observed in patients. To date, cBest retinopathy has been identified in thirteen dog breeds and results from one of three distinct mutations in the canine BEST1 ortholog (cBEST1 -c.73C>T/p.R25*, -c.482G>A/p.G161D, or -c.1388delC/P463fs) inherited in an autosomal recessive fashion. All three mutations lead to a consistent clinical phenotype in homozygous affected dogs, and model all major aspects of the disease-associated mutations as well as their molecular consequences described in man. The spectrum of clinical and molecular features recapitulated, including the salient predilection of lesions in the canine macular region, makes cBest an extremely attractive model system not only for addressing principles behind the molecular pathology of bestrophinopathies, but also for validating new therapeutic strategies.

Improvements in methods for treating BEST1-associated disorders caused by BEST1 gene mutations and for evaluating the effectiveness of potential treatments for bestrophinopathies are desired.

### SUMMARY OF THE INVENTION

In one aspect, provided herein is a method of assessing efficacy of treatment for a bestrophinopathy in a subject, the method comprising providing a subject having a treated eye, the treated eye having been administered a dose of a recombinant adeno-associated virus (rAAV) vector comprising a nucleic acid sequence encoding a human BEST1 protein or a functional fragment thereof, and wherein the subject has two mutant BEST1 alleles, and assessing retinal function in the treated eye of the subject by electroretinography (ERG), wherein improved and/or maintained ERG amplitude(s) is indicative of efficacy of the treatment.

In one aspect, provided herein is a method of assessing efficacy of treatment for a bestrophinopathy in a subject, the method comprising providing a subject having a treated eye, the treated eye having been administered a dose of a recombinant adeno-associated virus (rAAV) vector comprising a nucleic acid sequence encoding a human BEST1 protein, or a functional fragment thereof, wherein the subject has at least one mutant BEST1 allele, the method comprising assessing retinal function in the treated eye of the subject by ERG, wherein improved and/or maintained ERG amplitude(s) is indicative of efficacy of the treatment.

In one aspect, provided herein is a method of treatment for a bestrophinopathy in a subject having at least one mutant BEST1 allele, the method comprising assessing retinal function in an eye of the subject by electroretinography (ERG), and administering to the eye a dose of a recombinant adeno-associated virus (rAAV) vector comprising a nucleic acid sequence encoding a human BEST1 protein or a functional fragment thereof. In certain embodiments, the method further comprises assessing retinal function in the eye of the subject by ERG following the administration to the eye the rAAV vector.

In one aspect, provided herein is a method of treatment for a bestrophinopathy in a subject having two mutant BEST1 alleles, the method comprising assessing retinal function in an eye of the subject by ERG, and administering to the eye a dose of a rAAV vector comprising a nucleic acid sequence encoding a human BEST1 protein or a functional fragment thereof. In certain embodiments, the method further comprises assessing retinal function in the eye of the subject by ERG following the administration to the eye the rAAV vector.

In one aspect, provided herein is a method of assessing efficacy of treatment for a bestrophinopathy in a subject having at least one mutant BEST1 allele, the method comprising providing a subject having a treated eye, said treated eye having been administered a dose of a recombinant adeno-associated virus (rAAV) vector comprising a nucleic acid sequence encoding a human BEST1 protein, or a functional fragment thereof, assessing retinal function in the treated eye of the subject by ERG, and administering to the eye a dose of a recombinant adeno-associated virus (rAAV) vector comprising a nucleic acid sequence encoding a human BEST1 protein, or a functional fragment thereof. In certain embodiments, the method further comprises assessing retinal function in the eye of the subject by ERG following the administration to the eye the rAAV vector.

In one aspect, provided herein is a method of assessing efficacy of treatment for a bestrophinopathy in a subject having two mutant BEST1 alleles, the method comprising providing a subject having a treated eye, said treated eye having been administered a dose of a rAAV vector comprising a nucleic acid sequence encoding a human BEST1 protein, or a functional fragment thereof, assessing retinal function in the treated eye of the subject by ERG, and administering to the eye a dose of a rAAV vector comprising a nucleic acid sequence encoding a human BEST1 protein, or a functional fragment thereof. In certain embodiments, the method further comprises assessing retinal function in the eye of the subject by ERG following the administration to the eye the rAAV vector.

In certain aspects, the methods provided include assessing retinal function by obtaining ERG measurements in more than one region of the retina of the treated eye. In certain embodiments, assessing retinal function comprises obtaining ERG measurements within a treated region of the retina and in an untreated region of the retina. In certain embodiments, the treated region of the retina is a subretinal bleb at the site of administration. In certain embodiments, assessing retinal function comprises obtaining ERG measurements for a contralateral, untreated eye. In certain embodiments, the method includes assessing retinal function by measuring the amplitude(s) of a scotopic a-wave response, a scotopic b-wave response, a photopic b-wave response, and/or a photopic flicker response. In further embodiment, a) the scotopic a-wave response is measured at an intensity that produces a mixed rod-cone response; b) the scotopic a-wave response is measured at an intensity that produces a rod-only or a mixed rod-cone response; c) the photopic b-wave response is measured at an intensity that produces a cone response; and/or d) the photopic flicker response is measured at an intensity that produces a cone response. In certain embodiments, an amplitude difference is obtained by 1) comparing an ERG amplitude measurement obtained from the treated eye and an ERG amplitude measurement obtained from an untreated, contralateral eye; and/or 2) comparing an ERG amplitude measurement obtained in a region of the treated eye and an ERG amplitude measurement obtained from an untreated region of the treated eye.

In certain aspects, the methods provided include evaluating treatment by one or more of: performing in vivo retinal imaging to evaluate one or more of a longitudinal reflectivity profile (LRP), IS/OS to retinal pigment epithelium (RPE) distance in light-adapted and/or dark-adapted eyes, electrophysiology, dark-adapted kinetic perimetry and formation of light-potentiated subretinal microdetachments, wherein treatment efficacy is indicated by one or more of a rescue of retinal microarchitecture through restoration of RPE apical microvilli structure, and a reestablishment of proper apposition between RPE cells and photoreceptor (PR) outer segments (cytoarchitecture of RPE-PR interface).

Other aspects and advantages of the invention will be readily apparent from the following detailed description of the invention.

### BRIEF DESCRIPTION OF THE DRAWINGS

FIG. 1 shows confocal images illustrating the molecular pathology of cBest (R25*/R25*) mutant retinas compared to wild-type (WT) retinal tissue from a control subject. Retinal cryosections were immunolabeled with anti-EZRIN and human cone arrestin (red) combined with peanut agglutinin lectin (PNA, cyan) and DAPI to detail the structural alterations underlying loss of the native extracellular compartmentalization of cone photoreceptor outer segments and loss of interaction between RPE and the adjacent photoreceptor OS, resulting in subretinal microdetachment.

FIG. 2 shows a comparison of cross-sectional retina images of the retina for WT, cBest-Heterozygous (R25*), and cBest-R25*/P463fs mutant models obtained using either the Spectralis SD/OCT or Leica/Bioptigen Envisu R2210 SD-OCTUHR systems. Longitudinal reflectivity profiles (LRP) based on these UHR images are also shown to the right (Leica/Bioptigen Envisu R2210) compared to magnified images from Spectralis SD-OCT (in the center (Spectralis) and right (Leica/Bioptigen Envisu R2210) columns.

FIG. 3 shows results from ex vivo analyses of WT (top) and cBest heterozygous (R25*) (bottom) retinas in correlation to LRP images from UHR OCT and corresponding schematic drawings of retinal lamination.

FIG. 4 shows molecular pathology in cBest heterozygous (R25*) (top) and WT (bottom) retinas. Retinal cryosections from cBest-R25*-het and WT control retinas were assayed with anti-EZRIN, hCAR, and PNA to delineate RPE apical surface and associated microvilli, examine RPE-PR junction and IPM. Confocal micrographs were analyzed in comparison to generated LRP to determine the origin of LRP peaks and factors underlying the abnormal LRP in cBest-het mutant retina.

FIG. 5 shows a comparison of cross-sectional images from either the Spectralis SD-OCT or Leica/Bioptigen Envisu R2210 SD-UHR OCT system and corresponding immunolabeled sections from WT, cBest heterozygous, and cBest homozygous mutant retinas.

FIG. 6 shows rescue of the retinal microarchitecture at the RPE/PR interface following administration of AAV-mediated BEST1 gene augmentation therapy.

FIG. 7A-FIG. 7D demonstrate the retinal phenotype of cBest1-heterozygous, and cBest1-homozygous dog models compared with wild type (WT). FIG. 7A shows ultra-high resolution fiber-based Fourier domain optical coherence tomography of a wild type (WT) dog retina. The images show that the in vivo and ex vivo data correlate. FIG. 7B shows the retinal phenotype of a cBest1-heterozygous (cBest-het) dog model. The abnormal microarchitecture of the RPE-PR interface in cBest-het mutant model is shown. Elongation of both ROS & COS associated with increased external limiting membrane (ELM)-RPE distance, presence of L/MS-& RDS (PRPH2)-positive debris at the RPE apical surface indicating abnormal POS-RPE apposition and interaction in cBest-hets. FIG. 7C and FIG. 7D show a comparison of the 2-D (FIG. 7C) and 3-D (FIG. 7D) retinal imaging of WT and cBest-het models. FIG. 7C and FIG. 7D show significant lengthening of COS and ROS, as well as stretching and curving of the IS/OS.

FIG. 8A and FIG. 8B demonstrate that activation of Muller glia (MG) cells and reactive astrogliosis promote an inflammatory environment in cBest retina in both cBest-homozygous and cBest-heterozygous mutant models. Extension of Muller glia processes can be seen reaching RPE cells.

FIG. 8C demonstrates activation of Muller glia in cBest-het retinas. 40× (top) and 100× (bottom) confocal images show reactive gliosis in cBest-hets. Upregulation of glial fibrillary acid protein (GFAP) is an indicator of retinal stress. Also seen are fluctuation of ONL thickness (top panel), INL-ONL cell migration (top panel), and elevation of retinal surface (SS stretch—top panel).

FIG. 9 further demonstrates the retinal phenotype of the cBest1-heterozygous dog model as compared to WT.

FIG. 10 demonstrates that AAV-mediated BEST1 gene augmentation therapy restores retinal homeostasis and prevents gliotic changes in cBest mutant retina post AAV-BEST1 injection. The activation of Muller glia is limited to untreated retinal regions and is associated with subretinal microdetachment.

FIG. 11 shows a summary of cBest-AR rAAV2-hBest1-injected eyes enrolled in the study. All eyes receiving a dosage of 1.15×10¹¹ or higher showed rescue.

FIG. 12 shows assessment of cBest-AR treated subjects up to 74 weeks post injection.

FIG. 13 shows cBest eyes dosing in comparison to published cBest subjects.

FIG. 14A-FIG. 14D demonstrate RPE-photoreceptor interface structure in cBest mutant models and rescue of retinal microarchitecture post AAV-mediated BEST1 gene augmentation therapy. The panels show canine WT control retina (age: 71 weeks) (FIG. 14A), cBest-R25*-heterozygous mutant retina (age: 16 weeks) (FIG. 14B), cBest-R25*/P463fs mutant-untreated retina (116 weeks) (FIG. 14C), and cBest-R25*/P463fs mutant retina examined at 74 weeks post subretinal injection of AAV-BEST1-treated (Tx). Structural abnormalities at the RPE-PR interface associated with expansion of subretinal space (ELM to RPE distance) and compromised interphotoreceptor matrix (IPM) were detected in cBest mutant retina (FIG. 14B) cBest-het with monoallelic BEST1 mutation (arrow), and (FIG. 14C) cBest mutant harboring biallelic BEST1 mutation (bracket), assayed with PNA (peanut agglutinin lectin) marker. Note a remarkable restoration of the extracellular matrix in cBest AAV-BEST1 treated retina (FIG. 14D) comparable to the WT control (FIG. 14A). PNA: peanut agglutinin lectin known for its selective binding to the cone insoluble extracellular matrix microdomains of interphotoreceptor matrix (IPM). DAPI (4′, 6-diamidino-2-phenylindole) was used as a nuclear counterstain.

FIG. 15A and FIG. 15B demonstrate reestablishment of lipid homeostasis post AAV-mediated BEST1 gene therapy. Spatial distribution of unesterified (free) cholesterol visualized by sterol-binding probe filipin (in a normal and cBest1-R25*-mutant retina (FIG. 15A). Note the excess of autofluorescent RPE deposits in the diseased tissue. Histochemical detection of esterified cholesterol in a 12-month-old cBest vs age-matched control retina. Representative retinal cryosections from cBest and age-matched controls were stained with a fluorescent neutral lipids' tracer dye BODIPY 493/503 along with quantification of EC-BODIPY 493/503 signals in POS layer between WT and cBest-R25* mutant retinas. The observed difference was assessed as statistically significant using unpaired t-test (*p<0.05). EC distribution profile in canine wild-type and cBest1-affected retinas assayed with a lysochrome Oil Red O (ORO, rose). ORO-positive inclusions within the affected RPE (arrows) and in the subretinal space are shown (close-up). Anti-4-HNE labeling in the mutant vs control retina was measured. A scattered distribution of HNE-adducts within outer segments was observed in cBest retina outlining the apical contour of the hypertrophic RPE cells). Nuclei were counterstained with propidium iodide or DAPI. Restoration of subretinal space homeostasis in cBest-R25* mutant retina vs controls is depicted in FIG. 15B.

FIG. 16A-FIG. 16C show an OS+ thickness analysis from Spectralis OCT-derived maps at pre-dose and 12 weeks post-dose. Inter-eye comparison within treatment groups of the mean (±SD) OS+ thickness in the treated area of the injected (OS) eyes and the equivalent treated areas of the un-injected/contralateral (OD) eyes (OD) at pre-dose were analyzed by paired t-test. (FIG. 16A). Inter-eye comparison within treatment groups of the mean (±SD) OS+ thickness in the treated area of the injected (OS) eyes and the equivalent treated areas of the un-injected/contralateral (OD) eyes (OD) at 12 weeks post dose was performed was performed (FIG. 16C). Paired t-test: *=p≤0.05. Comparison across treatment groups of the normalized inter-eye difference [IED (OS-OD) at 12 weeks—IED (OS-OD) at pre-dose]. Boxed asterisks represent p values from the one-way ANOVA; colored asterisks represent p values of the Bonferroni post-hoc analysis: *=p≤0.05, **=p≤0.01.

FIG. 17 shows a comparison across treatment groups of the inter-eye differences in OS+ thickness (between treated and equivalent treated areas) at 12 weeks post-dose from Bioptigen OCT B-scans Boxed asterisk represents p value from the one-way ANOVA; colored asterisks represent p values of the Bonferroni post-hoc analysis: *=p≤0.05.

FIG. 18 shows a comparison across treatment groups of the inter-eye differences in ONL thickness (between treated and equivalent treated areas) at 12 weeks post-dose from Bioptigen OCT B-scans. Analyzed by one-way ANOVA.

FIG. 19A-FIG. 19C show ONL thickness analysis from Spectralis OCT-derived maps at pre-dose and 12 weeks post-dose. Inter-eye comparison within treatment groups of the mean (±SD) ONL thickness in the treated area of the injected (OS) eyes and the equivalent treated areas of the un-injected/contralateral (OD) eyes (OD) at pre-dose was analyzed by paired t-test (FIG. 19A). Inter-eye comparison within treatment groups of the mean (±SD) ONL thickness in the treated area of the injected (OS) eyes and the equivalent treated areas of the un-injected/contralateral (OD) eyes (OD) at 12 weeks post dose was assessed by paired t-test (FIG. 19B). Comparison across treatment groups of the normalized inter-eye difference [IED (OS-OD) at 12 weeks—IED (OS-OD) at pre-dose] was performed using one-way ANOVA test (FIG. 19C).

FIG. 20A-FIG. 20B show Intraocular Pressures (IOPs). Comparison of the mean (SD) IOP of the injected (OS) versus the un-injected (OD) eyes for each treatment group at pre-dose and Study Weeks 1, 4, 8, and 12 was performed (FIG. 20A). The dashed lines represent the 95% CI of IOPs measured on untreated dogs of 9 months of age in our colony. Paired t-test: *=p≤0.05 and **=p≤0.01. In FIG. 20B, comparison across treatment groups of the mean difference (OS-OD) in IOPs at each time point wasanalyzed statistically with one-way ANOVA; NS=non-significant.

FIG. 21 shows mean ERG amplitudes as a function of intensity of light stimulation in the vehicle treated group. Intensity response curves are shown for both the injected (OS) eyes and un-injected (OS) eyes at pre-dose (dotted lines) and at 11 weeks post-dose (continuous line). Paired t-test.

FIG. 22 shows mean ERG amplitudes as a function of intensity of light stimulation in the low-dose AAV2/2-BEST1 treated group. Intensity response curves are shown for both the injected (OS) eyes and un-injected (OD) eyes at pre-dose (dotted lines) and at 11 weeks post-dose (continuous lines). Comparison of mean amplitudes between injected (OS) and un-injected (OD) eyes at 11 wks post-dose are shown; paired t-test; *=p≤0.05, **=p≤0.01.

FIG. 23 shows mean ERG amplitudes as a function of intensity of light stimulation in the high-dose AAV2/2-BEST1 treated group. Intensity response curves are shown for both the injected (OS) eyes and un-injected (OD) eyes at pre-dose (dotted lines) and at 11 weeks post-dose (continuous lines). Comparison of mean amplitudes between injected (OS) and un-injected (OD) eyes at 11 wks post-dose are shown; paired t-test; *=p≤0.05; **=p≤0.01; ***=p≤0.001.

FIG. 24 shows a comparison of ERG amplitudes across treatment groups at pre-dose. Mean (±SD) differences in scotopic a- and b-wave, photopic b-wave and 29-Hz flicker amplitudes between the injected (OS) eyes and un-injected (OD) eyes as a function of intensity of light stimulus. One-way ANOVA.

FIG. 25 shows a comparison of ERG amplitudes across treatment groups at 11 weeks post-dose. Mean (±SD) differences in scotopic a- and b-wave, photopic b-wave and 29-Hz flicker amplitudes between the injected (OS) eyes and un-injected (OD) eyes as a function of intensity of light stimulus. Boxed asterisks represent p values from the one-way ANOVA; colored asterisks represent p value of Bonferroni post-hoc analysis: *=p≤0.05, **=p≤0.01, and ***=p≤0.001.

FIG. 26 shows a heat map summary of histopathological findings in the visual pathway in all treatment groups.

FIG. 27A and FIG. 27B show representative retinal histology and quantification of ONL thickness at 13 weeks post-dose in individual injected and un-injected eyes from all 3 treatment groups. (FIG. 27A) Photomicrographs of H&E-stained sections showing the retinal morphology in the treated area of the injected (OS) eye and the equivalent location of the contralateral un-injected (OD) eye. (FIG. 27B) Spidergraphs of ONL thickness measured in both eyes (OS/injected eye; OD/un-injected eye) that extend from the optic nerve head (ONH) to the peripheral ora serrata along both the inferonasal (Inf.—Nasal) and superotemporal (Sup.—Temp.) quadrants. The section was oriented so as to include the treated area in OS and equivalent area in OD. The bar under the x-axis of each spidergraph corresponds to the 5 locations within the treated area (and equivalent area in OD) that were selected for calculation of the mean ONL thickness in the treated area of OS and equivalent area in OD. The black arrows point to the location where the H&E images shown in FIG. 27A were taken. A=artefactual ONL separation during tissue processing.

FIG. 28A and FIG. 28B show quantitative analysis of the retention of ONL thickness in the treated area measured by histology at 13 weeks post-dose. Mean (±SD) ONL thickness of the treated area of the injected (OS) eyes and of the equivalent area of the un-injected (OD) eyes in the vehicle, low-, and high-dose treatment groups was performed (FIG. 28A). Paired t-tests; N.S.=non-significant. Comparison across treatment groups of the mean (±SD) difference in ONL thickness between the treated area of the injected (OS) eyes and the equivalent area of the un-injected (OD) eyes was assessed (FIG. 28B). One-way ANOVA; NS=non-significant.

FIG. 29A-FIG. 29C show scotopic a-wave amplitudes as a function of intensity of light stimulation for individual animals in each of the treatment groups (FIG. 29A: vehicle, FIG. 29B: low-dose, and FIG. 29C: high-dose). Intensity response curves are shown for both the injected (OS) eyes and un-injected (OD) eyes at pre-dose and at 11 weeks post-dose.

FIG. 30A-FIG. 31C show scotopic b-wave amplitudes as a function of intensity of light stimulation for individual animals in each of the treatment groups (FIG. 30A: vehicle, FIG. 30B: low-dose, and FIG. 30C: high-dose). Intensity response curves are shown for both the injected (OS) eyes and un-injected (OD) eyes at pre-dose and at 11 weeks post-dose.

FIG. 31A-FIG. 31C show photopic b-wave amplitudes as a function of intensity of light stimulation for individual animals in each of the treatment groups (FIG. 31A: vehicle, FIG. 31B: low-dose, and FIG. 31C: high-dose). Intensity response curves are shown for both the injected (OS) eyes and un-injected (OD) eyes at pre-dose and at 11 weeks post-dose.

FIG. 32A-FIG. 32C show photopic flicker (29 Hz) amplitudes as a function of intensity of light stimulation for individual animals in each of the treatment groups (FIG. 32A: vehicle, FIG. 32B: low-dose, and FIG. 32C: high-dose). Intensity response curves are shown for both the injected (OS) eyes and un-injected (OD) eyes at pre-dose and at 11 weeks post-dose.

### DETAILED DESCRIPTION OF THE INVENTION

In certain aspects, provided herein are methods for treating bestrophinopathies. Also provided herein are methods for assessing retinal phenotype in subjects, including those harboring BEST1 mutations. The methods are particularly suitable for evaluating the effectiveness of therapies in animal models used for research and development, as well as for diagnosing or assessing treatment of human subjects in a clinical setting. Accordingly, the subject being treated may be an animal model or a human subject having a mutation in a BEST1 allele.

In certain embodiments, provided herein are methods for treating, retarding, or halting progression of disease in a mammalian subject having an autosomal dominant (AD) BEST1-related ocular disease. In certain embodiments, the subject harbors a mutation in a BEST1 gene allele or has been identified as having or at risk of developing a bestrophinopathy, as described herein. The subject may be heterozygous for a specific mutation in the BEST1 gene, with one wild type allele, resulting in autosomal dominant (AD) bestrophinopathy. In certain embodiments, the AD bestrophinopathy may be Best vitelliform macular dystrophy (BVMD), adult-onset vitelliform macular dystrophy (AVMD), Vitreoretinochoroidopathy, Autosomal Dominant (ADVIRC), or retinitis pigmentosa (RP). The subject may have a homozygous mutation (presence of the identical mutation on both alleles) or compound heterozygous mutation (both alleles of the same gene harbor mutations, but the mutations are different). As used herein, the term “biallelic” or “Autosomal Recessive (AR)” covers both causes. In certain embodiments, the methods of treatment include providing a viral vector, as described herein. In certain embodiments, the bestrophinopathy is a result of a mutation that causes haploinsufficiency, where the lack of the amount of the wildtype protein rather than the presence of the mutant protein causes the disease.

A naturally occurring canine model of BEST1-associated retinopathies, canine Best (cBest), has been previously described (Guziewicz et al, Bestrophin gene mutations cause canine multifocal retinopathy: a novel animal model for best disease. Invest Ophthalmol Vis Sci. 2007, incorporated herein by reference). Briefly, the model utilizes dogs that are homozygous mutant for the canine BEST1 (cBEST1) gene, and may result from any of three mutations identified at that locus. The homozygous mutant dogs of the model exhibit all major aspects of the human homozygous recessive BEST1 disease-associated mutations as well as their molecular consequences described in humans.

As described herein, in vivo and ex vivo examination of cBEST1-heterozygous mutant (cBest-Het) dogs revealed an intermediate phenotype, indicating haploinsufficiency as a predominant mechanism underlying Best disease. As such, canine cBest-Het is the first spontaneous animal model for autosomal dominant Best vitelliform macular dystrophy (BVMD). The work described herein is the first identification of the cBest-Het phenotype, which enables use of the cBest-Het model for various diagnostic and therapeutic applications, as further described herein. The cBest-Het model may be useful in assessing potential efficacy of therapies, e.g., AAV mediated BEST1 gene augmentation therapies, for treatment of autosomal dominant BEST1-related ocular disorders such as BVMD. Moreover, the identification of phenotypical abnormalities in subjects harboring single copies of a mutant BEST1 allele potentially allows for improved methods of assessing therapies and evaluating treatment for bestrophinopathy in the human population, particularly in those with autosomal dominant disease. Furthermore, the observable and measurable features of the, at times, sub-clinical phenotype allow enhanced identification of individual subjects and patient populations that may be candidates for AAV mediated BEST1 gene augmentation therapies.

Also provided herein are compositions and methods for treating subjects having, or at risk of developing, autosomal dominant bestrophinopathy.

All scientific and technical terms used herein have their known and normal meaning to a person of skill in the fields of biology, biotechnology and molecular biology and by reference to published texts, which provide one skilled in the art with a general guide to many of the terms used in the present application. However, for clarity, certain terms are defined as provided herein.

The term “about” as used herein when referring to a measurable value such as an amount, a temporal duration, and the like, is meant to encompass variations of up to ±10% from the specified value; as such variations are appropriate to perform the disclosed method.

As used in this specification and the appended claims, the singular forms “a,” “an,” and “the” include plural referents unless the content clearly dictates otherwise. As such, the terms “a” (or “an”), “one or more,” and “at least one” are used interchangeably herein. Thus, for example, reference to “a vector” includes two or more of the vectors, and the like.

Various embodiments in the specification are presented using “comprising” language, which is inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention.

BEST1 belongs to the bestrophin family of anion channels, which includes BEST2 (607335), BEST3 (607337), and BEST4 (607336). Bestrophins are transmembrane (TM) proteins that share a homology region containing a high content of aromatic residues, including an invariant arg-phe-pro (RFP) motif. The bestrophin genes share a conserved gene structure, with almost identical sizes of the 8 RFP-TM domain-encoding exons and highly conserved exon-intron boundaries.

The OMIM DB (www.ncbi.nlm.nih.gov/omim) lists five phenotypes associated with hBEST1 gene mutations, collectively termed ‘bestrophinopathies’, with the first affection described in 1905 (by Friedrich Best) and the latest one recognized in 2006 (Autosomal recessive bestrophinopathy (ARB)). The autosomal recessive form (ARB) can be caused by homozygous mutation (presence of the identical mutation on both alleles) or compound heterozygous mutation (both alleles of the same gene harbor mutations, but the mutations are different). As used herein, the term “biallelic” or “Autosomal Recessive (AR)” covers both causes.

Burgess et al. (Biallelic mutation of BEST1 causes a distinct retinopathy in humans. Am J Hum Genet. 2008 January; 82(1):19-31) described a distinct retinal disorder they designated autosomal recessive bestrophinopathy (ARB). Characteristics of the disorder included central visual loss, a characteristic retinopathy, an absent electrooculogram (EOG) light peak rise, and a reduced electroretinogram (ERG). None of the patients showed the vitelliform lesions characteristic of Best disease, but showed a diffuse irregularity of the reflex from the retinal pigment epithelium (RPE), including dispersed punctate flecks. All patients showed an accumulation of fluid within and/or beneath the neurosensory retina in the macula region. All patients were hyperopic, and 3 from 2 families also had angle-closure glaucoma. The severe reduction in the EOG light peak rise seen in all patients was similar to that seen both in Best disease and ADVIRC.

Autosomal dominant forms of bestrophinopathies are caused by monoallelic mutations in in the bestrophin gene (bestrophin-1). As used herein the term “Autosomal Dominant (AD) Best disease” may refer to any disease caused by a heterozygous mutation in the BEST1 gene. Such mutations may include a mutation in the heterozygous state. Such conditions include Best vitelliform macular dystrophy, Autosomal dominant vitreoretinochoroidopathy, Adult-onset vitelliform macular dystrophy, and MRCS syndrome.

Best vitelliform macular dystrophy (BVMD or VMD2), also called Best disease, is an early-onset autosomal dominant disorder characterized by large deposits of lipofuscin-like material in the subretinal space, which creates characteristic macular lesions resembling the yolk of an egg (‘vitelliform’). Although the diagnosis of Best disease is often made during the childhood years, it is more frequently made much later and into the sixth decade of life. In addition, the typical egg yolk-like lesion is present only during a limited period in the natural evolution of the disease; later, the affected area becomes deeply and irregularly pigmented and a process called ‘scrambling the egg’ occurs, at which point the lesion may appear as a ‘bull's eye.’ The disorder is progressive and loss of vision may occur. A defining characteristic of Best disease is a light peak/dark trough ratio of the electrooculogram (EOG) of less than 1.5, without aberrations in the clinical electroretinogram (ERG). Even otherwise asymptomatic carriers of BEST1 mutations, as assessed by pedigree, will exhibit an altered EOG. Histopathologically, the disease has been shown to manifest as a generalized retinal pigment epithelium (RPE) abnormality associated with excessive lipofuscin accumulation, regions of geographic RPE atrophy, and deposition of abnormal fibrillar material beneath the RPE, similar to drusen. Occasional breaks in the Bruch membrane with accompanying neovascularization have also been reported, although Best disease is not noted for extensive choroidal neovascularization.

BVMD often presents in several stages, although all individuals may not progress beyond the early stages.

Stage 1 (pre-vitelliform stage) consists of normal macula or subtle RPE pigment changes, EOG is abnormal and visual acuity (VA) is 20/20.

Stage 2 (vitelliform stage) consists of well-circumscribed, 0.5-5 mm round, elevated, yellow or orange lesion(s) bearing an egg-yolk appearance; usually centered on the fovea; may be multifocal; rest of the fundus has a normal appearance. VA is 20/20 to 20/50.

Stage 3 (pseudohypopyon stage) consists of yellow material which accumulate in the subretinal space in a cyst with a fluid level. The yellow material shifts with extended changes in position (60-90 min). This stage has been described in individuals aged 8-38 years. VA is 20/20 to 20/50.

Stage 4 (vitelliruptive stage) consists of scrambled egg appearance due to break up of the uniform vitelliform lesion. Pigment clumping and early atrophic changes may be noted. Visual acuity may deteriorate moderately. VA is 20/20 to 20/100.

Stage 5 (atrophic stage) consists of disappearance of the yellow material over time and an area of RPE atrophy remains. This appearance is difficult to distinguish from other causes of macular degeneration. Visual acuity can deteriorate more markedly at this stage. VA may reduce to less than 20/200.

Stage 6 (CNVM/cicatricial stage) occurs after the atrophic stage, where choroidal neovascularisation may develop and leading to a whitish subretinal fibrous scar. See, e.g., Maggon et al, Best's Vitelliform Macular Dystrophy, Med J Armed Forces India. 2008 October; 64(4): 379-381, which is incorporated herein by reference.

Adult-onset vitelliform macular dystrophy (AVMD) is one of the most common forms of macular degeneration. The age of AVMD onset is highly variable, but patients have a tendency to remain asymptomatic until the fifth decade. The clinical characteristics of AVMD are relatively benign, including a small subretinal vitelliform macular lesion, a slower progression of disease, and a slight deterioration in electrooculography (EOG). In some cases, AVMD is associated with autosomal dominant inheritance, with mutations in PRPH2, BEST1, IMPG1, or FIPG2.

Autosomal dominant vitreoretinochoroidopathy (ADVIRC or VRCP) is a disorder that affects several parts of the eyes, including the clear gel that fills the eye (the vitreous), the light-sensitive tissue that lines the back of the eye (the retina), and the network of blood vessels within the retina (the choroid). The eye abnormalities in ADVIRC can lead to varying degrees of vision impairment, from mild reduction to complete loss, although some people with the condition have normal vision. ADVIRC is caused by heterozygous mutation in the bestrophin-1 gene.

Retinitis pigmentosa is a retinal dystrophy belonging to the group of pigmentary retinopathies. Retinitis pigmentosa is characterized by retinal pigment deposits visible on fundus examination and primary loss of rod photoreceptor cells followed by secondary loss of cone photoreceptors. Patients typically have night vision blindness and loss of midperipheral visual field. As their condition progresses, they lose their far peripheral visual field and eventually central vision as well. Retinitis pigmentosa-50 (RP50) is caused by heterozygous mutation in the BEST1 gene, while certain types of retinitis pigmentosa can be autosomal recessive.

MRCS syndrome (Microcornea, rod-cone dystrophy, cataract, and posterior staphyloma) is a rare, genetic retinal dystrophy disorder characterized by bilateral microcornea, rod-cone dystrophy, cataracts and posterior staphyloma, in the absence of other systemic features. Night blindness is typically the presenting manifestation and nystagmus, strabismus, astigmatism and angle closure glaucoma may be associated findings. Progressive visual acuity deterioration, due to pulverulent-like cataracts, results in poor vision ranging from no light perception to 20/400. MRCS is caused by heterozygous mutation in the BEST1 gene.

In certain embodiments, provided herein are methods for treating, retarding, or halting progression of blindness in a mammalian subject having an BEST1-related ocular disease. In certain embodiments, the subject harbors a mutation in a BEST1 gene allele or has been identified as having or at risk of developing a bestrophinopathy, as described herein. The subject may be heterozygous for a specific mutation in the BEST1 gene, with one wild type allele. In certain embodiments, the subject is heterozygous for a mutant BEST1 allele resulting in autosomal dominant bestrophinopathy. The AD bestrophinoapthy may be selected from BVMD, AVMD, ADVIRC, RP and MRCS. In certain embodiments, the methods of treatment include providing a viral vector, as described herein. In another embodiment, the subject has a biallelic form of bestrophinopathy. In one embodiment, the bestrophinopathy is ARB. In certain embodiments of this invention, the subject has an “ocular disease,” e.g., a BEST1-related ocular disease. Clinical signs of such ocular diseases include, but are not limited to, decreased peripheral vision, retinal degeneration, decreased central (reading) vision, decreased night vision, loss of color perception, reduction in visual acuity, decreased photoreceptor function, pigmentary changes, and ultimately blindness.

Retinal degeneration is a retinopathy which consists in the deterioration of the retina caused by the progressive death of its cells. There are several reasons for retinal degeneration, including artery or vein occlusion, diabetic retinopathy, R.L.F./R.O.P. (retrolental fibroplasia/retinopathy of prematurity), or disease (usually hereditary). Signs and symptoms of retinal degeneration include, without limitation, impaired vision, night blindness, retinal detachment, light sensitivity, tunnel vision, and loss of peripheral vision to total loss of vision. Retinal degeneration and remodeling encompass a group of pathologies at the molecular, cellular and tissue levels that are initiated by inherited retinal diseases such as those described herein and other insults to the eye/retina including trauma and retinal detachment. These retinal changes and apparent plasticity result in neuronal rewiring and reprogramming events that include alterations in gene expression, de novo neuritogenesis as well as formation of novel synapses, creating corruptive circuitry in bipolar cells through alterations in the dendritic tree and supernumerary axonal growth. In addition, neuronal migration occurs throughout the vertical axis of the retina along Muller cell columns showing altered metabolic signals, and retinal pigment epithelium (RPE) invades the retina forming the pigmented bone spicules that have been classic clinical observations of RP diseases. See, retinal degeneration, remodeling and plasticity by Bryan William Jones, Robert E. Marc and Rebecca L. Pfeiffer.

As used herein, the term “subject” means a mammalian animal, including a human, a veterinary or farm animal, a domestic animal or pet, and animals normally used for research. In certain embodiments, the subject of these methods is a human. In certain embodiments, the subject is a canine. In yet other embodiments, the subject is a non-human primate. Still other suitable subjects include, without limitation, murine, rat, feline, porcine, bovine, ovine, and others. As used herein, the term “subject” is used interchangeably with “patient.” In certain embodiments, the subject is a laboratory animal suitable for research purposes (including, but not limited to, mouse, rat, canine, and non-human primate) that has been genetically modified, for example, to introduce a mutation in an endogenous BEST1 gene or to introduce a transgene encoding a mutant BEST1. In certain embodiments, the animal subject has been modified to express a heterologous BEST1 gene, such as hBEST1 or a mutant hBEST1. In another embodiment, the animal subject is a cBEST1-heterozygous mutant. In certain embodiments, the subject is a cBest-heterozygous mutant model dog, as described herein. Transgenic animals can be generated produced by any method known to those of ordinary skill in the art (for example, a zinc finger nuclease, a TALEN and/or a CRISPR/Cas nuclease system).

In certain embodiments, the subject is a human at risk of developing bestrophinopathy (e.g., has a family history of bestrophinopathy) or has one or more confirmed BEST1 gene mutations. In one embodiment, the subject has biallelic BEST1 mutations. In yet another embodiment, the subject has shown clinical signs of a bestrophinopathy. In yet a further embodiment, the subject has shown signs of retinopathy that are also indicative of bestrophinopathy. In certain embodiments, the subject has been diagnosed with a bestrophinopathy. In yet another embodiment, the subject has not yet shown clinical signs of a bestrophinopathy. In one embodiment, the subject has, or is at risk of developing, an AD bestrophinopathy. In one embodiment, the bestrophinopathy is BVID. In another embodiment, the bestrophinopathy is AVID. In another embodiment, the bestrophinopathy is ADVIRC. In another embodiment, the bestrophinopathy is RP. In another embodiment, the bestrophinopathy is MRCS. In another embodiment, the bestrophinopathy is ARB.

Although the diagnosis of Best disease is often made during the childhood years, it is more frequently made much later and into the sixth decade of life, using traditional techniques such as fundus examination and electrooculogram (EOG). The subtle phenotypic changes identified herein are useful in diagnosing Best disease earlier, and in individuals lacking the gross retinal and visual changes previously used for identification. Thus, in certain embodiments, the techniques described herein are used to identify a subject as having, or at risk of developing, Best disease. In other embodiments, the techniques described here are used to identify a subject for suitability to receive gene replacement therapy for Best disease, such as the AAV mediated BEST1 gene augmentation therapies described herein.

The findings described herein allow, in one aspect, identification of areas of intact retina that are at risk of further degeneration. For example, while a Best-1 subject may have one or more obvious lesions, or areas of substantial retinal detachment, the entirety of the retina is affected by the disease. Thus, although some areas of the retina may show microdetachments, or even appear healthy, based on traditional analysis techniques, the advanced techniques and observations described herein allow for monitoring of these areas which lack gross clinical changes. Thus, in one embodiment, a method of assessing or monitoring a subject for areas of retinal degeneration are provided. The method includes assessing the retina for the sub-clinical phenotypic changes described herein. Such changes include dysregulation of lipid homeostasis; COS elongation, thinning, and/or curving; ROS elongation, thinning, and/or curving; glial activation; ELM-RPE distance elongation; accumulation of retinal debris; abnormal POS-RPE apposition and microarchitecture of RPE-PR interface; compromised IPM and defective ELM; fluctuation of ONL thickness associated with reactive gliosis and cell migration; schistic changes in the inner/outer retina; formation of subretinal & intraretinal scars; RPE monolayer hypertrophy; occasional severe deformation of individual RPE cells associated with ONL & INL thickness fluctuations.

In one embodiment, the subject is 10 years of age or less. In another embodiment, the subject is 15 years of age or less. In another embodiment, the subject is 20 years of age or less. In another embodiment, the subject is 25 years of age or less. In another embodiment, the subject is 30 years of age or less. In another embodiment, the subject is 35 years of age or less. In another embodiment, the subject is 40 years of age or less. In another embodiment, the subject is 45 years of age or less. In another embodiment, the subject is 50 years of age or less. In another embodiment, the subject is 55 years of age or less. In another embodiment, the subject is 60 years of age or less. In another embodiment, the subject is 65 years of age or less. In another embodiment, the subject is 70 years of age or less. In another embodiment, the subject is 75 years of age or less. In another embodiment, the subject is 80 years of age or less. In another embodiment, the subject is a neonate, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 years of age or greater.

As used herein, the term “treatment,” and variations thereof such as “treat” or “treating,” refer to clinical intervention in an attempt to alter the natural course of the individual being treated and can be performed either for prophylaxis or during the course of clinical pathology. “Treatment” can thus include one or more of reducing onset or progression of an ocular disease (such as bestrophinopathy), preventing disease, reducing the severity of the disease symptoms, or retarding their progression, including the progression of blindness, removing the disease symptoms, delaying onset of disease or monitoring progression of disease or efficacy of therapy in a given subject. In certain embodiments, improved and/or maintained ERG amplitude(s) is indicative of efficacy of treatment.

Thus, in certain embodiment, a therapy is administered before disease onset. In another embodiment, a therapy is administered prior to the initiation of vision impairment or loss. In another embodiment, a therapy is administered after initiation of vision impairment or loss. In yet another embodiment, a therapy is administered when less than 90% of the rod and/or cones or photoreceptors are functioning or remaining, as compared to a non-diseased eye.

In yet another embodiment, a therapy is administered when the subject being treated exhibits symptoms of stage I (the pre-vitelliform stage) to stage III (the vitelliruptive stage or the pseudo-hypopyon stage) of BVMD. In another embodiment, therapy is administered prior to exhibiting the symptoms of stage I. In another embodiment, therapy is administered after exhibiting the symptoms of stage I. In another embodiment, therapy is administered prior to exhibiting the symptoms of stage II. In another embodiment, therapy is administered after exhibiting the symptoms of stage II. In another embodiment, therapy is administered prior to exhibiting the symptoms of stage III. In another embodiment, therapy is administered after exhibiting the symptoms of stage III. In another embodiment, therapy is administered prior to exhibiting the symptoms of stage IV. In another embodiment, therapy is administered after exhibiting the symptoms of stage IV. In another embodiment, therapy is administered prior to exhibiting the symptoms of stage V. In another embodiment, therapy is administered after exhibiting the symptoms of stage V.

As used herein, “therapy” refers to any form of intervention intended to treat an existing disease condition in a subject or reduce, delay, inhibit or eliminate the onset or progression of disease or symptoms of disease in a subject. A therapy may be a gene augmentation therapy intended to supplement, restore, or enhance expression levels of a gene by providing a nucleic acid encoding a functional protein. Thus, in certain embodiments, the methods include administering a vector, in particular a gene therapy vector. In certain embodiments, the therapy is a recombinant AAV with a canine BEST1 (cBEST1) or human BEST1 (hBEST1). Suitable vectors may also encode components of a genome editing system (e.g, CRISPR/Cas) designed to, for example, insert a gene sequence, replace a gene sequence or part of a gene sequence, or correct a mutation in an endogenous BEST1 gene sequence.

The term “transgene” as used herein means an exogenous or engineered protein-encoding nucleic acid sequence that is under the control of a promoter or expression control sequence in an expression cassette, rAAV genome, recombinant plasmid or production plasmid, vector, or host cell described in this specification. In certain embodiments, the transgene is a BEST1 sequence, encoding a functional BEST1 protein, or a fragment thereof.

In certain embodiments, the methods include administering a viral vector to a subject. Suitable viral vectors are preferably replication defective and selected from amongst those which target ocular cells. Viral vectors may include any virus suitable for gene therapy wherein a vector includes a nucleic acid sequence encoding for protein intended mediate a therapeutic effect in the subject. Suitable gene therapy vectors include, but are not limited to adenovirus; herpes virus; lentivirus; retrovirus; parvovirus, etc. However, for ease of understanding, the adeno-associated virus is referenced herein as an exemplary viral vector.

Thus, in one aspect, a recombinant adeno-associated virus (rAAV) vector is provided. The rAAV compromises an AAV capsid, and a vector genome packaged therein. The vector genome comprises, in one embodiment: (a) an AAV 5′ inverted terminal repeat (ITR) sequence; (b) a promoter; (c) an optional enhancer; (d) a coding sequence encoding a human BEST1; (e) a polyA tail; and (f) an AAV 3′ ITR. In one embodiment, the BEST1 sequence encodes a full length bestrophin protein. In one embodiment, the BEST1 sequence is the protein sequence of Uniprot Accession No. 076090-1, which is incorporated herein by reference. (See, e.g., Guziewicz et al, PNAS. 2018 Mar. 20; 115(12):E2839-E2848, which is incorporated by reference herein).

In certain embodiments, the methods include delivery of a vector (e.g., a gene therapy vector) having a nucleic acid sequence encoding a normal BEST1 gene, or fragment thereof. The term “BEST1” as used herein, refers to the full-length gene itself or a functional fragment, as further defined below. The nucleic acid sequence encoding a normal BEST1 gene, or fragment thereof, may be derived from any mammal which natively expresses the BEST1 gene, or homolog thereof. In certain embodiments, the BEST1 gene sequence is derived from the same mammal that the subject is intended to treat. Thus, in certain embodiments, the BEST1 gene is derived from a human sequence (as provided, for example, in any of NM_001139443.1, NM_001300786.1, NM_001300787.1, NM_001363591.1 NM_001363592.1 NM_001363593.1, and NM_004183.4). In certain embodiments, the BEST1 sequence encodes a protein having an amino acid sequence of UniProtKB—076090-1, 076090-3, or 076090-4. In yet other embodiments, the BEST1 gene is derived from a canine sequence (as provided, for example, in NM_001097545.1). In certain embodiments, the BEST1 sequence encodes a protein having the amino acid sequence of UniProtKB—A5H7G8-1. In certain embodiments of the methods a human BEST1 (hBEST1) gene is delivered to a mammal other than a human (such as a canine, rat, mouse, or non-human primate model) to, for example, evaluate the efficacy of a therapy. In certain embodiment, the BEST1 sequence is the sequence of the full length human BEST1. By the term “fragment” or “functional fragment”, it is meant any fragment that retains the function of the full-length protein, although not necessarily at the same level of expression or activity. Functional fragments of human, or other BEST1 sequences may be determined by one of skill in the art. In certain embodiments, the BEST1 sequence is derived from a canine. In other embodiments, certain modifications are made to the BEST1 sequence in order to enhance the expression in the target cell. Such modifications include codon optimization, (see, e.g., U.S. Pat. Nos. 7,561,972; 7,561,973; and 7,888,112, incorporated herein by reference).

The term “adeno-associated virus,” “AAV,” or “AAV serotype” as used herein refers to the dozens of naturally occurring and available adeno-associated viruses, as well as artificial AAVs. Among the AAVs isolated or engineered from human or non-human primates (NHIP) and well characterized, human AAV2 is the first AAV that was developed as a gene transfer vector; it has been widely used for efficient gene transfer experiments in different target tissues and animal models. Unless otherwise specified, the AAV capsid, ITRs, and other selected AAV components described herein, may be readily selected from among any AAV, including, without limitation, AAV1, AAV2, AAV3, AAV4, AAV5, AAV6, AAV7, AAV8, AAV9, AAV8 bp, AAV2-7m8 and AAVAnc80, variants of any of the known or mentioned AAVs or AAVs yet to be discovered or variants or mixtures thereof. See, e.g., WO 2005/033321, which is incorporated herein by reference. In another embodiment, the AAV is selected from AAV10, AAV11, AAV12, LK01, LK02, LK03, AAV 4-1, AAV-2i8, Rh10, and/or Rh74. In another embodiment, the AAV capsid is an AAV8 bp capsid, which preferentially targets bipolar cells. See, WO 2014/024282, which is incorporated herein by reference. In another embodiment, the AAV capsid is an AAV2-7m8 capsid, which has shown preferential delivery to the outer retina. See, Dalkara et al, In Vivo-Directed Evolution of a New Adeno-Associated Virus for Therapeutic Outer Retinal Gene Delivery from the Vitreous, Sci Transl Med 5, 189ra76 (2013), which is incorporated herein by reference. In one embodiment, the AAV capsid is an AAV8 capsid. In another embodiment, the AAV capsid an AAV9 capsid. In another embodiment, the AAV capsid an AAV5 capsid. In another embodiment, the AAV capsid an AAV2 capsid.

As used herein, “artificial AAV” means, without limitation, an AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized AAV capsid. Pseudotyped vectors, wherein the capsid of one AAV is replaced with a heterologous capsid protein, are useful in the invention. In one embodiment, AAV2/5 and AAV2-7m8 are exemplary pseudotyped vectors.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector, indicates that the protein or the nucleic acid is present with another sequence or subsequence which with which the protein or nucleic acid in question is not found in the same relationship to each other in nature.

For packaging an expression cassette or rAAV genome or production plasmid into virions, the ITRs are the only AAV components required in cis in the same construct as the expression cassette. In one embodiment, the coding sequences for the replication (rep) and/or capsid (cap) are removed from the AAV genome and supplied in trans or by a packaging cell line in order to generate the AAV vector.

Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2]. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated, even if subsequently reintroduced into the natural system. Such polynucleotides could be part of a vector and/or such polynucleotides or polypeptides could be part of a composition, and still be isolated in that such vector or composition is not part of its natural environment.

In yet another system, the expression cassette flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety.

The methods used to construct any embodiment of this invention are known to those with skill in nucleic acid manipulation and include genetic engineering, recombinant engineering, and synthetic techniques. See, e.g., Green and Sambrook et al, Molecular Cloning: A Laboratory Manual, Cold Spring Harbor Press, Cold Spring Harbor, NY (2012). Similarly, methods of generating rAAV virions are well known and the selection of a suitable method is not a limitation on the present invention. See, e.g., K. Fisher et al, (1993) J. Virol., 70:520-532 and U.S. Pat. No. 5,478,745.

In certain embodiments, the rAAV expression cassette, the vector, and/or the virus comprises AAV inverted terminal repeat sequences, a nucleic acid sequence that encodes BEST1, and expression control sequences that direct expression of the encoded proteins in a host cell. In other embodiments, the rAAV expression cassette, the virus, and/or the vector further comprises one or more of an intron, a Kozak sequence, a polyA, post-transcriptional regulatory elements and others. In one embodiment, the post-transcriptional regulatory element is Woodchuck Hepatitis Virus (WHP) Posttranscriptional Regulatory Element (WPRE).

The expression cassettes, vectors and plasmids include other components that can be optimized for a specific species using techniques known in the art including, e.g, codon optimization, as described herein.

The components of the cassettes, vectors, plasmids and viruses or other compositions described herein include a promoter sequence as part of the expression control sequences. In one embodiment, the promoter is the native hVMD2 promoter. In another embodiment, the promoter is cell-specific. The term “cell-specific” means that the particular promoter selected for the recombinant vector can direct expression of the BEST1 coding sequence in a particular ocular cell type. In one embodiment, the promoter is specific for expression of the transgene in RPE. In one embodiment, the promoter is specific for expression of the transgene in photoreceptor cells. In another embodiment, the promoter is specific for expression in the rods and cones. In another embodiment, the promoter is specific for expression in the rods. In another embodiment, the promoter is specific for expression in the cones. In one embodiment, the photoreceptor-specific promoter is a human rhodopsin kinase promoter. The rhodopsin kinase promoter has been shown to be active in both rods and cones. See, e.g., Sun et al, Gene Therapy with a Promoter Targeting Both Rods and Cones Rescues Retinal Degeneration Caused by AIPL1 Mutations, Gene Ther. 2010 January; 17(1): 117-131, which is incorporated herein by reference in its entirety. In one embodiment, the promoter is modified to add one or more restriction sites to facilitate cloning.

In one embodiment, the promoter is the native hVMVD2 promoter or a modified version thereof. See, Guziewicz et al., PLoS One. 2013 Oct. 15; 8(10):e75666, which is incorporated herein by reference.

In one embodiment, the promoter is a human rhodopsin promoter. In one embodiment, the promoter is modified to include restriction on the ends for cloning. See, e.g, Nathans and Hogness, Isolation and nucleotide sequence of the gene encoding human rhodopsin, PNAS, 81:4851-5 (August 1984), which is incorporated herein by reference in its entirety. In another embodiment, the promoter is a portion or fragment of the human rhodopsin promoter. In another embodiment, the promoter is a variant of the human rhodopsin promoter.

Other exemplary promoters include the human G-protein-coupled receptor protein kinase 1 (GRK1) promoter (Genbank Accession number AY327580). In another embodiment, the promoter is a 292 nt fragment (positions 1793-2087) of the GRK1 promoter (See, Beltran et al, Gene Therapy 2010 17:1162-74, which is hereby incorporated by reference in its entirety). In another preferred embodiment, the promoter is the human interphotoreceptor retinoid-binding protein proximal (IRBP) promoter. In one embodiment, the promoter is a 235 nt fragment of the hIRBP promoter. In one embodiment, the promoter is the RPGR proximal promoter (Shu et al, IOVS, May 2102, which is incorporated by reference in its entirety). Other promoters useful in the invention include, without limitation, the rod opsin promoter, the red-green opsin promoter, the blue opsin promoter, the cGMP-β-phosphodiesterase promoter (Qgueta et al, IOVS, Invest Ophthalmol Vis Sci. 2000 December; 41(13):4059-63), the mouse opsin promoter (Beltran et al 2010 cited above), the rhodopsin promoter (Mussolino et al, Gene Ther, July 2011, 18(7):637-45); the alpha-subunit of cone transducin (Morrissey et al, BMC Dev, Biol, January 2011, 11:3); beta phosphodiesterase (PDE) promoter; the retinitis pigmentosa (RP1) promoter (Nicord et al, J. Gene Med, December 2007, 9(12):1015-23); the NXNL2/NXNL1 promoter (Lambard et al, PLoS One, October 2010, 5(10):e13025), the RPE65 promoter; the retinal degeneration slow/peripherin 2 (Rds/perph2) promoter (Cai et al, Exp Eye Res. 2010 August; 91(2):186-94); and the VMD2 promoter (Kachi et al, Human Gene Therapy, 2009 (20:31-9)). Each of these documents is incorporated by reference herein in its entirety. In another embodiment, the promoter is selected from human EFlα promoter, rhodopsin promoter, rhodopsin kinase, interphotoreceptor binding protein (IRBP), cone opsin promoters (red-green, blue), cone opsin upstream sequences containing the red-green cone locus control region, cone transducing, and transcription factor promoters (neural retina leucine zipper (Nrl) and photoreceptor-specific nuclear receptor Nr2e3, bZIP).

In another embodiment, the promoter is a ubiquitous or constitutive promoter. An example of a suitable promoter is a hybrid chicken β-actin (CBA) promoter with cytomegalovirus (CMV) enhancer elements. In another embodiment, the promoter is the CB7 promoter. Other suitable promoters include the human β-actin promoter, the human elongation factor-1α promoter, the cytomegalovirus (CMV) promoter, the simian virus 40 promoter, and the herpes simplex virus thymidine kinase promoter. See, e.g., Damdindorj et al, (August 2014) A Comparative Analysis of Constitutive Promoters Located in Adeno-Associated Viral Vectors. PLoS ONE 9(8): e106472. Still other suitable promoters include viral promoters, constitutive promoters, regulatable promoters [see, e.g., WO 2011/126808 and WO 2013/04943]. Alternatively a promoter responsive to physiologic cues may be utilized in the expression cassette, rAAV genomes, vectors, plasmids and viruses described herein. In one embodiment, the promoter is of a small size, under 1000 bp, due to the size limitations of the AAV vector. In another embodiment, the promoter is under 400 bp. Other promoters may be selected by one of skill in the art.

In a further embodiment, the promoter is selected from SV40 promoter, the dihydrofolate reductase promoter, and the phosphoglycerol kinase (PGK) promoter, rhodopsin kinase promoter, the rod opsin promoter, the red-green opsin promoter, the blue opsin promoter, the inter photoreceptor binding protein (IRBP) promoter and the cGMP-β-phosphodiesterase promoter, a phage lambda (PL) promoter, a herpes simplex viral (HSV) promoter, a tetracycline-controlled trans-activator-responsive promoter (tet) system, a long terminal repeat (LTR) promoter, such as a RSV LTR, MoMLV LTR, BIV LTR or an HIV LTR, a U3 region promoter of Moloney murine sarcoma virus, a Granzyme A promoter, a regulatory sequence(s) of the metallothionein gene, a CD34 promoter, a CD8 promoter, a thymidine kinase (TK) promoter, a B19 parvovirus promoter, a PGK promoter, a glucocorticoid promoter, a heat shock protein (HSP) promoter, such as HSP65 and HSP70 promoters, an immunoglobulin promoter, an MMTV promoter, a Rous sarcoma virus (RSV) promoter, a lac promoter, a CaMV 35S promoter, a nopaline synthetase promoter, an MND promoter, or an MNC promoter. The promoter sequences thereof are known to one of skill in the art or available publically, such as in the literature or in databases, e.g., GenBank, PubMed, or the like.

In another embodiment, the promoter is an inducible promoter. The inducible promoter may be selected from known promoters including the rapamycin/rapalog promoter, the ecdysone promoter, the estrogen-responsive promoter, and the tetracycline-responsive promoter, or heterodimeric repressor switch. See, Sochor et al, An Autogenously Regulated Expression System for Gene Therapeutic Ocular Applications. Scientific Reports, 2015 Nov. 24; 5:17105 and Daber R, Lewis M., A novel molecular switch. J Mol Biol. 2009 Aug. 28; 391(4):661-70, Epub 2009 Jun. 21 which are both incorporated herein by reference in their entirety.

Examples of suitable polyA sequences include, e.g., a synthetic polyA or from bovine growth hormone (bGH), human growth hormone (hGH), SV40, rabbit $-globin (RGB), or modified RGB (mRGB).

Examples of suitable enhancers include, e.g., the CMV enhancer, the RSV enhancer, the alpha fetoprotein enhancer, the TTR minimal promoter/enhancer, LSP (TH-binding globulin promoter/alphal-microglobulin/bikunin enhancer), an APB enhancer, ABPS enhancer, an alpha mic/bik enhancer, TTR enhancer, en34, ApoE amongst others.

As used in the methods described herein, “administering” means delivering a therapy to a subject for treatment of ocular disease. In one embodiment, the method involves administration via subretinal injection to the RPE, photoreceptor cells or other ocular cells. In one embodiment, the method involves administration via subretinal injection to the RPE. In another embodiment, intravitreal injection to ocular cells is employed. In still another method, injection via the palpebral vein to ocular cells may be employed. In still another embodiment, suprachoroidal injection to ocular cells may be employed. Still other methods of administration may be selected by one of skill in the art given this disclosure. By “administering” or “route of administration” is delivery of a therapy described herein (e.g. a rAAV comprising a nucleic acid sequence encoding BEST1), with or without a pharmaceutical carrier or excipient, of the subject. Routes of administration may be combined, if desired. In some embodiments, the administration is repeated periodically. Direct delivery to the eye (optionally via ocular delivery, subretinal injection, intra-retinal injection, intravitreal, topical), or delivery via systemic routes, e.g., intraarterial, intraocular, intravenous, intramuscular, subcutaneous, intradermal, and other parental routes of administration. In certain embodiments, the methods provide herein include administration of nucleic acid molecules and/or vectors described herein in a single composition or multiple compositions. Optionally, two or more different AAV may be delivered, or multiple viruses [see, e.g., WO20 2011/126808 and WO 2013/049493]. In another embodiment, multiple viruses may contain different replication-defective viruses (e.g., AAV and adenovirus), alone or in combination with proteins.

As used herein, the term “ocular cells” refers to any cell in, or associated with the function of, the eye. The term may refer to any one of photoreceptor cells, including rod, cone and photosensitive ganglion cells or retinal pigment epithelium (RPE) cells. In one embodiment, the ocular cells are the photoreceptor cells. In another embodiment, the ocular cells are the RPE.

Also provided herein are pharmaceutical compositions. The pharmaceutical compositions described herein are designed for delivery to subjects in need thereof by any suitable route or a combination of different routes. These delivery means are designed to avoid direct systemic delivery of the suspension containing the AAV composition(s) described herein. Suitably, this may have the benefit of reducing dose as compared to systemic administration, reducing toxicity and/or reducing undesirable immune responses to the AAV and/or transgene product.

In yet other aspects, these nucleic acid sequences, vectors, expression cassettes and rAAV viral vectors are useful in a pharmaceutical composition, which also comprises a pharmaceutically acceptable carrier, excipient, buffer, diluent, surfactant, preservative and/or adjuvant, etc. Such pharmaceutical compositions are used to express BEST1 in the host cells through delivery by such recombinantly engineered AAVs or artificial AAVs.

To prepare these pharmaceutical compositions containing the nucleic acid sequences, vectors, expression cassettes and rAAV viral vectors, the sequences or vectors or viral vectors are preferably assessed for contamination by conventional methods and then formulated into a pharmaceutical composition suitable for administration to the eye. Such formulation involves the use of a pharmaceutically and/or physiologically acceptable vehicle or carrier, particularly one suitable for administration to the eye.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline).

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In one example, the composition is formulated for intravitreal delivery. In one example, the composition is formulated for subretinal delivery. In another example, the composition is formulated for suprachoroidal delivery.

In the case of AAV viral vectors, quantification of the genome copies (“GC”), vector genomes (“VG”), or virus particles may be used as the measure of the dose contained in the formulation or suspension. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome (usually the transgene or the poly A signal). In another method the effective dose of a recombinant adeno-associated virus carrying a nucleic acid sequence encoding BEST1 is measured as described in S. K. McLaughlin et al, 1988 J. Virol., 62:1963, which is incorporated by reference in its entirety.

As used herein, the term “dosage” can refer to the total dosage delivered to the subject in the course of treatment, or the amount delivered in a single unit (or multiple unit or split dosage) administration. The pharmaceutical virus compositions can be formulated in dosage units to contain an amount of replication-defective virus carrying the nucleic acid sequences encoding BEST1 as described herein that is in the range of about 1.0×10⁹ vg (vector genomes)/mL to about 1.0×10¹⁵ vg/mL including all integers or fractional amounts within the range. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ vg/mL including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ vg/mL including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ vg/mL including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² vg/mL including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ vg/mL including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ vg/mL including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ vg/mL including all integers or fractional amounts within the range. In one embodiment, for human application the dose can range from 1×¹⁰1° to about 1×10¹² vg/mL including all integers or fractional amounts within the range. All dosages may be measured by any known method, including as measured by oqPCR or digital droplet PCR (ddPCR) as described in, e.g., M. Lock et al, Hum Gene Ther Methods. 2014 April; 25(2):115-25. doi: 10.1089/hgtb.2013.131, which is incorporated herein by reference.

In one embodiment, an aqueous suspension suitable for administration to patient having, or suspected of having, a bestrophinopathy, is provided. The suspension comprises an aqueous suspending liquid and about 1×10⁹ GC or viral particles to about 1×10¹² GC or viral particles per eye of a recombinant adeno-associated virus (rAAV) described herein useful as a therapeutic for bestrophinopathy. In one embodiment, about 1.5×10¹⁰ GC or viral particles are administered per eye.

It may also be desirable to administer multiple “booster” dosages of the pharmaceutical compositions of this invention. For example, depending upon the duration of the transgene within the ocular target cell, one may deliver booster dosages at 6-month intervals, or yearly following the first administration. The fact that AAV-neutralizing antibodies were not generated by administration of the rAAV vector should allow additional booster administrations.

Such booster dosages and the need therefor can be monitored by the attending physicians, using, for example, the retinal and visual function tests and the visual behavior tests described in the examples below. Other similar tests may be used to determine the status of the treated subject over time. Selection of the appropriate tests may be made by the attending physician. Still alternatively, the method of this invention may also involve injection of a larger volume of virus-containing solution in a single or multiple infection to allow levels of visual function close to those found in wildtype retinas.

In another embodiment, the amount of the vectors, the virus and the replication-defective virus described herein carrying the nucleic acid sequences encoding BEST1 are in the range of about 1.0×10⁷ VG per eye to about 1.0×10¹⁵ VG per eye including all integers or fractional amounts within the range. In one embodiment, the amount thereof is at least 1×10⁷, 2×10⁷, 3×10⁷, 4×10⁷, 5×10⁷, 6×10⁷, 7×10⁷, 8×10⁷, or 9×10⁷ VG per eye including all integers or fractional amounts within the range. In one embodiment, the amount thereof is at least 1×10⁸, 2×10⁸, 3×10⁸, 4×10⁸, 5×10⁸, 6×10⁸, 7×10⁸, 8×10⁸, or 9×10⁸ VG per eye including all integers or fractional amounts within the range. In one embodiment, the amount thereof is at least 1×10⁹, 2×10⁹, 3×10⁹, 4×10⁹, 5×10⁹, 6×10⁹, 7×10⁹, 8×10⁹, or 9×10⁹ VG per eye including all integers or fractional amounts within the range. In one embodiment, the amount thereof is at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ VG per eye including all integers or fractional amounts within the range. In one embodiment, the amount thereof is at least 1×10¹¹, 2×10¹¹, 3×10¹¹, 4×10¹¹, 5×10¹¹, 6×10¹¹, 7×10¹¹, 8×10¹¹, or 9×10¹¹VG per eye including all integers or fractional amounts within the range. In one embodiment, the amount thereof is at least 1×10¹², 2×10¹², 3×10¹², 4×10¹², 5×10¹², 6×10¹², 7×10¹², 8×10¹², or 9×10¹² VG per eye including all integers or fractional amounts within the range. In one embodiment, the amount thereof is at least 1×10¹³, 2×10¹³, 3×10¹³, 4×10¹³, 5×10¹³, 6×10¹³, 7×10¹³, 8×10¹³, or 9×10¹³ VG per eye including all integers or fractional amounts within the range. In one embodiment, the amount thereof is at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ VG per eye including all integers or fractional amounts within the range. In one embodiment, the amount thereof is at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ VG per eye including all integers or fractional amounts within the range. In one embodiment, the methods comprises dose ranging from 1×10⁹ to about 1×10¹³ VG per eye per dose including all integers or fractional amounts within the range. In another embodiment, the method comprises delivery of the vector in an aqueous suspension. In another embodiment, the method comprises administering the rAAV described herein in a dosage of from 1×10⁹ to 1×10¹³ VG in a volume about or at least 150 microliters, thereby restoring visual function in said subject.

These above doses may be administered in a variety of volumes of carrier, excipient or buffer formulation, ranging from about 25 to about 1000 microliters, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 75 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 L. In another embodiment, the volume is about 600 μL. In another embodiment, the volume is about 650 μL. In another embodiment, the volume is about 700 μL. In another embodiment, the volume is about 800 μL. In another embodiment, the volume is between about 150 and 800 μL. In another embodiment, the volume is between about 700 and 1000 μL. In another embodiment, the volume is between about 250 and 500 μL.

In one embodiment, the viral constructs may be delivered in doses of from at least 1×10⁹ to about least 1×10¹¹ GCs in volumes of about 1 μL to about 3 μL for small animal subjects, such as mice. For larger veterinary subjects having eyes about the same size as human eyes, the larger human dosages and volumes stated above are useful. See, e.g., Diehl et al, J. Applied Toxicology, 21:15-23 (2001) for a discussion of good practices for administration of substances to various veterinary animals. This document is incorporated herein by reference.

It is desirable that the lowest effective concentration of virus or other delivery vehicle be utilized in order to reduce the risk of undesirable effects, such as toxicity, retinal dysplasia and detachment. Still other dosages in these ranges may be selected by the attending physician, taking into account the physical state of the subject, preferably human, being treated, the age of the subject, the bestrophinopathy and the degree to which the disorder, if progressive, has developed.

In certain embodiments, treatment efficacy is determined by identifying an at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% improvement or change relative to a measurement in a control sample. The control sample may be a normal healthy control, a mutant disease control, a pre-treatment control, an earlier timepoint control, an untreated contralateral eye, or a retinal region outside of a treatment bleb. In certain embodiments, the mutant disease control is a sample from a subject with two mutant BEST1 alleles. In yet other embodiments, the mutant disease control is from a subject having one mutant BEST1 allele and a wildtype BEST1 allele.

In certain embodiments, provided herein are methods for evaluating a treatment for a BEST1-associated maculopathy in a subject. Accordingly, the subject harbors at least one mutant BEST1 gene. In certain embodiments, the subject is heterozygous for a BEST1 mutation (e.g., one mutant BEST1 allele and one wildtype, functional BEST1 allele or a carrier of alternative mutant BEST1 alleles). In certain embodiments, following administration of the therapy, the effectiveness of the treatment is determined by performing in vivo retinal imaging to evaluate one or more of a longitudinal reflectivity profile (LRP), IS/OS to retinal pigment epithelium (RPE) distance in light-adapted and/or dark-adapted eyes, and formation of light-potentiated subretinal microdetachments (as described, for example, in Guziewicz et al., PNAS. 2018 Mar. 20; 115(12):E2839-E2848, which is incorporated by reference herein). These parameters can be supplemented with additional methods known in the art for evaluating visual function and severity of ocular disease. The effectiveness of the therapy is evaluated following administration of a therapy at time points selected based on factors such as the severity of disease, parameter to be measured, or age or species of the subject, or nature of the therapy. Accordingly, in certain time points, the effectiveness of treatment is evaluated one or more intervals following administration of a therapy. In certain embodiments, treatment efficacy is evaluated within 24 hours, 36 hours, 48 hours, or 72 hours following administration of a therapy. In yet further embodiments, treatment efficacy is evaluated one or more times within 1 week, 2 weeks, 1 month, 2 months, 3 months, 4 months, 5 months, or 6 months of administering a therapy. In certain embodiments, the therapy is treatment with a viral vector, as described herein.

Canine bestrophinopathy arises as a focal detachment between retinal pigment epithelium (RPE) and the neural retina in the area centralis and can stay limited to the canine fovea-like region or develop extramacular satellite lesions, manifestations parallel to BVMD and ARB phenotype in patients. The typical cBest presents bilaterally, has an early onset (˜12 weeks of age), and progresses slowly following well-defined clinical stages described in BVMD: Stage I, pre-vitelliform with a discreet disruption between the RPE and neural retina within the canine fovea-like region; Stage II, vitelliform, characterized by a circular, yolk-like central lesion; Stage III, pseudohypopyon phase, Stage IV, vitelliruptive, and finally Stage V, atrophic—all highly comparable between BVMD patients and cBest dogs.

Thus, in certain embodiments, the methods provided herein include administering a therapy to a canine animal model for bestrophinopathy, wherein the canine harbors BEST1 mutation that recapitulates clinical, molecular, and/or histological features characteristic of human disease. Suitable mutations include previously identified spontaneous mutations, such as c.73C>T/p.R25*, -c.482G>A/p.G161D, and c.1388delC/P463fs. cBEST1-C73T/R25*-contains a premature stop codon, resulting in null phenotype; cBEST1-G482A/G161D which contains a missense change, affecting protein folding and trafficking; and cBEST1-C1388del/P463fs which contains a frameshift mutation, truncating the C-terminus of bestrophin-1 protein. In certain embodiments, the canine has a wildtype BEST1 allele and a mutated BEST1 allele. The mutated BEST1 allele may have one or more mutations. Additional BEST1 mutations can be identified by one of ordinary skill in the art to generate animal models to be used in the methods describe herein.

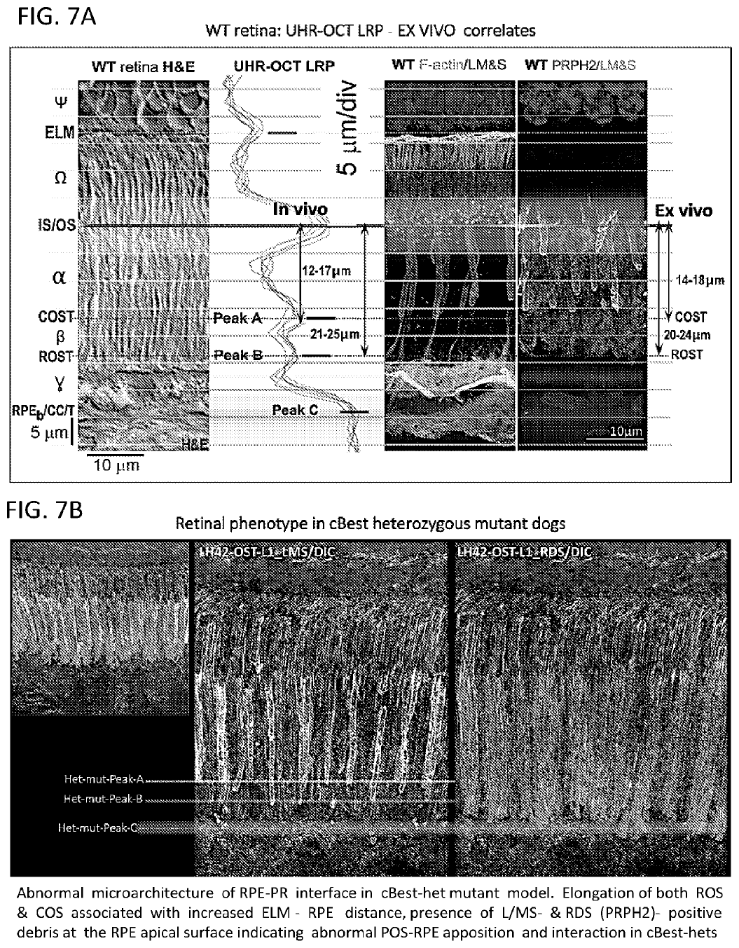

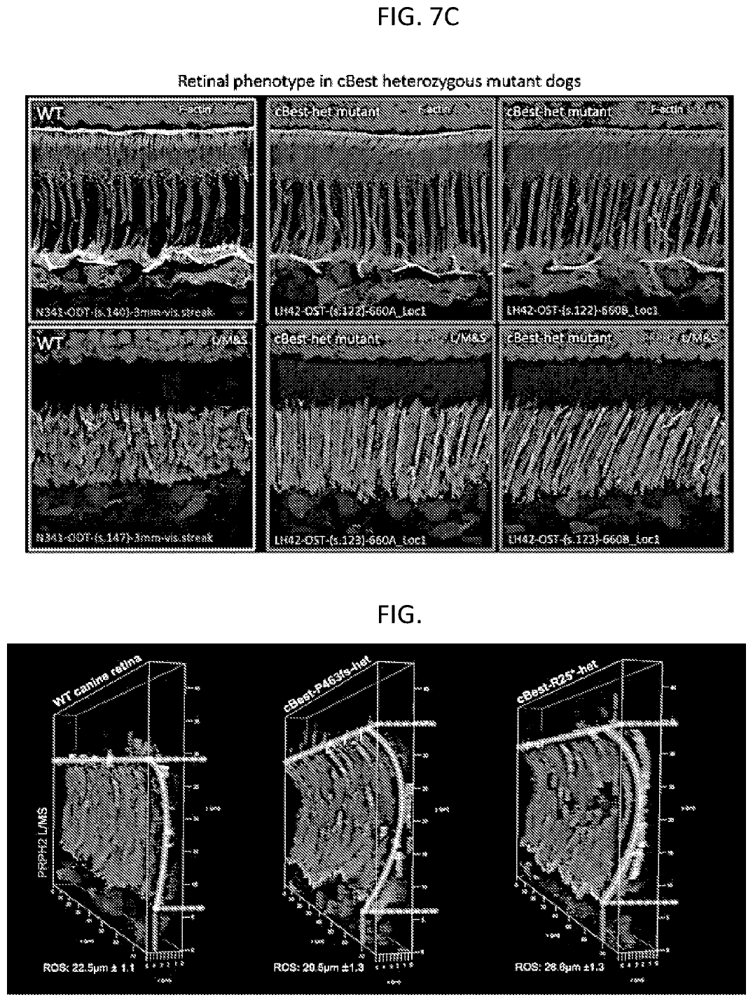

As described herein, for the first time, a previously undetected disease phenotype has been recognized in cBest heterozygote mutants. The cBest-Hets demonstrate a phenotype which shares overlapping disease aspects and pathogenesis with the cBest-homozygous mutant models previously described, but at a subtle, subclinical level. However, the subclinical manifestations observed in the cBest-Hets and described herein have not been previously identified or described, and are, identifiable only via testing with ultra-high resolution instrumentation, such as those described herein. The cBest-Het and cBest-homozygous models demonstrate retina-wide pathology of the RPE-photoreceptor interface. For example, FIG. 7A and FIG. 7B, looking at peak C, it can be seen that the RPE-PR interface of the cBest-Het model demonstrates abnormal microarchitecture due to elongation of both ROS and COS associated with increased ELM-RPE distance, the presence of L/MS- and RDS (PRPH2)—positive debris at the RPE apical surface indicating abnormal POS-RPE apposition and interaction in cBest-Hets. Furthermore, the cBest-Hets demonstrate thinning, elongation and curving of the ROS as compared to wild type retina (FIG. 7D), as well as increased formation of debris. In addition, the cBest-Het model demonstrates dysregulation of lipid homeostasis, similar to the cBest homozygous model. It is desirable that a therapeutic treatment ameliorate one or more of these phenotypic changes. In one embodiment, the treatment reduces COS elongation, thinning, and/or curving. In another embodiment, the treatment reduces ROS elongation, thinning, and/or curving. In another embodiment, the treatment reduces glial activation. In another embodiment, the treatment reduces ELM-RPE distance, in another embodiment, treatment reduces accumulation of retinal debris. In another embodiment, treatment reduces abnormal POS-RPE apposition and microarchitecture of RPE-PR interface. In another embodiment, treatment reduces subretinal debris at RPE apical surface, or within subretinal space. In another embodiment, treatment reduces compromised IPM and defective ELM. In another embodiment, treatment reduces fluctuation of ONL thickness associated with reactive gliosis and cell migration. In another embodiment, treatment reduces schistic changes in the inner/outer retina. In another embodiment, treatment reduces formation of subretinal & intraretinal scars. In another embodiment, treatment reduces RPE monolayer hypertrophy. In another embodiment, treatment reduces occasional severe deformation of individual RPE cells associated with ONL & INL thickness fluctuations. In another embodiment, treatment reduces and Muller Glial trunks/projections penetrating ONL layer. In one embodiment, treatment reduces gross macular lesion. In yet another embodiment, treatment reduces bullous detachment.

In certain embodiments, it is desirable to perform non-invasive retinal imaging and functional studies to identify areas of the rod and cone photoreceptors to be targeted for therapy. In certain embodiments, clinical diagnostic tests are employed to determine the precise location(s) for one or more subretinal injection(s). These tests may include electroretinography (ERG), perimetry, topographical mapping of the layers of the retina and measurement of the thickness of its layers by means of confocal scanning laser ophthalmoscopy (cSLO) and optical coherence tomography (OCT), topographical mapping of cone density via adaptive optics (AO), functional eye exam, etc., depending upon the species of the subject being treated, their physical status and health and treatment. In certain embodiments, the methods include performing functional measurements that include one or more of low-luminance visual acuity (LLVA), BCVA (best corrected visual acuity), light and dark adapted perimetry, and/or microperimetry, and ERG.

In a clinical setting, electroretinography is a useful, non-invasive procedure for determining spatial differences in retinal activity in which electrical potentials generated by the retina of the eye are measured upon exposing the retina to a light stimulus. In conducting an ERG, an electrode is positioned on the cornea of a patient's eye and a second electrode, usually referred to as an “indifferent” electrode is positioned to complete an electrical connection with the patient's upper anatomy. The indifferent electrode may be placed, for example, in the mouth or may be electrically coupled to the patient's ear or other convenient locus for such connection. The retina is then exposed to a light source and, in response, generates one or more electrical signals which are then studied. An electroretinogram is a record of the resulting electrical signals.

Illumination may be conducted in a number of ways. For example, a first set of ERG readings may be taken in normal room light. In a second step, the lights may be dimmed for a significantly long period of time (on the order of 20 minutes), and readings are taken while the patient's retina is exposed to a light source. That is, after prolonged relaxation in a dark environment, electrical retinal readings are taken at the onset of retinal exposure to light, and for a time period shortly thereafter. As a further step, after a sufficient time for relaxation has passed, a bright flash may be directed to the patient's retina with further ERG readings being taken. Each ERG reading will differ depending upon the light conditions to which the patient's retina is subjected. However, standard responses have been established for each type of test and various useful conclusions can be drawn from excursions from such standardized data. In each test, the retinal response to each illumination is typically in the form of voltage versus time waveforms. Different types of waves have been defined for normal retinal responses. It is expected in a healthy patient, for example, that an electroretinogram shows a-wave and b-wave patterns normal in shape and duration, with appropriate increases in electrical activity as the illumination intensity is increased.

It is understood that the electrodes measure the electrical responses of individual rods and cones which are constituents of the retina located at the back of the eye. The rods and cones comprise visual cells which “convert” or otherwise respond to illumination with electrical activity. This electrical activity is preferably measured with minimum invasion to the patient's anatomy, by placing an electrode on the patient's cornea. As indicated above, the electrode may be mounted on a contact lens for convenient application in an outpatient setting.

Such an electrode typically measures summed activity from the retina. In general, the electrical changes caused by the different major cell types comprising the retina (rod and cone photoreceptors, bipolar cells, horizontal cells, amacrine cells, ganglion cells, Muller cells) tend to overlap, thus the complex and varying waveform. The most prominent wave is the b-wave and the height of this wave can provide an indication of the patient's sensitivity to the illumination source. Tests can be conducted with illumination sources of different spectral content, intensity, kinetics, spatial patterns and spatial contrast, and the results can be studied to determine the patient's medical condition.

Many variations of ERG recording have been developed, which can be separated into two categories. In the first group, the dynamics of the stimulus are altered to exploit temporal response properties of specific functional retinal circuits or cell types. One example is the flicker ERG, in which light flashes are delivered in rapid succession (about 30 Hz), eliciting responses from fast-recovering cone-pathways in the retina (the rod pathways do not have sufficient time to recover between the rapid flashes, and are thus saturated by the time-averaged luminance of the source). A second example is the paired-flash ERG, in which the stimulus flash is followed at a defined time t by a bright probing flash, which drives retinal photoreceptors to saturation and thus titrates the level of response due to the stimulus flash at time t.

The second group includes ERG techniques in which the geometry of the stimulus is varied (from the typical full-field) in order to probe local areas of the retina. The two most common techniques in this category are focal ERG and multi-focal ERG. Focal ERG consists of a focal spot stimulus typically delivered via a hand-held ophthalmoscope with integral stimulus source. The spot is directed by the investigator or clinician to an area of interest, usually the fovea, and the response is recorded with a standard corneal electrode. The spot is illuminated in rapid succession (about 30- about 42 Hz), and the area of interest is surrounded by a ring of bright constant illumination to avoid contributions due to scattered light outside of the area of interest.

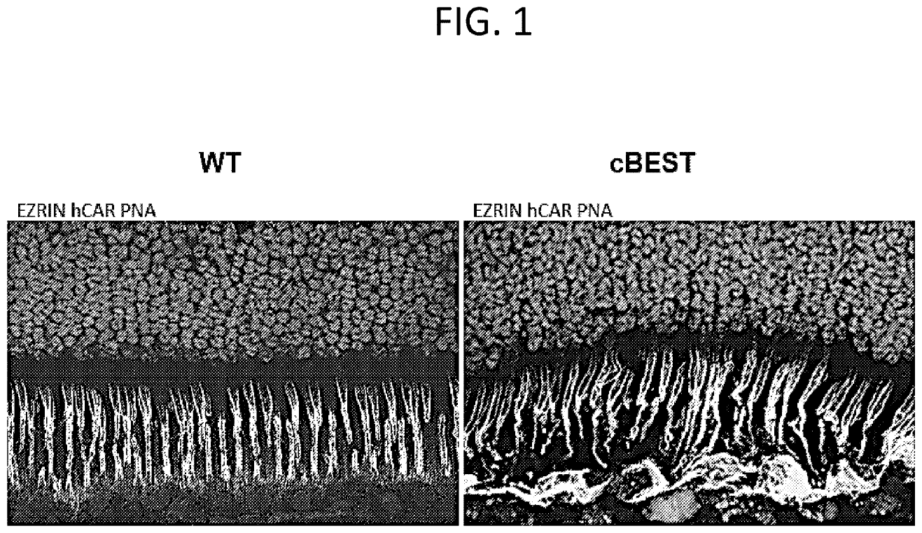

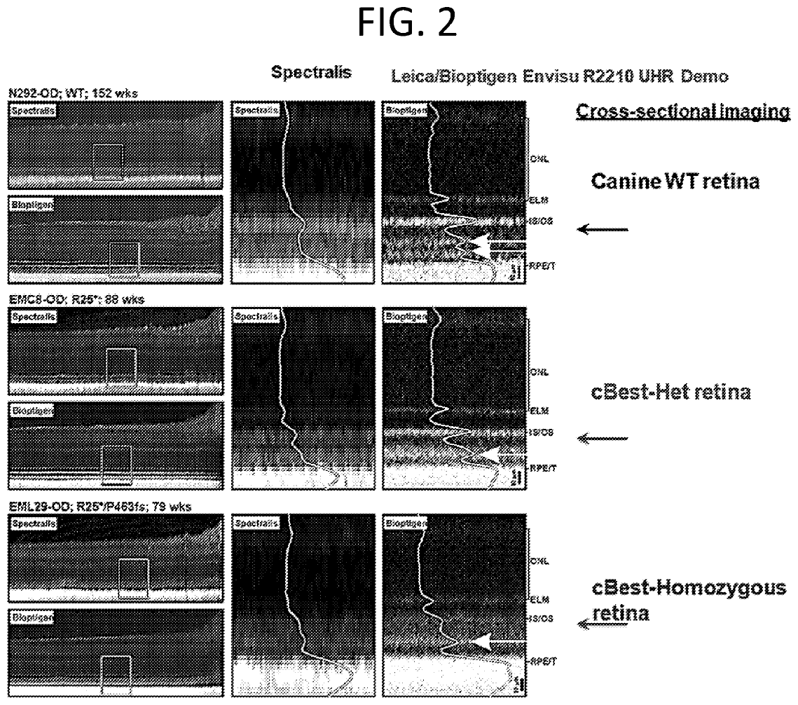

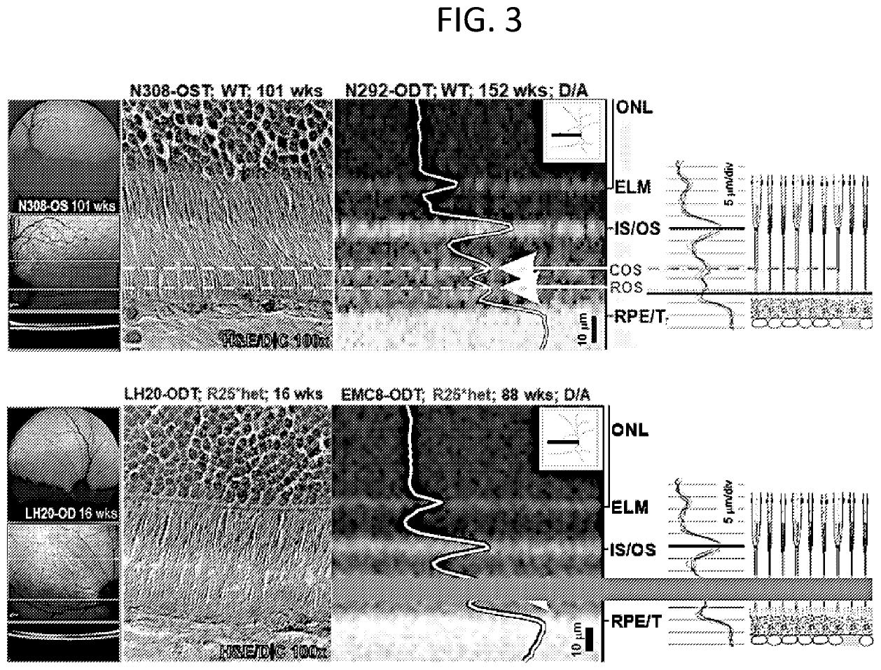

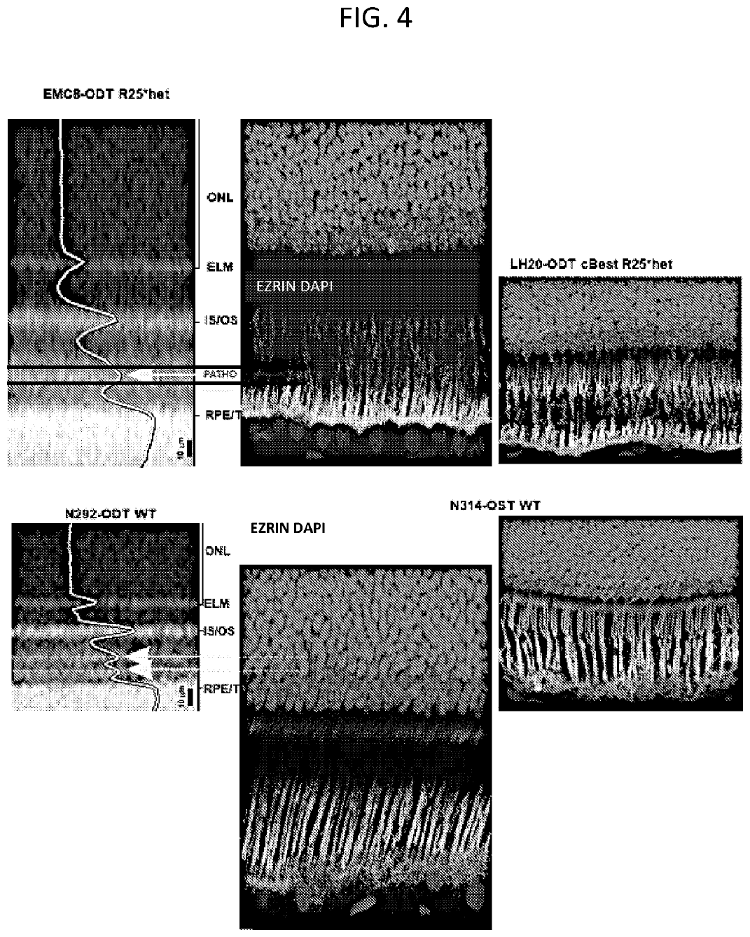

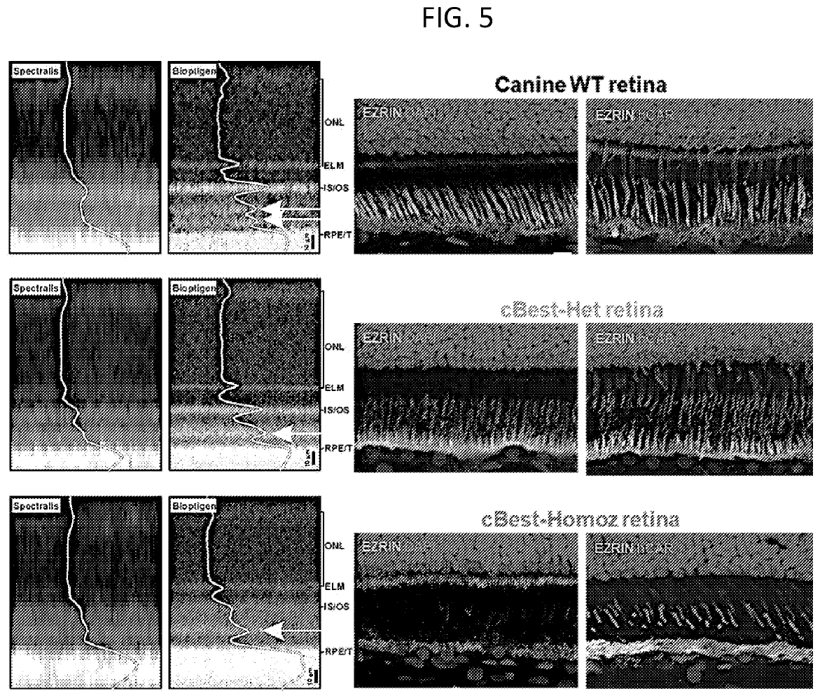

Multi-focal ERG (mfERG) has become more common than focal ERG in recent years. The mfERG stimulus is a field of contiguous hexagons (typically 103, subtending the central 50° of visual field), which are scaled to elicit approximately equal amplitude responses from a normal retina. FIG. 1a of U.S. Pat. No. 7,384,145, incorporated herein by reference, shows a typical stimulus arrangement for a multi-focal ERG. Each hexagon alternates between high and low luminance (e.g. 100 cd m⁻² and 2 cd m⁻², commonly described as white and black) in a predefined, pseudo-random temporal sequence called an m-sequence. Each hexagon follows the same sequence, but each starts at a different point in the m-sequence, and each has a probability of changing from white to black of 0.5 at each transition time. Transitions occur at approximately 75 Hz.

In certain embodiments, the methods provided herein include assessing a treatment using ERG. ERG can be used to assess toxicity of a treatment that includes delivery of an AAV vector encoding a BEST1 protein. However, described herein are unexpected findings based on ERG, including that ERG amplitudes can be measured in treated and untreated eyes to assess the efficacy of a treatment. In particular, data analysis showed that ERG amplitudes in low-dose and high-dose AAV2/2-BEST1 injected eyes were higher than in the uninjected contralateral eyes. As a reduction in ERG amplitudes has been reported in some patients affected with autosomal recessive bestrophinopathy (ARB) (Schatz et al. Invest Ophthalmol Vis Sci. 2010 September; 51(9):4754-65) and autosomal dominant Bestvitelliform macular dystrophy (BVMD) (Burgess et al. Am J Hum Genet. 2008 January; 82(1):19-31), improved ERG function can be a useful measure of efficacy of treatments.

Provided herein are methods for assessing treatment for a bestrophinopathy that include assessing retinal function in a treated eye of a subject by ERG. In certain embodiments, the subject has two mutant BEST1 alleles. In certain embodiments, the subject has at least one mutant BEST1 allele. In certain embodiments, ERG is full-field ERG, focal ERG, and/or multifocal ERG. As described herein, ERG amplitudes were unexpectedly higher, or maintained in treated eyes following AAV-BEST1 treatment relative to untreated, contralateral eyes (or vehicle treated eyes). In certain embodiments, the methods include obtaining ERG amplitude measurements for a treated eye and a contralateral untreated eye or vehicle-treated eye. In further embodiments, the methods include obtaining ERG amplitude measurements for a treated region of an eye (e.g., a subretinal injection bleb) for comparison with a untreated region of the same eye (e.g., outside of a subretinal injection bleb). In certain embodiments, ERG measurements are obtained performed at least 1 week, at least 2 weeks, at least 4 weeks, at least 8 weeks, or at least 10 weeks post-administration of the treatment. In embodiments, ERG measurements are obtained about 11 weeks post-administration of an AAV vector. In certain embodiments, the measurements are obtained at more than one time point post-administration of an AAV vector.

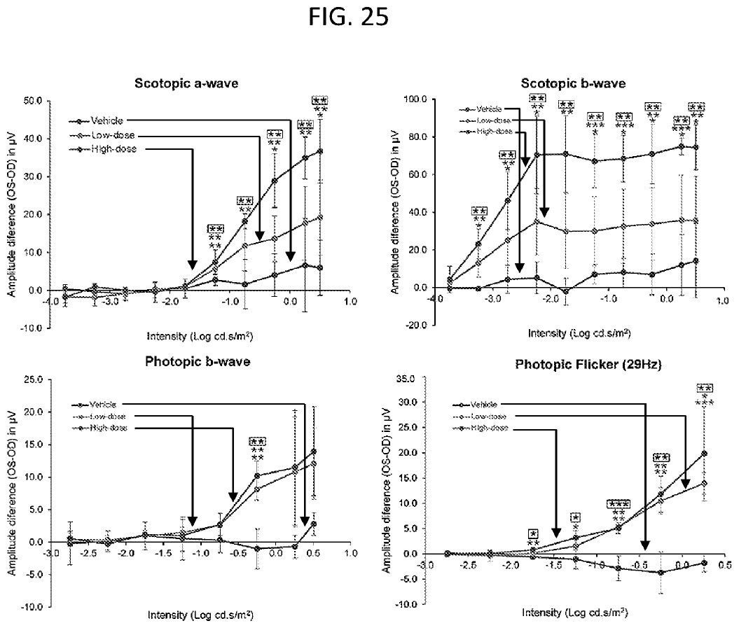

In certain embodiments, the methods include assessing retinal functional by obtaining at least one-type of ERG measurement for a treated eye. In further embodiments, the methods include a combination of ERG measurements obtained using different parameters at the same timepoint. In certain embodiments, ERG readings are obtained using the same parameters as at various time points post-treatment. In certain embodiments, the methods include obtaining ERG amplitude measurements for one or more of a scotopic a-wave response, a scotopic b-wave response, photopic b-wave response, and/or a photopic flicker response. In certain embodiments, the scotopic a-wave response is measured at an intensity that produces a mixed rod-cone response. In certain embodiments, the scotopic a-wave response is measured at an intensity that produces a rod-only or a mixed rod-cone response. In certain embodiments, the photopic b-wave response is measured at an intensity that produces a cone response. In certain embodiments, the photopic flicker response is measured at an intensity that produces a cone response. In certain embodiments, the photopic (1 Hz) b-wave response is a photopic (1 Hz) b-wave response. In certain embodiments, the photopic flicker response is a photopic 29 Hz flicker response. Suitable intensities for measurement for each of a scotopic a-wave response, a scotopic b-wave response, photopic b-wave response, and/or a photopic flicker response are described in Example 11, and depicted, for example, in FIG. 25. In certain embodiments, the amplitude of the scotopic a-wave response is measured at one or more intensities of at least about −2.0 Log cd·s/m². In a further embodiment, the amplitude of the scotopic a-wave response is measured at one or more intensities in a range from about −2.0 Log cd·s/m² to about 1.0 Log cd·s/m². In certain embodiments, the amplitude of the scotopic b-wave response is measured at one or more intensities of at least about −4.0 Log cd·s/m². In a further embodiments, the amplitude of the scotopic b-wave response is measured at one or more intensities in a range from about −4.0 Log cd·s/m² to about 1.0 Log cd·s/m². In certain embodiments, the amplitude of the photopic (1 Hz) b-wave response is measured at one or more intensities of at least about −1.0 Log cd·s/m². In further embodiments, the amplitude of the photopic (1 Hz) b-wave response is measured at one or more intensities in a range from about −1.0 Log cd·s/m2 to about 1.0 Log cd·s/m². In certain embodiments, the amplitude of the photopic 29 Hz flicker response is measured at one or more intensities of at least about −2.0 Log cd·s/m². In further embodiments, the amplitude of the photopic 29 Hz flicker response is measured at one or more intensities in a range from about −2.0 Log cd·s/m² to about 0.5 Log cd·s/m².

In certain embodiments, an amplitude difference for an ERG measurement is determined by comparing an ERG reading in a treated eye and the ERG reading in an untreated eye. In other embodiments, an amplitude an amplitude difference for an ERG measurement is determined by comparing an ERG reading in a treated region of an eye and an ERG reading obtained for an untreated region of the same eye. The amplitude difference can include a comparison of amplitude measurements at a particular intensity. Alternatively, amplitude measurement can be compared at more than one intensity. In certain embodiments, efficacy is indicated by an amplitude difference for an ERG measurement wherein the difference calculated is less than about 0 μV, about 0 μV, at least about 2.0 μV, at least about 5 μV, at least about 10 μV, at least about 15 μV, at least about 20 μV, at least about 25 μV, at least about 30 μV, at least about 40 μV, or at least about 50 μV. In certain embodiments, efficacy is indicated by fold change in the difference for an ERG measurement wherein the fold change is about 0, or a fold increase of at least about 1.1, at least about 1.2, at least about 1.3, at least about 1.4, at least about 1.5, at least about 1.6, at least about a 1.7, at least about a 1.8, at least about 1.9, or at least about 2.0.

In certain embodiments, the methods include generating a longitudinal reflectivity profile (LRP) using an optical coherence tomography (OCT) system. In certain embodiments, imaging of the retina is performed using an ultrahigh-resolution OCT (UHR-OCT) system, such as the Leica/Bioptigen Envisu OCT System or a system capable of similar high-resolution imaging). See, e.g., FIG. 7A demonstrating a LRP generated using an UHR-OCT system. In certain embodiments, ultrahigh resolution OCT is essential to generate a LRP used to evaluate a retinal phenotype. Accordingly, standard imaging systems (e.g., Spectralis HRA+OCT) are not sufficient to reveal retinal phenotypes for purposes of certain methods described herein. In certain embodiments, the LRP is further evaluated to assess parameters that indicate the effectiveness of a treatment. The effectiveness of a treatment can be evaluated, for example, based on examining cytoarchitecture at the RPE-photoreceptors (PRs) interface apposition between RPE and PRs. In certain embodiments, in vivo imaging is used to evaluate the extent of retina-wide RPE-PR macro- or microdetachment to determine the effectiveness of a treatment.

As described herein, and as discussed in the Examples below, the UHR-OCT LRP and generated LRP show the length of cone outer segments (IS/OS to cone outer segment tip (COST) as shown in FIG. 7A, Peak A) and length of rod outer segments (IS/OS to rod outer segment tip (ROST) as shown in FIG. 7A, Peak B) correlate with both in vivo and ex vivo histological analysis. See, e.g., FIG. 7A. Further, the cBest-Hets show elongation of the cone outer segments and rod outer segments. Further, as demonstrated in FIG. 7A and FIG. 7B, cBest model demonstrates abnormal microarchitecture of the RPE-PR interface. These described changes are measurable in both the cBest models, and subject patients. These measurements can be used to help determine efficacy of treatment, as well as identification of subjects requiring medical intervention for Best disease.

In certain embodiments, the COS and/or ROS are evaluated to determine if lengthening is present. In one embodiment, a COS measurement of greater than about 12 μm to about 17 μm is indicative of Best disease. In some embodiments, a COS measurement of greater than about 10, 11, 12, 13, 14, 15, 16, 17, 18, 19, or 20 μm is indicative of Best disease.

In one embodiment, a ROS measurement of greater than about 20 μm to about 27 μm is indicative of Best disease. In some embodiments, a ROS measurement of greater than about 20, 21, 22, 23, 24, 25, 26, 27, 28, 29, or 30 μm is indicative of Best disease.

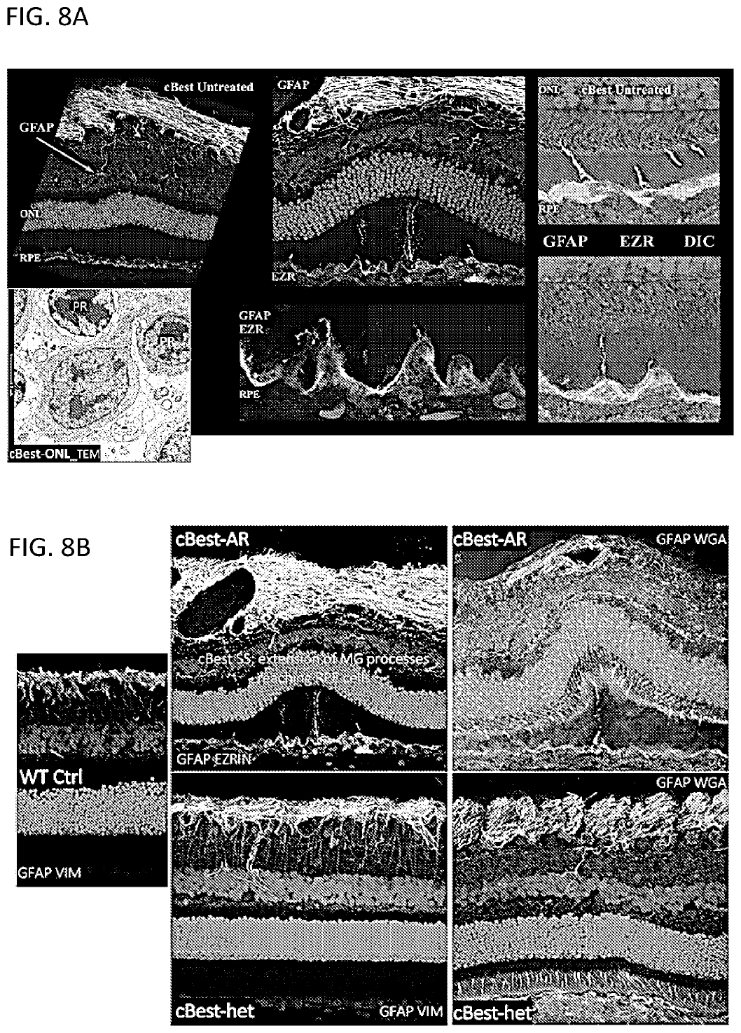

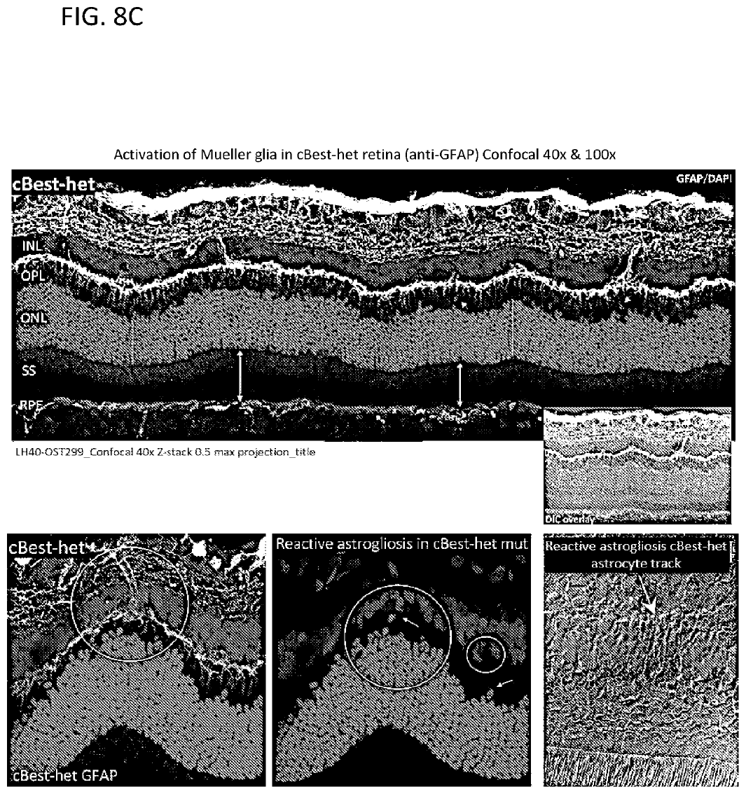

As demonstrated herein, gliotic changes are a hallmark of Best disease, in both the autosomal dominant and autosomal recessive disease. The gliotic changes are a result of constant insult and inflammation to the retina and are observed, inter alia, as Muller glia (MG) trunks or projections penetrating the ONL layer. For example, in FIGS. 8A and 8B it can be seen that the MG processes reach the RPE in the cBest-Het model.

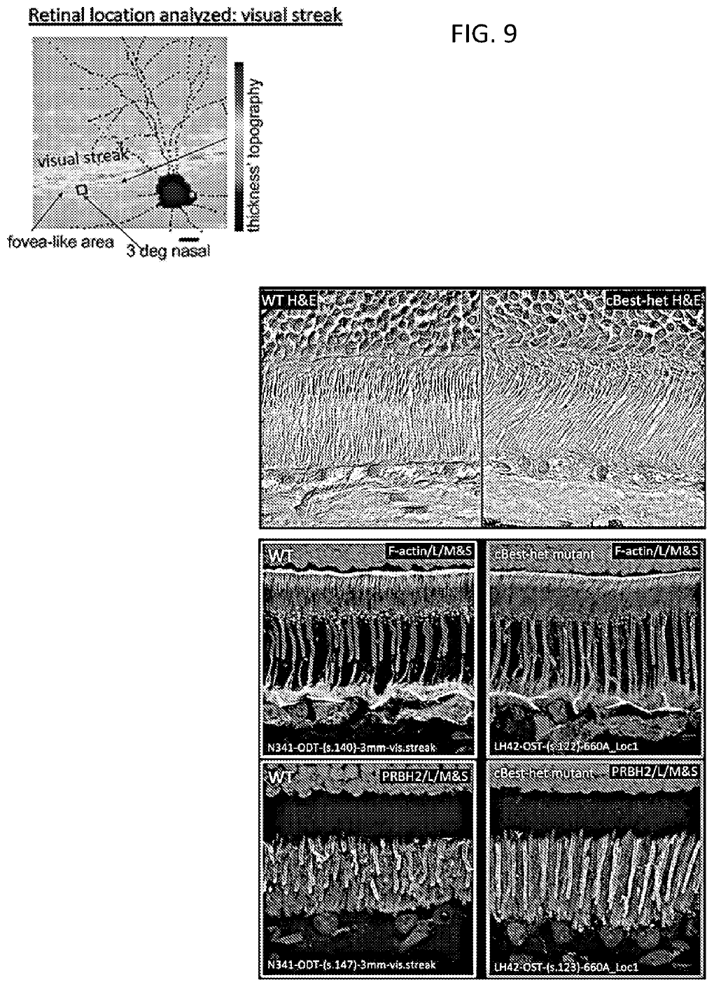

In one embodiment, retinal changes indicative of Best-1 disease include one or more of abnormal POS-RPE apposition and microarchitecture of RPE-PR interface (FIG. 7B); Elongation of both ROS & COS associated with increased ELM-RPE distance (FIG. 7B-FIG. 7D, FIG. 9); Accumulation of subretinal debris at RPE apical surface (FIG. 9), within subretinal space (FIG. 7B-FIG. 7D); Compromised IPM and defective ELM; Fluctuation of ONL thickness associated with reactive gliosis and cell migration; Schistic changes inner/outer retina; Formation of subretinal & intraretinal scars; RPE monolayer hypertrophy, occasional severe deformation of individual RPE cells associated with ONL & INL thickness fluctuations; MG trunks/projections penetrating ONL layer with astrogliosis as an indicator of chronic retinal stress (FIG. 8B).

In certain embodiments, provided herein are methods for detecting an autosomal dominant BEST1 mutation or diagnosing a subject as having autosomal dominant bestrophinopathy. In certain embodiments, the method includes performing retinal imaging using ultrahigh-resolution OCT to generate a longitudinal reflectivity profile (LRP), wherein an abnormal RPE-PR interdigitation zone results in an altered LRP profile indicating that the subject harbors an autosomal dominant BEST1 mutation.

In certain embodiments, the methods provided herein include obtaining a sample from a treated subject for examination ex vivo. Accordingly, an ocular tissue sample is examined by labeling with reagents that bind ocular cells and/or markers in the sample to evaluate a phenotype. The sample may be analyzed, for example, using fluorescence microscopy or immunohistochemistry. In certain embodiments, retinal lesions in a sample are evaluated for accumulation of autofluorescent material in RPE cells or the subretinal space. In yet other embodiments, the sample is evaluated to determine cytoskeletal rescue and restoration of restoration of RPE apical microvilli structure, a reestablishment of proper apposition between RPE cells and photoreceptor (PR) outer segments (cytoarchitecture of RPE-PR interface), and/or a restoration of the insoluble cone-specific interphotoreceptor matrix (IPM) to determine treatment efficacy (as described, for example, in Guziewicz et al., PLoS One. 2013 Oct. 15; 8(10):e75666 and Guziewicz et al, PNAS. 2018 Mar. 20; 115(12):E2839-E2848, each of which is incorporated by reference herein). In certain embodiments the sample is labeled with reagents that bind one or more of BEST1, RPE65, EZRIN, pEZRIN, MCT1, CRALBP, F-actin, hCAR, an L-opsin, an M-opsin, an S-opsin, and RHO.

The following examples are provided for the purpose of illustration only and the invention should in no way be construed as being limited to these examples but rather should be construed to encompass any and all variations that become evident as a result of the teaching provided herein.

Described herein is a sub-clinical phenotype in a canine cBest disease model associated with abnormal microarchitecture of RPE-PR interface and expose retinal pathways leading to chronic retinal stress, reactive Muller cells' gliosis and astrocytosis, both contributing to neuronal dysfunction in mono allelic BEST1 disease. Our findings support that these sub-clinical abnormalities are amenable to AAV-mediated BEST1 gene augmentation therapy, expanding the therapeutic landscape for Best patients.

The cBest-Het mutant model demonstrates various disease features which are observable by the skilled artisan including: Abnormal POS-RPE apposition and microarchitecture of RPE-PR interface; Elongation of both ROS & COS associated with increased ELM-RPE distance; Accumulation of subretinal debris at RPE apical surface, within subretinal space; Compromised IPM and defective ELM similar to UHR findings in human Best disease; Fluctuation of ONL thickness associated with reactive gliosis and cell migration; Schistic changes inner/outer retina; Formation of subretinal & intraretinal scars; RPE monolayer hypertrophy, occasional severe deformation of individual RPE cells associated with ONL & INL thickness fluctuations; MG trunks/projections penetrating ONL layer with astrogliosis as an indicator of chronic retinal stress.

### EXAMPLES

### Example 1: Methods

cBest Dogs

All cBest-mutant and control dogs are bred and maintained at the Retinal Disease Studies Facility (RDSF), Kennett Square, PA, USA. The studies are carried out in strict accordance with the recommendations in the Guide for the Care and Use of Laboratory Animals of the of the National Institutes of Health (NIH), and in compliance with the Association for Research in Vision & Ophthalmology (ARVO) Statement for the Use of Animals in Ophthalmic and Vision Research. The protocols were approved by the Institutional Animal Care and Use Committee of the University of Pennsylvania (IACUC #s 804956, 803422). All efforts are made to improve animal welfare and minimize discomfort.

### Genotyping

The genotypes of cBest dogs are determined using previously developed PCR-based assays with canine BEST1 (cBEST1) (GB #NM_001097545.1) gene specific primers (Guziewicz et al., 2007; Zangerl et al., 2010). To confirm cBEST1 heterozygous mutations (c.73C>T or c.482G>A or c.1388delC), PCR amplicons are purified (ExoSAP-IT, ThermoFisher Scientific, Waltham, MA, USA), submitted for direct Sanger sequencing (UPenn NAPCore Facility, The Children's Hospital of Philadelphia, PA, USA), and analyzed with the use of Sequencher v.5.2.4 software package (Gene Codes, Ann Arbor, MI, USA).

### Ophthalmic Examination and In Vivo Retinal Imaging

Ophthalmic examinations, including biomicroscopy, indirect ophthalmoscopy and fundus photography, are conducted on a regular basis, starting at 5 weeks of age, then biweekly before cSLO/OCT baseline evaluation, and every 4 weeks thereafter.

Non-invasive retinal imaging in cBest-mutant and control dogs is performed under general anesthesia after pupillary dilation and conducted according to methods similar to previously described (Cideciyan et al., 2005; Beltran et al., 2012; Guziewicz et al., 2018). Overlapping en face images of reflectivity with near-infrared illumination (820 nm) are obtained with 300 and 550 diameter lenses (Spectralis HRA+OCT, Heidelberg, Germany) to delineate fundus features such as optic nerve, retinal blood vessels, retinotomy post subretinal injection or other local changes. Custom programs (MatLab 7.5; The MathWorks, Natick, MA, USA) are used to digitally stitch individual photos into a retina-wide panorama. Imaging with an ultrahigh-resolution OCT system (Leica/Bioptigen).

Retinal cross-sectional images of cBest and control eyes were acquired with an Envisu R2210 UHR (Ultra-High Resolution) SD-OCT system (Bioptigen, Leica Microsystems, Morrisville, NC, USA) with methods similar to previously described (Aleman et al., 2011; Huang et al., 2012; Boye et al., 2014). ‘Rabbit’ lens was used, and the angular magnification was adjusted by matching features visible on the same canine eye scanned with Spectralis as well as Bioptigen/Envisu systems. The retinal location of interest centered at the canine fovea-like region was found under fast fundus mode. High-resolution scans (100 parallel raster scans of 1000 LRP each repeated three times) were acquired at this location. Each LRP had 1024 samples representing 1654 μm of retinal depth along the z-axis (1.615 μm/sample). Post-acquisition processing of OCT data was performed with custom programs (MatLab 7.5; The MathWorks, Natick, MA, USA). The LRPs of the OCT images were aligned by manually straightening the Bruch's membrane (BrM) and choriocapillaris (ChC) reflection. Thickness of the outer nuclear layer (ONL) was measured between the signal peaks defining the OPL and outer limiting membrane (OLM). Number of hyper-scattering peaks were identified between the IS/OS peak and the RPE/Tapetum (RPE/T) peak, and distance between the peaks was quantified.

### Ex Vivo Assessments

The retinal microarchitecture of cBest-Het eyes is studied in comparison to the wild-type controls with assessments methods similar to previously described (Beltran et al., 2006; Guziewicz et al., 2017; Guziewicz et al., 2018).

### Histological and Immunohistochemical Evaluations

Ocular tissues for ex vivo analyses are collected as described previously (Beltran et al., 2006; Beltran et al., 2014). The eyes are fixed in 4% paraformaldehyde or frozen, embedded in Optimal Cutting Temperature (OCT) media and processed as previously reported (Beltran et al., 2006; Guziewicz et al., 2017). Histological assessments using hematoxylin/eosin (H&E) staining, and immunohistochemical (IHC) experiments are performed on 10 μm-thick cryosections following established protocols (Beltran et al., 2006; Guziewicz et al., 2013; Guziewicz et al., 2017). Briefly, retinal cryosections are permeabilized with 1×PBS/0.25% TX-100, blocked for 1 hour at room temperature, and incubated overnight with a primary antibody. A set of RPE- and photoreceptor-specific markers (including BEST1, RPE65, EZRIN, pEZRIN, MCT1, CRALBP, F-actin, hCAR, L/M&S opsins, and RHO) is used to assay the RPE-photoreceptor interdigitation zone in cBest-Het and control retinas. For simultaneous assessment of the insoluble interphotoreceptor matrix (IPM), multicolor labeling is applied and primary antibodies combined with WGA-AF594 or PNA-AF647 (L32460; Molecular Probes, Eugene, OR, USA), followed by 1 hour incubation with a corresponding secondary antibody (Alexa Fluor®, ThermoFisher Scientific). The slides are examined by epifluorescence or transmitted light microscopy (Axioplan; Carl Zeiss Meditec GmbH Oberkochen, Germany), and digital images collected with a Spot4.0 camera (Diagnostic Instruments, Sterling Heights, MI, USA).

### Confocal Microscopy & Image Analysis

Microscopic images are acquired on a Leica TCS-SP5 Confocal Microscope System or Leica DM6000B Upright Microscope with DIC (Differential Interference Contrast) optics and DMC-2900 color camera (Leica Microsystems, Mannheim, Germany). To obtain high-resolution confocal photomicrographs, image stacks are acquired at 0.25 μm Z-steps with digital resolution of 2048×2048, then deconvolved with Huygens Deconvolution Software v.17.04 (Scientific Volume Imaging Inc., Hilversum, Netherlands). All deconvolved images are rendered in the Leica LAS X 3D-rendering module, and cone-associated RPE apical microvilli assessed from the maximum projection images. Data are analyzed and quantified using Prism software v.7 (Prism; GraphPad, San Diego, CA, USA).

### Example 2: Assessment of Retinal Phenotype in cBest Heterozygous Dogs

The goal of this study was to determine whether cBest heterozygous mutant dogs (cBest-Het) present a milder disease phenotype, which would support the use of the cBest-Het model for preclinical assessment of AAV2-BEST1 gene augmentation therapy for the autosomal dominant form of the disease. Accordingly, retinal imaging with an ultrahigh-resolution OCT system (Leica/Bioptigen) was performed to determine the presence of structural abnormalities at the RPE/PR interface below the resolution of the standard clinical systems (Spectralis HRA+OCT). cBest dogs (n=9; both sexes) harboring cBEST1—cmr1: c.73C>T/p.R25* or cmr2: -c.482G>A/p.G161D or cmr3: -c.1388delC/p.P463fs mutations in heterozygous state were evaluated.

The cBest heterozygous mutant dogs were bred at the UPenn RDSF, and housed under bright light (450 lux) cyclic conditions. Retinal phenotype was monitored at baseline (12-wks of age) and followed on a 6-wk basis by ophthalmoscopy and cSLO/SD-OCT using established protocols of incremental light exposure. Imaging with an ultrahigh-resolution OCT system (Bioptigen) was performed to determine the existence of structural abnormalities below the resolution of the standard clinical systems (Spectralis). Retinal pathology was assessed at 24-wks of age (Grp1 n=3) or at 36-wks of age (Grp2 n=3). Based on the intermediate phenotype in cBest-Het dogs identified at 24- or 36-wks of age, the remaining cBest-Het group (Grp3 n=3) was kept to test correction by gene therapy (Example 3). Ex vivo findings in the cBest-Het model indicate partial underdevelopment of the cytoskeleton associated with RPE apical aspect and RPE/PR interface, and suggest haploinsufficiency as the underlying cause of cBest-Het subclinical manifestation.

Serial in vivo imaging using targeted light exposure was used to determine the association between the milder cBest-Het phenotype and its sensitivity to light (light-potentiated formation of subretinal microdetachment quantified based on IS/OS-RPE/T distance measurements). Histology/IHC inform identified retinal morphological and molecular defects at the RPE-PR interdigitation zone in cBest-Het retinas. Expression of Ca-dependent molecules involved in Best1 pathway, accumulation of lipofuscin, and cytoarchitecture of RPE apical aspect (cone-MV quantification) were also examined. The characterization of cBest-Het mutant phenotype has yielded insight into the BEST1 haploinsufficiency mechanisms, and consequently, set the stage for gene augmentation therapy in patients affected with autosomal dominant bestrophinopathy.

Briefly, cmr1 mutation results in a premature stop codon in the first coding exon of cBEST1 gene, and no gene product (bestrophin-1 protein) was detected; cmr2 change is a point mutation (aka missense) in exon 5 resulting in amino acid substitution (Glycine residue ‘G’ to a polar, negatively charged Aspartic Acid ‘D’), leading to protein misfolding/ER retention/mistrafficking; cmr3 microdeletion (C1388del) initiates Pro463fs frameshift that results in a stop codon at amino acid 490 and protein truncation. All three cBEST1 mutations are naturally-occurring and lead to a highly consistent in vivo phenotype.

The cBest-Het mutant model demonstrates various disease features which are observable by the skilled artisan including: Abnormal POS-RPE apposition and microarchitecture of RPE-PR interface (FIG. 7B); Elongation of both ROS & COS associated with increased ELM-RPE distance (FIG. 7B-FIG. 7D, FIG. 9); Accumulation of subretinal debris at RPE apical surface (FIG. 9), within subretinal space (FIG. 7B-FIG. 7D); Compromised IPM and defective ELM supporting UHR findings in human Best disease; Fluctuation of ONL thickness associated with reactive gliosis and cell migration; Schistic changes inner/outer retina; Formation of subretinal & intraretinal scars; RPE monolayer hypertrophy, occasional severe deformation of individual RPE cells associated with ONL & INL thickness fluctuations; MG trunks/projections penetrating ONL layer with astrogliosis as an indicator of chronic retinal stress (FIG. 8B).

### Example 3: AAV-Mediated BEST1 Gene Augmentation

cBest-Het dogs (n=6) with established disease phenotype (Grp3, as described in Example 2) are injected unilaterally (n=6 eyes; age: 36-wks) with research-grade AAV-hBEST1 therapeutic vector (3.0E+11 vg/mL) targeting retinal areas previously exposed to the incremental light intensities. The contralateral eyes and retinal regions outside of the treatment bleb serve as controls. Treatment responses are monitored in vivo (fundus eye examination, cSLO, Bioptigen OCT) at 6-, 12-, and 24-wks post injection (p.i.), and assessed ex vivo 24-wks p.i. The reversal of the intermediate cBest-Het mutant phenotype provides baseline for determination of efficacy of correction relevant to a major proportion of patients affected with autosomal dominant form of bestrophinopathy.

### Example 4: Preclinical Assessment of an AAV-BEST1 Vector

The purpose of this study is to assess outcome measures, such as retinal preservation, vector tropism, and transgene expression resulting from administration of AAV-BEST1 vector in wildtype dogs for overexpression of BEST1 protein.

Pre-dosage: physical and eye examinations (n=12 dogs); 4 dose groups; 3 dogs/dose group. Subretinal injection (MedOne kit 25G/38G cannula) (150 uL) in one eye of 12 wild-type (WT) dogs with one of 3 vector doses (High-Dose: 3×10¹² vg/mL, Mid-Dose: 3×10¹¹ vg/mL, or Low-Dose: 3×10¹⁰ vg/mL), or vehicle. Termination at 10-wks post-dosage.

In vivo outcome measures of safety:
Physical examination at pre-dosage, wk1, then at termination (wk10).
Ocular examinations at pre-dosage, day1 and day2 post injection (p.i.), then weekly until termination at wk10.
Serum collection for AAV2 neutralizing antibody titration at pre-dosage, wk1 and wk6 p.i., then at termination (wk10).
cSLO/SD-OCT examination 10-wks post injection (end-evaluation wk10) and qualitative analysis.

Ex vivo outcome measures: assessment of retinal preservation, vector tropism, and transgene expression: Retinal histology (H&E)/IHC (BEST1 transgene expression, phosphorylated Ezrin (pEzrin) qualitative analysis) in treated vs non-treated areas of injected eyes at 10-wks p.i.

### Example 5: GLP-Like Dose Range Finding/Non-Clinical Toxicology Study

Purpose: To determine under GLP-like conditions the range of efficacious doses of research-grade AAV2-hVMD2-hBEST1 vector and evaluate its safety profile.
Subjects: cBest homozygous dogs.
Study Duration: In life: 12 wks (injection at ˜12-wks of age, termination at ˜24-wks of age).
Methods: 4 dose groups. Subretinal injection (150 uL) in one eye of cBest homozygous mutant dogs at ˜12-wks of age with one of 3 vector doses (High-Dose: 3×10¹² vg/mL, Mid-Dose: 3×10¹¹ vg/mL, or Low-Dose: 3×10¹⁰ vg/mL), or vehicle. Termination at 12 weeks post-dosage.

### Outcome Measures of Efficacy:

Assessment of retinal structure by cSLO-OCT at pre-dosage and before termination (˜12 weeks post dosage).
Retinal histology (H&E) and IHC for BEST1 transgene expression and cone MV structure in treated vs nontreated areas of ipsilateral and contralateral eyes.

### Outcome Measures of Safety:

Physical examination (incl. body weights) at pre-dosage, wk1, then weekly until termination (wk12).
Ocular examinations at pre-dosage, wk1, then monthly until termination at wk12.
Clinical pathology (CBC, Chemistry panel, Coagulation profile) at pre-dosage, then monthly until termination at wk12.
Whole blood collection (for biodistribution studies to be coordinated by Sponsor) at pre-dosage, wk1, then monthly until termination at wk12.
Serum collection (for AAV2 Nab testing to be coordinated by Sponsor) at pre-dosage, wk1, then monthly until termination at wk12.
Full necropsy, histopathology analysis, tissue collection for biodistribution studies.

Eye examinations: at pre-dose phase, and day 3-, weeks: 1-, 2-, 4-, 8-, and 12-post-injection. cSLO/SD-OCT examination: at pre-dose and 12wks p.i. Retinal histology (H&E)/IHC (BEST1 transgene expression; cone-MV structure) in treated vs non-treated areas. Outcomes: This study determines the range of effective and safe doses that can guide the design of a first-in-human clinical trial.

### Example 6: Photoreceptor Function and Structure in Autosomal Dominant Vitelliform Macular Dystrophy Caused by BEST1 Mutations

One of the more common inherited retinal diseases is autosomal dominant Best vitelliform macular dystrophy caused by BEST1 mutations. Patients have pathognomonic macular lesions surrounded by wide expanses of retina that appear functionally and structurally normal except for an electrophysiological defect. We evaluated rod and cone function within macular lesions, and retinal structure and function in extralesion areas. Within central lesions with large serous retinal detachments, rod function was severely reduced but cone function was relatively retained. In extralesion areas, there was a mild but detectable widening of the distance between IS/OS and the RPE in some patients and some retinal areas. Over long term followup, some eyes showed formation of de novo satellite lesions at retinal locations that previously demonstrated subretinal widening, and also there was evidence for slow kinetics of dark adaptation. Many patients demonstrated retinawide but mild thickening of the outer nuclear layer, which further thickened over long term followup. For future clinical trials, outcome measures should include evaluations of rod function, and quantitative measures of outer retinal structure.

Best vitelliform macular dystrophy (BVMD) is an autosomal dominant macular disease with a distinct ophthalmoscopic appearance caused by to monoallelic mutations in BEST1. Over the last decade it has become clear that BVMD is the most prevalent member of a spectrum of dominant and recessive inherited retinal pigment epithelium (RPE) diseases collectively named bestrophinopathies caused by both monoallelic as well as biallelic mutations in BEST1. Patients with BVMD tend to show pathognomonic macular lesions centered on or near the fovea, and disease is thought to progress through stages descriptively named based on their ophthalmoscopic appearances such as vitelliform, pseudohypopyon, or vitelliruptive. Common to the earlier stages of disease is a serous detachment of the retina from the RPE which is consistent with the understanding that the molecular defect acts primarily at the interface of photoreceptor outer segments and the RPE apical processes despite Bestrophin-1 being localized to the basolateral plasma membrane of the RPE. Later stages of BVMD can demonstrate substantial loss of photoreceptors and RPE that is localized to the macular area surrounded by wide expanses of retina that appear to demonstrate normal structure and normal function except for an electrophysiological defect detected by electrooculography (EOG).

BVMD is currently not curable or treatable. Gene augmentation therapy performed in dogs with naturally occurring biallelic BEST1 mutations has shown promise in treating both vitelliform lesions as well as peripheral subclinical abnormalities. Although gene therapy has not yet been tried in dogs heterozygous for BEST1 mutations, two independent investigations in patient cells have supported the hypothesis that at least a subset of monoallelic BVMD mutations may indeed be amenable to gene augmentation therapy. In addition, small molecules have been proposed as BVMD therapies. We evaluated rod- and cone-photoreceptor mediated function within macular lesions and investigated subcellular retinal structure of the clinically-uninvolved perilesion and peripheral retina.

### Methods

### Subjects

The study population consisted of 17 BVMD patients (ages 6-59 at first visit) from 7 families. Patients were heterozygous for BEST1 gene mutations. Subset of patients were evaluated 4 to 22 years after their initial visit; earlier results in a subset have been published. Procedures followed the Declaration of Helsinki, and the study was approved by the Institutional Review Board (IRB) of the University of Pennsylvania. Informed consent, assent, and parental permission were obtained, and the work was HIPAA-compliant.

| | | | First Visit | | | Last Visit | | |
|---|---|---|---|---|---|---|---|---|
| Patient # | Sex | Mutation | Age | VA/RE | VA/LE | Age | VA/RE | VA/LE |
| F1/P1 | F | T307I | 6 | 20/40 | 20/25 | | | |
| F1/P2 | F | T307I | 14 | 20/20 | 20/20 | | | |
| F1/P3 | M | T307I | 23 | 20/160 | 20/20 | 41 | 20/200 | 20/40 |
| F1/P4 | M | T307I | 24 | 20/20 | 20/200 | 43 | 20/50 | 20/100 |
| F1/P5 | M | T307I | 48 | 20/100 | 20/40 | 58 | 20/100 | 20/32 |
| F2/P1 | M | Q96R | 6 | 20/60 | 20/20 | | | |
| F2/P2 | F | Q96R | 34 | 20/20 | 20/20 | | | |
| F3/P1 | F | R218C | 29 | 20/16 | 20/40 | | | |
| F3/P2 | F | R218C | 59 | 20/50 | 20/40 | | | |
| F4/P1 | M | R218C | 20 | 20/20 | 20/32 | 42 | 20/50 | 20/20 |
| F4/P2 | M | R218C | 61 | 20/30 | 20/80 | | | |
| F5/P1 | M | R218C | 31 | 20/20 | 20/20 | | | |
| FS/P2 | M | R218C | 32 | 20/20 | 20/30 | | | |
| FS/P3 | M | R218C | 35 | 20/20 | 20/20 | | | |
| FS/P4 | M | R218C | 35 | 20/30 | 20/100 | 55 | 20/25 | 20/125 |
| F6/P1 | F | A243V | 42 | 20/25 | 20/32 | 56 | 20/50 | 20/80 |
| F7/P1 | M | R92H | 54 | 20/80 | 20/25 | 58 | 20/100 | 20/50 |

### Measures of Rod and Cone Function

Two-color dark-adapted perimetry was used to measure rod-mediated function across the visual field. Mediation of the 500 nm (blue-green) stimulus (1.7° diameter; 200 ms duration) sensitivity by rod photoreceptors was determined by comparison of sensitivities with a 650 nm (red) stimulus and taking advantage of the spectral sensitivity differences between rods and cones. Light-adapted perimetry with a 600 nm (orange) stimulus was used to measure cone-mediated function.

### Retinal Imaging

A confocal scanning laser ophthalmoscope (HRA2 or Spectralis HRA, Heidelberg Engineering, Heidelberg, Germany) was used to obtain near-infrared excited reduced-illuminance autofluorescence imaging (NIR-RAFI). Wide field image montage was assembled by manually specifying corresponding retinal landmark pairs in overlapping segments using custom-written software (MATLAB 6.5).

Optical coherence tomography (OCT) was performed mainly with a spectral-domain (SD) OCT system (RTVue-100, Optovue Inc., Fremont, CA). Earlier visits of some patients were obtained with time domain OCT (OCT1 and OCT3; Carl Zeiss Meditec, Dublin, CA).

### Results

### Retained Function Despite Chronic Retinal Detachment

Data from both eyes of 17 BVMD patients (7 families) were available. Ages at first visit ranged from 6 to 61 yr, and a subset of 7 patients were followed long term (15.5±6.4 yr). The majority (20 of 34, 59%) of eyes at the first visit had best corrected visual acuity (BCVA) of 0.2 log MAR (20/30 Snellen equivalent) or better, consistent with previous clinical impressions of relatively retained vision despite substantial foveal lesions. Over the long term, BCVA was lost slowly at the average rate of 0.02 log MAR/yr (corresponding to loss of 1 ETDRS letter per year) across the 14 eyes with available longitudinal data. Relatively retained acuity that showed slow progression would be counterintuitive considering the severe degenerative consequences of experimental retinal detachment on photoreceptors. Therefore, we investigated details of the visual function in eyes that showed subretinal fluid, but retained foveal fixation thus allowing use of perimetry.

A representative example of a BVMD eye with a large vitelliform lesion but normal visual acuity of 0 log MAR and foveal fixation is F1/P2 at age 14. En face imaging shows a pseudohypopyon stage with autofluorescent material accumulated at the inferior aspect of the lesion. Cross-sectional imaging along the horizontal meridian shows a large serous detachment extending from −11 deg temporal to ˜5 deg nasal to the fovea. There were 6 eyes with similar serous detachments and foveal fixation. To better understand the consequence of the chronic retinal detachment on foveal function, we measured light-adapted sensitivities at the fovea. Cone sensitivity loss (CSL) at the fovea ranged from 4-5 dB in the eyes with 0.2 log MAR or better acuity, to 10-16 dB in the eyes with worse than 0.2 log MAR acuity.

Considering there are at least four sources of the 11-cis-retinal chromophore required for rod and cone vision, we next asked whether rod and cone photoreceptor function are differentially affected in extrafoveal regions of eyes with chronic retinal detachment. In these regions, dark-adapted rod sensitivity losses (RSL) were substantially greater than CSL at the great majority of retinal locations. Greater rod than cone dysfunction associated with detached locations in BVMD was similar to findings in recessive bestrophinopathies and could be explained by the dominant source of visual chromophore originating from the retina for cones as opposed to the visual chromophore originating from the RPE for rods.

### Microstructural Defects in Clinically-Uninvolved Retina

To evaluate the retinal microstructure in the clinically-uninvolved retinal areas (outside of macular lesions and rare satellite lesions) of BVMD patients, two key subcellular photoreceptor features were initially considered: a) the outer nuclear layer (ONL) thickness where rod and cone photoreceptor nuclei reside, and b) the distance between the junction of inner and outer segments (IS/OS) and the RPE which would encompass the length of OS plus the subretinal space. Both features were mapped across an ultra-wide (˜80 deg) expanse of retina in each eye. As demonstrated in a representative normal eye, ONL thickness was largest at the fovea with a gradual fall-off with eccentricity. ONL topography in the BVMD patient F3/P2 was similar to normal but some retinal regions tended to show mild thickening. The normal IS/OS to RPE distance was also largest at the fovea with fast fall-off to parafovea and a nearly homogeneous distribution throughout the rest of the retina. In the representative BVMD patient F3/P2, IS/OS to RPE distance was larger than normal throughout the retina.

ONL thickness quantified across all BVMD eyes is shown along the horizontal and vertical meridians crossing the fovea. When measurable, ONL overlying macroscopically obvious lesions tended to be thinner than normal suggesting photoreceptor degeneration. In extralesion regions, on the other hand, ONL was either normal or mildly thickened. IS/OS to RPE distance is also shown along the two meridians. Majority of the BVMD eyes across the clinically-uninvolved retinal areas showed expansion of the distance from IS/OS to the RPE suggesting either elongation of OS or widening of subretinal space or both.

### Outer Segment Length and Subretinal Space

To differentiate between elongation of cone OS, rod OS, or expansion of the subretinal space with interdigitating OS and microvilli of the RPE, we measured corresponding markers from OCT scans at two locations at 16 deg eccentricity in the superior and inferior retina. Designation of the backscatter peaks on longitudinal reflectivity profiles (LRPs) corresponding to subcellular photoreceptor and RPE components are shown for a representative normal, and two BVMD patients F4/P1 and F3/P2. In most BVMD eyes, there was normal or near normal cone OS length in the superior and inferior retina. Rod OS length could be normal or elongated, and the distance to the RPE could be normal or extended both in the superior and inferior retina.

### Functional Consequences of an Expanded Subretinal Space

Rod sensitivities were normal across clinically-uninvolved regions of BVMD retinas; this implied that minor changes in the subretinal space do not result in detectable changes to the primary function of rods which is to signal dim lights under fully dark-adapted conditions. However, since the bulk of the chromophore for rod cells is thought to originate from RPE cells, we hypothesized that abnormalities at the interdigitation of the outer segments and RPE microvilli may slow down the kinetics of the resupply of chromophore and thus the rate of dark adaptation. In a subset of four BVMD patients at a macular region 10 deg nasal to the fovea we recorded dark-adaptation kinetics of the rod system. All four patients had near-normal ONL thickness and dark-adapted sensitivities ruling out retinal degeneration. Three of the four (F4/P1, F1/P4, and F7/P1), had normal or near normal rod and cone OS thicknesses and normal subretinal space and they demonstrated normal kinetics of dark-adaptation recovery. One patient (F3/P2) had elongated cone OS, rod OS and wider subretinal space and demonstrated a substantial slowing of the dark-adaptation recovery rate. To a first approximation, these data appear to support a relation between rod dark-adaptation recovery rate and the abnormality of the subretinal space. However, it is important to note the results from a fifth patient (F3/P1) demonstrating substantial thinning of the ONL but retaining apparently normal distances of IS/OS to COS, ROS and RPE. Dark-adapted sensitivities of F3/P1 were elevated by more than a log unit, and there was substantial slowing of the dark-adaptation rate. This retinal-degeneration-associated abnormality in dark-adaptation rate was similar to an effect previously described in patients with ABCA4-associated Stargardt disease and BEST1-associated autosomal recessive bestrophinopathy. Thus, it is important to note that dark-adaptation kinetic abnormalities could have multiple causes but the combination of normal ONL thickness, normal dark-adapted sensitivities and slowed kinetics measured in P3/P2 likely places the physiological defect at the abnormal RPE-PR interface.

### Long-Term Natural History of Retinal Structure

Among the outer retinal abnormalities in clinically-uninvolved retina was minor ONL thickening observed in some retinal locations of some BVMD patients. To better understand the temporal development of this finding, we took advantage of the long-term natural history of retinal structure in a subset of six BVMD patients who were followed with OCT over nearly two decades. Qualitatively, in 5 of 6 patients there were relatively small changes; central lesions showed minor changes, and clinically-uninvolved extra-lesion areas appeared to be unchanged. An important exception was F1/P3 who showed formation of an extra-macular satellite lesion temporal to the fovea. Next, we quantified the ONL thickness across the horizontal meridian crossing the fovea. Overlying the central lesions, ONL thickness was reduced implying a progressive photoreceptor degeneration. ONL thinning also occurred over the satellite lesion in F1/P3. Changes in extra-lesion regions were mostly within the variability observed with similar ultra-long-term evaluations; however, it was notable that there was a tendency towards ONL thickening over time. In some patients and some retinal locations (F1/P4-OD and F1/P5-OS ONL thickness moved from being near the upper limit of normal to being significantly hyperthick; in other patients and other retinal locations there was evidence for relative thickening of ONL albeit remaining within normal limits.

### Onset of Extra-Macular Satellite Lesions

In 9 eyes of 5 patients there were extra-macular satellite lesions at one or more visits. Unexpectedly, in both eyes of one patient, several de novo extra-macular satellite lesions was observed in retinal regions where there was no evidence of a lesion at an earlier visit. In F1/P4-OD, NIR-RAFI at ages 29 and 37 yrs showed only a macular lesion. At age 43 however, several small lesions had formed along the superior arcade and OCTs were consistent with development of local outer retinal deposits. IS/OS-RPE thickness topography performed across an ultra-wide extent of retina at ages 37 and 43 showed the existence of an arcuate region of subclinical abnormality at the superior arcade at age 37 where the serous detachments developed 6 years later. The region around the newly developed serous detachments showed widening of the IS/OS-RPE distance. In F1/P4-OS, NIR-RAFI at age 29 yrs showed only a macular lesion. At age 37 yrs, a satellite lesion had formed superior to the optic nerve head near the eccentricity of the vascular arcades. At age 43 yrs, a second satellite lesion formed supero-temporal to the macula. OCTs were consistent with the development of a local serous detachment where none existed previously. IS/OS-RPE thickness topography showed the existence of an arcuate region of subclinical abnormality along a band at the superior vascular arcades which appear to precede the onset of the lesion. These rare results support the hypothesis that clinically-obvious outer retinal deposits and serous detachments may be preceded by subclinical widening of the IS/OS-RPE distance.

### Discussion

### Visual Function in BVMD

Studies in BVDM during the pre-molecular era as well as large series performed more recently in molecularly confirmed patients have shown many patients over a substantial range of ages to have at least one eye with BCVA of 20/40 or better. This level of BCVA would be consistent with retained cone photoreceptor function in many patients with clinically obvious and often chronic serous detachments of the fovea. In our cohort there were BVMD eyes with 20/20 vision and fixation at the fovea which was chronically detached from the underlying RPE. Taking the previously published results together with the current findings, the only conclusion that can be reached is that in BVMD, survival and function of foveal cone photoreceptors are not dependent on their attachment to the RPE cells.

BCVA is a coarse measure of spatial vision and does not scale linearly with photoreceptor degeneration at least for the loss of the first ˜50%. Perimetric light sensitivity provides another measure of visual function and allows sampling of distinct retinal locations surrounding the fovea. Fundus-controlled microperimetry has shown foveal and parafoveal sensitivity losses with retinal lesions in BVMD patients; however, tests were performed under mesopic conditions and the relative contribution of rod versus cone photoreceptors losses were not known. In a subset of eyes retaining stable foveal fixation, we performed light- and dark-adapted perimetry and determined that at retinal loci with chronic serous detachments rod sensitivity losses tended to be substantially higher than cone sensitivity losses. Relatively greater involvement of rod sensitivity or low luminance visual acuity has previously been demonstrated in central serous retinopathy but we are not aware of investigations of direct co-localized comparison of rod and cone function in BVMD or central serous retinopathy (CSR). Publications reporting histopathology from BVMD eye donors have not quantitatively evaluated the comparative involvement of rod and cone photoreceptors.

### Thickening of the Outer Nuclear Layer

Human IRDs and their animal models involve progressive degeneration of the photoreceptors which is reflected by the thinning of the ONL readily measurable on histology. Soon after development of the OCT, our group was the first to recognize that ONL thickness could be measured noninvasively in humans and animals, and progression rates based on loss of photoreceptors could be quantitatively inferred in vivo from serial measurements of the ONL thickness over time. It is less appreciated that counterintuitive thickening can sometimes precede the ONL thinning. A mild ONL thickening can be chronic or a transient stage in a continuum and it has been detected at foveas of patients with choroideremia, periphery of patients with NPHP5- or CEP290-associated Leber congenital amaurosis (LCA), and surrounding the macular drusen in age-related macular degeneration (AMD). Thickened ONL has also been observed in animals as a consequence of interventions or mild disease states. More recently, thickening of ONL upon gene augmentation therapy was seen. Our current work showed that mild ONL thickening is detectable across the clinically normal-appearing retinas of many BVMD patients, and this feature can slowly be accentuated over decades in otherwise normal retinal areas. Our work is consistent with “apparent thickening” of the ONL outside lesion areas and in previtelliform disease stages previously observed in some BVMD patients. It is unlikely that thickened ONL observed across diverse IRDs and AMD represent the same exact underlying pathophysiological cause; however, involvement of subclinical edema due to abnormalities at the RPE-PR interface or sub-threshold signaling of photoreceptor stress can be postulated in BVMD.

### Outer Segment and RPE Interface in Clinically-Uninvolved Extralesion Areas

Clinically obvious serous detachments of the retina from the RPE have been well described in BVMD. This grossly detectable pathology is consistent with the recent understanding that the primary molecular defect acts at the RPE-PR interface in the canine model despite Bestrophin-1 being normally localized to the basolateral plasma membrane of the RPE. What remains less clear is the state of the photoreceptor outer segments and RPE across wide expanses of clinically-uninvolved extralesion areas of the retina in patients with BVMD. Previous investigations to evaluate the earliest disease features in BVMD have resulted in some controversy. Some qualitative studies have suggested subclinical abnormalities at the outer retina, but quantitative studies have come to opposing conclusions. One study measured “photoreceptor equivalent thickness” or “outer segment equivalent length” between the “IS/OS junction” and the “inner surface of the RPE” and found that distance to be on average 6.5 um wider in BVMD patients compared to controls. Another study measured the “length of outer segments” between the “IS/OS junction” and the “RPE1, outer segment/RPE interface” and found no difference between BVMD and controls and controversy ensued. Using our interpretation of OCT peaks in human eyes, measurements of the earlier study would closely relate to what we have labeled ROS length in the current study, and that of the latter study would correspond to what we have labeled COS length. At the superior and inferior perimacular regions we found greater numbers of eyes with lengthening of the ROS than those with lengthening of the COS in BVMD patients. Thus, our results confirm both previous conclusions and help clarify the source of the apparent controversy in the literature.

Understanding of the microscopic details of the minor structural abnormalities in some BVMD eyes well away from central lesions in clinically normal-appearing retina remains elusive. It has been suggested that RPE-PR interface is filled with extremely long and/or unphagocytosed photoreceptor outer segments and that may be true. Extrapolation from the results in dogs with biallelic BEST1 mutations would suggest a developmental abnormality of the RPE microvilli preceding detectable disease onset. Studies in dogs heterozygous for BEST1 mutations could potentially distinguish between these hypotheses in the future. More pragmatically, do these micron-level subclinical abnormalities have any consequence to the progression of disease or loss of vision that BVMD patients may experience? To answer this question is challenging due to the slow progression of disease and limited longitudinal studies with higher resolution cross-sectional techniques. However, in both eyes of one patient we showed de novo formation of clinically-obvious lesions in the exact area of the retina that 6 years earlier showed the largest subclinical RPE-PR abnormality but no lesions. In another patient, we showed dark-adaptation kinetic abnormalities corresponding to the location with a subclinical RPE-PR abnormality. If these anecdotal findings are representative of the progression and vision loss experienced in BVMD, it would be justifiable to treat such retinal areas with subclinical disease assuming a safe and effective treatment becomes available in the future.

There has been increasing evidence that measurable structural changes occur in the outer retina with light exposure. For the current work, all OCT imaging was performed in dark-adapted eyes.

### Outcome Measures for Clinical Trials of BEST1-BVMD

Our results support choices of outcome measures for clinical trials attempting to improve retinal structure and visual function. We showed relatively retained BCVA and relatively large losses of scotopic light sensitivity. In AMD with a scotopic deficit, low-luminance visual acuity (LLVA) has been found to be a simple but informative outcome. In the current study we did not record LLVA, and we are not aware of literature measuring LLVA in BVMD. However, patients with CSR have shown large reductions in LLVA despite retaining good BCVA. Assuming comparability of dysfunction resulting from serous retinal detachments in CSR and BVMD, it would make sense for future clinical trials in BVMD to record both standard BCVA as well as LLVA.

Perimetric methods provide topographic distribution of light sensitivity and can allow comparison of treated areas to neighboring untreated regions in localized interventions such as subretinal gene therapy. Use of light-adapted or dark-adapted conditions allows direct comparison of cone and rod function as was done in the current study. However standard perimetric methods require foveal fixation which may not be attainable in some patients with BVMD. Thus “microperimetry” methods performed with real time tracking of the retina is required. Measurement of cone function with microperimetry is challenging and we are aware of only one instrument that provides testing with the standard photopic background). Measurement of rod function with microperimetry is also challenging due to the limited dynamic range of stimuli available in all devices. Previous studies in BVMD have used microperimetry under mesopic conditions which do not allow distinction of rod and cone function. For a future clinical trial, it would be important to use a microperimetric method the results of which can be interpreted confidently in terms of the function of the underlying photoreceptor system.

En face imaging methods provide convenient analytics to measure changes in lesion appearance and extent as part of the natural history of disease or interventions. Historically, color fundus photographs were used to describe the evolution of BVMD lesions but advent of imaging with short-wavelength autofluorescence (SW-AF) showed a specific hyperintense signal within vitelliform lesions. However, conventional SW-AF is performed with a high intensity excitation light which could, at least in principle, accelerate retinal disease. Therefore, we developed a reduced illuminance autofluorescence imaging (RAFI) method using near-infrared (NIR) excitation. NIR-RAFI can provide en face information regarding RPE health with some similarities to the standard SW-AF results. In the case of BVMD, NIR-RAFI can be more sensitive to earliest disease features, but unlike the ABCA4 form of macular degeneration, extralesion areas in BVMD do not show abnormal increases in SW-AF and NIR-RAFI signals. In the current work, we used NIR-RAFI to delineate not only the gross macular lesions but also de novo development of small satellite lesions. Similarly, NIR-RAFI can form an important outcome measure to provide information regarding RPE health with comfortable lights and a short exam time without undue light hazard potential.

Cross sectional imaging with OCT is a key outcome for BVMD clinical trials. The extent of the vitelliform lesions can be quantified. Additionally, current results strongly suggest that hyperthickening of the ONL and widening of the distance between the IS/OS and the RPE may be the only subclinical abnormalities in paralesion retinal areas that can be evaluated quantitatively for efficacy and safety experimental interventions in clinical trials.

### Example 7: Light-Induced Acceleration of cBest Phenotype and AAV-BEST1 Therapy in Advanced cBest Disease after Light Stimulation

Purpose: To harness the light-modulated acceleration of cBest phenotype to assess AAV-hBEST1-gene therapy in advanced disease.
Subjects: 6 cBest homozygous affected dogs (n=2/mutation).
Study Duration: In life: 48 wks (n=6).
Methods: cBest homozygous dogs are housed under standard (120 lux; n=3) or bright light (450 lux; n=3) cyclic conditions. Both cBest homozygous-affected groups are followed by cSLO/SD-OCT imaging at 4-wk intervals (baseline at 12-wks of age) applying established protocols of targeted light stimulation.

Ophthalmological examination is performed on 3-wk basis and retinal phenotype documented by fundoscopy.

Disease progression rate and severity are addressed in comparison to already collected natural cBest history data of dogs not challenged with targeted light exposure protocols, and correlated with the light preconditioning paradigm set for the two groups.

If a more advanced stage of disease is achieved in these dogs following light exposure, then cBest homozygous dogs are injected bilaterally at 24-wks of age with research-grade AAV-hBEST1 lead therapeutic vector (3.0E+11 vg/mL). Subretinal injections are targeted to retinal areas with advanced disease, whereas retinal regions outside of the treatment bleb serve as internal controls. Treatment response is monitored in vivo for the next 24 wks p.i. (6-, 12-, and 24-wks p.i.), and the phenotype rescue in all 3 distinct cBest homozygous models is assessed by histology & IHC by the end-evaluation (24 wks p.i.).
Outcomes: Assessment of light-induced acceleration of cBest phenotype and its reversal provides critical insight into the disease metrics and development of outcome measures for clinical trial.

### Example 8: Photoreceptor Function and Structure in Retinal Degenerations Caused by Biallelic BEST1 Mutations

The only approved retinal gene therapy is for biallelic RPE65 mutations which cause a recessive retinopathy with a primary molecular defect located at the retinal pigment epithelium (RPE). Another recessive RPE disease is caused by biallelic BEST1 mutations for which pre-clinical proof-of-concept for gene therapy has been demonstrated in canine eyes. The current study was undertaken to consider potential outcome measures for a BEST1 clinical trial in patients demonstrating a classic autosomal recessive bestrophinopathy (ARB) phenotype. Spatial distribution of retinal structure showed a wide expanse of abnormalities including large intraretinal cysts, shallow serous retinal detachments, abnormalities of inner and outer segments, and an unusual prominence of the external limiting membrane. Surrounding the central macula extending from 7 to 30 deg eccentricity, outer nuclear layer was thicker than expected from a cone only retina and implied survival of many rod photoreceptors. Co-localized however, were large losses of rod sensitivity despite preserved cone sensitivities. The dissociation of rod function from rod structure supports a large treatment potential in the paramacular region for biallelic bestrophinopathies.

Retinal diseases caused by BEST1 mutations (bestrophinopathies) are a member of a group of complex monogenic conditions that are inherited both in autosomal dominant and autosomal recessive forms. Other retinopathies in this growing group include those caused by mutations in IMPG1 and IMPG2 which can result in phenotypes overlapping with bestrophinopathies, as well as those with distinctly different phenotypes caused by mutations in RHO, CRX, GUCY2D, RPE65, RP1, PROM1, SNRNP200, PRPH2, GNAT1, SAG, and RDH12. Monoallelic mutations in BEST1 can be non-disease-causing, or cause autosomal dominant Best vitelliform macular dystrophy (BVMD) or autosomal dominant adult-onset vitelliform macular dystrophy (AVMD); more rarely autosomal dominant forms of vitreoretinochoroidopathy (ADVIRC), microcornea, rod-cone dystrophy, cataract, and posterior staphyloma (MRCS), or retinitis pigmentosa (RP) have been described. Biallelic mutations in BEST1 cause the autosomal recessive bestrophinopathy (ARB) phenotype.

The classic examples of juvenile-onset monoallelic BEST1 disease show pathognomonic macular lesions centered on or near the fovea and disease is thought to progress through stages descriptively named based on their ophthalmoscopic appearances such as vitelliform, pseudohypopyon, or vitelliruptive. However retinal disease in so-called vitelliform macular dystrophy (VMD) phenotype is not always limited to the central macula considering reports of one or more satellite lesions forming in what has been named multifocal vitelliform dystrophy. Satellite lesions tend to be located approximately at the eccentricity of the optic nerve possibly corresponding to the ‘rod ring’—an annular region of high rod density in human retinas.

Initial recognition of the biallelic BEST1 disease was associated with a retinopathy that appeared substantially distinct from the classic lesions observed in the monoallelic forms. Biallelic BEST1 disease was thought to not have the foveal vitelliform lesions of the VMD phenotype and instead involve a larger retinal area that included the macula as well as the perimacular and midperipheral regions of the ARB phenotype. Within the involved areas, there were extensive cystic changes in the retina variably described as “macular edema”, “cystic edema”, “intraretinal cysts”, “cystoid intra-retina fluid”, “retinoschisis”, or “cystoid maculopathy”. Cystic spaces were often located within the inner nuclear layer and sometimes in the ganglion cell layer and the outer nuclear layer. Often a shallow subretinal serous detachment was found to extend across the macula. Later stages of disease with greater photoreceptor degeneration seemed to have less cystic changes as if functioning photoreceptors were required for the accumulation of intraretinal fluid.

Over the years it has become clear that not all bilallelic BEST1 disease results in ARB phenotype—some patients show a VMD phenotype normally associated with monoallelic disease. The exact genotype-phenotype relationship remains elusive, but it has been hypothesized that complete inactivation of the gene on both alleles results in ARB phenotype, whereas an allele with a dominant negative effect results in VMD phenotype.

Detailed visual function consequences co-localized with detailed photoreceptor structure abnormalities are not well understood in patients with biallelic BEST1 mutations. To plan for upcoming gene therapy trials, we evaluated rod- and cone-photoreceptor mediated function in patients exhibiting the more commonly encountered ARB phenotype.

### Methods

### Subjects

There were four ARB patients (ages 22-39) from three families. Patients were compound heterozygous for BEST1 gene mutations. Some results from two of the patients (P1 and P2) were previously published (Guziewicz, K. E. et al. Proc. Natl. Acad. Sci. U.S.A 115, E2839-E2848 (2018)). Procedures followed the Declaration of Helsinki, and the study was approved by the Institutional Review Board (IRB) of the University of Pennsylvania. Informed consent, assent, and parental permission were obtained, and the work was HIPAA-compliant.

| Patient | Sex | Allele1 | | Allele2 | |
|---|---|---|---|---|---|
| P1 | F | c.341T > C | p.(Val114Ala) | c.400C > G | p.(Leu134Val) |
| P2 ⁺ | M | c.95T > C | p.(Leu32Pro) | c.102C > T | p.(Glu35Trpfs*11) ⁺⁺ |
| P3 ⁺ | M | c.95T > C | p.(Leu32Pro) | c.102C > T | p.(Glu35Trpfs*11) ⁺⁺ |
| P4 | F | c.763C > T | p.(Arg255Trp) | c.948delG | p.(Val317Cysfs*52) |

⁺ siblings
⁺⁺ Synonymous codon variant predicted to alter splicing (Davidson et al., 2010)
^{§} IOL

### Measures of Rod and Cone Function

Dark-adapted chromatic perimetry (DACP) was used to measure rod-mediated function across the visual field. Mediation of the 500 nm (blue-green) stimulus (1.7° diameter; 200 ms duration) sensitivity by rod photoreceptors was determined by comparison of sensitivities with a 650 nm (red) stimulus and taking advantage of the spectral sensitivity differences between rods and cones. Light-adapted chromatic perimetry (LACP) with a 600 nm (orange) stimulus was used to measure cone-mediated function.

Dark-adaptation kinetics was evaluated similar to techniques previously described using a LED based dark-adaptometer (Roland Consult, Brandenburg a.d. Havel, Germany) and taking advantage of a short duration (30 s) moderate light exposure performed during standard of care from a short-wavelength autofluorescence imaging device (25% laser output; Spectralis HRA; Heidelberg Engineering, Heidelberg, Germany).

### Retinal Imaging

A confocal scanning laser ophthalmoscope (Spectralis HRA, Heidelberg Engineering, Heidelberg, Germany) was used in three patients (P1,P2,P3) to obtain short-wavelength excited reduced-illuminance autofluorescence imaging (SW-RAFI). Wide field image montage was assembled by manually specifying corresponding retinal landmark pairs in overlapping segments using custom-written software (MATLAB 6.5). In one patient (P4), Optos was used to obtain autofluorescence imaging.

Optical coherence tomography (OCT) was performed with a spectral-domain (SD) OCT system (RTVue-100, Optovue Inc., Fremont, CA) in three patients (P1,P2,P3), and a clinical ultrahigh resolution (UHR) SDOCT system (Bi-μ; Kowa Company, Ltd., Tokyo, Japan) was used in two patients (P3,P4) to better understand microscopic features. In three of the patients (P1,P2,P3), serial OCT studies performed in the referring clinic were available. Our recording and analysis techniques have been published.

### Results

### Clinical and Molecular Findings

Best corrected ETDRS visual acuities in the eight eyes ranged from −0.18 to 0.72 log MAR and were within the range published in other patients with biallelic BEST1 mutations and ARB or VMD phenotype. Fixation in the right eyes of P1-P3 was at or near the fovea and relatively stable (bivariate contour ellipse areas ranging from 0.6 to 1.9 deg²); fixation of left eyes of P1-P3 was not formally evaluated. The retained acuities of P4 implied foveal fixation but this was not formally evaluated. P1 had a history of angle closure glaucoma and cataract surgery in both eyes; P2 and P3 were phakic in both eyes. P4 had a history of angle-closure glaucoma and had undergone laser iridotomy in the left eye. Axial lengths were not measured.

P1 carried two missense mutations in BEST1 within the cytoplasmic domain of the protein; both parents were heterozygote carriers and reported to be unaffected. One allele would be predicted to replace the valine at codon 114 with an alanine, and the other allele the leucine at codon 134 with valine. Siblings P2 and P3 carried two missense mutations within the first transmembrane domain of the protein; both parents were heterozygote carriers and reported to be unaffected. One allele would be predicted to replace the leucine at codon 32 with a proline. The other allele is a synonymous variant at codon 34 that has been previously predicted to alter splicing, causing a frameshift and a downstream truncation. P4 carried a missense and a frame shift mutation.

### Prominence of External Limiting Membrane and Other Retinal Features

Qualitatively, all eyes showed substantial abnormalities of retinal cross-sectional structure that extended from foveal to mid-peripheral regions. Abnormalities included large intraretinal cysts located mostly in the INL but also sometimes in the ONL, shallow serous retinal detachments, and abnormalities of structures extending from the ELM to the RPE. There was an unusual prominence of the ELM peak which has been previously described in some disease stages of a different genetic form of macular degeneration but not to our knowledge in mono- or bi-allelic forms of BEST1 disease. In some locations there was local enhancement of IS/OS intensity. Extending into the serous subretinal detachments were stalactite-like reflective material that have been hypothesized to represent outer segments elongated due to impaired phagocytosis. It is also possible that these structures originated from glial cells considering the distinct prominence of the ELM.

### Photoreceptor Structure

ONL thickness in patients was abnormally reduced but detectable across the central 60-degree diameter region. In 4 of 6 eyes where such measures were available there was relative preservation of the parapapillary area as described previously in ARB, as well as in ABCA4-STGD and RDH12-LCA. At 35-40 degree eccentric in the nasal retina and at 25-30 degree eccentric in the temporal retina, there were distinct transitions associated with increases in the ONL thickness with greater eccentricity which often reached normal limits more peripherally. Retinal laminae distal to the ONL were evaluated in detail at a locus near 25 degrees in the nasal retina where ONL was substantially thinned in six eyes, as well as at a locus near 45 degrees in the nasal retina where ONL could be comparable to normal or only mildly thinned.

At 25 degrees eccentric in the nasal retina, best retained lamination was in P1-OS and P3-OD. There were distinct ELM and IS/OS peaks with a presumed IS length that approximated normal. ELM peak was unusually prominent. COST and ROST peaks could not be individually distinguished; distance from IS/OS to RPE was comparable to normal. P3-OS and P1-OD showed some lamination distal to the ELM but the identities of the peaks could not be confirmed. P2-OS and P2-OD showed an unusually prominent ELM peak the identity of which was confirmed by following the peak laterally to neighboring regions with greater preservation; there was no lamination apparent between ELM and RPE.

At 45 degrees eccentric in the nasal retina, best retained lamination was in P1-OS with outer retinal laminae comparable to normal despite a thinned ONL and a challenging ROST peak localization. Intensity of the ELM was enhanced, and the intensities of the IS/OS and COST bands were reduced compared to normal. Length of IS and COS appeared to be comparable to normal. P3-OD, P3-OS and P2-OS showed mostly interpretable lamination. ELM signal intensity appeared to be higher than normal and IS/OS signal lower than normal, with an IS length that was comparable to normal in P3-OD and P3-OS. For P1-OD and P2-OD there was greater noise and laminations were more tenuous but consistent with the other eyes. In all eyes ROST peak was difficult to distinguish but distance from IS/OS to RPE was comparable to normal except in P2-OD which showed mild reduction. It is important to note that in many cases there was an apparent hyposcattering between COST and RPE peaks and a microdetachment of the retina from the RPE similar to that seen in dogs with biallelic BEST1 mutations cannot be specifically ruled out.

SW-RAFI showed abnormalities consisting of spatial heterogeneity of signal extending to the midperiphery which was well correlated with the OCT abnormalities in six eyes. In the nasal periphery of each eye, there was a distinct transition to local homogeneity in SW-RAFI signal corresponding to greater thickness of ONL and less outer retinal laminar abnormalities observed on OCT. More limited data from both eyes of P4 were comparable.

### Longitudinal Changes in Retinal Structure

Additional imaging studies were performed in many eyes at different patient ages. Main changes detectable with the available data were regarding the fluctuation of intraretinal cysts. Specifically, there were examples where cysts visible within the ONL became undetectable at other visits or vice versa. There were also examples of INL cysts substantially changing in size and extent. In addition, subretinal fluid extent and localization could be seen to change. Some of the smaller fluctuations could be due to inexact matching of the scan locations at different visits, but some of the larger fluctuations could not be explained with scan location and must reflect physiological changes occurring in the retina. Evaluation of potential longitudinal changes in the outer retina relevant to progressive photoreceptor degeneration (such as ONL thickness or OS length) was not possible with the data available.

### Retinal Function—Evidence for Treatment Potential

Rod and cone sensitivities were sampled densely (every 2 deg) along the horizontal and vertical meridians in the central and mid-peripheral retina, and sparsely (12 deg grid) across the full visual field, and co-registered to retinal structure. In general, there were large losses of rod sensitivity across the swath of central and midperipheral retina that unsurprisingly showed OCT and SW-RAFI abnormalities. Beyond 30-50 degrees eccentric from the fovea, where retinal structure normalized, rod function also approached normal in the superior, inferior and temporal visual fields. The furthest tested eccentricity (48 deg) in the nasal visual field remained abnormal in all evaluated eyes. In four of the eyes (P1-OD, P1-OS, P3-OD, P3-OS), there was evidence of parapapillary region of relatively better rod function consistent with the parapapillary retention of retinal structure. Cone sensitivities were generally substantially better preserved as compared to rod sensitivities. At many loci within the central and mid-peripheral areas of clear-cut retinal structural defects, cone sensitivities were within or near normal limits, or showed mild losses of less than 1 log unit. Of interest, near normal cone sensitivities could include retinal regions with serous detachments (e.g., P3-OD, temporal to fovea) and large intraretinal cystic spaces (e.g., P2-OS, nasal to the fovea).

To determine whether large losses of rod sensitivity with retained cone sensitivity were due to severe degeneration of rod cells whilst cone cells survived, we evaluated co-localized measures of ONL thickness and sensitivity. By imaging methods available to date, rod and cone nuclei within the ONL are not directly distinguishable by their backscatter characteristics. However, we have previously estimated the fraction of the ONL containing cone nuclei as a function of eccentricity in normal eyes. Near the fovea, ONL thickness remaining in ARB eyes was well within the possibility of a cone-only retina and thus evidence for remnant but dysfunctional rod cells was not obtainable. Existence of a rod or cone treatment potential within the central ˜14 deg diameter region of the macula is currently indeterminate. Beyond ˜30-40 deg in the nasal retina both structure and function approached normal and thus there is no large treatment potential in terms of short term improvement of visual function. Between ˜7 and ˜30 deg eccentricity nasal to the fovea, retained ONL thickness in ARB was well beyond that expected from a pure cone retina. Similarly, beyond ˜7 deg eccentric in the temporal retina, there was ONL thickness likely including substantial rod nuclei. Both regions had rod function disproportionately reduced and strongly suggest remaining rod photoreceptors lacking function with a large magnitude treatment potential in terms of a short term improvement of visual function.

### Retinal Function—Kinetics of Rod Dark-Adaptation

All photoreceptor cells require a photolabile opsin molecule to signal light and the universal chromophore for all opsin molecules is 11-cis-retinal. There are now at least three known cycles that constantly regenerate 11-cis-retinal for continual vision. For rod photoreceptors, the bulk of the 11-cis-retinal originates from RPE cells within the so-called canonical visual cycle. We recorded dark-adaptation kinetics of the rod system to estimate the speed of the canonical visual cycle. All three patients studied demonstrated abnormally slow kinetics of dark-adaptation recovery.

### Discussion

### Genotype-Phenotype in Biallelic BEST1 Mutations—Review of Literature

Following the initial reports, there has been a growing list of publications describing autosomal recessive retinal disease caused by biallelic BEST1 mutations. The resulting retinal phenotype appears to fall into two distinct categories with some overlap. At one extreme is the ARB phenotype with retinal involvement extending from fovea to midperiphery demonstrating intraretinal cystic changes and subretinal serous detachment. The other extreme of biallelic BEST1 mutations is a VMD-like phenotype normally associated with monoallelic disease demonstrating a vitelliform lesion at the central macula surrounded by near-normal retina. To understand whether there is a genotype-phenotype relationship, we reviewed 139 patients in 23 publications in terms of their reported genotype and, when possible, we categorized their phenotype based on published OCT data and clinical descriptions. We found 72% (100/139) to have ARB-like phenotype, 12% (17/139) VMD-like phenotype and remaining patients were indeterminate or uncertain. Reported visual acuities were plotted and showed overlap between the phenotypes. In terms of genotype, having both alleles with predicted truncations was more likely to result in ARB phenotype but there were also examples with VMD phenotype. Homozygosity for missense mutations Arg255Trp, or Arg141His caused ARB whereas homozygosity for the missense mutation Arg47Cys caused VIVD phenotype. Frameshift, truncation and splice site mutations could be paired with missense mutations (e.g., Arg255Trp or Ala195Val or Arg141His) to cause ARB phenotype, or VMD phenotype. There was even a patient with compound heterozygous missense mutations who appeared to show VMD-like phenotype in one eye and ARB phenotype in the other eye. Thus, a clear genotype-phenotype relationship remains elusive. We are aware of the proposed hypothesis that a complete inactivation of BEST1 gene on both alleles results in ARB phenotype, whereas an allele with a dominant negative effect results in VMD phenotype. However, this hypothesis does not explain the unaffected heterozygous family members of patients with VMD-like phenotype and missense mutations or rare ARB phenotype associated with heterozygous missense mutations. Practically, among all reported patients with biallelic BEST1 mutations, the more severe ARB phenotype appears to be much more common than the milder VMD phenotype. Our detailed studies herein attempted to provide greater clarity to the ARB phenotype.

### Intraretinal and Subretinal Fluid

RPE cells are key in fluid transport/management as they transport fluid from the photoreceptors to the choroid. Retinopathies of diverse origins affecting the RPE function can result in serous retinal detachments where fluid accumulates between the photoreceptors and the RPE. Both ARB and VMD-like phenotypes of biallelic BEST1 mutations can show serous retinal detachments which is not surprising considering BEST1 is a primary RPE disease. An important distinction between ARB and VMD-like phenotypes, however, is the accumulation of intraretinal fluid only in the former. The combination of intra- and sub-retinal fluid is not common and typically observed in patients with chronic central serous retinopathy. Specifically, common forms of intraretinal fluid (cystoid macular edema) of inflammatory or ischemic origins do not typically show retinal detachment. Similarly, intraretinal fluid without retinal detachment is often seen in inherited retinal degenerations. It can be hypothesized that ARB phenotype reflects the chronicity of an earlier VMD-like phenotype after vitelliform lesions have been resorbed. However, this hypothesis is refuted by observations of ARB phenotype detectable in very young patients in the first decade of life. Alternatively, ARB phenotype reflects greater involvement of Muller glial cells in the disease process as compared to the VMD phenotype. Prominence of the ELM detected on imaging patients with ARB phenotype seems to provide some support for this alternative hypothesis.

### Rod Function Deficit

Dysfunction of the rod photoreceptor driven night vision is encountered commonly in a wide range of inherited retinal degenerations. It was generally assumed that loss of function was due to loss of cells. We now know that a functional deficit can exist in retained photoreceptor cells. Patients with biallelic BEST1 mutations and an ARB phenotype showed large retinal regions with substantial loss of dark-adapted rod sensitivity co-localized with retinal structure likely retaining rod nuclei. In addition, there was abnormal slowing of the kinetics of dark-adaptation rate implying a chromophore recycling defect. Correction of BEST1 pathophysiology with gene augmentation could improve both rod sensitivity and accelerate the kinetics of rod dark-adaptation. However, it is important to note that there were structural abnormalities at the photoreceptor-RPE interface at the level of cone and rod outer segments. Whether these long-standing abnormalities can be ameliorated by gene therapy remains to be evaluated.

### Example 9: Analysis of Long-Term Stability of AAV-BEST1 Treatment in cBest

Purpose: Assessment of long-term efficacy of human BEST1 transgene expression in cBest eyes is followed longitudinally.
Subjects: cBest dogs (n=10; both sexes), harboring R25*/R25* or P463fs/P463fs or R25*/P463fs cBEST1 mutations, injected with AAV2-hBEST1 (titers range: 0.5-5.0E+11 vg/mL), and followed by cSLO/SD-OCT imaging for 39-147 wks post injection (p.i.).
Methods: Comprehensive analysis of existing longitudinal in vivo imaging data and retinal histological analysis.
Assessments of cBest eyes (n=20) involves: generation of topographic maps of ONL thickness, quantification of IS/OS-RPE/T distance, comparative analysis of clinical stages in relation to patients, evaluation of phenotype rescue (reversal of macro- and micro-detachments) based on en face and cross-sectional recordings; retinal preservation is assayed in cryosections (H&E, IHC with RPE- and neuroretina-specific markers), and examined by confocal microscopy. Restoration of RPE-PR interface structure is assessed qualitatively and quantitatively (number of cone-MV/mm2) vs AAV-untreated control retinas.
Outcomes: Analyses of in vivo data assists in defining disease stages in patients sensible to approach with BEST1 gene augmentation therapy. Histology/IHC determines dose-response relationship with regard to correction of structural alterations at the RPE-PR interface.
SCOPE: cSLO/SD-OCT: topographic maps IS/OS-RPE/T distance & ONL thickness; H&E/IHC/cBest-AR eyes n=11 AAV-hBEST1-injected vs CTRLs

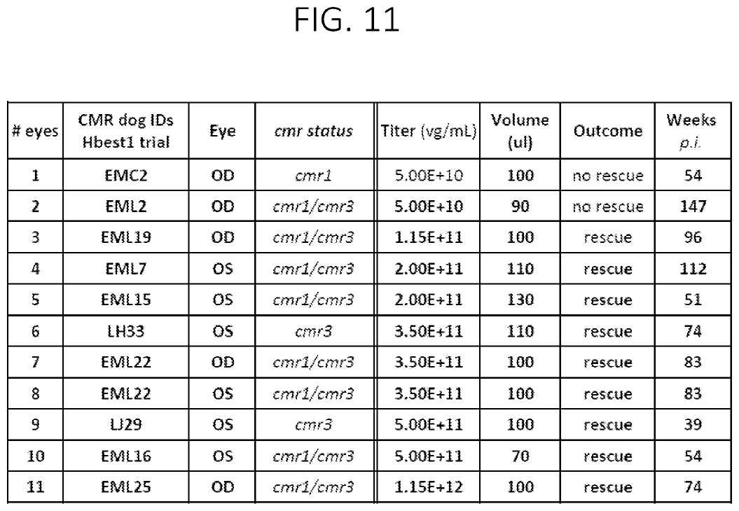

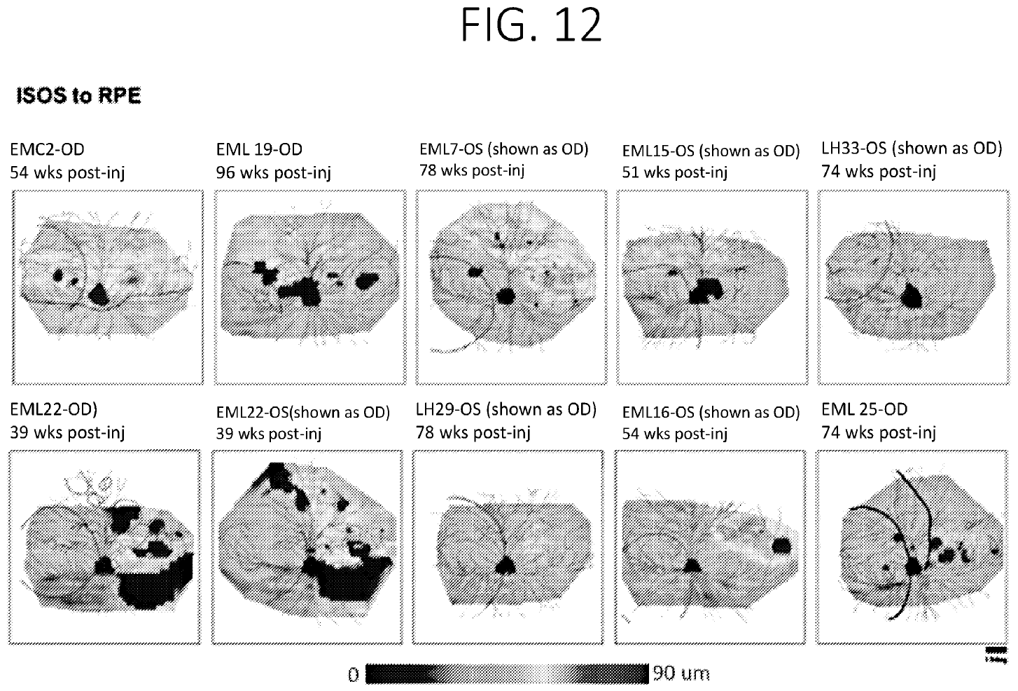

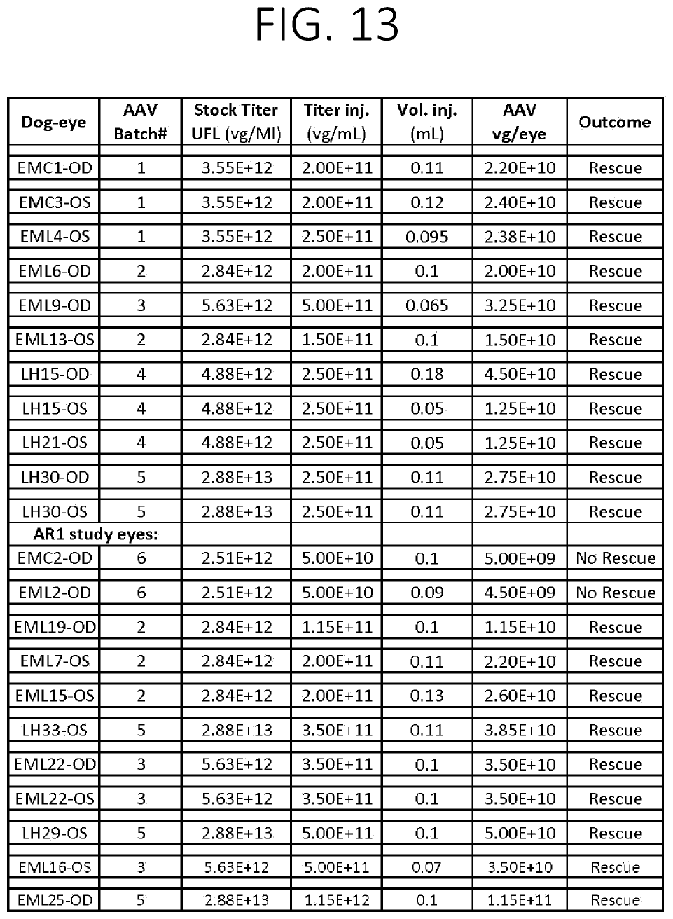

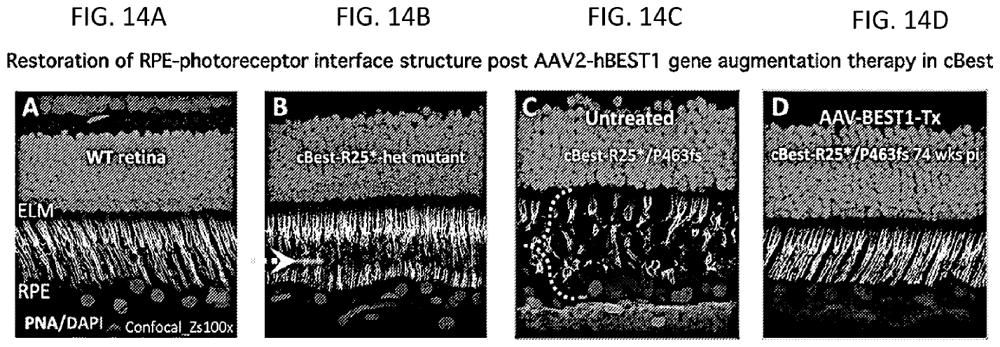

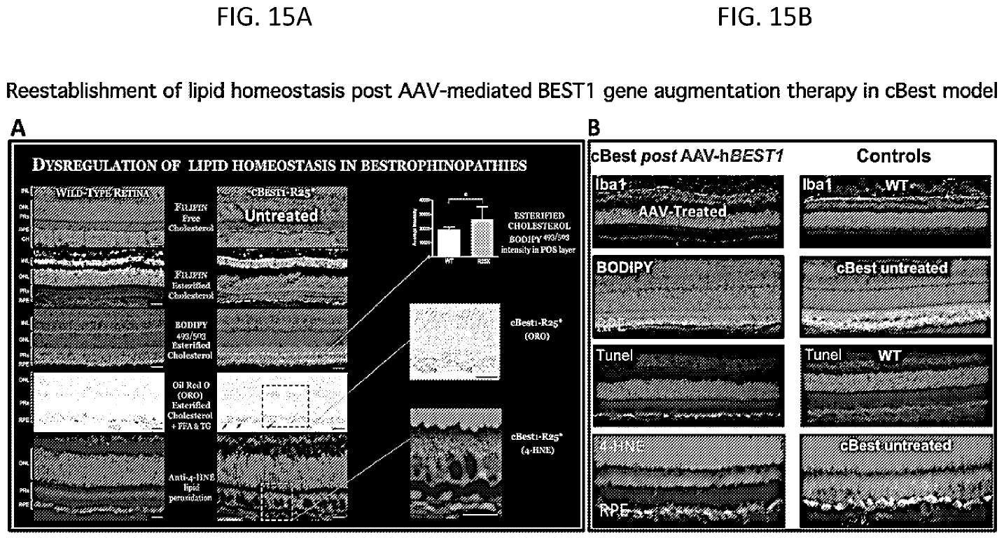

FIG. 11 shows a summary of cBest-AR rAAV2-hBest1-injected eyes enrolled in the study. All eyes receiving a dosage of 1.15×10¹¹ or higher showed rescue. FIG. 12 shows assessment of cBest-AR treated subjects up to 74 weeks post injection. FIG. 13 shows cBest eyes dosing in comparison to published cBest subjects.

### Example 10: Assessment of Treated cBest Mutant Dogs

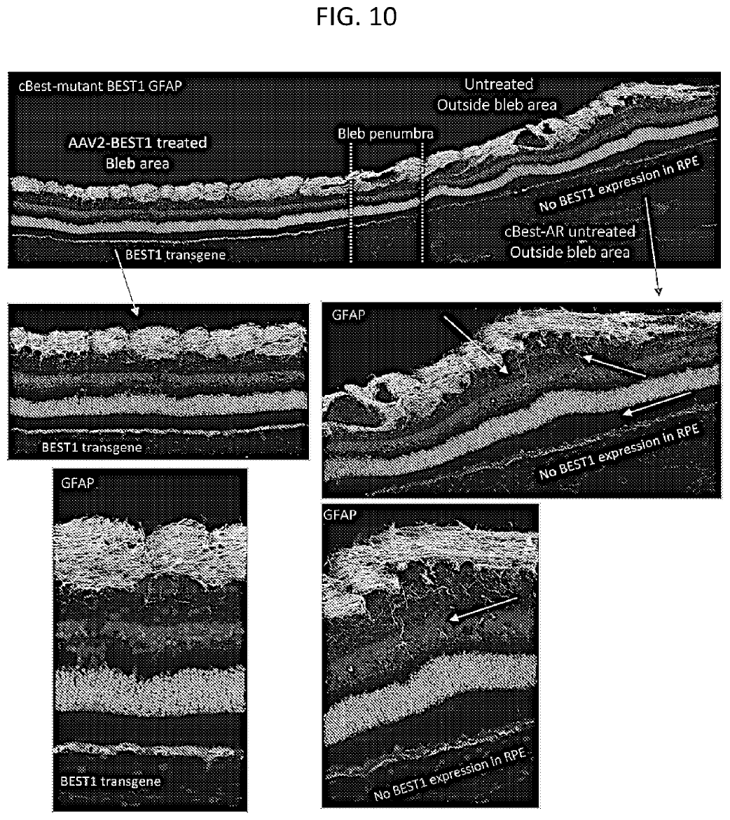

cBest mutant dogs were treated as previously described (Guziewicz et al, BEST1 gene therapy corrects a diffuse retina-wide microdetachment modulated by light exposure, Proc Natl Acad Sci USA. 2018 Mar. 20; 115(12): E2839-E2848. Published online 2018 Mar. 5, which is incorporated herein by reference). In view of newly observed phenotypic changes in cBest-Hets described herein, treated eyes were evaluated to determine whether the gliotic changes were observable in the cBest model. Retinas were evaluated for transgene expression, and using GFAP for gliosis and astrocytosis. As previously noted, Best1 expression was observed in RPE in treated bleb area, but not outside bleb. Increased MG gliosis and astrocytosis were observed in the untreated regions (outside bleb penumbra) of treated eyes (FIG. 10), but not in AAV2-Best1 treated areas.

### Example 11: AAV2/2-BEST1 Treatment of BEST1-Mutant Dogs

The objectives of this study were to conduct a safety and efficacy analysis in BEST1-mutant dogs of AAV2/2-BEST1, manufactured using transient plasmid DNA transfection. The AAV2/2-BEST1 vector or vehicle was administered by a single subretinal injection in one eye of BEST1-mutant dogs as outlined in the table below.

Efficacy endpoints in this study included masked analyses of: progression of disease by funduscopic examination, and changes in IS/OS to RPE/tapetum interface distance and changes in ONL thickness (by in vivo OCT imaging).

Safety endpoints in this study included masked analyses of: clinical examination, ophthalmic examination, retinal examination by in vivo cSLO/OCT imaging, ERG responses, clinical pathology assessment, immunological and biodistribution analysis, as well as gross pathology and microscopic pathology. Details of these procedures can be found in the respective Methods sections.

Investigators and evaluators involved in acquisition and analysis of efficacy and toxicity data were masked to the treatment group of each animal.

A total of 9 animals received the test article or vehicle by subretinal injection in the left eye. The contralateral right eye remained untreated. Animals age ranged from 16-194 weeks at the time of treatment. Both male and female dogs were allocated to each treatment group.

The subretinal surgical procedure in the 9 animals was performed over 3 consecutive days. On each day, both doses (high, low) of the test article and the vehicle were administered in the left eye of 3 different animals. To ensure similar representation in gender, ages, stages of disease, and genotypes, the animals were pre-assigned in a non-randomized manner to the different treatment groups. One animal, had a failed subretinal injection (reflux into the vitreous). This animal was removed from the study and replaced on that same day.

TABLE 1
Study Design

| | Dose Level | | | |
|---|---|---|---|---|
| | | Vector | | Injection |
| | Number of | Concentration | Total Dose | Volume |
| Dose | Animals | (vg/mL) | (vg per eye) | (mL) |
| High | 3 | 3.0 × 10¹⁰ | 4.5 × 10⁹ | ~0.15 |
| Low | 3 | 9.5 × 10⁹ | 1.4 × 10⁹ | ~0.15 |
| Vehicle | 3 | 0 | 0 | ~0.15 |

### Study Design

A total of 9 homozygote or compound-heterozygote BEST1-mutant dogs were allocated to 3 treatment groups. Three animals received a subretinal injection in the left eye of AAV2/2-BEST1 at the high-dose (150 μL, 3.0×10¹⁰ vg/mL, 4.5×10⁹ vg/eye, high-dose group). Three animals received a subretinal injection in the left eye of AAV2/2-BEST1 at the low-dose (150 μL, 9.5×10⁹ vg/mL, 1.4×10⁹ vg/eye, low-dose group), and three animals received a subretinal injection in the left eye of the vehicle control article (150 μL, Alcon BSS with 0.001% Poloxamer 188, vehicle group). The contralateral right eye remained un-injected in all the study dogs (9 eyes). All animals were sacrificed at 13 weeks post-injection.

Assessment of efficacy was based on assessment of progression of disease by funduscopic examination, non-invasive retinal imaging including confocal scanning laser ophthalmoscopy (cSLO), standard and high-resolution optical coherence tomography (OCT).

Assessment of safety was based on survival, clinical observations, clinical pathology, ophthalmic examinations, cSLO/OCT retinal imaging, full-field electroretinography (ERG), gross anatomic pathology and histopathology.

Both low and high doses of AAV2/2-BEST1 caused a reduction in the photoreceptor to RPE distance, and in some animals either prevented the onset of clinically-detectable BEST-1 lesions, or caused preexistent areas of focal retinal detachment to reattach. Unexpectedly, AAV2/2-BEST1-injected eyes also had a better ERG function than the contralateral un-injected eyes.

No mortality, systemic toxicity nor test article-related effects on body weight, clinical pathology parameters, organ weights, or macroscopic and histopathologic findings were seen during the 13-week in-life phase of the study with either the vehicle, low-dose of AAV2/2-BEST1, or high dose of AAV2/2-BEST1. No signs of ocular toxicity that could be unambiguously associated with either the vehicle control article or the test article (low-dose AAV2/2-BEST1, high-dose AAV2/2-BEST1) were seen in any of the treatment groups.

In summary, a single subretinal injection of a low- (1.4×10⁹ vg/eye) or a high-dose (4.5×10¹⁹ vg/eye) of AAV2/2-BEST1 led to improvement of the photoreceptor-RPE interface, and prevented the occurrence or reversed the progression of BEST1 disease in the naturally-occurring canine model. This was associated with a functional (ERG) benefit.

As no signs of systemic nor ocular toxicity were found in the high-dose treatment group, this study did not identify the no-adverse effect level (NOAEL) of AAV2/2-BEST1 in the naturally occurring BEST1-mutant dogs.

### Methods

### Test and Vehicle Control Articles

AAV2/2-BEST1 is a recombinant AAV2/2 vector that carries human BEST1 complementary DNA (cDNA) as a single-stranded construct. The plasmid contains a human VMD2 promoter (VMD2), driving the expression of human BEST1. The BEST1 cDNA is preceded by a simian virus (SV) 40 synthetic intron splice donor/splice acceptor (SV40 SD/SA) and followed by an SV40 polyadenylation sequence. The VMD2-BEST1 vector genome is packaged into an AAV2 capsid. The AAV2/2-BEST1 vector was by cotransfection of human embryonic kidney (HEK) 293 cells with three plasmids, the transgene plasmid VMD2-BEST1, the Rep2Cap2 plasmid and the helper plasmid pALD-X80, and it was subsequently purified by affinity and anion exchange chromatography, followed by cesium chloride ultracentrifugation, concentrated/buffer exchanged against Balanced Salt Solution (BSS) and supplemented with 0.001% Poloxamer 188, pH 7.0. The specific vector used in the study is a toxicity lot that was made under GMP conditions.

### Vehicle Control Article

The vehicle control article consisted of Balanced Salt Solution containing 0.001% Poloxamer 188, pH 7.0. It was used for dosing the vehicle treatment group and also to dilute AAV2/2-BEST1 to the concentration required for administration to the test article treatment groups.

### Test System

### Choice of Animal Model

The test system was the naturally-occurring canine model of BEST1-associated maculopathies, canine multifocal retinopathy (cmr), a.k.a. canine bestrophinopathy (cBest). This retinal disorder in the dog is caused by one of three distinct mutations in the canine BEST1 gene that include a premature stop mutation (R25X) (Guziewicz et al. Invest Ophthalmol Vis Sci. 2007; 48: 1959-1967) in the cmr1 line, a missense mutation (G161D) (Guziewicz et al. Invest Ophthalmol Vis Sci. 2007; 48: 1959-1967) in the cmr2 line, and a frameshift mutation (P463fs) (Zangerl B et al., Mol Vis. 2010; 16: 2791-2804) in the cmr3 line. Dogs that were homozygous mutant for the G161D mutation (cmr2/cmr2), and for the P463fs mutation (cmr3/cmr3), and dogs belonging to 3 lines of compound heterozygotes (R25X/G161D, a.k.a. cmr1/cmr2; R25X/P463fs, a.k.a. cmr1/cmr3; Gl61D/P463fs, a.k.a. cmr2/cmr3) were used in this study. All cBEST1-mutant genotypes result in a highly consistent clinical phenotype, fully recapitulating the human disease. cBest remains the only nonclinical model of human Best disease. cBest recapitulates all aspects of human BEST1-associated maculopathies, including clinical phenotypes, molecular and histological features. To validate the translational suitability of the cBest lines and design meaningful proof-of-concept studies, 18 cBest dogs (12 male/6 female) representing the cmr1/cmr1, cmr1/cmr3, and cmr3/cmr3 lines, and ranging in age from 6 to 297 weeks, were enrolled in a natural history study (Guziewicz K E et al. Proc Natl Acad Sci USA. 2018; 115: E2839-E2848). Regardless of genotype, focal RPE-PR detachments were noted by optical coherence tomography (OCT) assessments as early as 11 weeks of age. In the canine retinas, similar to humans, lesions develop in the macular region. Four stages of cBest disease were notable, capturing the full range of human phenotypes, including subclinical pre-vitelliform, vitelliform, pseudohypopyon and vitelliruptive lesions, corresponding to stages I-IV of human Best disease (Guziewicz K E et al. Proc Natl Acad Sci USA. 2018; 115: E2839-E2848). In each case, similar to humans, disease was bilateral and presented with remarkable symmetry. From the subclinical stage (stage I), the disease progressed to a macrodetachment (vitelliform stage; stage II) localized to the canine fovea-like region and surrounded by microdetachments. These detachments then expanded to encompass the entire fovea-like region. A pseudohypopyon stage, characterized by hyperautofluorescence in the inferior aspect of the lesion, also developed (stage III). The advanced stage of the disease (stage IV) was associated with significant thinning of the outer nuclear layer (Figure S4 in Guziewicz K E et al. Proc Natl Acad Sci USA. 2018; 115: E2839-E2848), as observed in patients, leading to vision loss.

### Prior Demonstration of the Responsiveness of the Model to Gene Therapy

A proof of concept study demonstrating that BEST1 gene augmentation corrects the diffuse retina-wide microdetachment phenotype in BEST1-mutant dogs has recently been published (Guziewicz K E et al. Proc Natl Acad Sci USA. 2018; 115: E2839-E2848). In this work, research grade AAV2/2 vectors carrying either the human or canine BEST1 cDNA under control of the VMD2 promoter were subretinally-injected (volume: 0.05 to 0.18 mL; titers: 0.1 to 5×10¹¹ vg/mL) in 12 dogs (15 eyes) that were either homozygous mutant (cmr1/cmr1; cmr3/cmr3) or compound heterozygous (cmr1/cmr3). Age at injection ranged from 27 to 69 weeks. Duration of follow-up ranged from 13 to 245 weeks. In vivo retinal imaging (cSLO/OCT) showed that microdetachments and/or focal lesions (stages II/III) in AAV-BEST1-treated regions resolved 4 to 12 weeks post-injection., and localized retinal reattachments remained stable thereafter. In eyes (n=7) that were injected with vehicle (BSS) lesions reappeared as early as 1-week post-injection and progressed. AAV-BEST1 treatment was also associated with reversal of the hyperthick ONL to normal values. Immunohistochemical analysis of retinal tissues showed restoration of the cytoarchitecture at the photoreceptor-RPE interface that included extension of cone microvilli and reorganization of the actin cytoskeleton.

In summary, this prior study showed that AAV-mediated BEST1 gene augmentation in canine bestrophinopathies promotes a sustained reversal of gross retinal detachments, reestablishment of a close contact between RPE and photoreceptors, and return of ONL thickness to normal values.

### Study Animals

The study design included use of BEST1-mutant dogs of both genders representing 5 different genotypes, and with ages ranging from 16 to 194 weeks at the dosing time-point so as to include animals with a spectrum of disease stages ranging from Stage I/II to Stage III/IV. Animals had a weight range of 5.1 to 16.8 kg at initiation of the study.

### Animal Care and Use Statement

All procedures in this protocol were conducted in compliance with the Animal Welfare Act, the Guide for the Care and Use of Laboratory Animals, and the Office of Laboratory Animal Welfare. In the opinion of the Sponsor and Study Director, the study did not unnecessarily duplicate any previous work, and no other model could have fulfilled the study requirements.

### Animal Husbandry

Housing: Animals were kept at the Retinal Disease Studies Facility on the New Bolton Center campus (Kennett Square, PA) of the School of Veterinary Medicine, University of Pennsylvania. This research animal facility houses exclusively dogs affected with inherited forms of retinal degeneration.
Quarantine Period: No quarantine was needed.
Food: Animals were fed as follows:
   From 3-4 weeks of age (pre-weaning; maternal feeding: “Purina Pro Plan Focus Chicken and Rice Dry Puppy Food” in the food hopper and a ½ can or more of the “Purina Pro Plan Focus Chicken and Rice Canned Puppy Food”, depending on the dam's nutritional requirements. Canned food was given twice a day, dry food once a day.
   From 4-12 weeks of age (weaning period): “Purina Pro Plan Focus Chicken and Rice Dry Puppy Food” mixed with the “Purina Pro Plan Focus Chicken and Rice Canned Puppy Food”. Dry food was given once a day. After about 10 weeks of age, canned food was reduced to once a day.
   From 3 to 8 months: “Purina Pro Plan Focus Chicken and Rice Dry Puppy Food” was given once a day.
   After 8 months: “Purina Pro Plan Focus Chicken and Rice Dry Adult Food” was given once a day.
Water: The animals had continuous free access to water.

### Ambient Temperature in the Kennel Areas:

In all rooms (except whelping & puppy rooms): the optimal temperature was kept at 74° F. (±4° F.).

### Hygrometry in Kennel Areas:

Mean weekly hygrometry was maintained in the 30-70% range, except the weeks of Nov. 2, 2020 and Jan. 18, 2021, when the hygrometer provide reading as “Hi” or “Lo”, and accurate humidity levels were not measured during those 2 weeks.

### Illumination:

Dogs were kept in kennel runs under cyclic light environment (6 AM on-6 PM off) with light intensities that varied between 190-217 lux at the level of the “standard” dog eye. In the Eye Exam Room, which was used infrequently, light intensities at “standard” dog eye level ranged from 45 to 85 lux. In the Exam Room 2 light intensities at “standard” dog eye level ranged from 185 to 240 lux. In the Spectralis Room, light intensities at “standard” dog eye level ranged from 180 to 430 lux. In the ERG Room, light intensities at “standard” dog eye level ranged from 56 to 270 lux. In the Visual Behavior Room, light intensities at “standard” dog eye level ranged from 95 to 198 lux. These ambient light levels do not cause retinal abnormalities in normal or BEST1-mutant dogs. Light intensities in the rooms were monitored monthly throughout the duration of the study using an illuminometer (IL-1700, International Light Technologies).
Health Status: Health status was monitored on a regular basis by a certified veterinarian and included: body condition score, weight, respiratory rate, heart rate, chest auscultation, abdominal palpation, lymph node palpation, rectal temperature, skin condition and pulse quality assessment.

### Animal Identification, and Assignment to Treatment Groups

An implantable microchip identification device and skin tattooing were used in the dogs assigned to the study as individual identification methods.

Due to the limited number of available mutant dogs that had diverse cBEST1 genotypes, ages and stages of disease at baseline, animals were pre-assigned to the treatment groups in a non-randomized manner, that assured that each treatment group included:
dogs of both genders, with ages that ranged from young (16-17 weeks) to older (>72 weeks) at pre-dosing.
dogs that were homozygous mutant for at least one of the 3 cBEST1 mutations, and dogs that were compound heterozygote mutants.
dogs with stages of disease that ranged from Stage I (pre-vitelliform with microdetachmemts) to Stage III (multifocal pseudohypopyon with autofluorescent material).

On each three consecutive days of dosing, the three animals that were treated on a given day, included one animal from each treatment group. To minimize bias, the order in which test or vehicle control article was to be administered was randomized prior to the day of dosing.

### Dosing Procedures

### Rationale for Dose Levels Selection

The dose levels were selected based on previous experience in gene therapy with this AAV2/2-BEST1 vector in wild type dogs and the research vector AAV2/2-hBEST1 in cBEST1-mutant dogs (Guziewicz K E et al. Proc Natl Acad Sci USA. 2018; 115: E2839-E2848). This toxicity/efficacy study was aimed at examining two dose levels of AAV2/2-BEST1 with the goal of identifying doses that can treat Best disease retinal lesions.

### Preparation of the Test Article

Test article AAV2/2-BEST1 was provided as a stock solution at a concentration of 1.81×10¹² vg/mL (measured by ddPCR) to allow preparation of all proposed doses. On each day of dose administration, the required number of fresh vials of test article was thawed and diluted to the required concentration (3.0×10¹⁰ vg/mL, and 9.5×10⁹ vg/mL) with the vehicle (BSS containing 0.001% Poloxamer 188, pH 7.0) using aseptic techniques. The formulations were prepared by diluting the stock solution with the appropriate volume of vehicle to reach the final target concentrations following dilution of viral vector and specific dose preparation protocols. Dosing formulations were prepared in sterile USP Type 1 glass vial(s) and stored at 2-8° C. and were used on the same day.

A MicroDose™ injection kit equipped with a PolyTip® cannulas (25G/38G) (MedOne, Sarasota, FL) were used to perform the subretinal injections. This subretinal injector device is currently being utilized for subretinal gene therapy in patients and has been successfully used by the veterinary surgeon (Dr. Beltran) in dogs.

After filling the dosing syringes, residual dose formulations (pre- and post-device) were stored in a freezer at a temperature of ≤−60° C. for future dose analysis.

### Dose Administration

Doses were administered at a volume of 0.15 mL/eye using the subretinal injector dosing apparatus described above.

The animals were dosed once via subretinal injection in the left eye.

Animals were anesthetized, and the left eye was cleaned with an approximately 1% povidone iodine solution (prepared with sterile saline and 10% povidone iodine). An adhesive sterile drape (as used in human surgeries) was applied on the eye and around it.

The subretinal injection was performed following a study-specific procedure, briefly described as follows: A retrobulbar injection of sterile saline was done to place the globe in primary gaze position, and stay sutures were placed. A lateral canthotomy was performed to increase exposure of the sclera. A 25-gauge trocar was placed. The subretinal injector's cannula was introduced into the vitreal cavity via the transcleral trocar, and the solution was delivered into the subretinal space under direct visualization while controlling the injection pressure with the vitrectomy console foot pedal. Visualization of the fundus was achieved through an operating microsope (Zeiss Lumera 700, Carl Zeiss Meditec, Inc. USA) with a Machemer magnifying vitrectomy lens (OMVI; Ocular Instruments Inc., Bellevue, WA, USA). Further details are provided in a subretinal injection SOP. Immediately following the subretinal injection, the dogs had a series of fundus photographs of the injected eye captured with a RetCam unit to document the location and size of the subretinal bleb.

The time of each dosing was recorded as the time of completion of each subretinal injection. A detailed description of the injection site (fundus photography), the delivered injection volume, presence of retinotomy, any potential leakage and cause of the leakage (e.g., device failure, etc.) was documented in individual reports for each animal.

On the day prior to surgery, dogs received oral administration of antibiotics (amoxicillin trihydrate/clavulanate potassium; 12.5-20 mg/kg; Dechra Veterinary Products, Overland Park, KS) in the morning and afternoon, as well as oral administration of a corticosteroid (prednisone; 1 mg/kg; Lannett Company, Inc. Philadelphia, PA) in the morning and afternoon; topical corticosteroid was applied twice a day (prednisolone acetate 1% suspension; 1 drop; Allergan, Irvine, CA). On the morning of surgery, dogs received oral antibiotic (amoxicillin trihydrate/clavulanate potassium; 12.5-20 mg/kg) and topical antibiotic (gentamicin sulfate solution; 1 drop; Allergan, Irvine, CA) and anti-inflammatory agents (prednisone; 1 mg/kg/prednisolone acetate; 1% suspension; 1 drop/flurbiprofen 0.03%; 1 drop; Bausch and Lomb, Tampa, FL). In addition, topical mydriatics (atropine sulfate 1%; Akorn, Inc. Lake Forest, IL, or Wedgewood Pharmacy, Swedesboro, NJ), tropicamide 1%; Akorn, Inc. Lake Forest, IL, and phenylephrine 10%; Paragon Bioteck, Portland, OR; 1 drop 3 times, 30 minutes apart) were given prior to dosing. Immediately after the end of the surgical procedure, a subconjunctival injection of 4 mg of triamcinolone acetonide (Kenalog 40 mg/mL; Bristol-Myers Squibb, Montreal), and topical application of antibiotic plus steroid ointment (NeoPolyDex ointment, ¼ inch strip, consisting of neomycin sulfate 3.5 mg, polymyxin B sulfate 10,000 units and dexamethasone 0.1%; Bausch and Lomb, Bridgewater, NJ) was given in the injected eye only. In the afternoon of the procedure, the animals received a second dose of oral antibiotics (amoxicillin trihydrate/clavulanate potassium; 12.5-20 mg/kg) and corticosteroid (prednisone; 1 mg/kg) medications. Topical application of atropine sulfate 1% ointment (¼′ strip) was applied once a day in the treated eye for 1 week post-injection. Corticosteroid suspension (prednisolone acetate; 1% suspension; 1 drop) was applied twice a day in the treated eye for 2 weeks post-injection and then once daily for the following 2 weeks. Oral administration of antibiotics (amoxicillin trihydrate/clavulanate potassium; 12.5-20 mg/kg) was given twice a day for 5 weeks. Oral administration of corticosteroid (prednisone) was given twice a day for 2 weeks at a dose of 1 mg/kg, then twice a day for 2 weeks at a dose of 0.5 mg/kg, then once a day for 2 weeks at a dose of 0.5 mg/kg, and finally every other day for 2 weeks at a dose of 0.5 mg/kg. At 4 weeks post-injection a second subconjunctival injection of 4 mg of triamcinolone acetonide (Kenalog; 40 mg/mL) was given in the treated eye under topical anesthesia (proparacaine 0.5%; 1 drop; Bausch and Lomb, Bridgewater, NJ) and gentle restraint.

### Dosimetry Analysis

Residual dose formulations collected before and after exposure to the device and cannula from each animal were aliquoted, frozen at −60° C. or colder and later transferred on dry ice for assessment of the vector concentration in the formulated material. The residual dose formulation volume exposed to the device was collected immediately prior to dosing (post-device).

### Observations and Measurements

Investigators involved in acquisition and analysis of all data were masked to the treatment group of each animal. The investigator involved in dose preparation and personnel involved in QC assessment were not masked.

### Efficacy Evaluation

Efficacy endpoints for this study included analysis by funduscopic examination of the progression of disease-related lesions, changes in IS/OS to RPE/tapetum interface distance, and changes in ONL thickness (by in vivo OCT imaging). Investigators and evaluators involved in acquisition and analysis of efficacy data were masked to the treatment group of each animal.

### Funduscopic Examinations

A board-certified veterinary ophthalmologist familiar with the clinical phenotype of canine bestrophinopathies performed in-life funduscopic examinations by indirect ophthalmoscopy at pre-dose, and 1 week (±3 days), 4 weeks (±3 days), 8 weeks (±3 days), 12 weeks (±3 days), post-dose. Fundus photography of both eyes was performed at these same time-points to document the presence of disease-associated retinal lesions, their location, stage of disease, and persistence during the course of the study.

Confocal Scanning Laser Ophthalmoscopy (cSLO) and Optical Coherence Tomography (OCT)

cSLO and OCT imaging was performed with a Spectralis HRA/OCT2 (Heidelberg) unit at pre-dose, 1 week, 4 weeks, 8 weeks, and 12 weeks post-dose.

Qualitative assessment of cSLO and single OCT B-scans was performed at all time-points and progression of disease was documented.

At the pre-dose, and 12-week post-dose time-points, more extensive semi-automated segmentation was performed in light-adapted eyes to produce topographical maps of the distance between inner and outer segments (IS/OS) of photoreceptors and the RPE-tapetum (RPE/T) interface (denominate hereafter OS+), as well as topographical maps of ONL thickness. This was done in test-article injected eyes, vehicle-injected eyes and the un-injected contralateral eyes as previously reported. Results of ONL and OS+ thicknesses within the AAV2/2-BEST1 injected/treated area was compared to results from the corresponding retinal location on the contralateral un-injected eyes, and to vehicle-injected eyes

In addition, ultra-high-resolution (UHR) OCT analysis of treatment effect was assessed in all test-article injected eyes, vehicle-injected eyes, and in the un-injected contralateral eyes. This was performed with a Bioptigen OCT unit at the 12-week post dose time point as follows. Each 44 degree×44 degree raster image obtained in each eye was registered to the corresponding topographic ONL and OS+ thicknesses maps performed with standard resolution OCT. In the injected left eyes, a single b-scan was selected based on the location of the bleb, lesions, treatment related changes so that the selected scan went through the bleb and fovea-like regions (or at least through the visual streak). Major lesions were avoided when possible. An equivalently located b-scan was selected in the un-injected contralateral eyes.

### Safety Evaluations

Safety endpoints for this study included: Clinical observations, ophthalmic examinations, in vivo cSLO/OCT retinal imaging, ERG, clinical pathology panels, immunological and biodistribution studies, as well as gross pathology and microscopic pathology. Investigators and evaluators involved in acquisition and analysis of safety data were masked to the treatment group of each animal.

### Clinical Observations

For each animal enrolled into the study, veterinary records (e.g., body weights, clinic, general health condition, etc.) and comprehensive baseline data documenting the disease condition were collected.

Cage-side observations: An animal caretaker conducted daily cage-side observations for each animal, except on days of detailed observations. Abnormal findings, including any observed eye abnormality (e.g., ocular irritation), were recorded.

Physical examinations: A veterinarian conducted an evaluation of the general health of each animal at least once during the pre-dose phase, prior to dosing on Study Day 0 and weekly (±3 days) thereafter (based on Study Day 0). Abnormal findings, including any observed eye abnormality, were recorded.

Body weight: Measurements were taken at least once during the pre-dose phase, prior to dosing on Study Day 0 and weekly (±3 days) thereafter (based on Study Day 0).

### Ophthalmic Examinations

A board-certified veterinary ophthalmologist performed in-life ophthalmic examinations, including slit lamp biomicroscopy, tonometry, and indirect ophthalmoscopy. Inflammatory changes (conjunctival hyperemia, chemosis, or discharge, anterior chamber flare and cellularity/precipitates in vitreous) and changes in transparency of ocular media (cornea/lens/vitreous) were graded as none, mild, moderate or severe. Intraocular pressure (IOP) measurements were recorded for each eye with a rebound tonometer (iCare Tonovet®, Vantaa, Finland). Examinations were conducted at pre-dose, and 1 week (±3 days), 4 weeks (±3 days), 8 weeks (±3 days), 12 weeks (±3 days), post-dose, and all abnormalities were noted. Fundus photography of both eyes was performed to document the retinal appearance at pre-dose, immediately after the subretinal injection, and at 1 week (±3 days), 4 weeks (±3 days), 8 weeks (±3 days), 12 weeks (±3 days) post-dose.

Confocal Scanning Laser Ophthalmoscopy (cSLO) and Optical Coherence Tomography (OCT)

cSLO/OCT retinal imaging was performed with a Spectralis HRA/OCT2 (Heidelberg) unit at pre-dose, 1 week, 4 weeks, 8 weeks, and 12 weeks post-dose. Images were examined for detection of retinal lesions that could be associated with the surgical procedure, and/or test-article.

### Electroretinography (ERG)

Recordings were conducted at pre-dose and at 11 weeks post-dose utilizing an Espion E3 electroretinography unit (Diagnosys LLC, Lowell, MA). In brief, pupils were dilated with topical atropine sulfate (1%), tropicamide (1%) and phenylephrine (10%). After induction with intravenous propofol, dogs were maintained under general inhalation anesthesia (isoflurane). Full-field flash electroretinography was performed on both eyes using a custom-built Ganzfeld dome fitted with the LED stimuli of a Color Dome stimulator (Diagnosys LLC, Lowell, MA). After 20 minutes of dark adaptation, rod- and mixed rod-cone-mediated responses to single 4-ms white flash stimuli of increasing intensities were recorded. Following 5 minutes of white light adaptation, cone-mediated signals to a series of single flashes and to 29.4-Hz flicker stimuli were recorded. Amplitudes of the b-wave of the dark-adapted rod response, of the a- and b-waves of the dark-adapted rod-cone mixed response, and the trough-to-peak amplitudes of the light-adapted single flash and 29.4 Hz flicker stimuli were measured. The results of the AAV2/2-BEST1 injected eyes were compared to the vehicle-treated eyes, and to the un-injected contralateral eyes.

### Clinical Pathology

Investigators and evaluators involved in the collection of blood samples and analysis of the clinical pathology data were masked to the treatment group of each animal.

Blood samples for hematology, coagulation, and clinical chemistry panels were obtained at pre-dose, 1 week (±3 days), 4 weeks (±3 days), 8 weeks (±3 days), and 12 weeks (3 days) post-dose. Animals were fasted overnight for scheduled collections. Blood samples were collected via the jugular vein; an alternate vein was used if necessary. The site of blood collection was documented.

Anticoagulants used were sodium citrate for the coagulation sample and tripotassium-EDTA for hematology samples. Samples for clinical chemistry were collected without an anticoagulant. The serum and the plasma were stored frozen at −80° C. until analysis, while the EDTA samples for hematology were stored in the refrigerator at 4° C. until analysis.

The minimum volumes collected were:
For hematology: 1 mL of blood
For routine coagulation profile: 1.3 mL of blood (1:9 ratio of citrate to blood)
Serum chemistry: ˜1 ml of blood to obtain ˜0.5 mL of serum

### Clinical Pathology Tests Performed are Listed Below

### Hematology:

Red blood cell (erythrocyte) count
Hemoglobin
Hematocrit
Mean corpuscular volume
Mean corpuscular hemoglobin
Mean corpuscular hemoglobin concentration
Reticulocyte count
Platelet count
White blood cell (leukocyte) count
Differential blood cell count
Blood smear evaluation

### Coagulation:

Prothrombin time activated
Partial thromboplastin time

### Clinical Biochemistry:

Glucose
Urea nitrogen
Creatinine
Total protein
Albumin
Globulin
Albumin:globulin ratio
Cholesterol
Total bilirubin
Alanine aminotransferase
Aspartate aminotransferase
Alkaline phosphatase
Gamma-glutamyltransferase
Calcium
Inorganic phosphorus
Sodium
Potassium
Chloride
Triglycerides

### Immunological Studies

Serum samples were obtained at pre-dose, 1 week (±3 days), 4 weeks (±3 days), 8 weeks (±3 days), 12 weeks (±3 days) post dose, and immediately prior termination at 13 weeks post-dose. Two aliquots (0.5 mL each) were collected for each time point and stored at ≤−60° C. One aliquot from each animal and time point was shipped to the Sponsor's test site for determination of AAV2 antibody titers.

Viral shedding Biodistribution Studies A validated viral shedding/biodistribution assay utilizing qPCR and targeting a distinct sequence of the human BEST1 transgene was developed at CRL by the Sponsor.

Samples listed in Table 2 were collected during the in-life period for viral shedding (tears, saliva, urine) and biodistribution (whole blood, aqueous humor) studies.

TABLE 2
Fluids Collected for Determination of Viral
Shedding and Biodistribution Analysis

| | |
|---|---|
| Tears (both eyes) | Pre-dose, 1 week (±3 days), 4 weeks |
| | (±3 days), 8 weeks (±3 days) |
| | post-dose, and immediately prior |
| | termination (13 weeks post-dose). |
| Saliva | Pre-dose, 1 week (±3 days), 4 weeks |
| | (±3 days), 8 weeks (±3 days) |
| | post-dose, and immediately prior |
| | termination (13 weeks post-dose). |
| Urine | Pre-dose, 1 week (±3 days), 4 weeks |
| | (±3 days), 8 weeks (±3 days) |
| | post-dose, and immediately prior |
| | termination (13 weeks post-dose). |
| Whole blood | Pre-dose, 1 week (±3 days), 4 weeks |
| | (±3 days), 8 weeks (±3 days) |
| | post-dose, and immediately prior |
| | termination (13 weeks post-dose). |
| Aqueous humor | Pre-dose (OU), immediately post-dose |
| | (OS), 4 weeks ± 3 days (OU), and |
| | immediately prior termination at 13 |
| | weeks post-dose (OU). |

Other tissues collected at the time of termination for biodistribution analysis are listed in Table 3 below.

TABLE 3
Tissues Collected for Biodistribution Analysis

| | | |
|---|---|---|
| Eyelid and | Pancreas* | Skin |
| periocular tissues | | (abdomen) |
| (pair) | | |
| Bulbar conjunctiva | Jejunum | Mandibular lymph |
| (pair) | | nodes (pair) |
| Lacrimal | Testes or | Parotid |
| glands | ovaries | gland |
| (pair) | (pair)* | (pair) |
| Heart* | Liver* | Brain* |
| | | Optic tract (pair)** |
| | | Lateral geniculate nucleus |
| | | (pair)** |
| | | Cortex (pair)** |
| | | Cerebellum (pair)** |
| Lungs (pair)* | Spleen* | Gross lesions |
| Kidneys (pair)* | Skeletal muscle | |
| | (R. quadriceps) | |

*Samples weighed.
**Collected once the brain was fixed.
For paired organs, samples of both left and right were collected and stored for potential future analysis.

### Gross Pathology and Microscopic Pathology

### Terminal Sacrifice

Animals were sacrificed at 13 weeks post-dose. Animals were euthanized with an overdose of a commercial pentobarbital-based euthanasia solution (Euthasol; Virbac USA) that was administered via intravenous injection after exsanguination. The procedure of exsanguination was conducted by ultrasonic-guided intracardiac puncture and blood extraction. The necropsy procedure started as soon as death was confirmed by the absence of a heartbeat on chest auscultation and ultrasound imaging.

Necropsy Procedures For all scheduled deaths, comprehensive gross pathology examinations were performed, and tissues were collected in appropriate fixative for histopathology evaluation, including eyes (with optic nerve), other peri-ocular tissues and any gross lesions from all animals, as well as selected systemic organs/tissues, which are listed in Table 4.

### Core Tissues for Histopathology Examination

Tissues listed in Table 5. Additional Tissues Collected for Potential Histopathology Examination were collected and preserved in appropriate fixative. Samples were labeled with the following information: Study number, date, dog ID, tissue name, purpose (for qPCR or histopathology), and side (left or right) for potential future evaluation.

Tissues for biodistribution analysis were collected according to a study-specific procedure in a manner to avoid cross contamination using ultra-clean techniques. With the exception of the ocular globes that were collected within a few minutes following euthanasia to avoid any autolysis of the retinal tissues, the organs that are less likely to contain vector DNA were sampled at the beginning of the necropsy, while those tissues more likely to contain vector DNA were collected towards the end of the procedure.

The tissues were washed with fresh phosphate-buffered saline, which reduces contamination with transduced circulating blood cells, especially peripheral blood mononuclear cells (PBMC). Tissue samples were flash-frozen in liquid nitrogen and then stored at ≤−60° C.

The 1.8-mL sample collection tubes were labeled and used for collection of tissues for biodistribution analysis. All tissue samples collected for biodistribution analysis were weighed in the collection tubes (excluding the tared weight of the empty sample tube for each sample), with the exception of liquid samples such as urine, saliva, tears and plasma.

TABLE 4
Core Tissues for Histopathology Examination

| | | |
|---|---|---|
| Injected eye with | Pancreas* | Skin |
| optic nerve | | (abdomen) |
| Uninjected eye with | Jejunum | Mandibular lymph |
| optic nerve | | nodes (pair) |
| Eyelid and periocular | Testes or | Parotid |
| tissues | ovaries (pair)* | gland (pair) |
| (pair) | | |
| Heart* | Liver* | Brain* |
| | | Optic chiasma |
| | | Optic tract (pair)** |
| | | Lateral geniculate |
| | | nucleus (pair)** |
| | | Cortex (pair)** |
| | | Cerebellum (pair)** |
| Lungs (pair)* | Spleen* | Gross lesions |
| Kidneys (pair)* | Skeletal muscle | |
| | (R. quadriceps) | |

*Organs weighed.
**Collected once the brain was fixed.
For paired organs, samples of both left and right were collected and stored for potential future examination.

TABLE 5
Additional Tissues Collected for Potential
Histopathology Examination

| | | |
|---|---|---|
| Adrenal gland (pair)* | Gall bladder | Stomach |
| Aorta | Ileum | Tongue |
| Bone marrow | Lymph node | Thymus |
| smear | (mesenteric) | |
| Cecum | Peripheral | Thyroid/ |
| | nerve (sciatic) | parathyroid gland |
| Colon | Pituitary gland | Urinary bladder |
| Diaphragm | Prostate | Ureters (pair) |
| Duodenum | Rectum | Uterine horns (pair) |
| Epididymis (pair) | Spinal cord (cervical) | Vagina |
| Esophagus | Spinal cord (lumbar) | |
| Femur with bone marrow | Spinal cord (thoracic) | |

*Organs weighed.

Terminal body weights were recorded post exsanguination and prior to necropsy.

Macroscopic examinations were conducted by a board-certified veterinary pathologist.

Necropsies included an examination of the external features of the carcass; external body orifices; abdominal, thoracic, and cranial cavities; organs and tissues.

Eyes were enucleated a few minutes after euthanasia. Part of the intra-orbital optic nerve and an aqueous humor paracentesis from each eye were collected and frozen for future qPCR analysis. Ocular globes with the proximal optic nerve were fixed in an alcohol Bouin's solution for 72 hours before being transferred to 70% ethanol until paraffin embedding.

Paired organs and the liver with gall bladder were weighed together. Organs from which samples were taken for qPCR analysis were weighed following qPCR sample collection, with the exception of the brain. Tissues designated for biodistribution analyses were collected with a fresh disposable or sterile DNA-free set of instruments as soon as possible following sacrifice (except brain samples).

Samples from the following tissues were collected (prior to fixation) for potential biodistribution analysis (approximate 5 mm³, when possible): eyelid and periocular tissues (2), bulbar conjunctiva (2), lacrimal glands (2), heart, lung (2), kidney (2), pancreas, jejunum, ovaries (2) or testis (2), liver, spleen, skeletal muscle (R. quadriceps), skin (abdomen), mandibular lymph node (2), parotid gland (2). Samples of the same tissues were collected for histology (except the aqueous humor, bulbar conjunctiva and lacrymal gland). The tissues (when present) in the above tables from each animal were preserved in an appropriate fixation solution.

Tissue sampling for biodistribution assessment was performed using fresh disposable or sterile DNA-free sets of material and containers and changing of gloves for each sample to minimize cross contamination.

Brains were removed at the end of the procedure and weighed (using a clean weigh dish). The optic chiasma and the pituitary gland were collected for histopathology only, and the brains were sectioned sagittally (along the longitudinal fissure) with a new DNA-free blade for each animal and collected into neutral-buffered 10% formalin and allowed to fix for 24-72 hours.

The following regions of the brain were dissected to obtain samples for histopathology and biodistribution analysis: optic tract (left), lateral geniculate nucleus (LGN [left]), occipital cortex (left), cerebellum (left), optic tract (right), LGN (right), occipital cortex (right), and cerebellum (right). The size of each sample was approximately 5 mm³ (size was not documented). Samples for qPCR were transferred to 70% ethanol, and samples for histopathology were paraffin-embedded sectioned and stained with H&E.

Samples in 70% ethanol collected for qPCR analysis from fixed tissues (brain samples) were stored under ambient conditions until shipped (under ambient conditions) for analysis.

Additional tissue samples for potential histopathology were also collected and preserved in the appropriate fixation solution.

Lesions identified during gross pathology examination were fixed in desired fixative and stained with H&E.

Histopathology Tissues from each animal were collected in an appropriate fixative, paraffin-embedded and sectioned, and slides were prepared and stained with hematoxylin and eosin. At least two sections per tissue were examined microscopically for anatomic pathology. The remaining tissues were preserved in fixative for future potential examination.

Paraffin-embedded H&E-stained section of ocular globes were examined for any potential histological lesions of inflammation and/or toxicity, and measurements of ONL thickness were analyzed as described.

### Mortality and Ophthalmic Findings

Categorical outcome measures such as ophthalmic findings were summarized by treatment group in a frequency table.

For continuous outcome measures (e.g., body weights, IOPs), descriptive analysis [mean and standard deviation (SD)] was performed. The comparison of means across all groups was performed using one-way analysis of variance (ANOVA) followed by linear trend analysis. If there was a significant difference, post-hoc pairwise comparisons (of the 2 dose groups vs. vehicle group) with correction for multiple comparisons will be made. Within each treatment group, a paired t-test was performed to compare the mean value of IOP, ERG amplitudes, ONL thickness, and OS+ thickness between injected eyes, and the un-injected contralateral eyes

All statistical analyses were performed in SAS v9.4 (SAS Institute Inc, Cary, NC).

### Body and Organ Weights

Group means and standard deviations (SD) were calculated for terminal body weight (recorded immediately after euthanasia and before exsanguination), organ weight, organ-to-body weight ratios and organ-to-brain weight ratios. The comparisons of means across all groups were performed using one-way analysis of variance (ANOVA) followed by linear trend analysis. When there was a significant difference, post-hoc pairwise comparisons (of the 2 dose groups vs. vehicle control group) with correction for multiple comparisons were made using the Bonferroni test.

All statistical analyses were performed in SAS v9.4 (SAS Institute Inc, Cary, NC).

### Clinical Pathology

For these continuous outcome measures (clinical pathology values), descriptive analyses (mean, SD) were performed. The comparisons of means across all treatment groups were performed using one-way analysis of variance (ANOVA) followed by linear trend analysis. When there was a significant difference, post-hoc pairwise comparisons (of the 2 dose groups vs. vehicle group) with correction for multiple comparisons were made using the Bonferroni test.

All statistical analyses were performed in SAS v9.4 (SAS Institute Inc, Cary, NC).

### Gross Pathology, Microscopic Pathology

Abnormal findings were described, graded in severity and reported for each individual animal. Findings that could be associated with test article-related toxicity were summarized in a tabular format using a heat map to document severity of the lesions.

### ERG

For the continuous ERG outcome measures (amplitudes of ERG waves) measured at pre-dose and 11 weeks post-dose, descriptive analysis (mean, SD) was performed. To account for potential animal-to-animal variability of ERG amplitudes, the mean difference between the injected (OS) and un-injected (OD) eyes was calculated for each of the three treatment groups. The comparison of means for the inter-eye difference (between injected eye and un-injected fellow eye) across all treatment groups was performed using one-way analysis of variance (ANOVA) followed by a linear trend analysis. When there was a significant difference, post-hoc pairwise comparisons (of the 2 dose groups vs. vehicle group) with correction for multiple comparisons were made using the Bonferroni test. For each treatment group, the paired t-test test was performed to compare the means between injected eyes and the un-injected contralateral eyes.

All statistical analyses were performed in SAS v9.4 (SAS Institute Inc, Cary, NC).

cSLO and OCT data

Qualitative post acquisition analysis was performed on cSLO and OCT data collected at pre-dose, 1 week, 4 weeks, 8 weeks, and 12 weeks post-dose included staging of disease within the treated and untreated areas of injected eyes, and of equivalent areas of contralateral injected eyes.

Standard resolution OCT data collected at pre-dose and at 12 weeks post-dose underwent more extensive analysis and included generation of ultra-wide-angle composite images from the infrared images, registration of each of approximately 1500 to 1800 B-scans to their corresponding retinal location across each retina, resampling of the registered OCT data into regularly spaced bins of a coordinate system centered on the optic nerve, as previously reported²⁹. All longitudinal reflectivity profiles (LRPs) collected in each bin were aligned and averaged before performing manual segmentation of four boundaries: two boundaries at OPL and ELM that define outer nuclear layer (ONL) thickness, and an additional two boundaries at IS/OS and RPE/T to define the combined thickness of the outer segments and the subretinal space (termed OS+). ONL and OS+ thicknesses were assigned to a pseudocolor scale to generate topographic images. Injection blebs from the post-surgery images, location of the fovea-like area and the boundary of the tapetum were overlaid on resulting maps. ONL and OS+ thicknesses results at pre-dose and at 12 weeks post-dose within the AAV2/2-BEST1 treated area were sampled and compared to results from vehicle-injected eye, as well as from an equivalent area of the contralateral un-injected eye (OD). Specifically, for each treatment group, the inter-eye difference (IED; OS-OD) in ONL and OS+ thicknesses in the treated and untreated areas at 12 weeks post-dose were calculated and normalized by the IED (OS-OD) in ONL and OS+ thicknesses at pre-dose.

For UHR-OCT data collected only at 12 weeks post-dose, the OPL, ELM, IS/OS, and RPE/T layers were segmented in the selected b-scans and plotted as a function of distance from the fovea-like region along the horizontal meridian. ONL thickness was calculated as the difference between OPL and ELM layers, and OS+ thickness as the difference between IS/OS and RPE/T layers. The mean (+/−SD) of the inter-eye difference (IED) of the two thicknesses within the bleb region was provided as a quantitative measure of the intervention for each animal. In addition, qualitative examination of the outer segments and subretinal space was performed and compared between injected and un-injected eyes.

For these continuous outcome measures (ONL thickness, and OS+ thickness by OCT), descriptive analysis was performed using mean, and standard deviation (SD). To account for potential animal to animal variability of ONL thickness, the mean difference between the injected (OS) and un-injected (OD) eyes was calculated for each of the three treatment groups. The comparison of means for the inter-eye difference (injected eye—un-injected fellow eye) across all treatment groups was performed using one-way analysis of variance (ANOVA) followed by linear trend analysis. If there was a significant difference, post-hoc pairwise comparisons (of the 2 dose groups vs. vehicle group) with correction for multiple comparisons was made. Within each treatment group, the paired t-test test was performed to compare the means between AAV2/2-BEST1 injected eyes, the vehicle-injected eyes, and their un-injected fellow eyes.

All statistical analyses were performed in SAS v9.4 (SAS Institute Inc, Cary, NC).

### ONL Thickness Assessed by Histology

H&E-stained paraffin sections of the ocular globes that included the optic nerve head were sectioned through the bleb/treated area (or equivalent area in contralateral un-injected eyes) and were digitally scanned (Aperio digital pathology scanner, Leica). Manual measurements of the ONL thickness were performed using the Aperio ImageScope software at regular (1-mm) intervals extending from the edge of the optic nerve head to the ora serrata. Linear graphs of ONL thickness were constructed for both eyes, representing the measurements acquired in the superior and inferior plane of the previously defined sections. Based on the location of the bleb, the mean ONL thickness measured from five (5) locations within the bleb/treated area were calculated. Similarly, five (5) equivalent locations were selected in the contralateral (OD) un-injected eyes to calculate the mean ONL thickness in these eyes. To account for potential animal to animal variability of ONL thickness, the mean difference between the injected (OS) and un-injected (OD) eyes was calculated for each of the three groups treated with the test article or vehicle and compared using one-way analysis of variance (ANOVA) followed by linear trend analysis. When there was a significant difference, post-hoc pairwise comparisons (of the 2 dose groups vs. vehicle group) with correction for multiple comparisons were made using the Bonferroni test. Within each treatment group, the paired t-test was performed to compare the means between injected eyes and their un-injected contralateral eyes.

All statistical analyses were performed in SAS v9.4 (SAS Institute Inc, Cary, NC).

### Results

### Progression of Disease by Funduscopic Examination

BEST1-associated lesions observed by indirect ophthalmoscopy and cSLO were staged and their progression in the treated area of the injected eyes compared to the progression of lesions observed in the untreated area of the injected eyes, and equivalent treated area of the contralateral un-injected eyes. Note: the earliest stage that can be diagnosed by funduscopic examination or cSLO is Stage II.

### Un-Injected Eyes

In 3 out of 9 dogs (Animal IDs No. EML34-OD, LH39-OD, CTL1-OD), BEST1-associated lesions (Stage III and/or II) that were detected at pre-dose did not progress during the course of the study. In 2 out of 9 dogs (Animal ID Nos LH37, CT5) stage II lesions appeared during the course of the study. No BEST1-associated lesions appeared in the remaining 4 out of 9 dogs.

### Vehicle-Injected Eyes

In the single dog (Animal ID No. EML34-OS) from this treatment group that had funduscopically visible lesions (stage II and III) at pre-dose, lesions persisted in the treated area. No lesions appeared in the treated area in the two remaining dogs (CT4-OS, and LH37-OS).

Low Dose: 1.4×10⁹ vg/Eye AAV2/2-BEST1 Treated Eyes

In the single dog (Animal ID No. LH39-OS) from this treatment group that had funduscopically visible lesions (stage III) at pre-dose, lesions persisted in the untreated area but reverted to a stage II appearance in the treated area. No lesions appeared in the two remaining dogs (CTL3-OS, and ECT2-OS). Thus, evidence for some potential efficacy of low dose AAV2/2-BEST1 could be detected in 1 out of 3 dogs.

High Dose: 4.5×10⁹ vg/Eye AAV2/2-BEST1 Treated Eyes

In Animal ID No. CT5-OS, numerous multifocal Stage II lesions appeared at 8 weeks post-dose but this was only seen in the untreated area. No lesions developed in the treated area. In Animal ID No. CTL1-OS a single focal Stage III lesion at pre-dose in the treated area disappeared following treatment. However, all stage II and III lesions in the untreated area persisted. No lesions appeared in the remaining dog (EIL35-OS). Thus, evidence for some potential efficacy of high dose AAV2/2-BEST1 could be detected in 2 out of 3 dogs.

### OS+ Thickness Assessed by Optical Coherence Tomography

Results from two different approaches (measurements from standard resolution OCT-derived maps, and measurements from single HR OCT B-scans) used to independently assess restoration of the RPE-PR interface following treatment are presented below. Results from individual animals are also available in Table 6 and Table 7.

TABLE 6
Individual mean OS+ thickness (in μm) from Spectralis OCT maps in the treated (Tx) and
untreated (UnTx) areas of the injected eyes (OS) and equivalent treated (Tx-eq) and equivalent
untreated (UnTx-eq) in the contralateral un-injected (OD) eyes at pre-dose and 12 weeks post-dose.

| | | OS (Injected) | | | | OD (Un-injected) | | | |
|---|---|---|---|---|---|---|---|---|---|
| | | Tx | Tx | UnTx | UnTx | Tx-eq | Tx-eq | UnTx-eq | UnTx-eq |
| Dog ID | Treatment | Pre-dose | 12 wks PD | Pre-dose | 12 wks PD | Pre-dose | 12 wks PD | Pre-dose | 12 wks PD |
| LH37 | Vehicle | 50.7 | 49.6 | 49.5 | 47.2 | 46.4 | 46.8 | 46.9 | 45.0 |
| CT4 | | 42.6 | 44.9 | 42.4 | 47.8 | 41.6 | 44.6 | 44.0 | 46.7 |
| EML34 | | 79.4 | 72.3 | 69.9 | 63.5 | 70.7 | 61.3 | 64.1 | 54.1 |
| CTL3 | Low-dose | 49.1 | 46.6 | 47.9 | 53.4 | 52.7 | 60.6 | 50.3 | 57.7 |
| ECT2 | AAV2/2- | 52.1 | 43.1 | 52.6 | 58.4 | 53.7 | 53.3 | 51.1 | 50.7 |
| LH39 | BEST1 | 59.2 | 37.7 | 55.7 | 42.7 | 53.5 | 45.2 | 47.7 | 42.0 |
| CT5 | High-dose | 46.0 | 40.6 | 45.7 | 53.2 | 51.9 | 58.9 | 49.4 | 49.8 |
| EML35 | AAV2/2- | 43.8 | 43.0 | 42.4 | 48.4 | 44.9 | 49.1 | 43.8 | 46.0 |
| CTL1 | BEST1 | 62.1 | 43.0 | 60.9 | 57.0 | 60.4 | 56.5 | 66.6 | 57.5 |

PD: post-dose

TABLE 7
Individual mean OS+ thickness (in μm) from Bioptigen
UHR OCT-b scans in the treated (Tx) area of the injected
(OS) eyes and in the equivalent treated (Tx-eq) of the
contralateral un-injected (OD) eyes at 12 weeks post-dose.

| | | Injected (OS) | Un-injected (OD) |
|---|---|---|---|
| Dog ID | Treatment | Tx | Tx-eq |
| LH37 | Vehicle | 46.3 | 42.5 |
| CT4 | | 41.3 | 39.2 |
| EML34 | | 83.9 | 73.2 |
| CTL3 | Low-dose | 37.4 | 50.0 |
| ECT2 | AAV2/2-BEST1 | 29.7 | 44.9 |
| LH39 | | 30.4 | 70.5 |
| CT5 | High-dose | 32.4 | 62.3 |
| EML35 | AAV2/2-BEST1 | 33.8 | 40.1 |
| CTL1 | | 35.5 | 52.5 |

### Un-Injected Eyes

### OCT Map Analysis of OS+ Thickness:

In 2 out of 9 un-injected eyes (Animal ID Nos LH37-OD, ECT2-OD) no changes in OS+ thickness were seen between the pre-dose and 12 weeks post-dose time points. In 3 out of 9 eyes un-injected eyes (Animal ID Nos EML34-OD, LH39-OD) there was a decrease in OS+ thickness, while an increase was seen in the remaining 4 out of 9 dogs (Animal ID Nos CT4-OD, CTL3-OD, CT5-OD, EML25-OD). Variability of the directional changes in OS+ thickness across these animals could be explained by differences in disease progression, differences in duration of light exposure during image acquisition which has been shown to affect the photoreceptor-RPE interface²⁹, or a combination of the above.

### HR OCT Single B-Scan Analysis of OS+ Thickness:

In 3 out of 9 un-injected eyes (Animal ID Nos. LH37-OD, CT4-OD, EML35-OD), OS+ thickness at 12 weeks post-dose was within or slightly above the 95% CI of normal dogs. In all other 6 out of 9 dogs, OS+ thickness was significantly higher than in normal dogs.

### Vehicle-Injected Eyes

### OCT Map Analysis of OS+ Thickness:

In 2 out of 3 vehicle-injected eyes (Animals ID Nos LH37-OS and CT4-OS) there were no changes in OS+ thickness seen between the pre-dose and 12 weeks post-dose time points. In EML34-OS a reduction was seen in both treated and untreated areas.

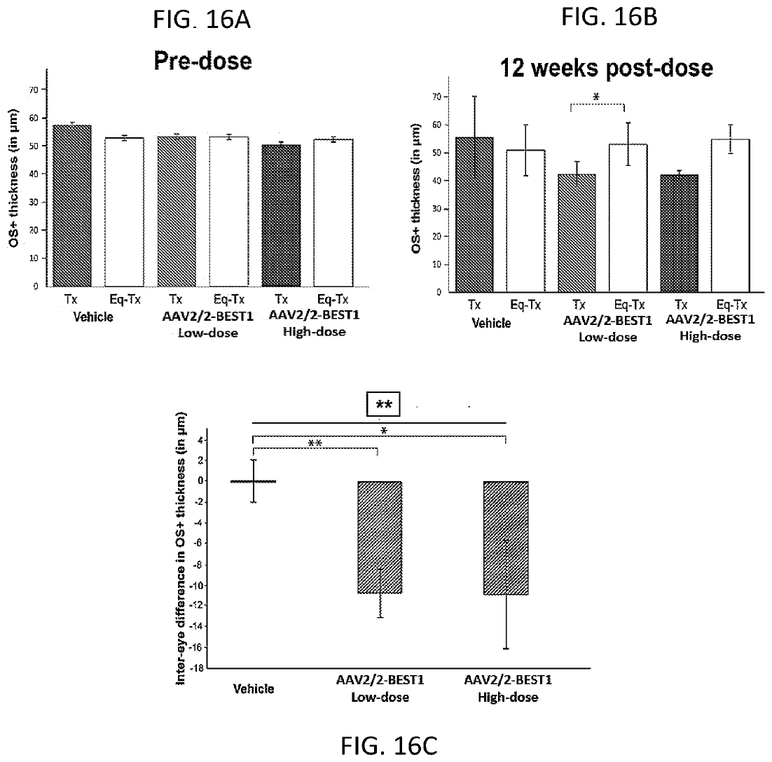

No significant differences were seen when comparing the mean ONL thickness in the treated area of the vehicle-injected (OS) eyes to that of the equivalent treated area of the contralateral un-injected (OD) eyes at both pre-dose and 12 weeks-post dose (FIG. 16A).

### HR OCT Single B-Scan Analysis of OS+ Thickness:

In 3 out of 3 vehicle-injected eyes, OS+ thickness in the treated area at 12 weeks post-dose was prominently above or slightly above the 95% CI of normal dogs. No significant differences were seen when comparing the mean OS+ thickness in the treated area of the vehicle-injected (OS) eyes to that of the equivalent treated area of the contralateral un-injected (OD) eyes.

In summary, taken together, the combined results of both methods did not show any efficacy with the vehicle control article.

$$Low Dose : 1.4 \times {10}^{9} vg / eye AAV 2 / 2 - BEST 1 Treated Eyes$$

### OCT Map Analysis of OS+ Thickness:

In 1 out of 3 low-dose AAV2/2-BEST1-treated eyes (Animal ID No. CTL3-OS) OS+ thickness remained stable in the treated area but increased in the untreated area between the pre-dose and 12 weeks post-dose time points. In 1 out of 3 low-dose AAV2/2-BEST1-treated eyes (Animal ID No. ECT2-OS) OS+ thickness decreased in the treated area but increased in the untreated area. Finally, in 1 out of 3 eyes (Animal ID No. LH39-OS) OS+ thicknesses decreased in both the treated and untreated area but the effect was lesser in the later. This could have been the result of expansion of the bleb beyond its initial border due to the generalized microdetachment present in this eye at pre-dose.

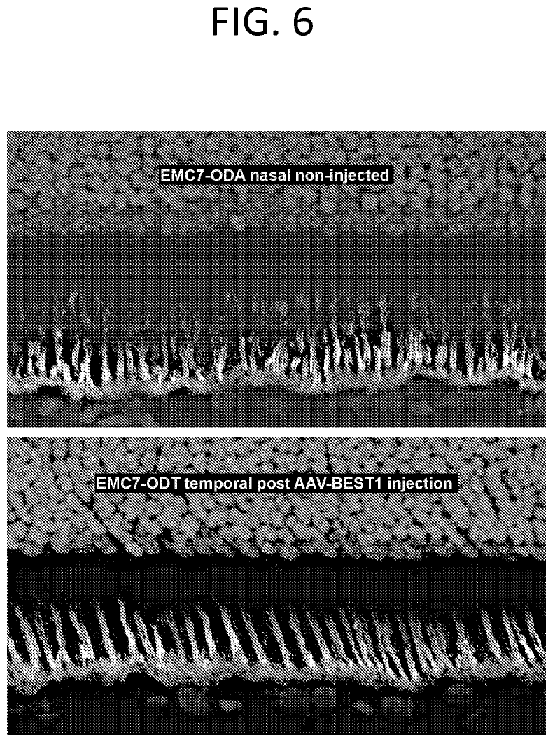

No significant differences were seen when comparing the mean OS+ thickness in the treated area of the low-dose AAV2/2-BEST1-injected (OS) eyes to that of the equivalent treated area of the contralateral un-injected (OD) eyes at pre-dose (FIG. 6A). Similarly, no inter-eye differences were found between untreated and equivalent untreated areas at pre-dose and 12 weeks post-dose. However, at 12 weeks post-dose OS+ thickness in the treated area was significantly (p=0.03) lower (42.5±4.5 μm) than in the equivalent-treated area of the contralateral eye (53±7.7 μm) (FIG. 6B).

### HR OCT Single B-Scan Analysis of OS+ Thickness:

In 3 out of 3 low-dose AAV2/2-BEST1-treated eyes, OS+ thickness in the treated area at 12 weeks post-dose was within the 95% CI of normal dogs.

A quantitatively large difference in OS+ thickness was found when comparing the treated area of the low-dose AAV2/2-BEST1-injected (OS) eyes (32.5±4.2 m) to that of the equivalent treated area of the contralateral un-injected (OD) eyes (55.1±13.6 μm), however the small group size may have precluded reaching statistical significance.

In summary, taken together, the combined results of both methods indicate that AAV2/2-BEST1 at a low dose of 1.4×10⁹ vg/eye stabilized or reduced OS+ thickness to normal values, thus suggesting a positive therapeutic effect.

$$High Dose : 4.5 \times {10}^{9} vg / eye AAV 2 / 2 - BEST 1 Treated Eyes$$

### OCT Map Analysis of OS+ Thickness:

In 1 out of 3 high-dose AAV2/2-BEST1-treated eyes (Animal ID No. EML35-OS) OS+ thickness remained stable in the treated area but increased in the untreated area between the pre-dose and 12 weeks post-dose time points. In 1 out of 3 high-dose AAV2/2-BEST1-treated eyes (Animal ID No. CT5-OS) OS+ thickness decreased in the treated area but increased in the untreated area. Finally, in 1 out of 3 eyes (Animal ID No. CTL1-OS) OS+ thicknesses decreased in both the treated and untreated area but the effect was lesser in the later. This could have been the result of expansion of the bleb beyond its initial border due to the generalized microdetachment present in this eye at pre-dose.

No significant differences were seen when comparing the mean OS+ thickness in the treated area of the high-dose AAV2/2-BEST1-injected (OS) eyes to that of the equivalent treated area of the contralateral un-injected (OD) eyes at pre-dose (FIG. 6A). Similarly, no inter-eye differences were found between untreated and equivalent untreated areas at pre-dose and 12 weeks post-dose. However, at 12 weeks post-dose OS+ thickness in the treated area was lower (42.2±1.4 μm) than in the equivalent-treated area of the contralateral eye (54.8±5.1 μm) (FIG. 6B) but the difference was not statistically significant. The small group size may have precluded reaching statistical significance.

### HR OCT Single B-Scan Analysis of OS+ Thickness:

In 3 out of 3 high-dose AAV2/2-BEST1-treated eyes, OS+ thickness in the treated area at 12 weeks post-dose was within the 95% CI of normal dogs. A quantitatively large difference in OS+ thickness was found when comparing the treated area of the high-dose AAV2/2-BEST1-injected (OS) eyes (33.9±1.6 μm) to that of the equivalent treated area of the contralateral un-injected (OD) eyes (51.7±11.1 μm), however the small group size may have precluded reaching statistical significance.

In summary, taken together, the combined results of both methods indicate that AAV2/2-BEST1 at a high dose of 4.5×10⁹ vg/eye stabilized or reduced OS+ thickness to normal values suggesting a positive therapeutic effect.

### Comparison of OS+ Thickness Across Treatment Groups

### OCT Map Analysis of OS+ Thickness:

Quantitative comparison across treatment groups of the mean inter-eye differences in OS+ thickness at 12 weeks post-dose (normalized to the IED at pre-dose) between the treated area of the injected eyes (OS) and the equivalent treated area of the un-injected contralateral eyes (OD) showed an overall statistical difference across all groups (p=0.01), and post-hoc comparison showed a statistically significant reduction in the low-dose AAV2/2-BEST1 (−10.7±2.3 μm; p=0.004) and in the high-dose AAV2/2-BEST1 (−10.9±5.2 μm; p=0.004) groups when compared to vehicle (FIG. 6C).

### HR OCT Single B-Scan Analysis of OS+ Thickness:

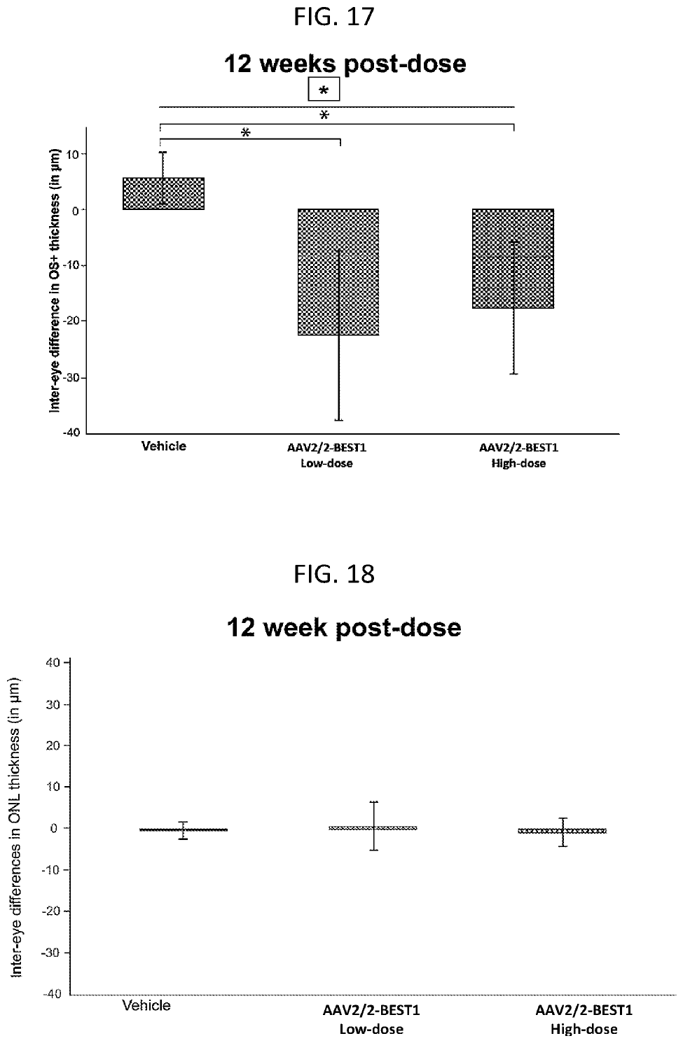

Quantitative comparison across treatment groups of the mean inter-eye differences in OS+ thickness at 12 weeks post-dose between the treated area of the injected eyes (OS) and the equivalent treated area of the un-injected contralateral eyes (OD) showed an overall statistically significant (p=0.049) difference, and post-hoc comparison showed a statistically significant reduction in the low-dose AAV2/2-BEST1 (−22.6±15.2 μm; p=0.04) and in the high-dose AAV2/2-BEST1 (−17.7±11.8 μm; p=0.03) groups when compared to vehicle (FIG. 17).

In summary, both methods confirmed that both doses of AAV2/2-BEST1 (low and high) result in reduction of OS+ thickness.

### ONL Thickness Assessed by Optical Coherence Tomography

Results from two different approaches (measurements from standard resolution OCT-derived maps, and measurements from single HR OCT-B scans) used to independently assess restoration of ONL thickness following treatment are presented below.

TABLE 8
Individual mean ONL thickness (in μm) from Spectralis OCT maps in the
treated (Tx) and untreated (UnTx) areas of the injected eyes (OS) and
equivalent treated (Tx-eq) and equivalent untreated (UnTx-eq) in the
contralateral un-injected (OD) eyes at pre-dose and 12 weeks post-dose.

| | | OS (Injected) | | | | OD (Un-injected) | | | |
|---|---|---|---|---|---|---|---|---|---|
| | | Tx | Tx | UnTx | UnTx | Tx-eq | Tx-eq | UnTx-eq | UnTx-eq |
| Dog | | Pre- | 12 wks | Pre- | 12 wks | Pre- | 12 wks | Pre- | 12 wks |
| ID | Treatment | dose | PD | dose | PD | dose | PD | dose | PD |
| LH37 | Vehicle | 58.5 | 57.2 | 55.2 | 54.6 | 57.6 | 57.0 | 55.6 | 54.8 |
| CT4 | | 65.4 | 56.8 | 66.8 | 53.7 | 67.0 | 61.6 | 64.3 | 55.2 |
| EML34 | | 54.1 | 56.9 | 51.3 | 51.1 | 55.9 | 55.5 | 53.7 | 55.8 |
| CTL3 | Low-dose | 62.5 | 58.0 | 60.8 | 49.7 | 61.8 | 54.7 | 61.2 | 50.4 |
| ECT2 | AAV2/2- | 51.3 | 50.3 | 47.7 | 45.1 | 48.6 | 51.3 | 43.1 | 46.6 |
| LH39 | BEST1 | 47.1 | 46.2 | 41.2 | 43.4 | 44.9 | 48.6 | 45.7 | 46.1 |
| CT5 | High-dose | 61.1 | 56.3 | 60.2 | 48.2 | 59.8 | 58.9 | 57.9 | 52.4 |
| EML35 | AAV2/2- | 65.2 | 52.9 | 60.9 | 50.7 | 62.6 | 54.1 | 61.8 | 50.3 |
| CTL1 | BEST1 | 51.1 | 51.2 | 54.7 | 48.8 | 45.6 | 50.3 | 57.5 | 56.7 |

PD: post-dose

TABLE 9
Individual mean ONL thickness (in μm) from Bioptigen
UHR OCT-b scans in the treated (Tx) area of the injected
(OS) eyes and in the equivalent treated (Tx-eq) of the
contralateral un-injected (OD) eyes at 12 weeks post-dose

| | | Injected (OS) | Un-injected (OD) |
|---|---|---|---|
| Dog ID | Treatment | Tx | Tx-eq |
| LH37 | Vehicle | 63.8 | 64.4 |
| CT4 | | 59.2 | 61.7 |
| EML34 | | 50.7 | 49.1 |
| CTL3 | Low-dose | 58.7 | 62.3 |
| ECT2 | AAV2/2-BEST1 | 54.7 | 56.7 |
| LH39 | | 47.3 | 39.9 |
| CT5 | High-dose | 56.5 | 57.8 |
| EML35 | AAV2/2-BEST1 | 56.7 | 61.1 |
| CTL1 | | 55.0 | 52.4 |

### Un-Injected Eyes

### OCT Map Analysis of ONL Thickness:

In 4 out of 9 un-injected eyes (Animal ID Nos CT4-OD, CTL3-OD, CT5-OD, and EML35-OD) the ONL thickness decreased, between the pre-dose and 12 weeks post-dose time points. All four were younger animals (12-13 weeks of age at dosing) and thus thinning of the ONL is likely age-related. In all 1 out of 9 remaining dog (Animal ID No. LH37-OD) ONL thickness remained stable, while in the remaining 4 out of 9 dogs an increase in ONL thickness possibly associated with the disease was observed.

### HR OCT Single B-Scan Analysis of ONL Thickness:

In 9 out of 9 un-injected eyes, ONL thickness at 12 weeks post-dose was within or slightly above the 95% CI of normal dogs, except in areas of a focal BEST1 lesion with retinal detachment where ONL thinning was seen (Animal ID nos. EML34-OD, LH39-OD, CT5-OD, and CTL1-OD).

### Vehicle-Injected Eyes

### OCT Map Analysis of ONL Thickness:

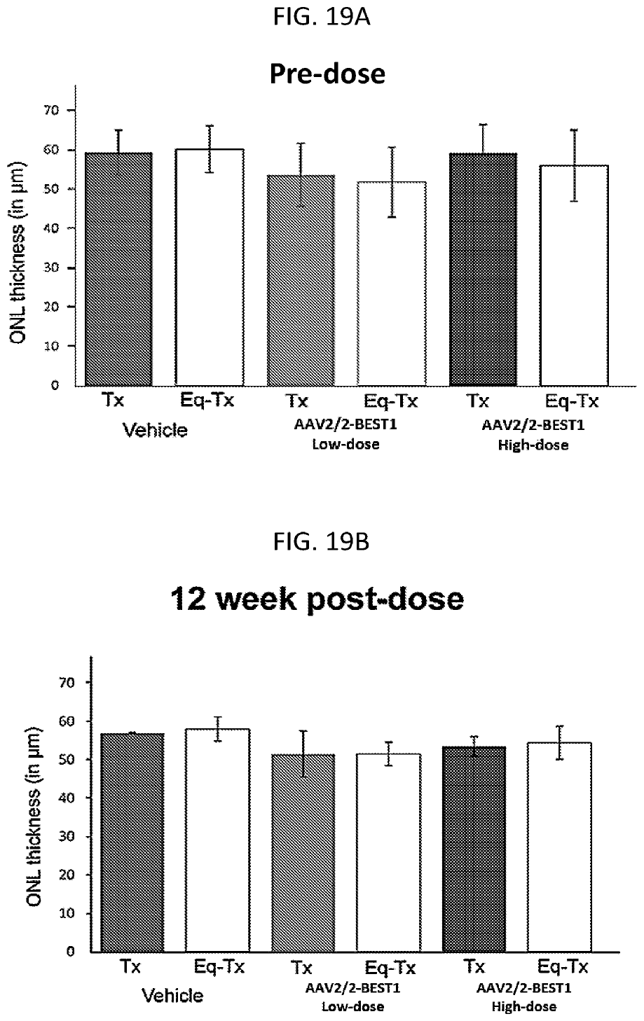

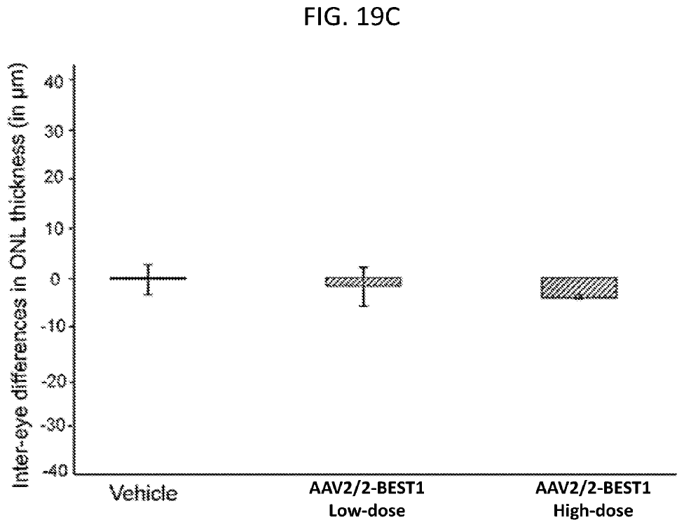

In 1 out of 3 vehicle-injected eyes (CT4-OS) the ONL thickness decreased in both the treated and untreated areas, between the pre-dose and 12 weeks post-dose time points. This was also seen in the contralateral un-injected eye and was likely age-related. No significant differences were seen when comparing the mean ONL thickness in the treated area of the vehicle-injected (OS) eyes to that of the equivalent treated area of the contralateral un-injected (OD) eyes either at pre-dose or 12 weeks post-dose (FIG. 19A and FIG. 19B). Similarly, no inter-eye differences were found between untreated and equivalent untreated areas.

### HR OCT Single B-Scan Analysis of ONL Thickness:

In 3 out of 3 vehicle-injected eyes, ONL thickness in the treated area at 12 weeks post-dose was within or slightly above the 95% CI of normal dogs, except in a BEST1-associated area of focal retinal detachment in Animal ID No. EML34-OS where the ONL was below the 95% CI. No significant differences were seen when comparing the mean ONL thickness in the treated area of the vehicle-injected (OS) eyes to that of the equivalent treated area of the contralateral un-injected (OD) eyes.

In summary, taken together, the combined results of both methods confirmed that the vehicle control article did not impact negatively nor positively any changes in ONL thickness.

$$Low Dose : 1.4 \times {10}^{9} vg / eye AAV 2 / 2 - BEST 1 Treated Eyes$$

### OCT Map Analysis of ONL Thickness:

In 1 out of 3 low-dose AAV2/2-BEST1-injected eyes (CTL3-OS) the ONL thickness decreased in both the treated and untreated areas, between the pre-dose and 12 weeks post-dose time points. This was also seen in the contralateral un-injected eye and was likely age-related. In the remaining 2 out of 3 dogs ONL thickness remained overall stable. No significant differences were seen when comparing the mean ONL thickness in the treated area of the low-dose AAV2/2-BEST1-injected (OS) eyes to that of the equivalent treated area of the contralateral un-injected (OD) eyes either at pre-dose or 12 weeks post-dose (FIG. 19A and FIG. 19B). Similarly, no inter-eye differences were found between untreated and equivalent untreated areas.

### HR OCT Single B-Scan Analysis of ONL Thickness:

In 3 out of 3 low-dose AAV2/2-BEST1-injected eyes, ONL thickness in the treated area at 12 weeks post-dose was within the 95% CI of normal dogs, except in a BEST1-associated area of focal retinal detachment in Animal ID No. LH39-OS where the ONL was below the 95% CI. No significant differences were seen when comparing the mean ONL thickness in the treated area of the low-dose AAV2/2-BEST1-injected (OS) eyes to that of the equivalent treated area of the contralateral un-injected (OD) eyes.

In summary, taken together, the combined results of both methods confirmed that AAV2/2-BEST1 at a low dose of 1.4×10⁹ vg/eye did not impact negatively nor positively any changes in ONL thickness.

$$High - Dose : 4.5 \times {10}^{9} vg / eye Treated Eyes$$

### OCT Map Analysis of ONL Thickness:

In 2 out of 3 high-dose AAV2/2-BEST1-injected eyes (CT5-OS and EML35-OS) the ONL thickness decreased in both the treated and untreated areas, between the pre-dose and 12 weeks post-dose time points. This was also seen in the contralateral un-injected eye and was likely age-related. In the remaining 1 out of 3 dogs ONL thickness remained overall stable. No significant differences were seen when comparing the mean ONL thickness in the treated area of the high-dose AAV2/2-BEST1-injected (OS) eyes to that of the equivalent treated area of the contralateral un-injected (OD) eyes either at pre-dose or 12 weeks post-dose (FIG. 19A and FIG. 19B). Similarly, no inter-eye differences were found between untreated and equivalent untreated areas.

### HR OCT Single B-Scan Analysis of ONL Thickness:

In 3 out of 3 high-dose AAV2/2-BEST1-injected eyes, ONL thickness in the treated area at 12 weeks post-dose was within the 95% CI of normal dogs, except in a BEST1-associated area of focal retinal detachment in Animal ID No. CTL1-OS where the ONL was below the 95% CI. No significant differences were seen when comparing the mean ONL thickness in the treated area of the high-dose AAV2/2-BEST1-injected (OS) eyes to that of the equivalent treated area of the contralateral un-injected (OD) eyes.

In summary, taken together, the combined results of both methods confirmed that AAV2/2-BEST1 at a high dose of 4.5×10⁹ vg/eye did not impact negatively nor positively any changes in ONL thickness.

### Comparison of ONL Thickness Across Treatment Groups

### OCT Map Analysis of ONL Thickness:

Quantitative comparison across treatment groups of the mean inter-eye differences in ONL thickness at 12 weeks post-dose (normalized to the IED at pre-dose) between the treated area of the injected eyes (OS) and the equivalent treated area of the un-injected contralateral eyes (OD) showed no significant differences (FIG. 19C).

### HR OCT Single B-Scan Analysis of ONL Thickness:

Quantitative comparison across treatment groups of the mean inter-ocular differences in ONL thickness at 12 weeks post-dose between the treated area of the injected eyes (OS) and the equivalent treated area of the un-injected contralateral eyes (OD) showed no significant differences.

In summary, both methods confirmed that either dose (low and high) of AAV2/2-BEST1 did not have any effect on ONL thickness.

### Safety Evaluations

### Clinical Observations and Body Weights

Daily clinical observations did not reveal any systemic or ocular effect of the test article. Clinical signs related to the use of a corticosteroid medication are detailed below.

Comparison of body weights across treatment groups throughout the in-life portion of the study did not show any effect of the test article.

### Vehicle-Injected Dogs

Daily clinical observation and weekly physical examinations were unremarkable.

In summary, no clinical signs of toxicity that could be associated with the vehicle were observed in any of the 3 injected dogs.

$$Low - Dose : 1.4 \times {10}^{9} vg / eye AAV 2 / 2 - BEST 1 Treated Dogs$$

Daily clinical observation and weekly physical examinations were unremarkable with the exception of a superficial corneal ulcer observed in 1 of 3 dogs immediately post-surgery (Animal ID No. ECT2) and a focal area of alopecia observed in 1 of 3 dogs (Animal ID No. CTL3). The corneal ulcer was noted 2 days post-dose and persisted to 5 days post-dose. The diagnosis was established by slit lamp examination with fluorescein staining and monitored daily. The ulcer was successfully treated with a short course of triple (neomycin-polymyxin B-bacitracin) antibiotic topical ointment (NeoPolyBac, Bausch & Lomb). The focal area of alopecia was seen at 12 weeks post-dose and persisted to 13 weeks post-dose. Microscopic examinations of a deep skin scrapings at each physical exam were non diagnostic but differentials include; contact alopecia, atopic dermatitis, and demodicosis. Contact alopecia is not uncommon in kennel housed dogs, atopic dermatitis is an inflammatory skin disorder associated with topical allergies, and demodicosis is a common parasitic skin disease in dogs that can be exacerbated following a course of corticosteroid medication.

In summary, no clinical signs of toxicity that could be associated with the low-dose of IC-100 were observed in any of the 3 treated dogs.

$$High - Dose : 4.5 \times {10}^{9} vg / eye AAV 2 / 2 - BEST 1 Treated Dogs$$

Daily clinical observation and weekly physical examinations were unremarkable with the exception of a focal area of alopecia observed in 1 of 3 dogs (Animal ID No. EML35), digital papillomas in 1 of 3 dogs (Animal ID No. EML35) and multifocal epidermal collarettes observed in 1 of 3 dogs (Animal ID No. CTL1). The alopecia was seen at 12 weeks post-dose and persisted to 13 weeks post-dose. Microscopic examinations of a deep skin scrapings at each physical exam were non diagnostic but differentials included contact alopecia, atopic dermatitis, and demodicosis. Contact alopecia is not uncommon in kennel housed dogs, atopic dermatitis is an inflammatory skin disorder associated with topical allergies, and demodicosis is a common parasitic skin disease in dogs that can be exacerbated following a course of corticosteroid medication. The epidermal collarettes were noted at 13 weeks post-dose and have a similar list of differentials with the additional of bacterial dermatitis secondary to contact alopecia. The digital papillomas were noted at 13 weeks post-dose. The most common cause of papillomas in dogs is canine papilloma virus-1 (CPV1), which is transmitted through direct contact with the virus and generally requires an immature immune system to establish clinical signs.

In summary, no clinical signs of toxicity that could be associated with the high-dose of AAV2/2-BEST1 were observed in any of the 3 treated dogs.

### Ophthalmic Examinations

Records of individual ophthalmic examinations for all animals at all time points and a summary of individual findings can be found in Tables 10-12.

TABLE 10
Summary of Ophthalmic Findings - Vehicle Control

| | | | Week 1 | Week 4 | Week 8 | Week 12 |
|---|---|---|---|---|---|---|
| Dog | Eye | Pre-dose | post-dose | post-dose | post-dose | post-dose |
| LH37 | OS | NF | Conj. Hyperemia | Retinotomy scar | Retinotomy scar | Retinotomy scar |
| | | | (mild) | | | |
| | | | Retinotomy scar | | | |
| | | | SR bleb border | | | |
| | | | Debris in SR bleb | | | |
| | OD | NF | NF | NF | NF | BEST1 stage |
| | | | | | | II |
| CT4 | OS | NF | Retinotomy scar | Conj. Hyperemia | Retinotomy scar | Retinotomy scar |
| | | | SR bleb border | (mild) | | BEST1 stage |
| | | | | Retinotomy scar | | II (UnTx) |
| | OD | NF | NF | NF | NF | NF |
| EML34 | OS | BEST1 stage | Retinotomy scar | Retinotomy scar | Retinotomy scar | Retinotomy scar |
| | | II (Tx) | BEST1 stage | BEST1 stage | BEST1 stage | BEST1 stage |
| | | BEST1 stage | II (Tx) | II (Tx) | II (Tx) | II (Tx) |
| | | III (Tx) | BEST1 stage | BEST1 stage | BEST1 stage | BEST1 stage |
| | | BEST1 stage | II (UnTx) | II (UnTx) | II (UnTx) | II (UnTx) |
| | | II (UnTx) | BEST1 stage | BEST1 stage | BEST1 stage | BEST1 stage |
| | | BEST1 stage | III (UnTx) | III (UnTx) | III (UnTx) | III (UnTx) |
| | | III (UnTx) | | | | |
| | OD | BEST1 stage | BEST1 stage | BEST1 stage | BEST1 stage | BEST1 stage |
| | | II | II | II | II | II |
| | | BEST1 stage | BEST1 stage | BEST1 stage | BEST1 stage | BEST1 stage |
| | | III | III | III | III | III |

NF = No finding;
Conj. = conjunctiva;
SR = subretinal;
BEST1 stage II = vitelliform lesion;
BEST1 stage III = pseudohypopion lesion;
Tx = treated area;
UnTx = untreated area

TABLE 11
Summary of Ophthalmic Findings - Low-Dose AAV2/2-BEST1

| | | | Week 1 | Week 4 | Week 8 | Week 12 |
|---|---|---|---|---|---|---|
| Dog | Eye | Pre-dose | post-dose | post-dose | post-dose | post-dose |
| CTL3 | OS | NF | Conj. Hyperemia | Retinal fold | Conj. Hyperemia | Retinal fold |
| | | | (mild) | (Tx) | (mild) | (Tx) |
| | | | Retinal fold | Hyperreflectivity | Retinal fold | Hyperreflectivity |
| | | | (Tx) | (Tx) | (Tx) | (Tx) |
| | | | Hyperreflectivity | | Hyperreflectivity | |
| | | | (Tx) | | (Tx) | |
| | | | Retinal detach. | | | |
| | | | (partial, Tx) | | | |
| | OD | Focal | Focal | Focal | Focal | Focal |
| | | pigmentation | pigmentation | pigmentation | pigmentation | pigmentation |
| ECT2 | OS | Conj. Hyperemia | Blepharospasm | Vit. Strand | Retinotomy Scar | Retinotomy Scar |
| | | (mod.) | (mild) | Retinotomy Scar | | |
| | | Periocular | Conj. Hyperemia | | | |
| | | erythema (mild) | (mild) | | | |
| | | | Vit. Strand | | | |
| | | | Retinotomy Scar | | | |
| | OD | Conj. Hyperemia | NF | NF | NF | NF |
| | | (mod.) | | | | |
| | | Periocular | | | | |
| | | erythema (mild) | | | | |
| LH39 | OS | Conj. Hyperemia | Conj. Hyperemia | BEST1 stage | BEST1 stage | Retinotomy Scar |
| | | (mild) | (mild) | III (Tx) | II (Tx) | BEST1 stage |
| | | BEST1 stage | Vit. Strand | BEST1 stage | BEST1 stage | II (Tx) |
| | | III (Tx) | BEST1 stage | III (UnTx) | III (UnTx) | BEST1 stage |
| | | BEST1 stage | III (Tx) | | | III (UnTx) |
| | | III (UnTx) | BEST1 stage | | | |
| | | | III (UnTx) | | | |
| | OD | Conj. Hyperemia | BEST1 stage | BEST1 stage | BEST1 stage | BEST1 stage |
| | | (mild) | III | III | III | III |
| | | BEST1 stage | | | | |
| | | III | | | | |

NF = No finding;
Conj. = conjunctiva;
SR = subretinal;
Retinal detach. = retinal detachment;
BEST1 stage II = vitelliform lesion;
BEST1 stage III = pseudohypopion lesion;
Tx = treated area;
UnTx = untreated area;
Vit = Vitreal;
Mod = Moderate;
R = Retinal

TABLE 12
Summary of Ophthalmic Findings - High-Dose AAV2/2-BEST1

| | | | Study | Study | Study | Study |
|---|---|---|---|---|---|---|
| Dog | Eye | Pre-dose | Week 1 | Week 4 | Week 8 | Week 12 |
| CT5 | OS | NF | Conj. Hyperemia | Conj. Hyperemia | Conj. Hyperemia | Conj. Hyperemia |
| | | | (mild) | (mild) | (mild) | (mild) |
| | | | Retinotomy Scar | Retinotomy Scar | Retinotomy Scar | Retinotomy Scar |
| | | | | | BEST1 stage | BEST1 stage |
| | | | | | II (UnTx) | II (UnTx) |
| | | | | | | Sig. Inc in |
| | | | | | | BEST1 (UnTx) |
| | OD | NF | NF | NF | BEST1 stage | BEST1 stage |
| | | | | | II | II |
| EML35 | OS | NF | Retinotomy Scar | Retinotomy Scar | Conj. Hyperemia | Retinotomy Scar |
| | | | | | (mild) | |
| | | | | | Retinotomy Scar | |
| | OD | NF | NF | NF | NF | NF |
| CTL1 | OS | BEST1 stage | Conj. Hyperemia | Retinotomy Scar | Conj. Hyperemia | Retinotomy Scar |
| | | III (Tx) | (mod.) | BEST1 stage | (mod.) | Reflectivity |
| | | BEST1 stage | Retinotomy Scar | II (UnTx) | Retinotomy Scar | change (Tx) |
| | | II (UnTx) | Retinal folds | BEST1 stage | BEST1 stage | BEST1 stage |
| | | BEST1 stage | BEST1 stage | III (UnTx) | II (UnTx) | II (UnTx) |
| | | III (UnTx) | II (UnTx) | | BEST1 stage | BEST1 stage |
| | | | BEST1 stage | | III (UnTx) | III (UnTx) |
| | | | III (UnTx) | | | |
| | OD | BEST1 stage | BEST1 stage | BEST1 stage | BEST1 stage | BEST1 stage |
| | | II | II | III | II | II |
| | | BEST1 stage | BEST1 stage | | BEST1 stage | BEST1 stage |
| | | III | III | | III | III |

NF = No finding;
Conj. = conjunctiva;
SR = subretinal;
BEST1 stage II = vitelliform lesion;
BEST1 stage III = pseudohypopion lesion;
Tx = treated area;
UnTx = untreated area;
Sig = Significant;
Inc = Increase;
Mod = Moderate

### Un-Injected Eyes

Table summarizes the ophthalmic findings seen during the course of the study in the 9 un-injected (OD) eyes. Conjunctival hyperemia was observed in two eyes (Animal ID Nos. LH39-OD and ECT2-OD) at the pre-dose time point and was most likely the result of the placement of stay sutures that were used to immobilize the globe during the pre-dose ERG that was conducted a few days prior. Periocular erythema present at pre-dose in Animal TD No. ECT2-OD was not observed at further time points. Fundus examination revealed at pre-dose a focal area of pigmentation that persisted during the course of the study in Animal ID No. CTL3-OD, as well as classic lesions of BEST1 disease (Stage II in Animal ID Nos. EMVL34-OD and CTL1-OD; Stage III in Animal ID Nos. EML34-OD, LH39-OD, and CTL1-OD). Lesions persisted in these eyes, and at 12 weeks post-dose were also seen in two additional eyes (Stage II in Animal ID Nos. LH37-OD, and CT5-OD).

TABLE 16
Important Ophthalmic Examination Findings of Un-injected Eyes

| | | Study | Study | Study | Study |
|---|---|---|---|---|---|
| | | Week | Week | Week | Week |
| Finding | Pre-dose | 1 | 4 | 8 | 12 |
| Conjunctival hyperemia | | | | | |
| Mild | 1/9 | 0/9 | 0/9 | 0/9 | 0/9 |
| Moderate | 1/9 | 0/9 | 0/9 | 0/9 | 0/9 |
| Periocular erythema | 1/9 | 0/9 | 0/9 | 0/9 | 0/9 |
| Fundus Exam | | | | | |
| Focal areas of | 1/9 | 1/9 | 1/9 | 1/9 | 1/9 |
| pigmentation | | | | | |
| BEST1 Stage II- | 2/9 | 2/9 | 1/9 | 3/9 | 4/9 |
| Vitelliform | | | | | |
| BEST1 Stage III- | | | | | |
| Pseudohypopyon | 3/9 | 3/9 | 3/9 | 3/9 | 4/9 |

### Vehicle-Injected Eyes

Table summarizes the ophthalmic findings seen during the course of the study in the 3 vehicle-injected eyes. Mild conjunctival hyperemia was seen in 2 out of 3 eyes at 1-week post-dose (Animal ID Nos. EML34-OS and LH37-OS) and 1 of the 3 eyes at 4 weeks post-dose (Animal ID No. CT4-OS). This was likely the result of the placement of stay sutures. Mild conjunctival hyperemia was seen in 1 of the 3 eyes at 4 weeks post-dose (Animal ID No. CT4) at the site of subconjunctival injection of triamcinolone acetonide. In 3 out of 3 eyes a focal area of pigmentation in the tapetal fundus was seen at the site of the retinotomy; this “scar” lesion is a common finding in dogs following subretinal injection. Classic lesions of BEST1 disease (Stages II and III were seen in the treated and untreated areas) of Animal ID No. EML34-OS during the course of the study, or at 12 weeks post-dose in the untreated area of Animal ID No. CT4-OS.

In summary no clinical signs of toxicity were seen in any of the vehicle-injected eyes, and ocular findings were related either to the surgical procedure or to natural course of disease in this model.

TABLE 17
Important Ophthalmic Examination Findings
of Eyes Injected with Vehicle

| | | Study | Study | Study | Study |
|---|---|---|---|---|---|
| | | Week | Week | Week | Week |
| Finding | Pre-dose | 1 | 4 | 8 | 12 |
| Conjunctival hyperemia | | | | | |
| Mild | 0/3 | 2/3 | 1/3 | 0/3 | 0/3 |
| Fundus Exam | | | | | |
| Retinotomy scar | 0/3 | 3/3 | 3/3 | 3/3 | 3/3 |
| Visible border of subretinal | 0/3 | 2/3 | 0/3 | 0/3 | 0/3 |
| bleb | | | | | |
| Within treated area | | | | | |
| BEST1 Stage II- Vitelliform | 1/3 | 1/3 | 1/3 | 1/3 | 1/3 |
| BEST1 Stage III- | | | | | |
| Pseudohypopyon | 1/3 | 0/3 | 0/3 | 0/3 | 0/3 |
| Other: White material in | 0/3 | 1/3 | 0/3 | 0/3 | 0/3 |
| inferior bleb area | | | | | |
| Within untreated area | | | | | |
| BEST1 Stage II- Vitelliform | 1/3 | 1/3 | 1/3 | 1/3 | 2/3 |
| BEST1 Stage III- | | | | | |
| Pseudohypopyon | 1/3 | 1/3 | 1/3 | 1/3 | 1/3 |

$$Low Dose : 1.4 \times {10}^{9} vg / eye AAV 2 / 2 - BEST 1 Treated Eyes$$

Table summarizes the ophthalmic findings seen during the course of the study in the 3 low-dose AAV2/2-BEST1-injected eyes. One eye (Animal ID. No. ECT2-OS) showed mild blepharospasm at 1 week post dose, which resolved shortly after. This was associated 1-5 days post-dose with a superficial corneal ulcer that was completely healed by 1-week post-dose. Cause of the ulcer was likely due to insufficient lubrication of the cornea post-surgery. Mild to moderate conjunctival hyperemia observed pre dose in 2 out of 3 eyes (Animal ID Nos. LH39-OS and ECT2-OS), in all 3 out of 3 eyes at week 1, and 1 out of 3 eyes at week 8 (Animal ID No. CTL3-OS) is likely the consequence of stay suture placement or subconjunctival triamcinolone acetonide injection. The presence of vitreal strands and/or fibrin was seen in 2 out of 3 eyes (Animal ID Nos. LH39-OS, and ECT2-OS) at 1 week post dose, and persisted in only ECT2-OS at 4 weeks post-dose. This is a frequent observation in dogs with this surgical (transvitreal) approach used to deliver the test-article subretinally without performing a vitrectomy, thus it cannot be unambiguously related to the test article. A common focal area of pigmentation in the tapetal fundus was seen at the site of the retinotomy in 2 out of 3 eyes (Animal ID Nos. ECT2-OS, and LH39-OS). Classic lesions of BEST1 disease (Stages II and III were seen in the treated and untreated areas) of Animal ID No. LH39-OS during the course of the study. In Animal ID No. CTL3-OS the retina in the treated area had not fully reattached by 1 week post dose. In this area, a localized site of hyperreflectivity and retinal folds was seen that persisted until 12 weeks-post dose.

In summary no clinical signs of toxicity were seen in 2 out 3 low-dose AAV2/2-BEST1-injected eyes, and ocular findings were related either to the surgical procedure or to the natural course of disease in this model. Retinal findings seen within the treated area of 1 out of 3 eyes (Animal ID No. CTL3-OS) could have been caused by the surgical procedure, and thus cannot be unambiguously related to the test article.

TABLE 18
Important Ophthalmic Examination Findings
of Eyes Injected with Low Dose AAV2/2-BEST1

| | | Study | Study | Study | Study |
|---|---|---|---|---|---|
| Finding | Pre-dose | Week 1 | Week 4 | Week 8 | Week 12 |
| Mild blepharospasm | 0/3 | 1/3 | 0/3 | 0/3 | 0/3 |
| Conjunctival | | | | | |
| hyperemia | | | | | |
| Mild | 1/3 | 3/3 | 0/3 | 1/3 | 0/3 |
| Moderate | 1/3 | 0/3 | 0/3 | 0/3 | 0/3 |
| Periocular erythema | 1/3 | 0/3 | 0/3 | 0/3 | 0/3 |
| Vitreous | | | | | |
| Vitreal strand/fibrin | 0/3 | 2/3 | 1/3 | 0/3 | 0/3 |
| Fundus Exam | | | | | |
| Retinotomy scar | 0/3 | 1/3 | 1/3 | 1/3 | 2/3 |
| Within treated area | | | | | |
| BEST1 Stage II- | 0/3 | 0/3 | 0/3 | 1/3 | 1/3 |
| Vitelliform | | | | | |
| BEST1 Stage III- | | | | | |
| Pseudohypopyon | 1/3 | 1/3 | 1/3 | 0/3 | 0/3 |
| Within untreated | | | | | |
| area BEST1 | | | | | |
| Stage III- | | | | | |
| Pseudohypopyon | 1/3 | 1/3 | 1/3 | 1/3 | 1/3 |
| Other: retinal fold, | 0/3 | 1/3 | 1/3 | 1/3 | 1/3 |
| hyperreflectivity | | | | | |
| Other: bleb | 0/3 | 1/3 | 0/3 | 0/3 | 0/3 |
| incompletely | | | | | |
| reattached | | | | | |

$$High - Dose : 4.5 \times {10}^{9} vg / eye AAV 2 / 2 - BEST 1 Treated Eyes$$

Table 16 summarizes the ophthalmic findings seen during the course of the study in the 3 high-dose AAV2/2-BEST1-injected eyes. Mild to moderate conjunctival hyperemia was seen in up to 3 out of 3 during the course of the study. This was likely the result of the placement of stay sutures and/or the subconjunctival triamcinolone acetonide injection performed immediately post-dose and at week 5 post-dose. A common focal area of pigmentation in the tapetal fundus was seen at the site of the retinotomy in 3 out of 3 eyes (Animal ID Nos. CT5-OS, CTL1-OS and EML35). Classic lesions of BEST1 disease (Stages II and III were seen in the treated and untreated areas) of Animal ID No. CTL1-OS.

In summary no clinical signs of toxicity were seen in any of the high-dose AAV2/2-BEST1-injected eyes, and ocular findings were related either to the surgical procedure or to natural course of disease in this model.

TABLE 16
Important Ophthalmic Examination Findings of
Eyes Injected with High Dose AAV2/2-BEST1

| | | Study | Study | Study | Study |
|---|---|---|---|---|---|
| | | Week | Week | Week | Week |
| Finding | Pre-dose | 1 | 4 | 8 | 12 |
| Conjunctival hyperemia | | | | | |
| Mild | 0/3 | 1/3 | 1/3 | 2/3 | 1/3 |
| Moderate | 0/3 | 1/3 | 0/3 | 1/3 | 0/3 |
| Fundus Exam | | | | | |
| Retinotomy scar | 0/3 | 3/3 | 3/3 | 3/3 | 3/3 |
| Within treated area | | | | | |
| BEST1 Stage III- | 1/3 | 0/3 | 0/3 | 0/3 | 0/3 |
| Pseudohypopyon | | | | | |
| Other: Retinal folds | 0/3 | 1/3 | 0/3 | 0/3 | 0/3 |
| Other: Change in reflectivity | 0/3 | 0/3 | 0/3 | 0/3 | 1/3 |
| Within untreated area | | | | | |
| BEST1 Stage II- Vitelliform | 1/3 | 1/3 | 1/3 | 2/3 | 2/3 |
| BEST1 Stage III- | | | | | |
| Pseudohypopyon | 1/3 | 1/3 | 1/3 | 1/3 | 1/3 |

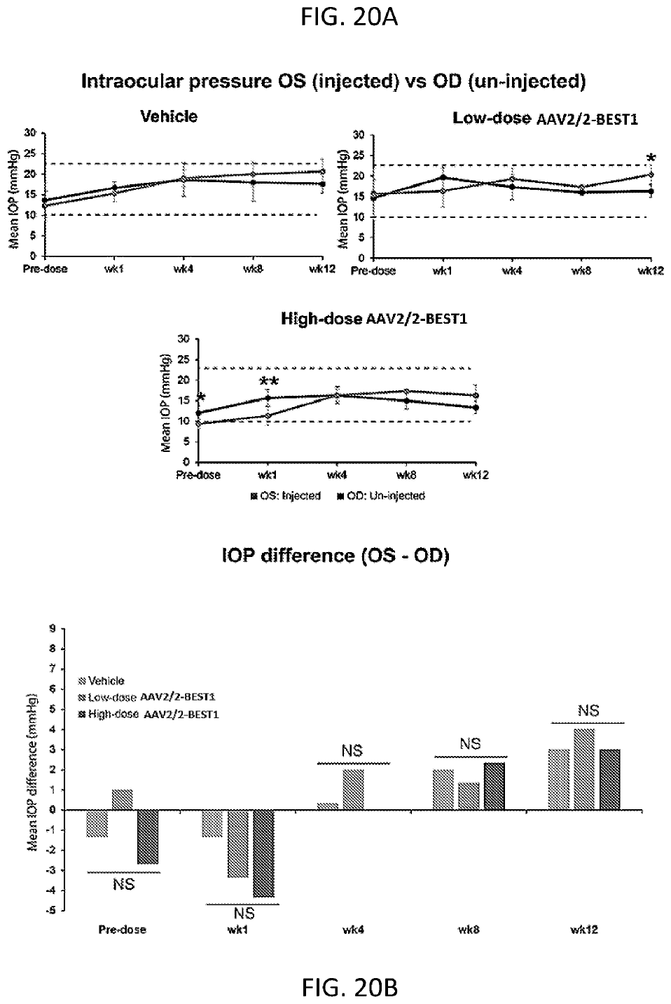

Mean IOP values in the low- and high-dose AAV2/2-BEST1-treated eyes (OS) were found to be statistically significantly different than that of the un-injected contralateral eyes (OD) at some time points, yet values remained within the 95% confidence interval (CI) limit of WT un-injected dogs, and thus were not considered to be clinically significant (FIG. 20A, upper left panel). In the case of the high-dose group a lower IOP was observed in OS at pre-dose thus was clearly not test article-related.

Inter-ocular IOP differences were not found to be significantly different across treatment groups at any time-point (FIG. 20B).

Retinal Findings by Confocal Scanning Laser Ophthalmoscopy (cSLO) and Optical

### Coherence Tomography

Besides its use to monitor the efficacy of the test article on the progression of BEST1 disease-related retinal lesions, cSLO/OCT imaging was also used to detect any potential signs of retinal toxicity.

### Un-Injected Eyes

No abnormalities other than those associated with the disease were seen in the 9 un-injected eyes.

### Vehicle-Injected Eyes

A commonly seen surgically-induced focal pigmented lesion was observed at the site of retinotomy as early as 1 week post-dose in 3 out of the 3 eyes and persisted thereafter. In Animal ID No. EML34 a localized area of retinal detachment increased over time at the site of retinotomy. This was likely surgery/disease-related.

In summary, no signs of toxicity that could be associated with the vehicle were observed by cSLO/OCT in any of the 3 treated eyes.

$$Low - Dose : 1.4 \times {10}^{9} vg / eye AAV 2 / 2 - BEST 1 Treated Eyes$$

A commonly seen surgically-induced focal pigmented lesion was observed at the site of retinotomy as early as 1 week post-dose in all 3 eyes and persisted thereafter. In Animal ID No. CTL3 a localized region with reflectivity changes was seen by cSLO (IR Reflectance mode) which corresponded to minimal alterations in ONL, and IS/OS lamination by OCT. These lesions could have been surgically-induced

In summary, no signs of toxicity that could be unambiguously associated with the low dose of AAV2/2-BEST1 were observed by cSLO/OCT in any of the 3 treated eyes.

$$High - Dose : 4.5 \times {10}^{9} vg / eye AAV 2 / 2 - BEST 1 Injected Eyes$$

A commonly seen surgically-induced focal pigmented lesion was observed at the site of retinotomy as early as 1 week post-dose in all 3 eyes and persisted thereafter.

In summary, no signs of toxicity that could be associated with the high dose of AAV2/2-BEST1 were observed by cSLO/OCT in any of the 3 treated eyes.

### Retinal Function Assessed by Electroretinography (ERG)

All animals underwent retinal functional assessment by ERG at two time points (pre-dose and 11 weeks post-dose), and results per group are summarized below. Amplitudes of recorded scotopic a-wave, scotopic b-wave, photopic (1 Hz) b-wave, and photopic 29 Hz flicker individual responses can be found in Table 17-24.

TABLE 17
Individual Scotopic a-Wave ERG Amplitudes (in μV) at Pre-Dose.

| | Dog | | Intensity (log cd · s/m²) | | | | | | | | | |
|---|---|---|---|---|---|---|---|---|---|---|---|---|
| Treatment | ID | Eye | −3.74 | −3.25 | −2.74 | −2.24 | −1.74 | −1.24 | −0.74 | −0.25 | 0.26 | 0.51 |
| Vehicle | LH37 | OS | 0.872 | 1.317 | 0.388 | 0.74 | 4.884 | 12.05 | 56.03 | 102.6 | 140.7 | 156.7 |
| | | OD | 0.552 | 0.557 | 0.691 | 0.564 | 3.065 | 6.684 | 31.35 | 69.32 | 101.8 | 117.1 |
| | CT4 | OS | 2.525 | 0.02 | 3.264 | 0.111 | 3.217 | 10.21 | 58.56 | 105.7 | 156.9 | 177.2 |
| | | OD | 1.081 | 0.948 | 2.646 | 1.328 | 5.996 | 23.95 | 92.61 | 173.8 | 249.6 | 276.5 |
| | EML34 | OS | 1.221 | 1.46 | 0.511 | 1.313 | 4.56 | 13.69 | 39.63 | 68.39 | 93.69 | 101 |
| | | OD | 1.37 | 0.652 | 0.06 | 2.133 | 0.534 | 10.06 | 36.54 | 64.78 | 85.79 | 95.72 |
| Low- | CTL3 | OS | 1.628 | 1.845 | 0.478 | 11.39 | 7.39 | 9.748 | 34 | 58.85 | 88.81 | 98.22 |
| Dose | | OD | 1.603 | 0.406 | 5.244 | 6.391 | 9.208 | 6.623 | 23.05 | 53.65 | 82.97 | 89.56 |
| AAV2/2- | ECT2 | OS | 2.287 | 0.108 | 0.79 | 0.65 | 1.567 | 5.835 | 35.66 | 58.62 | 72.53 | 83.31 |
| BEST1 | | OD | 3.878 | 0.991 | 1.046 | 0.464 | 1.267 | 6.557 | 29.24 | 56.71 | 74.76 | 83.2 |
| | LH39 | OS | 1.983 | 1.21 | 2.634 | 4.718 | 0.77 | 4.005 | 23.13 | 46.98 | 65.8 | 72.78 |
| | | OD | 3.772 | 1.999 | 2.073 | 2.835 | 0.497 | 6.512 | 28.5 | 59.83 | 80.44 | 90.71 |
| High- | CT5 | OS | 2.398 | 3.819 | 9.087 | 15.07 | 7.369 | 16 | 58.46 | 111.7 | 158.1 | 177.8 |
| Dose | | OD | 2.078 | 5.211 | 8.725 | 15.71 | 7.897 | 17.31 | 66.75 | 132.7 | 188.1 | 206.3 |
| AAV2/2- | EML35 | OS | 0.037 | 1.261 | 5.802 | 22.07 | 7.075 | 6.127 | 41.26 | 97.35 | 136.8 | 142.9 |
| BEST1 | | OD | 0.282 | 0.887 | 2.653 | 8.445 | 2.651 | 13.53 | 49.71 | 98.45 | 135.4 | 147.5 |
| | CTL1 | OS | 5.853 | 1.371 | 1.818 | 2.367 | 1.208 | 13.04 | 52.74 | 87.17 | 111.8 | 124.3 |
| | | OD | 2.027 | 1.577 | 1.759 | 1.902 | 0.64 | 9.58 | 32.64 | 54.79 | 81.55 | 85.07 |

TABLE 18
Individual Scotopic a-Wave ERG Amplitudes (in μV) at 11 Weeks Post-Dose.

| | Dog | | Intensity (log cd · s/m²) | | | | | | | | | |
|---|---|---|---|---|---|---|---|---|---|---|---|---|
| Treatment | ID | Eye | −3.74 | −3.25 | −2.74 | −2.24 | −1.74 | −1.24 | −0.74 | −0.25 | 0.26 | 0.51 |
| Vehicle | LH37 | OS | 0.582 | 0.104 | 1.381 | 0.143 | 3.527 | 11.39 | 41.23 | 73.1 | 97.45 | 111.6 |
| | | OD | 1.424 | 0.705 | 0.61 | 0.614 | 2.936 | 12.61 | 47.03 | 78.41 | 98.67 | 114.2 |
| | CT4 | OS | 10.71 | 2.853 | 9.026 | 8.162 | 15.7 | 40.81 | 102.4 | 154.3 | 167 | 195.8 |
| | | OD | 9.914 | 1.918 | 7.35 | 8.411 | 19.04 | 45 | 107.4 | 163.2 | 187.7 | 210.1 |
| | EML34 | OS | 1.437 | 1.389 | 0.371 | 0.377 | 4.471 | 9.116 | 47.65 | 72.66 | 92.71 | 98.99 |
| | | OD | 2.583 | 0.645 | 0.198 | 0.48 | 3.157 | 11.9 | 41.73 | 70.54 | 90.67 | 99.94 |
| Low-Dose | CTL3 | OS | 0.139 | 0.535 | 0.882 | 1.779 | 5.07 | 14.36 | 49.93 | 82.39 | 108.8 | 119.9 |
| AAV2/2- | | OD | 0.035 | 1.378 | 0.475 | 1.528 | 4.31 | 11.08 | 45.55 | 75.52 | 100.6 | 110.6 |
| BEST1 | ECT2 | OS | 5.815 | 1.015 | 6.494 | 8.781 | 14.92 | 38.56 | 78.09 | 99.93 | 117.8 | 126.1 |
| | | OD | 8.628 | 5.428 | 9.394 | 10.84 | 14.09 | 31.36 | 60.94 | 81.32 | 90.17 | 96.99 |
| | LH39 | OS | 0.026 | 1.596 | 0.011 | 2.811 | 6.016 | 27.01 | 69.53 | 110.8 | 130.1 | 137.2 |
| | | OD | 2.593 | 1.842 | 0.254 | 0.929 | 4.617 | 20.42 | 55.67 | 95.46 | 112.8 | 117.9 |
| High-Dose | CT5 | OS | 0.254 | 2.353 | 1.258 | 2.351 | 6.601 | 19.14 | 65.45 | 113 | 144 | 157 |
| AAV2/2- | | OD | 2.947 | 1.408 | 0.433 | 0.133 | 4.998 | 8.127 | 47.03 | 75.85 | 102.8 | 110.6 |
| BEST1 | EML35 | OS | 10.22 | 0.232 | 2.142 | 1.774 | 5.332 | 23.98 | 63.91 | 96.65 | 125 | 135.6 |
| | | OD | 13.92 | 0.205 | 2.046 | 2.124 | 4.118 | 17.26 | 43.78 | 71.12 | 92.35 | 103.2 |
| | CTL1 | OS | 1.418 | 2.132 | 0.005 | 0.171 | 6.214 | 16.66 | 64.03 | 101.9 | 128.9 | 137.9 |
| | | OD | 0.5 | 0.342 | 0.974 | 3.368 | 5.457 | 12.16 | 47.89 | 77.71 | 98.04 | 106.6 |

TABLE 19
Individual Scotopic b-Wave ERG Amplitudes (in μV) at Pre-Dose.

| | Dog | | Intensity (log cd · s/m²) | | | | | | | | | |
|---|---|---|---|---|---|---|---|---|---|---|---|---|
| Treatment | ID | Eye | −3.74 | −3.25 | −2.74 | −2.24 | −1.74 | −1.24 | −0.74 | −0.25 | 0.26 | 0.51 |
| Vehicle | LH37 | OS | 9.868 | 42.34 | 108.8 | 192.9 | 225.9 | 239.3 | 244.5 | 281.9 | 301.6 | 300 |
| | | OD | 3.69 | 21.38 | 62.39 | 130.6 | 182.8 | 190.1 | 183.4 | 208 | 232.2 | 236.6 |
| | CT4 | OS | 7.907 | 46.36 | 106.8 | 191.5 | 250.1 | 240 | 251.6 | 276.1 | 336.9 | 352.8 |
| | | OD | 7.951 | 70.17 | 156.8 | 297.1 | 358.7 | 348.2 | 369.9 | 407.9 | 510 | 526.2 |
| | EML34 | OS | 1.568 | 9.29 | 34.08 | 96.81 | 135.3 | 136.4 | 145.9 | 174.2 | 183.2 | 202 |
| | | OD | 3.596 | 7.824 | 33.96 | 89.31 | 116.4 | 120.8 | 131 | 156.4 | 169.3 | 180.8 |
| Low- | CTL3 | OS | 8.572 | 28.85 | 90.46 | 150.9 | 202.6 | 221.5 | 242.4 | 243.5 | 287 | 292.8 |
| Dose | | OD | 2.068 | 14.67 | 64.2 | 131.9 | 179.9 | 195 | 208.5 | 225.3 | 268.1 | 275.1 |
| AAV2/2- | ECT2 | OS | −2.982 | 20.02 | 27.51 | 73.57 | 106.2 | 96.6 | 90.48 | 120.4 | 131.6 | 133.8 |
| BEST1 | | OD | 1.049 | 4.018 | 22.58 | 66 | 93.74 | 82.37 | 89.31 | 109.6 | 121.5 | 123.5 |
| | LH39 | OS | 0.901 | 10.12 | 26.56 | 71.18 | 122.3 | 125 | 133.5 | 151.6 | 167.9 | 165.1 |
| | | OD | 1.352 | 7.407 | 27.45 | 76.68 | 130.9 | 134.5 | 140.6 | 164 | 180.8 | 185.2 |
| High- | CT5 | OS | 2.714 | 36.05 | 104.5 | 235.1 | 309.7 | 300.1 | 330.8 | 383.8 | 424.3 | 431.7 |
| Dose | | OD | 0.989 | 28.07 | 108.9 | 262.1 | 364.3 | 348.4 | 381.5 | 450.9 | 496.5 | 498.9 |
| AAV2/2- | EML35 | OS | 2.032 | 10.81 | 54.43 | 132.5 | 169 | 167.2 | 187 | 213 | 263.4 | 278 |
| BEST1 | | OD | 5.744 | 19.75 | 77.34 | 161.9 | 215 | 205.3 | 229.4 | 251.5 | 306.7 | 317.2 |
| | CTL1 | OS | 3.497 | 31.36 | 89.38 | 164.1 | 205.8 | 216.5 | 232.4 | 292.8 | 315.2 | 322.8 |
| | | OD | 2.607 | 12.28 | 54.95 | 108.7 | 152.4 | 161.3 | 172.7 | 209.2 | 232.4 | 235.7 |

TABLE 20
Individual Scotopic b-Wave ERG Amplitudes (in μV) at 11 Weeks Post-Dose

| | Dog | | Intensity (log cd · s/m²) | | | | | | | | | |
|---|---|---|---|---|---|---|---|---|---|---|---|---|
| Treatment | ID | Eye | −3.74 | −3.25 | −2.74 | −2.24 | −1.74 | −1.24 | −0.74 | −0.25 | 0.26 | 0.51 |
| Vehicle | LH37 | OS | 4.919 | 16.02 | 61.8 | 131.4 | 165.7 | 165.1 | 176.6 | 199 | 209.6 | 219.5 |
| | | OD | 4.211 | 15.04 | 62.53 | 138.7 | 171.7 | 176.3 | 186.5 | 206.4 | 216.1 | 227 |
| | CT4 | OS | 10.95 | 64.21 | 160 | 277.8 | 301.1 | 297.6 | 278.9 | 301.9 | 299.3 | 344.8 |
| | | OD | 10.51 | 63.32 | 172.9 | 290.2 | 294.1 | 306.3 | 293.3 | 319.5 | 329.5 | 381.4 |
| | EML34 | OS | 2.541 | 13 | 52.74 | 106.4 | 124.1 | 129.3 | 146.8 | 163 | 170.8 | 185.3 |
| | | OD | 2.629 | 13.28 | 52.09 | 102.1 | 118.6 | 130.5 | 146.5 | 158.9 | 170 | 184.3 |
| Low- | CTL3 | OS | 10.01 | 47.7 | 115.2 | 195.8 | 211.3 | 221.1 | 237 | 258.3 | 274.7 | 279.9 |
| Dose | | OD | 9.828 | 43.26 | 105.3 | 180.6 | 204.2 | 212.3 | 226.4 | 246.2 | 265.7 | 270.3 |
| AAV2/2- | ECT2 | OS | 4.135 | 37.56 | 96.83 | 175.4 | 193.7 | 197.9 | 213.2 | 220.4 | 222.3 | 230.7 |
| BEST1 | | OD | 3.051 | 19.84 | 58.2 | 126.4 | 153.8 | 157.8 | 164.6 | 172 | 166.8 | 175.8 |
| | LH39 | OS | 8.266 | 47.04 | 120.7 | 204 | 225 | 237.7 | 243.3 | 266.3 | 276.6 | 278.9 |
| | | OD | 1.976 | 30.25 | 93.9 | 163.3 | 182.4 | 196.5 | 204.9 | 225.5 | 234.1 | 236.5 |
| High- | CT5 | OS | 23.07 | 69.04 | 146.9 | 245.3 | 276.9 | 270.7 | 271.7 | 294.6 | 334 | 341.9 |
| Dose | | OD | 10.85 | 39 | 85.91 | 151.9 | 183.3 | 187.8 | 194.2 | 209.3 | 254.6 | 253.8 |
| AAV2/2- | EML35 | OS | 5.691 | 39.64 | 97.35 | 173.9 | 216.3 | 222.7 | 252.5 | 292.8 | 322.4 | 325.9 |
| BEST1 | | OD | 3.616 | 14.66 | 50.44 | 109.2 | 151.1 | 167.2 | 179.2 | 219.4 | 247.2 | 257.3 |
| | CTL1 | OS | 4.141 | 45.35 | 111.5 | 197.1 | 237.1 | 244.9 | 251.8 | 273 | 318 | 321.7 |
| | | OD | 5.281 | 30.96 | 81.08 | 144 | 183.1 | 181.9 | 197.3 | 219 | 247.7 | 255.2 |

TABLE 21
Individual Photopic b-Wave ERG Amplitudes (in μV) at Pre-Dose.

| | Dog | | Intensity (log cd · s/m²) | | | | | | | |
|---|---|---|---|---|---|---|---|---|---|---|
| Treatment | ID | Eye | −2.74 | −2.24 | −1.74 | −1.24 | −0.74 | −0.25 | 0.26 | 0.51 |
| Vehicle | LH37 | OS | 1.334 | 0.446 | 2.012 | 8.565 | 18.24 | 27.41 | 35.6 | 38.07 |
| | | OD | 2.476 | 0.302 | 1.432 | 4.945 | 11.17 | 18.62 | 22.68 | 25.02 |
| | CT4 | OS | 2.25 | 1.204 | 4.853 | 7.764 | 15.28 | 33.13 | 55.5 | 56.11 |
| | | OD | 0.762 | 1.415 | 3.14 | 10.44 | 21.33 | 50.22 | 80.87 | 82.89 |
| | EML34 | OS | 16.07 | 11.74 | 12.47 | 14.38 | 22.46 | 26.62 | 30.98 | 29.63 |
| | | OD | 6.983 | 3.466 | 10.08 | 26.04 | 44.71 | 27.37 | 26.93 | 28.11 |
| Low-Dose | CTL3 | OS | 3.603 | 5.341 | 2.486 | 1.958 | 12.12 | 29.78 | 39.12 | 38.29 |
| AAV2/2- | | OD | 0.507 | 0.509 | 1.834 | 1.021 | 8.79 | 20.43 | 31.39 | 29.88 |
| BEST1 | ECT2 | OS | 3.752 | 1.582 | 1.468 | 2.65 | 7.489 | 16 | 33.99 | 29.11 |
| | | OD | 5.512 | 5.255 | 6.615 | 6.018 | 10.29 | 20.47 | 34.87 | 34.62 |
| | LH39 | OS | 2.191 | 4.019 | 4.372 | 3.958 | 5.681 | 9.969 | 21.03 | 21.68 |
| | | OD | 1.345 | 0.888 | 2.111 | 2.161 | 6.128 | 10.96 | 20.52 | 23.46 |
| High-Dose | CT5 | OS | 0.911 | 0.466 | 3.568 | 7.185 | 15.87 | 37.74 | 56.2 | 54.47 |
| AAV2/2- | | OD | 0.827 | 1.565 | 4.088 | 8.122 | 21.46 | 50.32 | 72.95 | 68.48 |
| BEST1 | EML35 | OS | 0.519 | −1.059 | 1.123 | 6.748 | 11.07 | 22.38 | 33.89 | 36.8 |
| | | OD | 1.066 | 0.889 | 2.366 | 6.491 | 11.63 | 25.93 | 37.65 | 33.19 |
| | CTL1 | OS | 7.223 | 4.87 | 4.528 | 7.557 | 10.85 | 17.38 | 34.84 | 32.23 |
| | | OD | 1.151 | 2.137 | 1.893 | 2.607 | 5.83 | 10.48 | 21.11 | 23.11 |

TABLE 19
Individual Photopic b-Wave ERG Amplitudes (in μV) at 11 Weeks Post-Dose

| | Dog | | Intensity (log cd · s/m²) | | | | | | | |
|---|---|---|---|---|---|---|---|---|---|---|
| Treatment | ID | Eye | −2.74 | −2.24 | −1.74 | −1.24 | −0.74 | −0.25 | 0.26 | 0.51 |
| Vehicle | LH37 | OS | 5.239 | 4.929 | 8.546 | 11.43 | 15.02 | 20.9 | 25.78 | 31.82 |
| | | OD | 2.132 | 4.211 | 5.435 | 7.108 | 13.05 | 19.91 | 28.1 | 28.23 |
| | CT4 | OS | 0.945 | 1.06 | 3.965 | 6.417 | 14.12 | 40.84 | 65.19 | 60.73 |
| | | OD | 1.127 | −0.264 | 3.109 | 7.192 | 15.92 | 45.38 | 63.98 | 56.74 |
| | EML34 | OS | 2.021 | 1.95 | 2.753 | 4.338 | 9.888 | 15.77 | 29.52 | 30.3 |
| | | OD | 5.526 | 3.651 | 3.9 | 6.234 | 9.139 | 15.33 | 30.49 | 29.51 |
| Low- | CTL3 | OS | 2.305 | 2.679 | 4.462 | 6.385 | 12.31 | 30.83 | 32.69 | 40.84 |
| Dose | | OD | 0.852 | 1.777 | 2.727 | 5.699 | 9.827 | 24.4 | 30.77 | 31.86 |
| AAV2/2- | ECT2 | OS | 1.085 | 1.681 | 2.155 | 5.455 | 12.55 | 34.06 | 43.56 | 35.22 |
| BEST1 | | OD | 1.67 | 2.045 | 2.207 | 4.043 | 10.24 | 24.32 | 31.86 | 26.45 |
| | LH39 | OS | 1.578 | 2.774 | 2.751 | 3.722 | 8.026 | 17.27 | 35.9 | 40.12 |
| | | OD | 1.274 | 2.309 | 1.742 | 1.381 | 5.008 | 9.058 | 17.1 | 21.73 |
| High- | CT5 | OS | 2.823 | 2.427 | 4.21 | 5.983 | 12.69 | 31.38 | 54.7 | 56.15 |
| Dose | | OD | 3.445 | 2.928 | 3.019 | 5.775 | 9.565 | 18.71 | 33.25 | 34.29 |
| AAV2/2- | EML35 | OS | 1.777 | 2.14 | 3.42 | 12.26 | 10.12 | 24.31 | 29.52 | 32.95 |
| BEST1 | | OD | 1.17 | 1.419 | 2.508 | 8.487 | 5.963 | 14.6 | 24.76 | 23.71 |
| | CTL1 | OS | 6.757 | 3.345 | 8.786 | 4.914 | 9.849 | 18.06 | 26.01 | 28.5 |
| | | OD | 5.113 | 4.431 | 7.456 | 5.815 | 9.03 | 9.862 | 17.83 | 17.64 |

TABLE 20
Individual Photopic Flicker (29 Hz) ERG Amplitudes (in μV) at Pre-Dose.

| | Dog | | Intensity (log cd · s/m²) | | | | | | |
|---|---|---|---|---|---|---|---|---|---|
| Treatment | ID | Eye | −2.74 | −2.24 | −1.74 | −1.24 | −0.74 | −0.25 | 0.26 |
| Vehicle | LH37 | OS | 0.741 | 2.023 | 5.065 | 11.83 | 19.62 | 32.62 | 49.91 |
| | | OD | 0.616 | 1.219 | 2.981 | 7.856 | 13.25 | 21.7 | 35.19 |
| | CT4 | OS | −0.149 | 1.296 | 3.625 | 5.653 | 15.2 | 43.94 | 69.67 |
| | | OD | −0.664 | 1.791 | 4.359 | 8.385 | 21.82 | 70.56 | 109.5 |
| | EML34 | OS | 3.975 | 2.741 | 3.794 | 9.932 | 23.29 | 30.47 | 35.99 |
| | | OD | 2.414 | 3.767 | 5.515 | 14.15 | 37.38 | 42.63 | 49.27 |
| Low- | CTL3 | OS | 0.588 | 1.442 | 2.226 | 8.296 | 15.13 | 33.03 | 45.97 |
| Dose | | OD | 0.664 | 0.969 | 1.584 | 5.341 | 12.08 | 26.75 | 38.14 |
| AAV2/2- | ECT2 | OS | 0.072 | 0.92 | 1.964 | 3.788 | 9.52 | 24.43 | 36.72 |
| BEST1 | | OD | 0.319 | 0.918 | 2.484 | 4.916 | 11.51 | 27.98 | 41.74 |
| | LH39 | OS | 0.04 | 0.623 | 1.775 | 4.461 | 7.973 | 19.18 | 34.98 |
| | | OD | 0.05 | 1.124 | 2.369 | 6.004 | 10.34 | 23.46 | 41.28 |
| | CT5 | OS | 1.669 | 1.575 | 4.631 | 7.541 | 9.733 | 45.58 | 61.98 |
| High- | | OD | 1.261 | 3.294 | 5.877 | 10.29 | 25.21 | 60.03 | 79.64 |
| Dose | EML35 | OS | 1.051 | 1.662 | 4.479 | 5.897 | 13.03 | 32.23 | 45.64 |
| AAV2/2- | | OD | 0.212 | 1.679 | 4.973 | 7.692 | 15.41 | 34.76 | 49.66 |
| BEST1 | CTL1 | OS | 0.952 | 2.99 | 2.388 | 5.149 | 11.46 | 22.55 | 35.03 |
| | | OD | 0.665 | 1.008 | 1.461 | 5.064 | 7.196 | 17.21 | 29.15 |

TABLE 24
Individual Photopic Flicker (29 Hz) ERG
Amplitudes (in μV) at 11 Weeks Post-Dose.

| | Dog | | Intensity (log cd · s/m²) | | | | | | |
|---|---|---|---|---|---|---|---|---|---|
| Treatment | ID | Eye | −2.74 | −2.24 | −1.74 | −1.24 | −0.74 | −0.25 | 0.26 |
| Vehicle | LH37 | OS | 1.389 | 2.125 | 4.968 | 9.933 | 16.81 | 30.28 | 47.59 |
| | | OD | 1.183 | 2.773 | 5.305 | 9.902 | 16.9 | 32.09 | 47.4 |
| | CT4 | OS | 0.852 | 1.243 | 2.866 | 7.006 | 18.24 | 51.71 | 94.92 |
| | | OD | 0.601 | 1.233 | 3.844 | 7.021 | 22.12 | 60.15 | 97.48 |
| | EML34 | OS | 0.563 | 0.909 | 2.481 | 4.922 | 8.185 | 25.37 | 44.5 |
| | | OD | 0.533 | 1.178 | 2.977 | 8.17 | 12.85 | 26.29 | 47.56 |
| Low- | CTL3 | OS | 0.653 | 1.584 | 3.435 | 7.553 | 20.45 | 40.1 | 53.34 |
| Dose | | OD | 0.813 | 1.351 | 3.185 | 6.339 | 15.26 | 30.92 | 41.7 |
| AAV2/2- | ECT2 | OS | 0.686 | 1.271 | 2.511 | 4.65 | 16.27 | 43.67 | 63.27 |
| BEST1 | | OD | 0.677 | 0.954 | 1.798 | 3.634 | 10.44 | 30.15 | 47.36 |
| | LH39 | OS | 0.327 | 0.447 | 1.119 | 5.007 | 11.98 | 33.05 | 63.42 |
| | | OD | 0.617 | 2.033 | 1.712 | 2.615 | 7.017 | 24.31 | 49.08 |
| High- | CT5 | OS | 0.503 | 2.662 | 2.935 | 7.988 | 15.54 | 46.13 | 79.94 |
| Dose | | OD | 0.276 | 2.01 | 1.854 | 4.012 | 9.431 | 30.15 | 50.17 |
| AAV2/2- | EML35 | OS | 1.012 | 1.609 | 3.263 | 6.091 | 13.52 | 27.01 | 35.99 |
| BEST1 | | OD | 0.694 | 1.346 | 2.614 | 4.521 | 8.325 | 17.27 | 24.78 |
| | CTL1 | OS | 1.354 | 1.649 | 3.721 | 7.749 | 11.07 | 29.01 | 47.67 |
| | | OD | 1.781 | 2.371 | 3.133 | 3.704 | 7.105 | 19.25 | 29.22 |

### Un-Injected Eyes

All 9 out of 9 un-injected eyes had, at both time points, rod, mixed rod-cone, and cone-mediated normal appearing ERG traces with amplitudes that were comparable or slightly lower (Animal ID Nos. EML34-OD), ECT2-OD, LH39-OD, CTL1-OD) than that of WT dogs measured using the same ERG system and protocol. See, Beltran W A et al. Mol Ther. 2017; 25: 1866-1880.

### Vehicle-Injected Eyes

All 3 out of 3 vehicle-injected eyes had, at both time points, normal appearing ERG traces with amplitudes that were comparable or slightly lower (Animal IDs No. EMVL34-OS) than that of WT dogs.

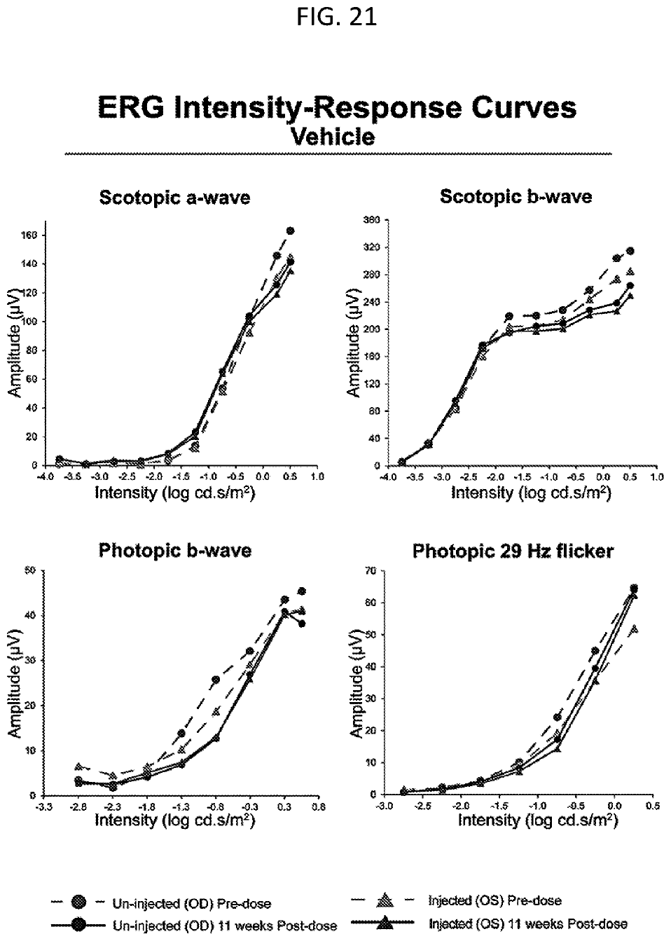

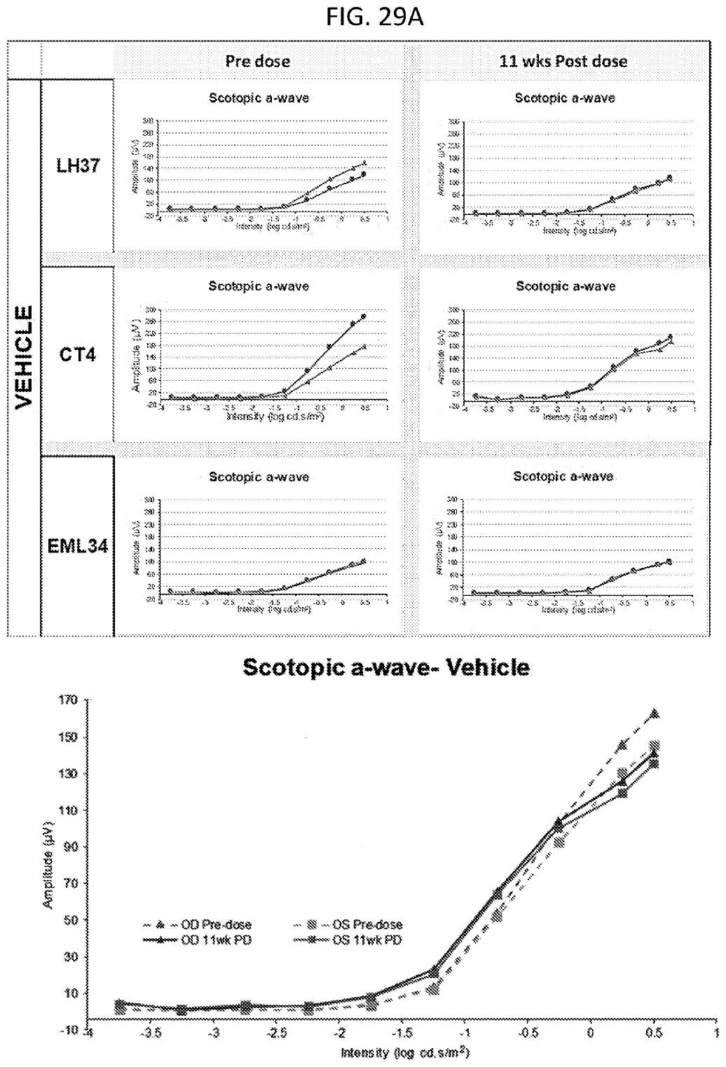

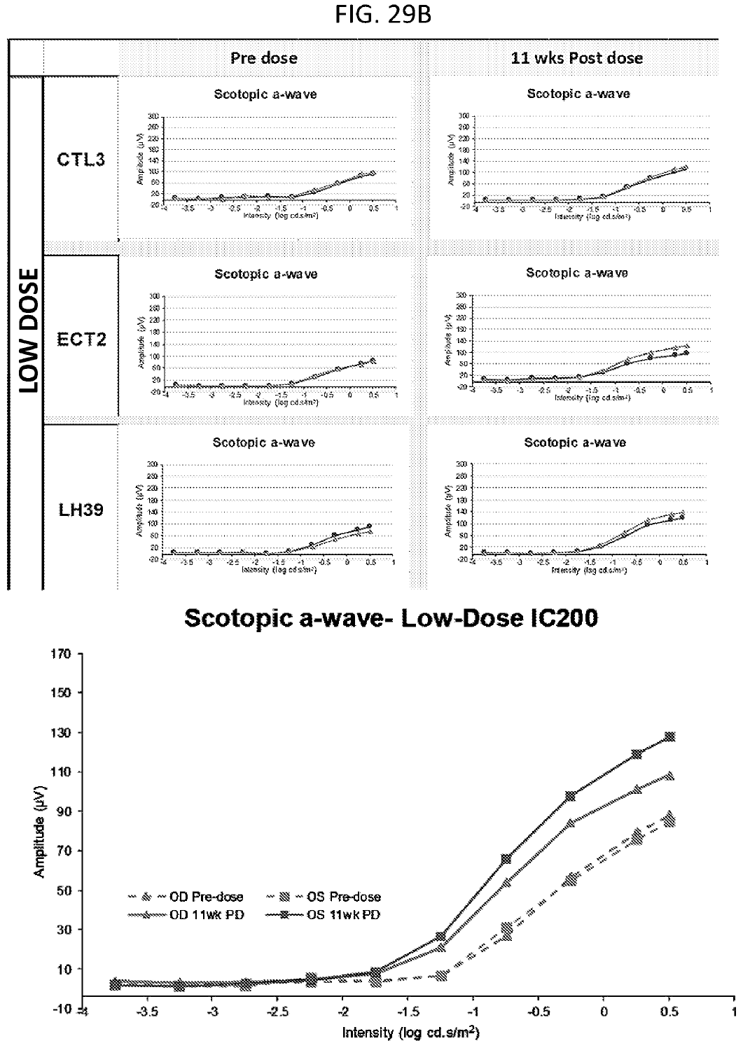

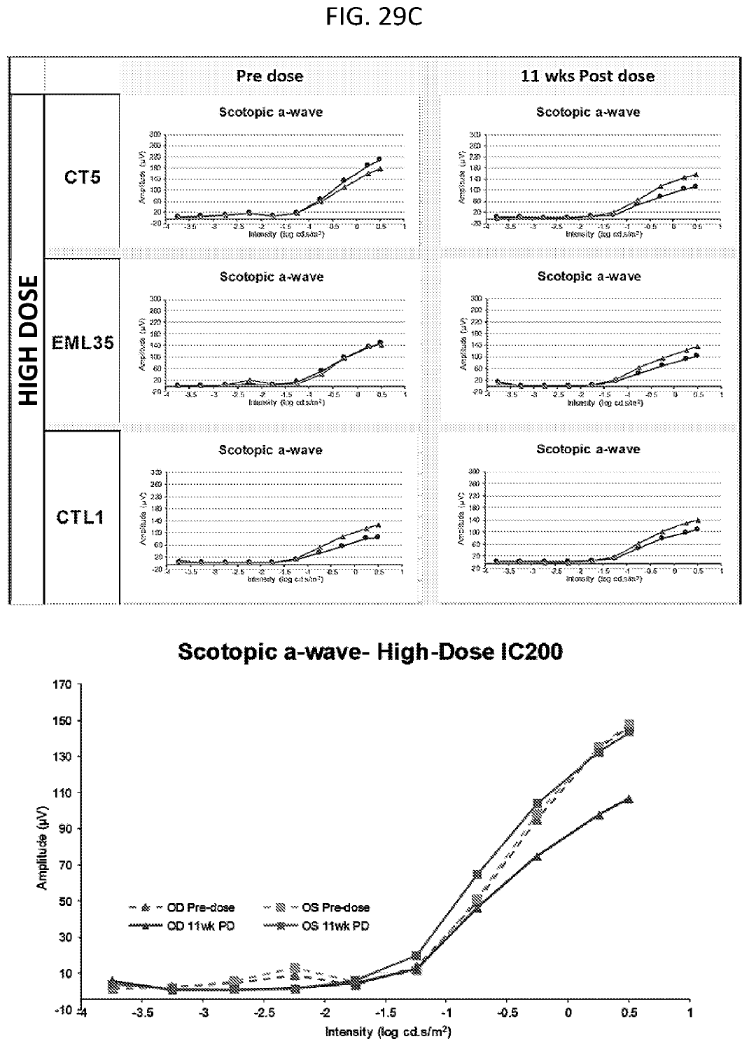

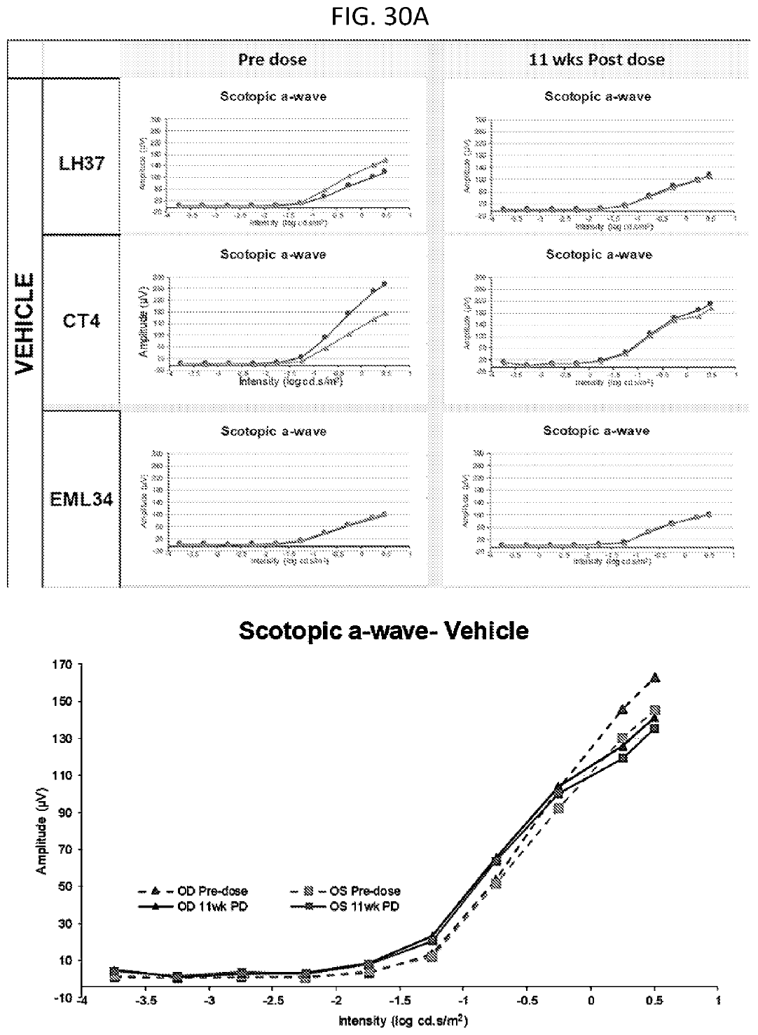

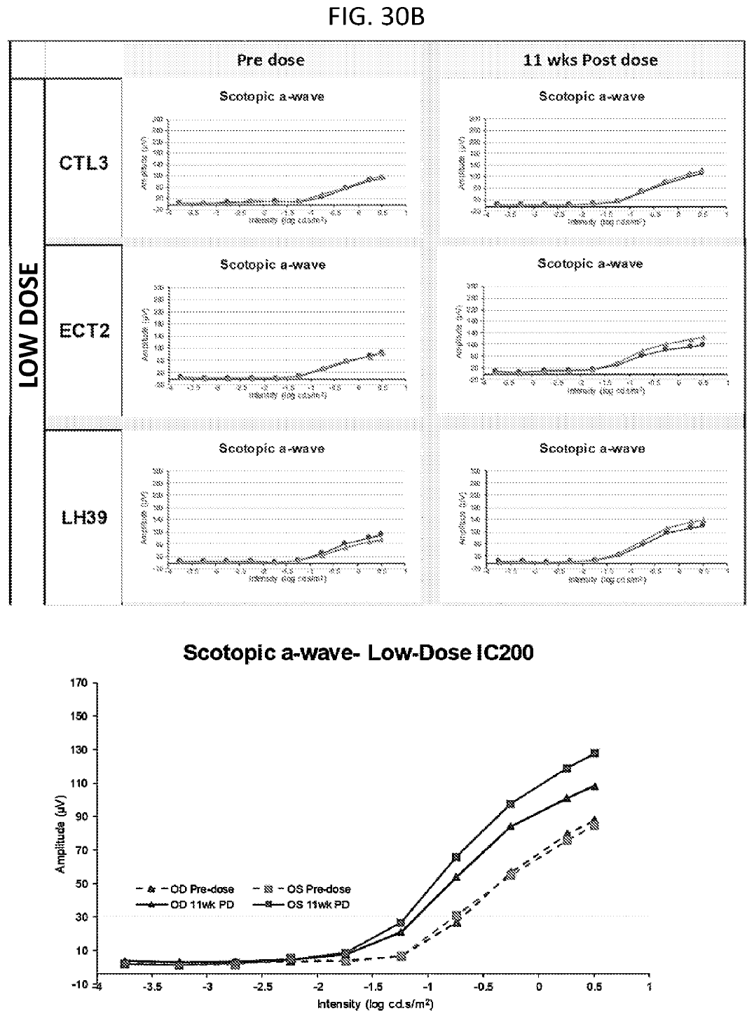

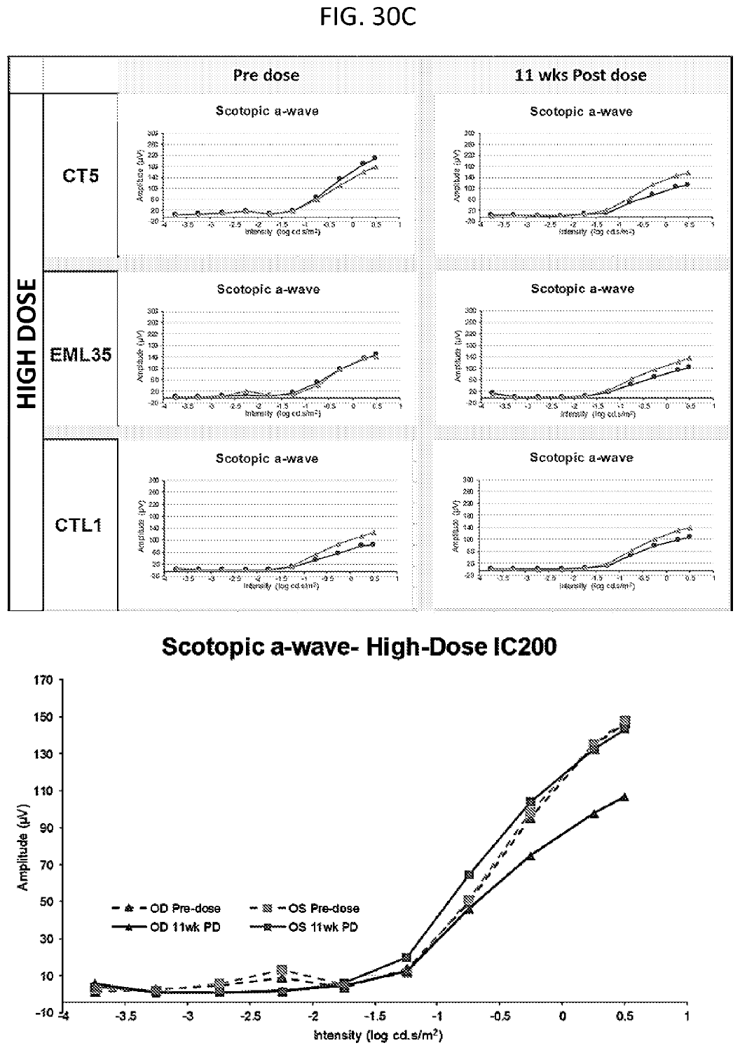

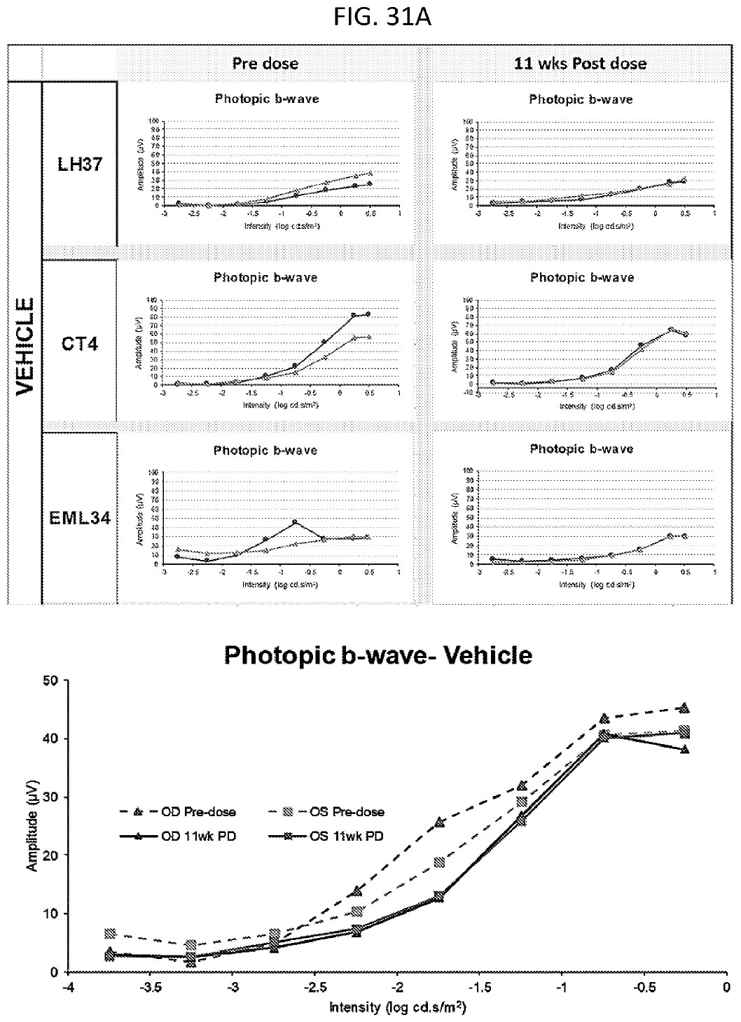

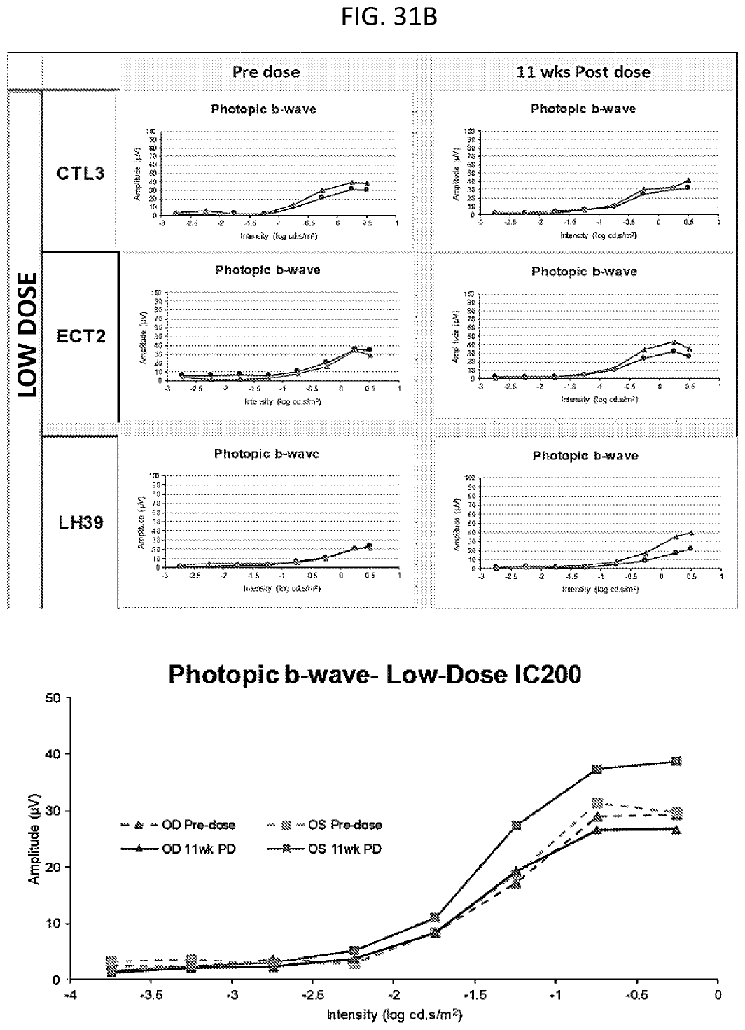

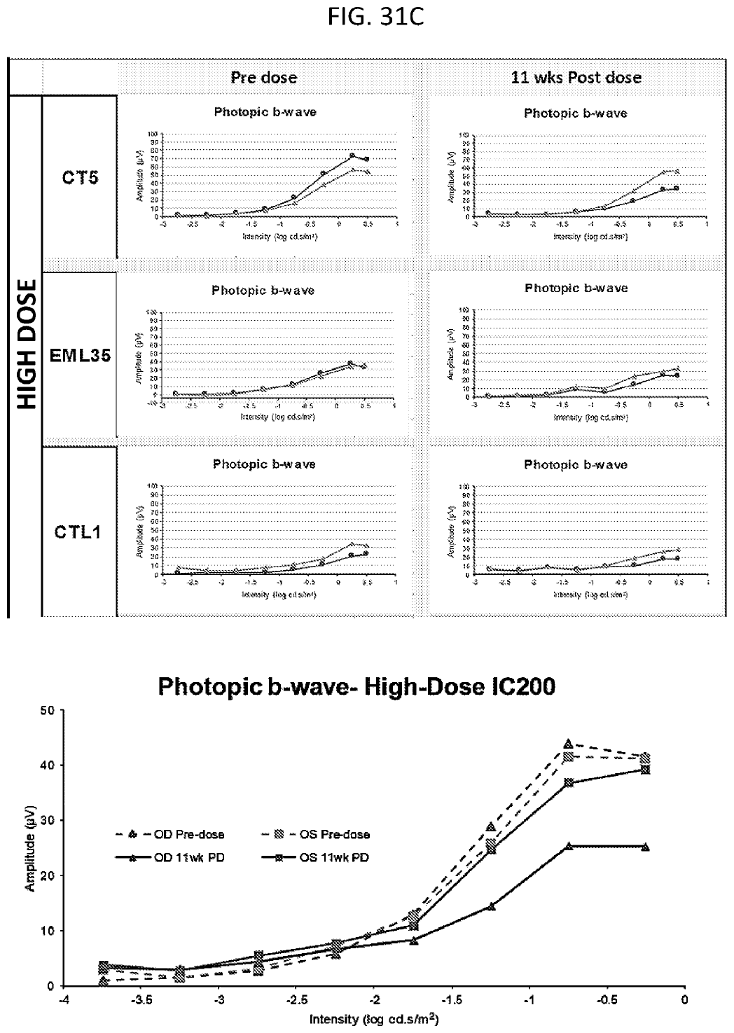

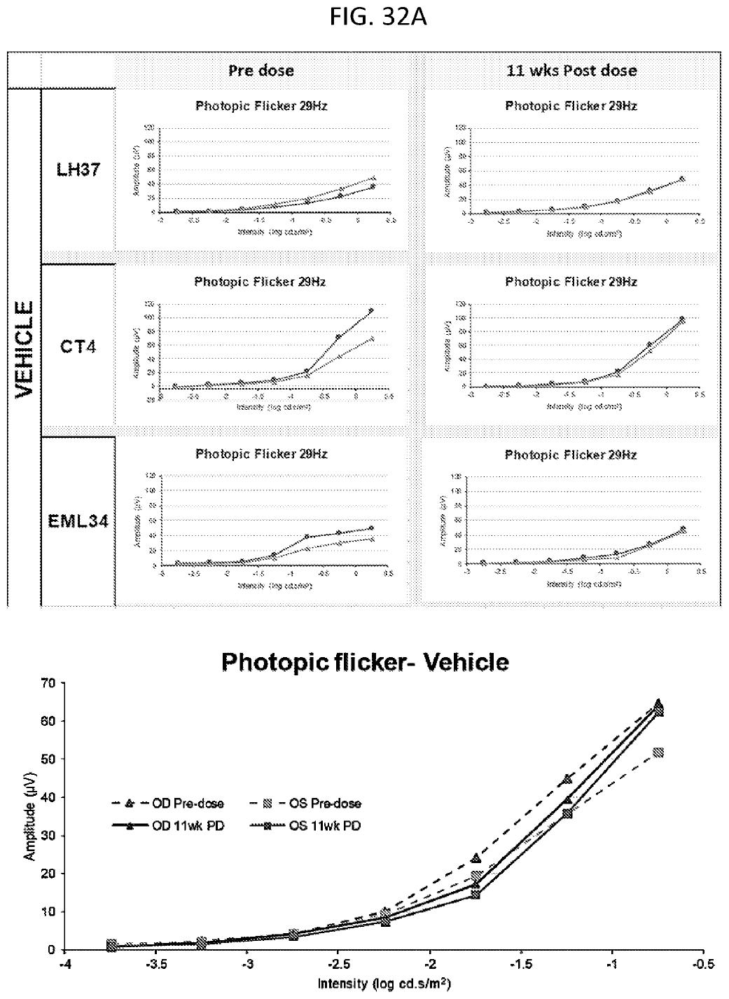

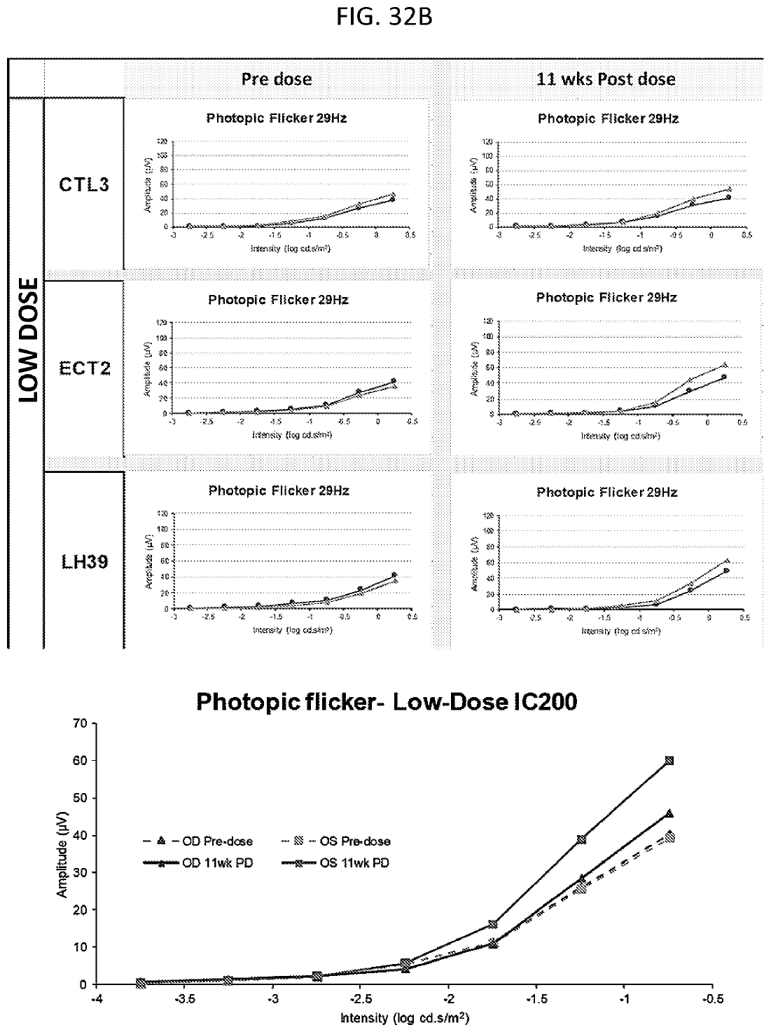

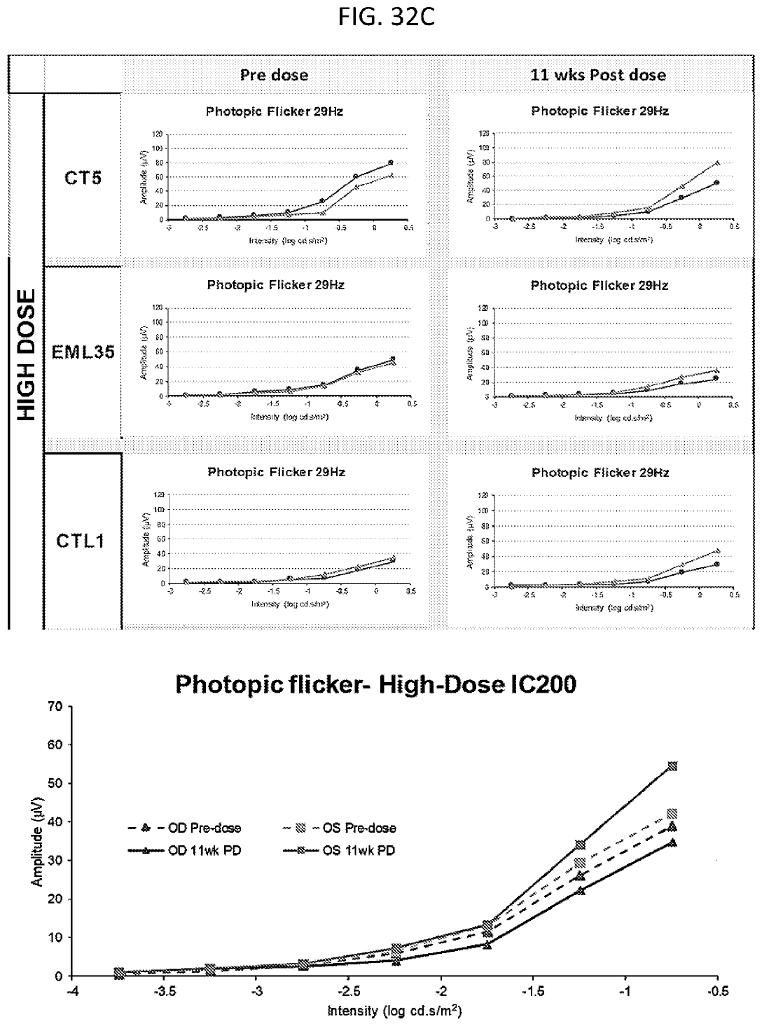

Quantitative analysis did not show any significant differences in mean scotopic a-wave, scotopic b-wave, photopic b-wave, and 29 Hz photopic flicker amplitudes between vehicle-injected eyes (OS) and contralateral un-injected eyes (OD) at pre-dose and 11 weeks post-dose (FIG. 21 and FIG. 29-FIG. 32)

In summary, the vehicle control article did not negatively alter the ERG function in the injected eyes, nor did it confer any beneficial effect in 3 out of 3 animals.

$$Low Dose : 1.4 \times {10}^{9} vg / eye AAV 2 / 2 - BEST 1 Treated Eyes$$

All 3 out of 3 low-dose AAV2/2-BEST1-injected eyes had, at both time points, normal appearing ERG traces with amplitudes that were comparable or slightly lower (ECT2-OS, LH39-OS) than that of WT dogs.

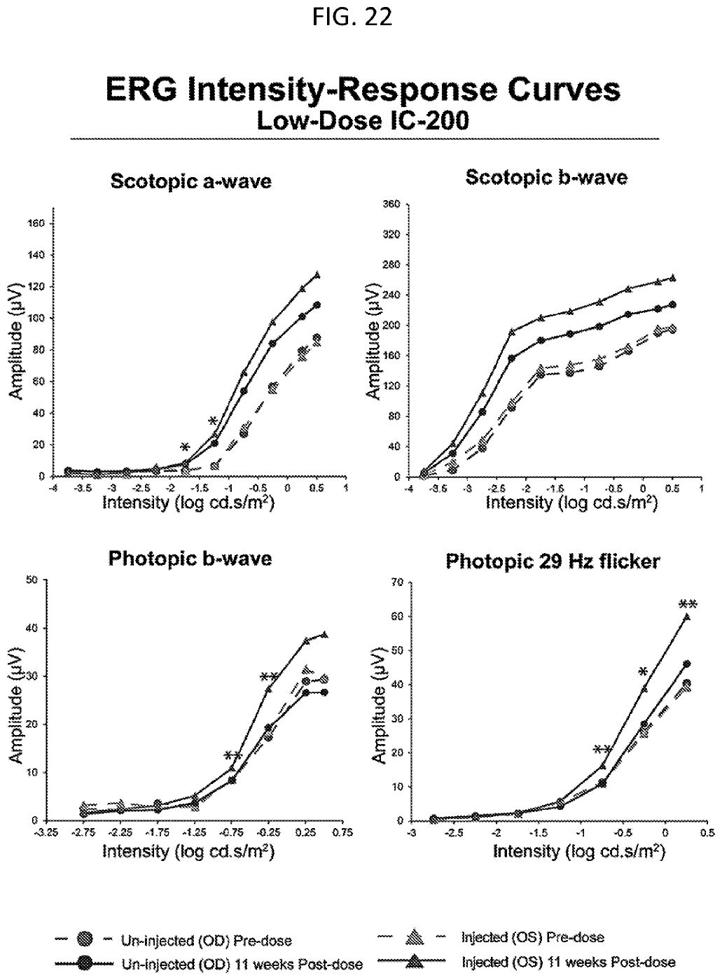

Quantitative analysis showed that the scotopic a-wave amplitudes were, at 11 weeks post-dose, higher in the injected (OS) eyes than in the un-injected (OD) eyes at all light intensities that produce a mixed rod-cone response, and the differences reached statistical significance under 2 intensities (FIG. 22).

Quantitative analysis showed that the mean amplitudes of the scotopic b-waves were, at 11 weeks post-dose, higher in the injected (OS) than in the un-injected (OD) eyes at all light intensities that produce either a rod-only, or a mixed rod-cone response, yet the differences did not reach statistical significance (FIG. 22).

Quantitative analysis showed that the photopic b-wave amplitudes were, at 11 weeks post-dose, higher in the injected (OS) eyes than in the un-injected (OD) eyes at all light intensities that produce a cone response, and the differences reached statistical significance under 2 intensities (FIG. 22).

Quantitative analysis showed that the photopic 29-Hz flicker amplitudes were, at 11 weeks post-dose, higher in the injected (OS) eyes than in the un-injected (OD) eyes at all light intensities that produce a cone response, and the differences reached statistical significance under the 3 highest intensities (FIG. 22).

In summary, low-dose AAV2/2-BEST1 did not negatively alter the ERG function in the injected eyes, but on the contrary, was associated with a trend towards improved rod-, mixed rod-cone-, and cone-mediated responses.

$$High Dose : 4.5 \times {10}^{9} vg / eye AAV 2 / 2 - BEST 1 Treated Eyes$$

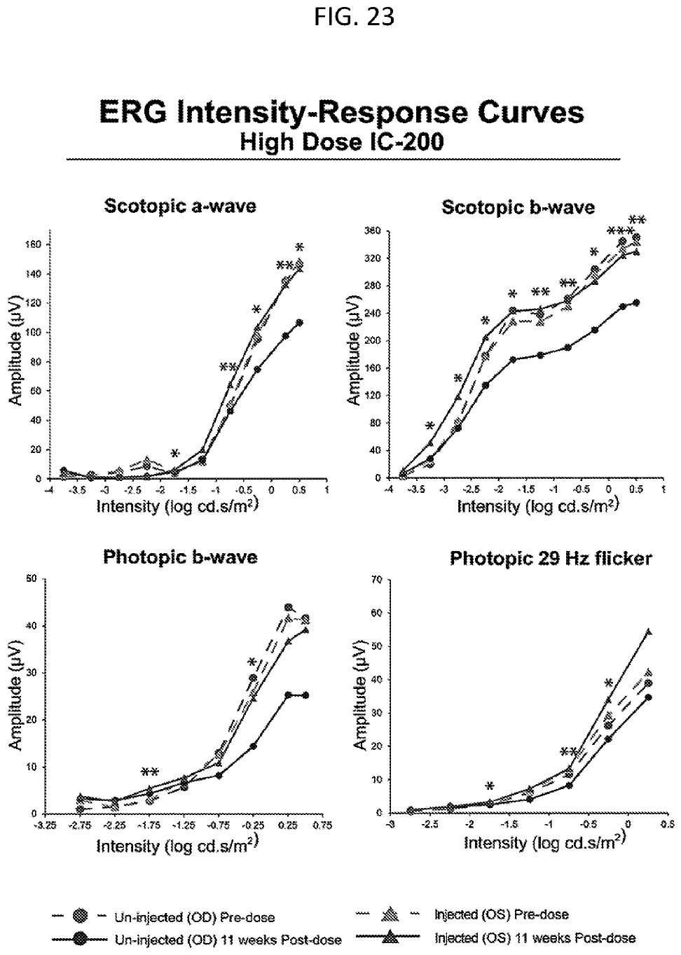

All 3 out of 3 high-dose AAV2/2-BEST1-injected eyes had, at both time points, normal appearing ERG traces with amplitudes (FIG. 23) that were comparable or slightly lower (CTL1-OS) than that of WT dogs.

Quantitative analysis showed that the scotopic a-wave amplitudes were, at 11 weeks post-dose, higher in the injected (OS) eyes than in the un-injected (OD) eyes at all light intensities that produce a mixed rod-cone response, and the differences reached statistical significance under 5 intensities (FIG. 23).

Quantitative analysis showed that the mean amplitudes of the scotopic b-waves were, at 11 weeks post-dose, higher in the injected (OS) than in the un-injected (OD) eyes at all light intensities that produce either a rod-only, or a mixed rod-cone response, and the differences reached statistical significance (FIG. 23).

Quantitative analysis showed that the photopic b-wave amplitudes were, at 11 weeks post-dose, higher in the injected (OS) eyes than in the un-injected (OD) eyes at all light intensities that produce a cone response, and the differences reached statistical significance under 2 intensities (FIG. 23).

Quantitative analysis showed that the photopic 29-Hz flicker amplitudes were, at 11 weeks post-dose, higher in the injected (OS) eyes than in the un-injected (OD) eyes at all light intensities that produce a cone response, and the differences reached statistical significance under 3 intensities (FIG. 23).

### Comparison of ERG Responses Across Treatment Groups

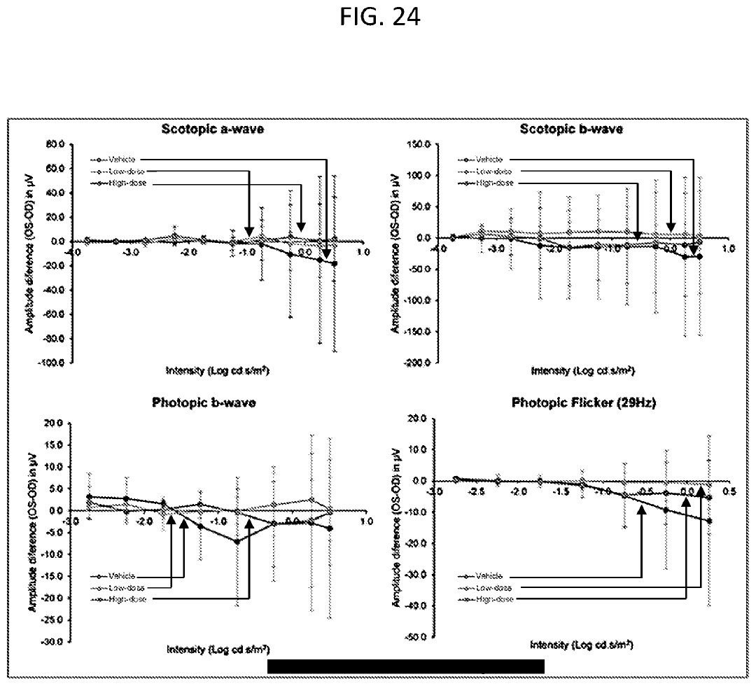

To account for any potential inter-animal variability in ERG responses, the differences in mean amplitudes between the injected (OS) and un-injected (OD) eyes were calculated and compared across treatment groups (FIG. 24 and FIG. 25). At pre-dose, the mean inter-ocular amplitude differences were minimal and there was no significant differences across treatment groups (FIG. 24). At 11 weeks post-dose, there was overall a dose-dependent statistically significant difference across treatment groups in mean inter-ocular amplitude differences (FIG. 25). This was observed under conditions that elicit a rod, cone, and mixed rod-cone response.

The inter-ocular differences in ERG amplitudes observed at 11 weeks post-dose in the low-dose AAV2/2-BEST1 group were found to be the result of an improvement in rod, mixed rod-cone, and cone-mediated responses following treatment with AAV2/2-BEST1 in the injected eyes (FIG. 22). The inter-ocular differences in ERG amplitudes observed at 11 weeks post-dose in the high-dose AAV2/2-BEST1 group were found to be the result of a combination of improved rod- and cone-mediated responses following treatment with AAV2/2-BEST1 in the injected (OS) eyes, and a worsening rod and cone-mediated responses between pre-dose at 11 weeks post-dose time points in the un-injected (OD) eyes (FIG. 23).

In summary, no signs of retinal toxicity that could be associated with either the low-dose or high-dose of AAV2/2-BEST1 were detected by ERG. Results showed, on the contrary, a dose-dependent improvement in rod and cone ERG function following treatment with AAV2/2-BEST1.

### Clinical Pathology

### Vehicle-Injected Eyes

Ages at pre-dose included a 3.5 month old dog (Animal ID No. CT4), a 1 year, 8.5 month old dog (Animal ID No. EIL34), and a 2 year, 8 month old dog (Animal ID No. LH37).

CBC: At 1-week post-dose, there was evidence of acute inflammation, supported by a mild neutrophilia and numbers and degree of neutrophil toxic change, in one dog (Animal ID No. EIL34). There was also evidence of inflammation at 4 weeks post-dose in one dog (Animal ID No. LH37). Inflammation may have been secondary to corticosteroid effects, potentially on the gastrointestinal tract.

Chemistry: There was evidence for dehydration, supported by mild hyperalbuminemia, over several weeks in varying dogs: 1-week post-dose in two dogs (Animal ID Nos. LH37, EIL34), 4 weeks post-dose in two dogs (Animal Nos. CT4, EHL34), and 8 weeks post-dose in one dog (Animal ID No. EIL34). Clinical assessment of dehydration is more valid as biologic variation and the thirst drive can vary in animals. Given the increase in albumin from the prior measurement, this may further support some gastrointestinal effects of corticosteroids.

There was a mild ALT increase in one dog (Animal ID No. LH37) and a moderate ALT increase in one dog (Animal No. EML34) 4 weeks post-dose; increases in ALT support some degree of hepatocellular injury. ALT, a transaminase enzyme, is specific for hepatocyte leakage, often secondary to injury, albeit this can be highly variable. These two dogs also had clinicopathologic evidence of inflammation, so it is possible the inflammation was resulting in some reactive hepatopathy, e.g. if the gastrointestinal tract is the site of the inflammation. This would be attributed to corticosteroid rather than vehicle effects. Steroid effects, i.e. due to hepatocyte glycogen accumulation and cell swelling, can sometimes also result in mild and, less often, moderate ALT increases.

There was evidence of muscle injury 4 weeks post-dose in one dog (Animal ID No. EML34); CK is specific for myocyte injury/leakage. This can be a result of an inflammatory process or trauma, more commonly. A specific cause is unknown and values normalized by 8 weeks post-dose.

Some analytes, outside their respective reference ranges, were a result of age and bone growth in Animal ID No. CT4, i.e. anemia in 4 weeks post-dose, and increases in calcium, phosphorous, and ALP throughout the study with values appropriately decreasing over time.

Some analyte changes can be attributed to corticosteroid effects, from exogenous corticosteroid administration, and are expected and not clinically relevant, i.e., these do not reflect disease/tissue abnormality. These include stress leukograms and increases in ALP due to induction of the corticosteroid-ALP isoform.

Electrolyte and acid base changes, were variable with frequent metabolic alkalosis supported in all dogs at various time points, including pre-dose. In the absence of ileus or another intestinal motility issue and given corticosteroid administration, increased hydrogen ion secretion is suspected. Some age-related changes may also play a role as this is seen pre-dose in the youngest dog (Animal ID No. CT4). Given this acid base disturbance is also noted pre-dose in an older animal (Animal ID No. EML4), this repeated abnormality is unlikely to be clinically relevant and is unrelated to vehicle administration.

### Coagulation:

No clinically relevant abnormalities were seen in coagulation testing in the Vehicle group.

In summary there were no changes in clinical pathology parameters that could be attributed to the vehicle.

$$Low - dose : 1.4 \times {10}^{9} vg / eye AAV 2 / 2 - BEST 1 - Injected Eyes$$

Ages at pre-dose included a 3.5-month dog (Animal ID No. CTL3), an 11 month old dog (Animal ID No. ECT2), and a 2 year, 0.5 month old dog (Animal ID No. LH39).

CBC: There was a mild neutrophilia in one dog (Animal ID No. LH39) 1 week post dose with a concurrent hyperglobulinemia. While this would typically be interpreted as secondary to inflammation, the hyperglobulinemia was present pre-dose, so the neutrophilic could be a result of inflammation and/or a corticosteroid-mediated effect, unrelated to inflammation.

Chemistry: There was evidence for dehydration, supported by mild hyperalbuminemia, for all dogs 1 week post dose and for two dogs 4 weeks post dose (Animal ID Nos. CTL3, ECT2). For ECT2, the elevated albumin was also present pre-dose and persisted past 4 weeks post dose, so it is possible this elevation reflected an animal outside of the normal reference range, which can be seen approximately 5% of the time. Clinical assessment of dehydration is more valid as biologic variation and the thirst drive can vary in animals. Given the increase in albumin from the prior measurement, this may further support some gastrointestinal effects of corticosteroids.

There was a moderate ALT increase in one dog (Animal ID No. ECT2) 1-week post-dose; increases in ALT support some degree of hepatocellular injury. ALT, a transaminase enzyme, is specific for hepatocyte leakage, often secondary to injury, albeit this can be highly variable. Obvious inflammation was not identified via the CBC, but the possibility of a reactive hepatopathy remains, given the potential corticosteroid effects on the intestine. Steroid effects, i.e. due to hepatocyte glycogen accumulation resulting in cell swelling, can sometimes also result in mild and, less often, moderate ALT increases.

Some analytes, outside their respective reference ranges, were a result of age and bone growth in Animal ID No. CTL3, i.e., increases calcium, phosphorous, and ALP throughout the study with values appropriately decreasing over time.

Some analyte changes can be attributed to corticosteroid effects, from exogenous corticosteroid administration, and are expected and not clinically relevant, i.e. these do not reflect disease/tissue abnormality. These include stress leukograms, hyperglycemia, increases in ALP due to induction of the corticosteroid-ALP isoform.

Electrolyte and acid base changes, were variable with frequent metabolic alkalosis supported in all dogs at various time points, including pre-dose. In the absence of ileus or another intestinal motility issue and given corticosteroid administration, increased hydrogen ion secretion is suspected. Given this acid base disturbance was also noted pre-dose in both older dogs (Animal ID Nos. ECT2, LH39), this repeated abnormality is unlikely to be clinically relevant and is unrelated to administration of the low dose of IC200.

Dietary intake can greatly influence potassium values. Additionally, two of the dogs in this group (Animal ID Nos. CTL3, ECT2) are suspected, at 8 weeks post dose to have had pre-analytical errors impacting the results of calcium and sodium. It is possible, sample collection/handling issues could also have influenced potassium.

In two dogs (Animal ID Nos. CTL3 and ECT2), there was likely pre-analytical error resulting in hypocalcemia and hypernatremia 8 weeks post dose. Given the degree of changes and given that concurrent coagulation testing was performed, the potential for citrate contamination of a specimen during collection is possible, as there is not a likely clinical explanation for it, in light of other changes, or lack of other analyte changes, in these dogs.

Coagulation: No clinically relevant abnormalities were seen in coagulation testing in the Low dose group.

In summary, the abnormal clinical pathology findings were similar to those observed in dogs that were treated with the vehicle, and are most likely unrelated to administration of the low-dose of AAV2/2-BEST1.

$$High - dose : 4.5 \times {10}^{9} vg / eye AAV 2 / 2 - BEST 1 - Injected Eyes$$

CBC: Inflammation, supported by a neutrophilia and monocytosis was apparent at 1 week post dose in only the older dog (Animal ID No CTL1). Inflammation may have been secondary to corticosteroid effects, potentially on the gastrointestinal tract. There was also a mild neutrophilia at 8- and 12-weeks post-dose in this dog. This could have resulted from corticosteroid-effects, unrelated to inflammation, and/or a mild component of inflammation.

Age-related CBC changes were noted in both of the younger dogs (CTL5, EML35). This included anemia and hypoglobulinemia earlier in the study; the increasing HCT over time supports a normal change.

Chemistry: All dogs demonstrated clinicopathological support for dehydration, given the mildly elevated albumin at 1 week post-dose. In two dogs (Animal ID Nos. CT5, CTL1), there was also support for dehydration at 4 weeks post-dose.

Some biochemistry analytes, outside their respective reference ranges, were a result of age and bone growth in the younger dogs (Animal ID Nos. CT5, EML35); this included increases in calcium, phosphorous, and ALP throughout the study with values appropriately decreasing over time. ALP changes noted in the older dog (Animal ID No. CTL1) could be attributed to the corticosteroid isoform of ALP (CS-ALP) given the administration of systemic and topical corticosteroids.

There was a mild ALT elevation only in the older dog (Animal ID No. CTL1) at 1 and 4 weeks post dose. Increases in ALT support some degree of hepatocellular injury. ALT, a transaminase enzyme, is specific for hepatocyte leakage, often secondary to injury, albeit this can be highly variable. This dog also had clinicopathologic evidence of inflammation at the same time, so it is possible the inflammation was resulting in some reactive hepatopathy, e.g. if the gastrointestinal tract is the site of the inflammation. This would be attributed to corticosteroid rather than test article effects. Steroid effects, i.e. due to hepatocyte glycogen accumulation and cell swelling, can sometimes also result in mild and, less often, moderate ALT increases.

There was evidence of muscle injury 4 weeks post-dose in one dog (Animal ID No. EML35) given a moderate to marked increase in CK; CK is specific for myocyte injury/leakage. This could be a result of an inflammatory process or trauma, more commonly. A specific cause is unknown and values normalized by 8 weeks post-dose.

Electrolyte and acid base changes, were variable with frequent metabolic alkalosis supported in all dogs at various time points, including pre-dose. In the absence of ileus or another intestinal motility issue and given corticosteroid administration, increased hydrogen ion secretion was suspected. Given this acid base disturbance was also noted pre-dose in two dogs (Animal ID Nos. CTL5, CTL1), this repeated abnormality was unlikely to be clinically relevant and was unrelated to administration of the high dose of AAV2/2-BEST1.

Coagulation: No clinically relevant abnormalities were seen in coagulation testing in the High dose group.

In summary, the abnormal clinical pathology findings were similar to those observed in dogs treated with the vehicle, and are most likely unrelated to administration of the high-dose of AAV2/2-BEST1.

### Comparison of Clinical Pathology Results Across Treatment Groups

At 4 weeks post dose, there is a statistically significant difference between RBC parameters of vehicle and low dose and vehicle and high dose groups with the vehicle group having a relative decrease in RBC parameters, i.e. PCV, Hemoglobin concentration, and Hematocrit; alternatively, it could be stated that the low and high dose groups have a relative increase in these parameters, compared to the vehicle group. This is unlikely to be clinically relevant and is more likely a result of a biologic variation and small group size, given the known variability in RBC parameter ranges. It also seems unrelated to dehydration given the presence of dehydration in all groups.

The lower mean cholesterol in the Low dose group vs. the Vehicle group at 8 weeks post dose is statistically significant but unlikely to be clinically relevant, as cholesterol has a wide normal reference range and biologic and day-to-day variability (given dietary influences); the small group size likely exacerbates this difference.

The statistical difference in creatinine at 12 weeks post-dose for High vs. Low group when compared to the Vehicle group is unlikely to be clinically relevant or related to administration of the high-dose of AAV2/2-BEST1.

In summary, there were no statistically significant or biologically relevant changes noted between groups that could be attributed to the AAV2/2-BEST1 test article.

### Terminal Evaluations for Safety

### Organ Weights (Week 13 Necropsy)

Differences in organ weights, organ-to-body weight ratios, and organ-to-brain weights across treatment groups were not statistically significant and were otherwise not suggestive of test article effects for all collected organs, with the exception of ovaries. For the ovaries, the interpretation of the results of the statistical analysis was limited by the low number of females (1) in each treatment group.

### Non-Ocular Macroscopic and Microscopic Pathology (Week 13 Necropsy)

### Macroscopic Pathology Findings (Week 13 Necropsy)

No AAV2/2-BEST1 related macroscopic findings were noted in any of the dogs included in this study.

Across all treatment groups (Tables 23-27), some dogs presented gross lesions at Week 13 during necropsy that were most likely attributable to the administration of corticosteroids, such as the hepatic discoloration with an enhanced lobular pattern and the atrophy of the adrenal glands. Other findings, such as the multifocal chronic dermatitis, cutaneous papillomas, splenic choristomas, abdominal hernia and cardiac changes are considered as non-significant background findings in dogs unrelated to the experimental treatments.

### Non-Ocular Microscopic Pathology Findings

No definitive AAV2/2-BEST1-related histologic findings were seen in the organs examined.

In a single dog (Animal ID No. EML34) from the Vehicle treatment group, a thin perivascular cuff composed of mononuclear cells (mostly lymphocytes) was noted within the thalamus. This changes was not associated with obvious neuroaxonal alterations and is of unclear significance. The same dog also exhibited inflammatory cell infiltrates comprised of lymphocytes, plasma cells and macrophages centered around mesenteric lymphatic vessels and within the lamina propria of the jejunum. These findings are reminiscent of a mild form of lipogranulomatous lymphangitis, an uncommon cause of gastrointestinal disease in dogs.

The changes noted in the liver (i.e. hepatocellular swelling and clearing) and in the testes (i.e. decreased spermatogenesis) were attributed to the corticosteroid administration.

Similarly, while some Herring bodies can be seen in the hypothalamus of normal dogs, there was an increased number in the study dogs that likely resulted from the corticosteroid administration.

The changes in the cranial lung lobes seen in Animal ID No. EM512 from the low dose-treated group are non-specific. Given the cranioventral distribution, a minor aspiration event is suspected.

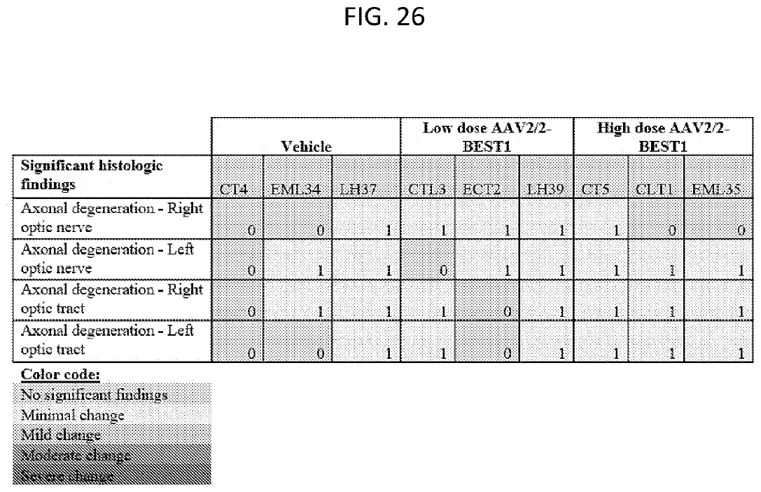

Most dogs exhibited axonal degeneration in the optic nerves and optic tracts (FIG. 26), characterized by dilated myelin sheaths, swollen and hyper-eosinophilic axons (spheroids) and/or myelinomacrophages within digestion chambers. These changes remained minimal and were often seen bilaterally throughout all experimental groups, including the vehicle-injected group. The cause for the axonal degeneration is difficult to ascertain and may have been caused by the focal retinal disruption at the site of retinotomy, the advanced stage of retinal degenerative disease with secondary effect on the ganglion cells, or a combination of both. The bilateral nature of this finding supports the latter hypothesis.

All dogs exhibited one or more of the following spontaneous background lesions:
Perivascular and perifollicular dermal inflammatory cell infiltrates with occasional intralesional Demodex mites.
Inflammatory cell aggregates in the lung interstitium.
Hepatic inflammatory cell aggregates.
Renal tubular basophilia.
Lymph node sinus histiocytosis and cortical and paracortical hyperplasia.
Salivary gland interstitial inflammatory cell infiltrates.
Splenic choristoma within the omentum.
Ovarian teratoma.

None of the above lesions exhibited an incidence related to test article dose level.

TABLE 21
Summary of Histopathology Findings in the Vehicle Control Group

| | Pathology Findings | | |
|---|---|---|---|
| | Gross | Histopathology | |
| DOG ID | Pathology | Finding | Organ/Tissue |
| LH37 | Small adrenal glands | Inflammatory cell infiltrate | Liver (minimal) |
| | Dental calculus | | R Limbus (mild) |
| | | | L Limbus (minimal) |
| | | Steroid hepatopathy | Liver (mild) |
| | | Tubular vacuolation | R/L Kidney (mild) |
| | | Teratoma | L Ovary (focal) |
| | | Chronic drainage reaction | R/L Mandibular LN (mild) |
| | | Axonal degeneration | R/L Optic Nerve (minimal) |
| | | | R Optic Tract (minimal) |
| | | Herring bodies | Hypothalamus (moderate) |
| | | RPE cell vacuolation | R Retina (mild) |
| | | | L Retina (minimal) |
| CT4 | Small adrenal glands | Inflammatory cell infiltrate | Liver (minimal) |
| | Focal Hemocyst | | L Eyelid (minimal) |
| | | | L Limbus (minimal) |
| | | Steroid hepatopathy | Liver (moderate) |
| | | Reduced spermatogenesis | R/L Testis (severe) |
| | | Drainage reaction | R/L Mandibular LN (mild) |
| | | Herring bodies | Hypothalamus (moderate) |
| | | RPE hypertrophy | L Retina (minimal) |
| | | RPE cell vacuolation | R/L Retina (mild) |
| | | Retinal folds | L Retina (minimal) |
| | | Retinal detachment | L Eye (minimal) |
| | | Hemocyst | Tricuspid valve (focal) |
| EML34 | Bilateral cryptorchidism | Valvular endocardiosis | Mitral valve (mild) |
| | Small adrenal glands | Steroid hepatopathy | Liver (mild) |
| | Siderofibrotic plaques | Inflammatory cell infiltrate | R Lung (minimal) |
| | Valvular endocardiosis | | R/L Eyelid (minimal) |
| | | | Liver (minimal) |
| | | | L Limbus (minimal) |
| | | Siderofibrotic plaque | Spleen (regional) |
| | | Enteritis | Jejunum (mild) |
| | | Lymphangitis | Jejunum (mild) |
| | | Steroid hepatopathy | Liver (minimal) |
| | | Reduced spermatogenesis | R/L Testis (severe) |
| | | Chronic drainage reaction | R/L Mandibular LN (mild) |
| | | Perivascular cuff | Thalamus (minimal) |
| | | Axonal degeneration | L Optic nerve (minimal) |
| | | | R Optic tract (minimal) |
| | | Herring bodies | Hypothalamus (moderate) |
| | | Multifocal detachment | R/L Retina (mild) |
| | | RPE hypertrophy | R/L Retina (mild) |
| | | RPE cell vacuolation | R Retina (mild to moderate) |
| | | | L Retina (moderate) |

TABLE 22
Summary of Histopathology Findings in the Low-Dose AAV2/2-BEST1 Group

| | Pathology Findings | | |
|---|---|---|---|
| | Gross | Histopathology | |
| DOG ID | Pathology | Finding | Organ/Tissue |
| CTL3 | Small adrenal glands | Inflammatory cell infiltrate | Liver (minimal) |
| | Chronic dermatitis | | L Lung (minimal) |
| | Steroid hepatopathy | Demodicosis | Skin (moderate) |
| | Splenic choristoma | Steroid hepatopathy | Liver (mild) |
| | | Reduced spermatogenesis | R/L Testis (mild to moderate) |
| | | Alveolar histiocytosis | R Lung (minimal) |
| | | Chronic drainage reaction | R/L Mand. LN (mild) |
| | | Axonal degeneration | R Optic nerve (minimal) |
| | | | L Optic tract (minimal) |
| | | Herring bodies | Hypothalamus (low) |
| | | RPE cell vacuolation | R Retina (mild to moderate) |
| | | | L Retina (mild) |
| | | Splenic choristoma | Omentum (focal) |
| ECT2 | Small adrenal glands | Inflammatory cell infiltrate | Liver (minimal) |
| | Septal defect (heart) | | L Lung (minimal) |
| | | | L Limbus |
| | | Steroid hepatopathy | Liver (moderate) |
| | | Tubular vacuolation | R/L kidney (mild) |
| | | Alveolar histiocytosis | R Lung (minimal) |
| | | Chronic drainage reaction | R/L Mand. LN (mild) |
| | | Axonal degeneration | R/L Optic nerve (minimal) |
| | | Herring bodies | Hypothalamus (moderate) |
| | | RPE cell vacuolation | R Retina (minimal) |
| | | | L Retina (mild) |
| LH39 | Small adrenal glands | Steroid hepatopathy | Liver (moderate) |
| | Dental Calculus | Inflammatory cell infiltrate | Liver (minimal) |
| | Unilateral Cryptorchid (L) | | R Lung (minimal) |
| | | | L Parotid gland (minimal) |
| | | | L Limbus |
| | | Reduced spermatogenesis | R Testis (mild) |
| | | | L Testis (severe) |
| | | Tubular vacuolation | L Kidney (mild) |
| | | Chronic drainage reaction | R/L Mandibular LN (mild) |
| | | Axonal degeneration | R/L Optic nerve (minimal) |
| | | | R/L Optic tract (minimal) |
| | | Herring bodies | Hypothalamus (high) |
| | | RPE cell vacuolation | R/L Retina (minimal) |

TABLE 23
Summary of Histopathology Findings in the High-Dose AAV2/2-BEST1 Group

| | Pathology Findings | | |
|---|---|---|---|
| | Gross | Histopathology | |
| DOG ID | Pathology | Finding | Organ/Tissue |
| CT5 | Small adrenal glands | Inflammatory cell | Liver (mild) |
| | Bilateral | infiltrate | R/L Lung (mild) |
| | cryptorchidism | | |
| | Splenic choristoma | | L Eyelid (minimal) |
| | | Steroid hepatopathy | Liver (mild) |
| | | Reduced | R/L Testis (severe) |
| | | spermatogenesis | |
| | | Chronic drainage | R/L Mandibular LN (mild) |
| | | reaction | |
| | | Demodicosis | L Eyelid |
| | | Axonal degeneration | R/L Optic Nerve (minimal) |
| | | | R Optic Tract (minimal) |
| | | Herring bodies | Hypothalamus (moderate) |
| | | Splenic choristoma | Omentum (focal) |
| | | RPE hypertrophy | R Retina (minimal) |
| | | RPE cell vacuolation | R/L Retina (mild) |
| | | Retinal fold | L Retina (mild) |
| EML35 | Small adrenal glands | Inflammatory cell | Skin (minimal) |
| | Abdominal hernia | infiltrate | Liver (minimal) |
| | Chronic dermatitis | | R Lung (minimal) |
| | Cutaneous papillomas | | L Kidney (minimal) |
| | Red pachymeninges | | R Eyelid (mild) |
| | | | R/L Limbus |
| | | Steroid hepatopathy | Liver (mild) |
| | | Tubular basophilia | L Kidney |
| | | Chronic drainage | R Mandibular LN (mild to |
| | | reaction | moderate) |
| | | | L Mandibular LN (mild) |
| | | Axonal degeneration | L Optic Nerve (minimal) |
| | | | R/L Optic Tract (minimal) |
| | | Herring bodies | Hypothalamic (moderate) |
| | | Viral papillomas | Skin (multifocal) |
| | | RPE cell vacuolation | R/L Retina (mild) |
| | | Layering disruption | L Retina (focal) |
| CTL1 | Small adrenal glands | Steroid hepatopathy | Liver (moderate) |
| | Chronic dermatitis | Reduced | R/L Testis (minimal) |
| | | spermatogenesis | |
| | | Inflammatory cell | R/L eyelid (mild) |
| | | infiltrate | Liver (minimal) |
| | | | R Limbus (minimal) |
| | | Chronic drainage | R/L Mandibular LN (mild) |
| | | reaction | |
| | | Axonal degeneration | L Optic nerve (minimal) |
| | | | L/R Optic Tracts (minimal) |
| | | Herring bodies | Hypothalamus (low) |
| | | Dermatitis | Skin (mild) |
| | | Retinal detachment | R Eye (regional) |
| | | RPE cell vacuolation | R Retina (minimal to mild) |
| | | | L Retina (mild) |
| | | RPE hypertrophy | R/L Eye (minimal to mild) |

### Microscopic Ocular Pathology (Week 13 Necropsy)

### Un-Injected Eyes

No histologic evidence of intraocular hemorrhage, inflammation, or necrosis were observed in any of the un-injected (right) eyes. Two eyes (Animal ID Nos. EML34-OD and CTL1-OD) presented mild multifocal retinal detachment with hypertrophy of the RPE, and accumulation of cytoplasmic lipofuscin (Animal ID No. CTL1-OD), which are classic features in dogs of BEST1 disease-related abnormalities at the photoreceptor-RPE interface.

### Vehicle-Injected Eyes

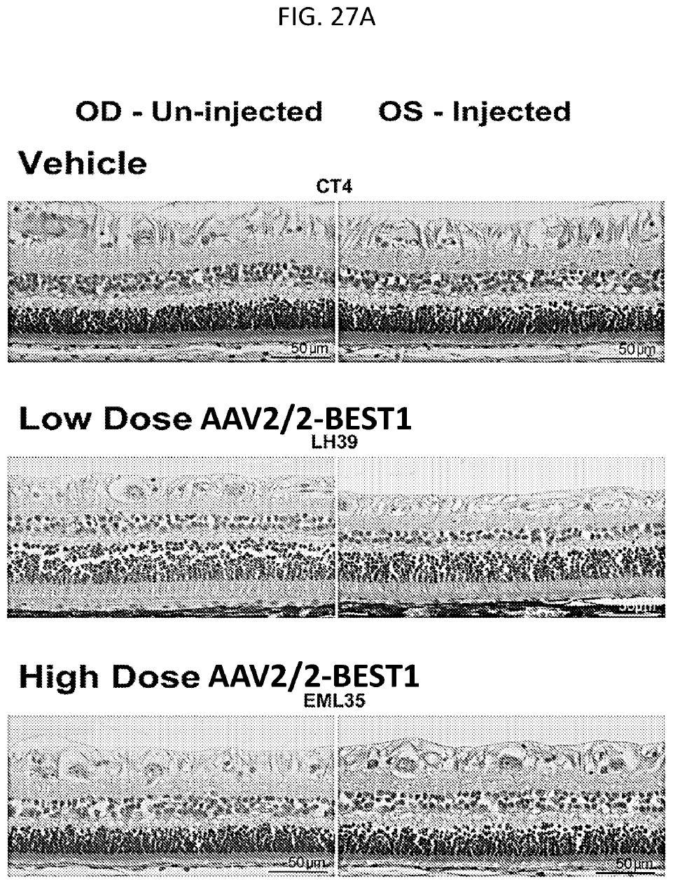

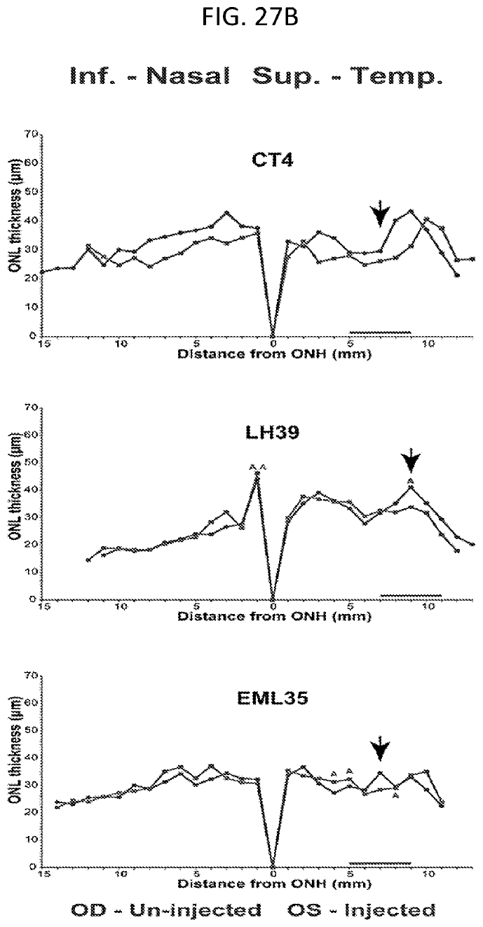

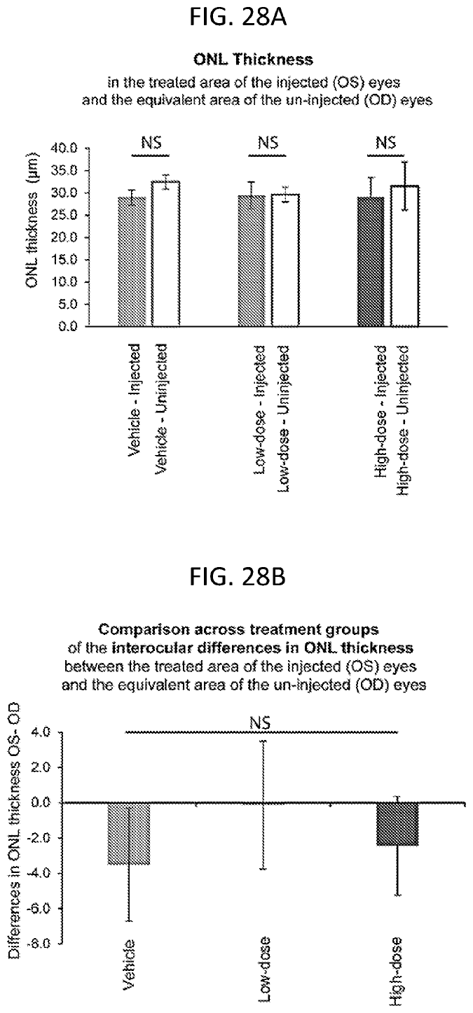

No histologic evidence of intraocular hemorrhage, inflammation, or necrosis were observed in all 3 vehicle-treated eyes. Two of the 3 eyes (Animal ID Nos. CT4-OS and EML34-OS) exhibited minimal to mild multifocal retinal detachment with RPE hypertrophy. Dog EML34 also had lipofuscin accumulation in the cells of the RPE. These are classic features in dogs of BEST1 disease-related abnormalities at the photoreceptor-RPE interface. All 3 right eyes in this treatment group showed preservation of the ONL and inner and outer segments in the treated area, with a mean ONL thickness of 28.9 μm that was comparable to that of the equivalent area (32.5 m) of the un-injected contralateral eyes (FIG. 27A and FIG. 27B, FIG. 28A).

In summary, no ocular abnormalities related to administration of the vehicle were observed by light microscopy.

$$Low - Dose : 1.4 \times {10}^{9} vg / eye AAV 2 / 2 - BEST 1 Injected Eyes$$

No histologic evidence of intraocular hemorrhage, inflammation or necrosis were observed in all 3 low dose-treated eyes (Animal ID Nos CTL3-OS, ECT2-OS, LH39-OS). No other findings were seen in any of the 3 eyes in this treatment group. All 3 right eyes in this treatment group showed preservation of the ONL and inner and outer segments in the treated area, with a mean ONL thickness of 29.4 μm that was comparable to that of the equivalent area (29.6 μm) of the un-injected contralateral eyes (FIG. 27A and FIG. 27B, FIG. 28A).

In summary, no ocular abnormalities related to administration of low-dose AAV2/2-BEST1 were observed by light microscopy.

$$High - Dose : 4.5 \times {10}^{9} vg / eye AAV 2 / 2 - BEST 1 Injected Eyes$$

No histologic evidence of intraocular hemorrhage, inflammation, or necrosis were observed in all 3 high dose-treated eyes. One eye (Animal ID No. CTL1-OS) presented minimal to mild multifocal hypertrophy of the RPE with accumulation of lipofuscin without obvious retinal detachment. These are classic features in dogs of BEST1 disease-related abnormalities of the RPE.

All 3 right eyes in this treatment group showed preservation of the ONL and inner and outer segments in the treated area, with a mean ONL thickness of 29.1 μm that was comparable to that of the equivalent area (31.5 m) of the un-injected contralateral eyes (FIG. 27A and FIG. 27B, FIG. 28A).

In summary, no ocular abnormalities related to administration of high-dose AAV2/2-BEST1 were observed by light microscopy.

### Comparison of ONL Thickness Across Treatment Groups

To account for any potential inter-animal variability in ONL thickness due in particular to age, the differences in mean ONL thickness between the injected (OS) and un-injected (OD) eyes were calculated and compared across treatment groups. Results did not show any significant difference in mean (OS-OD) ONL thickness across treatment groups (FIG. 28A and FIG. 28B show).

Taken together, these results suggest the absence of adverse effects detectable by light microscopy in the eyes injected with the AAV2/2-BEST1 test article.

TABLE 26
ONL thickness (in μm) measured by histology at each of the
5 loci selected within the treated area of the injected (OS)
eyes and the equivalent area of the un-injected (OD) eyes.

| | Dog | | ONL thickness (μm) at each locus | | | | |
|---|---|---|---|---|---|---|---|
| Treatment | ID | Eye | 1 | 2 | 3 | 4 | 5 |
| Vehicle | LH37 | OS - | 32.87 | 32.78 | 29.61 | 29.52 | 29.55 |
| | | Injected | | | | | |
| | | OD - | 36.35 | 33.33 | 28.85 | 25.6 | 31.82 |
| | | Un-injected | | | | | |
| | CT4 | OS - | 28.04 | 24.93 | 26.16 | 27.29 | 31.35 |
| | | Injected | | | | | |
| | | OD - | 29.09 | 28.95 | 29.61 | 40.31 | 43.5 |
| | | Un-injected | | | | | |
| | EML34 | OS - | 28.73 | 28.7 | 28.74 | 28.3 | 27.41 |
| | | Injected | | | | | |
| | | OD - | 33.02 | 39.71 | 28.82 | 29.45 | 28.38 |
| | | Un-injected | | | | | |
| Low-dose | CTL3 | OS - | 25.46 | 27.29 | 24.93 | 33.87 | 26.83 |
| AAV2/2- | | Injected | | | | | |
| BEST1 | | OD - | 23.54 | 24.06 | 25.15 | 28.17 | 37.93 |
| | | Un-injected | | | | | |
| | ECT2 | OS - | 29.89 | 31.91 | 30.92 | 29.75 | 26.44 |
| | | Injected | | | | | |
| | | OD - | 25.68 | 26.4 | 25.55 | 28.75 | 25.22 |
| | | Un-injected | | | | | |
| | LH39 | OS - | 32.5 | 32.02 | 33.84 | 31.82 | 23.77 |
| | | Injected | | | | | |
| | | OD - | 31.75 | 35.26 | 41.12 | 35.35 | 29.4 |
| | | Un-injected | | | | | |
| High-dose | CT5 | OS - | 28.09 | 27.61 | 31.87 | 32.16 | 37.42 |
| AAV2/2- | | Injected | | | | | |
| BEST1 | | OD - | 37.99 | 35.71 | 34.68 | 37.34 | 39.81 |
| | | Un-injected | | | | | |
| | EML35 | OS - | 32.26 | 26.83 | 28.47 | 29.05 | 33.71 |
| | | Injected | | | | | |
| | | OD - | 29.68 | 28.31 | 34.53 | 29.53 | 33.08 |
| | | Un-injected | | | | | |
| | CTL1 | OS - | 23.26 | 21.76 | 22.04 | 29.86 | 31.39 |
| | | Injected | | | | | |
| | | OD - | 22.67 | 26.39 | 23.76 | 29.65 | 29.39 |
| | | Un-injected | | | | | |

TABLE 27
Mean ONL thickness measured on Histological Sections
in Treated (and equivalent-treated) Areas.

| | | | | | Low-dose | | | High-dose | | |
|---|---|---|---|---|---|---|---|---|---|---|
| | | Vehicle | | | 1.4 × 10⁹ vg/eye | | | 4.5 × 10⁹ vg/eye | | |
| | | CT4 | EML34 | LH37 | CTL3 | ECT2 | LH39 | CT5 | CTL1 | EML35 |
| Mean ONL | OS | 27.6 | 28.4 | 30.9 | 27.7 | 29.8 | 30.8 | 31.4 | 25.7 | 30.1 |
| thickness in | OD | 34.3 | 31.9 | 31.2 | 27.8 | 26.3 | 34.6 | 37.1 | 26.4 | 31 |
| the treated | | | | | | | | | | |
| (OS) or | | | | | | | | | | |
| equivalent- | | | | | | | | | | |
| treated | | | | | | | | | | |
| (OD) areas | | | | | | | | | | |
| (μm) | | | | | | | | | | |

### CONCLUSIONS

This nonclinical study was conducted in a manner that addressed the key components of the principles of Good Laboratory Practice (GLP) regulations as set forth in 21 CFR Part 58, GLP for Nonclinical Laboratory Studies to evaluate the efficacy and safety of AAV2/2-BEST1 in BEST1-mutant dogs, a naturally-occurring large animal model of bestrophinopathy.

Evidence of a significantly lower OS+ thickness (=reduced IS/OS to RPE/T distance) was seen in the treated area of the injected eyes in both (low-dose, and high-dose) AAV2/2-BEST1 treatment groups at 12 weeks post-dose when compared by standard resolution and high-resolution OCT imaging to an equivalent area of the contralateral un-injected eyes. These results confirm previously published findings that showed structural improvement of the photoreceptor to RPE interface following AAV-mediated BEST1 gene augmentation with a research grade vector.

Although a hyperthick ONL has recently been reported as a feature of canine BEST1 disease, dogs in this study in which a prominent thinning of the ONL occurred during the pre-dose and the 12 weeks-post-dose period, were young animals, and a similar change was seen in untreated areas of the injected eye as well as in the contralateral un-injected eye. Thus, in these dogs ONL thinning was likely the result of aging rather than therapeutic intervention.

Mutant BEST1 dogs that were used in this study were of different ages with variable stages of disease. Thus, clinical and OCT evidence of clear retinal reattachment of Stage III (pseudohypopyon) lesions following dosing was only seen in a single dog (out of 3) in both the low-dose AAV2/2-BEST1 and high-dose AAV2/2-BEST1 groups that had preexistent advanced disease at pre-dose. Similarly, evidence of the efficacy of AAV2/2-BEST1 at preventing the occurrence of stage II (vitelliform lesions) disease in the treated area was observed in a single animal within the high-dose group.

No mortality or any clinical signs of poor health occurred in any animals, and all 9 dogs remained successfully enrolled until the end of the 13-week in life phase of the study.

Clinical pathology results did not reveal any findings that could be associated with AAV2/2-BEST1 treatment.

In vivo retinal examination by indirect ophthalmoscopy, and cSLO/OCT did not reveal any lesions that could be unambiguously related to AAV2/2-BEST1 in the eyes injected with the low-, or high-dose.

No electroretinographic abnormalities were seen in any of the AAV2/2-BEST1 or vehicle-injected eyes. While ERG was primarily conducted to identify any potential retinal toxicity, data analysis unexpectedly showed that ERG amplitudes in low-dose and high-dose AAV2/2-BEST1 injected eyes were higher than in the uninjected contralateral eyes. This may be the functional readout of an improved retinoid visual cycle as a result of restored structural integrity at the photoreceptor-RPE interface.

Histopathologic examinations of non-ocular tissues in the vehicle, low-, and high-dose groups did not reveal any findings that could be clearly associated with the test-article. Ocular histopathology in the vehicle, low-dose AAV2/2-BEST1, and high-dose AAV2/2-BEST1 was unremarkable. No signs of inflammation, nor toxicity were detected, and measurements of ONL thickness within the treated area was normal and similar to that of the equivalent treated area of the un-injected contralateral eyes.

In summary, a single subretinal injection of either doses (1.4×10⁹ and 4.5×10⁹ vg/eye) of AAV2/2-BEST1 led to structural and functional improvement in the retinas of BEST1-mutant dogs, and did not cause any in-life adverse findings at 13 weeks after injection. The study results do not identify the NOAEL of AAV2/2-BEST1 which is likely above 4.5×10⁹ vg/eye (150 μL, 3.0×10¹⁰ vg/mL) in the naturally occurring BEST1-mutant dogs.

### REFERENCES

Aleman T S, Cideciyan A V, Aguirre G K, et al. Human CRB1-associated retinal degeneration: comparison with the rd8 Crbl-mutant mouse model. Invest Ophthalmol Vis Sci 2011; 52:6898-6910.
Beltran W A, Hammond P, Acland G M, Aguirre G D. A frameshift mutation in RPGR exon ORF15 causes photoreceptor degeneration and inner retina remodeling in a model of X-linked retinitis pigmentosa. Invest Ophthalmol Vis Sci 2006; 47(4):1669-1681.
Beltran W A, Cideciyan A V, Lewin A S, Iwabe S, Khanna H, et al. Gene therapy rescues X-linked photoreceptor blindness in dogs and paves the way for treating RPGR form of human retinitis pigmentosa. Proc Natl Acad Sci USA 2012; 109:2132-2137.
Beltran W A, Cideciyan A V, Guziewicz K E, Iwabe S, et al. Canine retina has a primate fovea-like bouquet of cone photoreceptors which is affected by inherited macular degenerations. PLoS One 2014; 9:e90390.
Boye S E, Huang W C, Roman A J, Sumaroka A, Boye S L, et al. Natural history of cone disease in the murine model of Leber congenital amaurosis due to CEP290 mutation: determining the timing and expectation of therapy. PLoS One. 2014; 9(3):e92928.
Cideciyan A V, Jacobson S G, Aleman T S, Gu D, Pearce-Kelling S E, et al. In vivo dynamics of retinal injury and repair in the rhodopsin mutant dog model of human retinitis pigmentosa. Proc Natl Acad Sci USA 2005; 102:5233-5238.
Davidson A E, Millar I D, Urquhart J E, Burgess-Mullan R, et al. Missense mutations in a retinal pigment epithelium protein, bestrophin-1, cause retinitis pigmentosa. American journal of human genetics. 2009; 85:581-592.
Guziewicz K E, Zangerl B, Lindauer S J, Mullins R F, et al. Bestrophin gene mutations cause canine multifocal retinopathy: a novel animal model for Best disease. Invest Ophthalmol Vis Sci 2007; 48:1959-1967.
Guziewicz K E, Zangerl B, Komáromy A M, Iwabe S, et al. Recombinant AAV-mediated BEST1 transfer to the retinal pigment epithelium: analysis of serotype-dependent retinal effects. PLoS One 2013; 8:e75666.
Guziewicz K E, Sinha D, Gómez N M, Zorych K, et al. Bestrophinopathy: An RPE-photoreceptor interface disease. Prog Retin Eye Res 2017; 58:70-88.
Guziewicz K E, Cideciyan A V, Beltran W A, Komiromy A M, et al. BEST1 gene therapy corrects a diffuse retina-wide microdetachment modulated by light exposure. Proc Natl Acad Sci USA 2018; 115(12):E2839-E2848.
Huang W C, Wright A F, Roman A J, Cideciyan A V, Manson F D, et al. RPGR-associated retinal degeneration in human X-linked R P and a murine model. Invest Ophthalmol Vis Sci 2012; 53: 5594-608.
Zangerl B, Wickstrom K, Slavik J, Lindauer S J, Ahonen W, Schelling C, Lohi H, Guziewicz K E, Aguirre G D. Assessment of canine BEST1 variations identifies new mutations and establishes an independent bestrophinopathy model (cmr3). Mol Vis 2010; 16:2791-2804.

All publications cited in this specification are incorporated herein by reference. U.S. Provisional Patent Application No. 63/237,751, filed Sep. 1, 2021, International Patent Application No. PCT/US21/20171, filed Feb. 28, 2021, and International Patent Application No. PCT/US21/20169, filed Feb. 28, 2021, are incorporated by reference in their entireties. While the invention has been described with reference to particular embodiments, it will be appreciated that modifications can be made without departing from the spirit of the invention. Such modifications are intended to fall within the scope of the appended claims.

What is claimed is:

1. A method of assessing efficacy of treatment for a bestrophinopathy in a subject, the method comprising
providing a subject having a treated eye, said treated eye having been administered a dose of a recombinant adeno-associated virus (rAAV) vector comprising a nucleic acid sequence encoding a human BEST1 protein or a functional fragment thereof, and
wherein the subject has two mutant BEST1 alleles, and
assessing retinal function in the treated eye of the subject by electroretinography (ERG),
wherein improved and/or maintained ERG amplitude(s) is indicative of efficacy of the treatment.

2. A method of assessing efficacy of treatment for a bestrophinopathy in a subject, the method comprising
providing a subject having a treated eye, said treated eye having been administered a dose of a recombinant adeno-associated virus (rAAV) vector comprising a nucleic acid sequence encoding a human BEST1 protein, or a functional fragment thereof,
wherein the subject has at least one mutant BEST1 allele,
assessing retinal function in the treated eye of the subject by ERG,
wherein improved and/or maintained ERG amplitude(s) is indicative of efficacy of the treatment.

3. A method of treatment for a bestrophinopathy in a subject having at least one mutant BEST1 allele, the method comprising
assessing retinal function in an eye of the subject by electroretinography (ERG), and
administering to the eye a dose of a recombinant adeno-associated virus (rAAV) vector comprising a nucleic acid sequence encoding a human BEST1 protein or a functional fragment thereof.

4. The method of claim 3, further comprising assessing retinal function in the eye of the subject by ERG following the administration to the eye the rAAV vector.

5. A method of treatment for a bestrophinopathy in a subject having two mutant BEST1 alleles, the method comprising
assessing retinal function in an eye of the subject by ERG, and
administering to the eye a dose of a rAAV vector comprising a nucleic acid sequence encoding a human BEST1 protein or a functional fragment thereof.

6. The method of claim 5, further comprising assessing retinal function in the eye of the subject by ERG following the administration to the eye the rAAV vector.

7. A method of assessing efficacy of treatment for a bestrophinopathy in a subject having at least one mutant BEST1 allele, the method comprising
providing a subject having a treated eye, said treated eye having been administered a dose of a recombinant adeno-associated virus (rAAV) vector comprising a nucleic acid sequence encoding a human BEST1 protein, or a functional fragment thereof,
assessing retinal function in the treated eye of the subject by ERG, and
administering to the eye a dose of a recombinant adeno-associated virus (rAAV) vector comprising a nucleic acid sequence encoding a human BEST1 protein, or a functional fragment thereof.

8. The method of claim 7, further comprising assessing retinal function in the eye of the subject by ERG following the administration to the eye the rAAV vector.

9. A method of assessing efficacy of treatment for a bestrophinopathy in a subject having two mutant BEST1 alleles, the method comprising
providing a subject having a treated eye, said treated eye having been administered a dose of a rAAV vector comprising a nucleic acid sequence encoding a human BEST1 protein, or a functional fragment thereof,
assessing retinal function in the treated eye of the subject by ERG, and
administering to the eye a dose of a rAAV vector comprising a nucleic acid sequence encoding a human BEST1 protein, or a functional fragment thereof.

10. The method of claim 9, further comprising assessing retinal function in the eye of the subject by ERG following the administration to the eye the rAAV vector.

11. The method according to any one of claims 1 to 10, wherein the subject is a canine, mouse, rat, non-human primate, or human.

12. The method according to any one of claims 1 to 11, wherein the subject is a human.

13. The method according to any one of claims 1 to 12, wherein the bestrophinopathy is Best Vitelliform Macular Dystrophy (BVMD), Autosomal dominant vitreoretinochoroidopathy (ADVIRC), Adult-onset vitelliform macular dystrophy (AVMD), retinitis pigmentosa (RP), microcornea, rod-cone dystrophy, or cataract.

14. The method of any of claims 1 to 13, wherein the rAAV vector is administered to the retina of the subject.

15. The method of any one of claims 1 to 14, wherein the rAAV vector is administered via subretinal, intravitreal, or suprachoroidal injection.

16. The method of claim 15, wherein the rAAV vector is administered via subretinal injection.

17. The method of any one of claims 1 to 16, wherein the nucleic acid sequence expresses the human BEST1 protein, or functional fragment thereof, in the retinal pigment epithelium (RPE) of the eye.

18. The method according to any one of claims 1 to 17, wherein the ERG is full-field ERG, focal ERG, and/or multifocal ERG.

19. The method according to any one of claim 1 to 18, wherein assessing retinal function comprises obtaining ERG measurements in more than one region of the retina of the treated eye.

20. The method according to any one of claim 1 to 19, wherein assessing retinal function comprises obtaining ERG measurements within a treated region of the retina and in an untreated region of the retina.

21. The method according to claim 20, wherein the treated region of the retina is a subretinal bleb at the site of administration.

22. The method according to any one of claims 1 to 21, wherein assessing retinal function comprises obtaining ERG measurements for a contralateral, untreated eye.

23. The method according to any one of claims 1 to 22, wherein assessing retinal function is performed at least 1 week, at least 2 weeks, at least 4 weeks, at least 8 weeks, or at least 10, or at least 11 weeks post-administration of the rAAV vector.

24. The method according to any one of claims 1 to 23, wherein assessing retinal function is performed about 11 weeks post-administration of the rAAV vector.

25. The method according to any one of claims 1 to 24, wherein assessing retinal function comprises measuring the amplitude(s) of a scotopic a-wave response, a scotopic b-wave response, a photopic b-wave response, and/or a photopic flicker response.

26. The method according to claim 25, wherein:
a) the scotopic a-wave response is measured at an intensity that produces a mixed rod-cone response;
b) the scotopic a-wave response is measured at an intensity that produces a rod-only or a mixed rod-cone response;
c) the photopic b-wave response is measured at an intensity that produces a cone response; and/or
d) the photopic flicker response is measured at an intensity that produces a cone response.

27. The method according to claim 26, wherein the amplitude of the scotopic a-wave response is measured at one or more intensities of at least about −2.0 Log cd·s/m².

28. The method according to claim 26 or 27, wherein the amplitude of the scotopic a-wave response is measured at one or more intensities in a range from about −2.0 Log cd·s/m² to about 1.0 Log cd·s/m².

29. The method according to any one of claims 26 to 28, wherein the amplitude of the scotopic b-wave response is measured at one or more intensities of at least about −4.0 Log cd·s/m².

30. The method according to any one of claims 26 to 29, wherein the amplitude of the scotopic b-wave response is measured at one or more intensities in a range from about −4.0 Log cd·s/m² to about 1.0 Log cd·s/m².

31. The method according to any one of claims 26 to 30, wherein the amplitude of the photopic (1 Hz) b-wave response is measured at one or more intensities of at least about −1.0 Log cd·s/m².

32. The method according to any one of claims 26 to 31, wherein the amplitude of the photopic (1 Hz) b-wave response is measured at one or more intensities in a range from about −1.0 Log cd·s/m² to about 1.0 Log cd·s/m².

33. The method according to any one of claims 26 to 32, wherein the amplitude of the photopic 29 Hz flicker response is measured at one or more intensities of at least about −2.0 Log cd·s/m².

34. The method according to any one of claims 26 to 33, wherein the amplitude of the photopic 29 Hz flicker response is measured at one or more intensities in a range from about −2.0 Log cd·s/m² to about 0.5 Log cd·s/m².

35. The method according to any one of claims 26 to 34, wherein an amplitude difference is obtained by:
1) comparing an ERG amplitude measurement obtained from the treated eye and an ERG amplitude measurement obtained from an untreated, contralateral eye; and/or
2) comparing an ERG amplitude measurement obtained in a region of the treated eye and an ERG amplitude measurement obtained from an untreated region of the treated eye.

36. The method according to claim 35, wherein the amplitude difference for one or more measurements is 0 μV, at least about 2.0 μV, at least about 5 μV, at least about 10 μV, at least about 15 μV, at least about 20 μV, at least about 25 μV, at least about 30 μV, at least about 40 μV, or at least about 50 μV.

37. The method according to any one of claims 1 to 36, wherein the dose of the rAAV vector is administered at a concentration of about 1.0×10⁹ vg/ml to about 3.0×10¹² vg/ml.

38. The method of claim 37, wherein the dose of rAAV vector is administered at a concentration of about 1.5×10¹⁰ vg/ml.

39. The method of claim 37, wherein the dose of rAAV vector is administered at a concentration of about 3.0×10¹⁰ vg/ml.

40. The method of claim 37, wherein the dose of rAAV vector is administered at a concentration of about 9.5×10⁹ vg/ml.

41. The method according to any one of claims 1 to 36, wherein the dose of rAAV vector is administered at a concentration of about 1.0×10¹¹ vg/ml to about 7.5×10¹¹ vg/ml.

42. The method according to claim 41, wherein the dose of rAAV vector is administered at a concentration of about 3.0×10¹¹ vg/ml.

43. The method according to claim 41, wherein the dose of rAAV vector is administered at a concentration of about 6.0×10¹¹ vg/ml.

44. The method according to any one of claims 1 to 36, wherein the dose of rAAV vector is administered at a concentration of about 7.5×10¹¹ vg/ml to about 1.0×10¹³ vg/ml.

45. The method according to claim 44, wherein the dose of rAAV vector is administered at a concentration of about 3.5×10¹² vg/ml.

46. The method according to any one of claims 1 to 45, wherein the dose of rAAV vector is administered in a volume in the range of about 50 ul to about 500 ul.

47. The method according to claim 46, wherein the dose of rAAV vector is administered in a volume of about 150 ul.

48. The method according to claim 46, wherein the dose of rAAV vector is administered in a volume of about 300 ul.

49. The method according to claim 46, wherein the dose of rAAV vector is administered in a volume of about 500 ul.

50. The method according to any one of claims 1 to 49, wherein the dose of rAAV vector administered is about 5.0×10⁸ vg per eye to about 1.5×10¹⁰ vg per eye.

51. The method according to claim 50, wherein the dose of rAAV vector administered is about 7.5×10⁸ vg per eye.

52. The method according to claim 50, wherein the dose of rAAV vector administered is about 4.5×10⁹ vg per eye.

53. The method of claim 50, wherein the dose of rAAV vector administered is about 1.4×10⁹ vg per eye.

54. The method according to any one of claims 1 to 49, wherein the dose of rAAV vector administered is about 1.0×10¹⁰ vg per eye to about 1.0×10¹¹ vg per eye.

55. The method according to claim 54, wherein the dose of rAAV vector administered is about 4.5×10¹⁰ vg per eye.

56. The method according to any one of claims 1 to 49, wherein the dose of rAAV vector administered is about 1.0×10¹¹ vg per eye to about 5.0×10¹² vg per eye.

57. The method of claim 56, wherein the dose of rAAV vector administered is about 1.0×10¹² vg per eye

58. The method according to any one of claims 1 to 57, further comprising evaluating treatment by one or more of:
performing in vivo retinal imaging to evaluate one or more of a longitudinal reflectivity profile (LRP), IS/OS to retinal pigment epithelium (RPE) distance in light-adapted and/or dark-adapted eyes, electrophysiology, dark-adapted kinetic perimetry and formation of light-potentiated subretinal microdetachments,
wherein treatment efficacy is indicated by one or more of a rescue of retinal microarchitecture through restoration of RPE apical microvilli structure, and a reestablishment of proper apposition between RPE cells and photoreceptor (PR) outer segments (cytoarchitecture of RPE-PR interface).

59. The method according to claim 58, wherein the performing in vivo retinal imaging comprises one or more of fundus examination, cSLO/SD-OCT, measurement of rod outer segments, cone outer segments, ONL thickness, and ELM-RPE distance.

60. The method according to claim 59, wherein the performing in vivo retinal imaging comprises evaluation for reactive gliosis.

61. The method according to any one of claims 58 to 60, further comprising evaluation for Muller glial trunks/projections penetrating ONL layer with astrogliosis.

62. The method according to any one of claims 58 to 61, wherein said retinal imaging is performed using an ultrahigh-resolution optical coherence tomography (OCT) to generate said LRP.

63. The method according to any one of claims 58 to 62, further comprising comparing a measurement of a selected parameter to a measurement in a normal control, mutant disease control, pre-treatment control, earlier timepoint control, an untreated contralateral eye, or a retinal region outside of a treatment bleb.
